SEQUENCE LISTING (816 sequences)

SEQ ID NO: 1 - DNA, 1956 bp, synthetic construct
  misc_feature 1..1956: note = synthetic polymer
  source 1..1956: mol_type = other DNA
taatacgactcactataaggaagcttcttgttctttttgcagaagctcagaataaacgctcaactttggccgccaccatggcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattactagaaccagcctcaagaacacccgaatggagtctctaagctacataataccaacttacactttacaaaatgttgtcccccaaaatgtagccattcgtatctgctcctaataaaaagaaagtttcttcacaaaaaaaaaaaaaaaaaaaaaaaaaaaaaaaaaa

SEQ ID NO: 2 - DNA, 398 bp, synthetic construct
  misc_feature 1..398: note = synthetic polymer
  misc_feature 14: note = n is a, c, g, or t
  misc_feature 21: note = n is a, c, g, or t
  misc_feature 59: note = n is a, c, g, or t
  misc_feature 65: note = n is a, c, g, or t
  misc_feature 70..71: note = n is a, c, g, or t
  misc_feature 81: note = n is a, c, g, or t
  misc_feature 85..86: note = n is a, c, g, or t
  misc_feature 88..89: note = n is a, c, g, or t
  misc_feature 137..139: note = n is a, c, g, or t
  misc_feature 143: note = n is a, c, g, or t
  misc_feature 156: note = n is a, c, g, or t
  misc_feature 171: note = n is a, c, g, or t
  misc_feature 188: note = n is a, c, g, or t
  misc_feature 211: note = n is a, c, g, or t
  misc_feature 214: note = n is a, c, g, or t
  misc_feature 218: note = n is a, c, g, or t
  misc_feature 223: note = n is a, c, g, or t
  misc_feature 231: note = n is a, c, g, or t
  misc_feature 245: note = n is a, c, g, or t
  misc_feature 261: note = n is a, c, g, or t
  misc_feature 271: note = n is a, c, g, or t
  misc_feature 275: note = n is a, c, g, or t
  misc_feature 304: note = n is a, c, g, or t
  misc_feature 307: note = n is a, c, g, or t
  misc_feature 366: note = n is a, c, g, or t
  misc_feature 386: note = n is a, c, g, or t
  misc_feature 397: note = n is a, c, g, or t
  source 1..398: mol_type = other DNA
mahsdyddradksnyscdsdnastsddssddvmvrrtrrrrsssssdssdggrwshvdnvdghgntdavnndavkgddvsnryynnrnnkskkskwkdtmkkgvmgvrkdrrddywttwttygktmtrdrrwkawhnnnadvnsdrckvrvdyvknykhsdgvwrgrrvynagkvkygvrcsdtgycnmycggkrttvvsytdswyhymdnyynsvancamknkrcgtrknrgkdtskkgtkrkndvwskkvysshsamsndrtskkkvknadynkhmkgvdradysyysrrtvkwtkramymncansyavyksvrrkmgkmktahwtdddmdvdvstsgmraktsdcrsmdmrkhtavgsgkkknrrcrvcsvhkrstrymckcnhkgackyhtkny

SEQ ID NO: 3 - DNA, 157 bp, synthetic construct
  misc_feature 1..157: note = synthetic polymer
  source 1..157: mol_type = other DNA
ttaacacttggattgcgggaaacgagttaagtcggctcgcgtgaattgcgcgtactccgcgggagccgtcttaactcggttcatatagatttgcggtggagtgcgggaaacgtgtaaactcgggccgattgtaactgcgtattaccaaatatttgtt

SEQ ID NO: 4 - DNA, 212 bp, synthetic construct
  misc_feature 1..212: note = synthetic polymer
  source 1..212: mol_type = other DNA
aattatttatgtactgaatagataaaaaaatgtctgtgattgaataaattttcattttttacacaagaaaccgaaaatttcatttcaatcgaacccatacttcaaaagatataggcattttaaactaactctgattttgcgcgggaaacctaaataattgcccgcgccatcttatattttggcgggaaattcacccgacaccgtagtgttaa

SEQ ID NO: 5 - DNA, 4104 bp, synthetic construct
  misc_feature 1..4104: note = synthetic polymer
  source 1..4104: mol_type = other DNA
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctggaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 6 - protein, 1368 aa, synthetic construct
  REGION 1..1368: note = synthetic polymer
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7-8 - skipped (2 entries)

SEQ ID NO: 9 - protein, 572 aa, synthetic construct
  REGION 1..572: note = synthetic polymer
MAQHSDYSDDEFCADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 10 - DNA, 1719 bp, synthetic construct
  misc_feature 1..1719: note = synthetic polymer
  source 1..1719: mol_type = other DNA
atggcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattactag

SEQ ID NO: 11 - DNA, 1719 bp, synthetic construct; identical to SEQ ID NO: 10
  misc_feature 1..1719: note = synthetic polymer
  source 1..1719: mol_type = other DNA

SEQ ID NO: 12 - protein, 508 aa, synthetic construct
  REGION 1..508: note = synthetic polymer
MRNFPVPYSNELIYSTIARAGVYQGIVSPKQLLDEVYGNRKVVATLGLPSHLGVIARHLHQTGRYAVQQLIYEHTLFPLYAPFVGKERRDEAIRLMEYQAQGAVHLMLGVAASRVKSDNRFRYCPDCVALQLNRYGEAFWQRDWYLPALPYCPKHGALVFFDRAVDDHRHQFWALGHTELLSDYPKDSLSQLTALAAYIAPLLDAPRAQELSPSLEQWTLFYQRLAQDLGLTKSKHIRHDLVAERVRQTFSDEALEKLDLKLAENKDTCWLKSIFRKHRKAFSYLQHSIVWQALLPKLTVIEALQQASALTEHSITTRPVSQSVQPNSEDLSVKHKDWQQLVHKYQGIKAARQSLEGGVLYAWLYRHDRDWLVHWNQQHQQERLAPAPRVDWNQRDRIAVRQLLRIIKRLDSSLDHPRATSSWLLKQTPNGTSLAKNLQKLPLVALCLKRYSESVEDYQIRRISQAFIKLKQEDVELRRWRLLRSATLSKERITEEAQRFLEMVYGEE

SEQ ID NO: 13-22 - skipped (10 entries)

SEQ ID NO: 23 - DNA, 19 bp, synthetic construct
  misc_feature 1..19: note = synthetic polymer
  source 1..19: mol_type = other DNA
tggccggcctgaccactgg

SEQ ID NO: 24 - DNA, 19 bp, synthetic construct
  misc_feature 1..19: note = synthetic polymer
  source 1..19: mol_type = other DNA
tgaaggcctggccggcctg

SEQ ID NO: 25 - DNA, 19 bp, synthetic construct
  misc_feature 1..19: note = synthetic polymer
  source 1..19: mol_type = other DNA
tgagcactgaaggcctggc

SEQ ID NO: 26 - DNA, 19 bp, synthetic construct
  misc_feature 1..19: note = synthetic polymer
  source 1..19: mol_type = other DNA
tccactgagcactgaaggc

SEQ ID NO: 27 - DNA, 19 bp, synthetic construct
  misc_feature 1..19: note = synthetic polymer
  source 1..19: mol_type = other DNA
tggtttccactgagcactg

SEQ ID NO: 28 - DNA, 19 bp, synthetic construct
  misc_feature 1..19: note = synthetic polymer
  source 1..19: mol_type = other DNA
tggggaaaatgacccaaca

SEQ ID NO: 29 - DNA, 19 bp, synthetic construct
  misc_feature 1..19: note = synthetic polymer
  source 1..19: mol_type = other DNA
taggacagtggggaaaatg

SEQ ID NO: 30 - DNA, 19 bp, synthetic construct
  misc_feature 1..19: note = synthetic polymer
  source 1..19: mol_type = other DNA
tccagggacacggtgctag

SEQ ID NO: 31 - DNA, 19 bp, synthetic construct
  misc_feature 1..19: note = synthetic polymer
  source 1..19: mol_type = other DNA
tcagagccaggagtcctgg

SEQ ID NO: 32 - DNA, 19 bp, synthetic construct
  misc_feature 1..19: note = synthetic polymer
  source 1..19: mol_type = other DNA
tccttcagagccaggagtc

SEQ ID NO: 33 - DNA, 19 bp, synthetic construct
  misc_feature 1..19: note = synthetic polymer
  source 1..19: mol_type = other DNA
tcctccttcagagccagga

SEQ ID NO: 34 - DNA, 19 bp, synthetic construct
  misc_feature 1..19: note = synthetic polymer
  source 1..19: mol_type = other DNA
tccagcccctcctccttca

SEQ ID NO: 35 - DNA, 19 bp, synthetic construct
  misc_feature 1..19: note = synthetic polymer
  source 1..19: mol_type = other DNA
tccgagcttgacccttgga

SEQ ID NO: 36 - DNA, 19 bp, synthetic construct
  misc_feature 1..19: note = synthetic polymer
  source 1..19: mol_type = other DNA
tggtttccgagcttgaccc

SEQ ID NO: 37 - DNA, 19 bp, synthetic construct
  misc_feature 1..19: note = synthetic polymer
  source 1..19: mol_type = other DNA
tggggtggtttccgagctt

SEQ ID NO: 38 - DNA, 19 bp, synthetic construct
  misc_feature 1..19: note = synthetic polymer
  source 1..19: mol_type = other DNA
tctgctggggtggtttccg

SEQ ID NO: 39 - DNA, 19 bp, synthetic construct
  misc_feature 1..19: note = synthetic polymer
  source 1..19: mol_type = other DNA
tgcagagtatctgctgggg

SEQ ID NO: 40 - DNA, 14 bp, synthetic construct
  misc_feature 1..14: note = synthetic polymer
  source 1..14: mol_type = other DNA
ccaatcccctcagt

SEQ ID NO: 41 - DNA, 15 bp, synthetic construct
  misc_feature 1..15: note = synthetic polymer
  source 1..15: mol_type = other DNA
cagtgctcagtggaa

SEQ ID NO: 42 - DNA, 18 bp, synthetic construct
  misc_feature 1..18: note = synthetic polymer
  source 1..18: mol_type = other DNA
gaaacatccggcgactca

SEQ ID NO: 43 - DNA, 19 bp, synthetic construct
  misc_feature 1..19: note = synthetic polymer
  source 1..19: mol_type = other DNA
tcgcccctcaaatcttaca

SEQ ID NO: 44 - DNA, 19 bp, synthetic construct
  misc_feature 1..19: note = synthetic polymer
  source 1..19: mol_type = other DNA
tcaaatcttacagctgctc

SEQ ID NO: 45 - DNA, 19 bp, synthetic construct
  misc_feature 1..19: note = synthetic polymer
  source 1..19: mol_type = other DNA
tcttacagctgctcactcc

SEQ ID NO: 46 - DNA, 19 bp, synthetic construct
  misc_feature 1..19: note = synthetic polymer
  source 1..19: mol_type = other DNA
tacagctgctcactcccct

SEQ ID NO: 47 - DNA, 19 bp, synthetic construct
  misc_feature 1..19: note = synthetic polymer
  source 1..19: mol_type = other DNA
tgctcactcccctgcaggg

SEQ ID NO: 48 - DNA, 19 bp, synthetic construct
  misc_feature 1..19: note = synthetic polymer
  source 1..19: mol_type = other DNA
tcccctgcagggcaacgcc

SEQ ID NO: 49 - DNA, 19 bp, synthetic construct
  misc_feature 1..19: note = synthetic polymer
  source 1..19: mol_type = other DNA
tgcagggcaacgcccaggg

SEQ ID NO: 50 - DNA, 19 bp, synthetic construct
  misc_feature 1..19: note = synthetic polymer
  source 1..19: mol_type = other DNA
tctcgattatgggcgggat

SEQ ID NO: 51 - DNA, 19 bp, synthetic construct
  misc_feature 1..19: note = synthetic polymer
  source 1..19: mol_type = other DNA
tcgcttctcgattatgggc

SEQ ID NO: 52 - DNA, 19 bp, synthetic construct
  misc_feature 1..19: note = synthetic polymer
  source 1..19: mol_type = other DNA
tgtcgagtcgcttctcgat

SEQ ID NO: 53 - DNA, 19 bp, synthetic construct
  misc_feature 1..19: note = synthetic polymer
  source 1..19: mol_type = other DNA
tccatgtcgagtcgcttct

SEQ ID NO: 54 - DNA, 19 bp, synthetic construct
  misc_feature 1..19: note = synthetic polymer
  source 1..19: mol_type = other DNA
tcgcctccatgtcgagtcg

SEQ ID NO: 55 - DNA, 19 bp, synthetic construct
  misc_feature 1..19: note = synthetic polymer
  source 1..19: mol_type = other DNA
tcgtcatcgcctccatgtc

SEQ ID NO: 56 - DNA, 19 bp, synthetic construct
  misc_feature 1..19: note = synthetic polymer
  source 1..19: mol_type = other DNA
tgatctcgtcatcgcctcc

SEQ ID NO: 57 - DNA, 14 bp, synthetic construct
  misc_feature 1..14: note = synthetic polymer
  source 1..14: mol_type = other DNA
gcttcagcttccta

SEQ ID NO: 58 - DNA, 14 bp, synthetic construct
  misc_feature 1..14: note = synthetic polymer
  source 1..14: mol_type = other DNA
ctgtgatcatgcca

SEQ ID NO: 59 - DNA, 16 bp, synthetic construct
  misc_feature 1..16: note = synthetic polymer
  source 1..16: mol_type = other DNA
acagtggtacacacct

SEQ ID NO: 60 - DNA, 15 bp, synthetic construct
  misc_feature 1..15: note = synthetic polymer
  source 1..15: mol_type = other DNA
ccaccccccactaag

SEQ ID NO: 61 - DNA, 14 bp, synthetic construct
  misc_feature 1..14: note = synthetic polymer
  source 1..14: mol_type = other DNA
cattggccgggcac

SEQ ID NO: 62 - DNA, 16 bp, synthetic construct
  misc_feature 1..16: note = synthetic polymer
  source 1..16: mol_type = other DNA
gcttgaacccaggaga

SEQ ID NO: 63 - DNA, 17 bp, synthetic construct
  misc_feature 1..17: note = synthetic polymer
  source 1..17: mol_type = other DNA
acacccgatccactggg

SEQ ID NO: 64 - DNA, 14 bp, synthetic construct
  misc_feature 1..14: note = synthetic polymer
  source 1..14: mol_type = other DNA
gctgcatcaacccc

SEQ ID NO: 65 - DNA, 16 bp, synthetic construct
  misc_feature 1..16: note = synthetic polymer
  source 1..16: mol_type = other DNA
gccacaaacagaaata

SEQ ID NO: 66 - DNA, 15 bp, synthetic construct
  misc_feature 1..15: note = synthetic polymer
  source 1..15: mol_type = other DNA
ggtggctcatgcctg

SEQ ID NO: 67 - DNA, 16 bp, synthetic construct
  misc_feature 1..16: note = synthetic polymer
  source 1..16: mol_type = other DNA
gatttgcacagctcat

SEQ ID NO: 68 - DNA, 15 bp, synthetic construct
  misc_feature 1..15: note = synthetic polymer
  source 1..15: mol_type = other DNA
aagctctgaggagca

SEQ ID NO: 69 - DNA, 13 bp, synthetic construct
  misc_feature 1..13: note = synthetic polymer
  source 1..13: mol_type = other DNA
ccctagctgtccc

SEQ ID NO: 70 - DNA, 14 bp, synthetic construct
  misc_feature 1..14: note = synthetic polymer
  source 1..14: mol_type = other DNA
gcctagcatgctag

SEQ ID NO: 71 - DNA, 15 bp, synthetic construct
  misc_feature 1..15: note = synthetic polymer
  source 1..15: mol_type = other DNA
atgggcttcacggat

SEQ ID NO: 72 - DNA, 14 bp, synthetic construct
  misc_feature 1..14: note = synthetic polymer
  source 1..14: mol_type = other DNA
gaaactatgcctgc

SEQ ID NO: 73 - DNA, 14 bp, synthetic construct
  misc_feature 1..14: note = synthetic polymer
  source 1..14: mol_type = other DNA
gcaccattgctccc

SEQ ID NO: 74 - DNA, 14 bp, synthetic construct
  misc_feature 1..14: note = synthetic polymer
  source 1..14: mol_type = other DNA
gacatgcaactcag

SEQ ID NO: 75 - DNA, 14 bp, synthetic construct
  misc_feature 1..14: note = synthetic polymer
  source 1..14: mol_type = other DNA
acaccactaggggt

SEQ ID NO: 76 - DNA, 14 bp, synthetic construct
  misc_feature 1..14: note = synthetic polymer
  source 1..14: mol_type = other DNA
gtctgctagacagg

SEQ ID NO: 77 - DNA, 15 bp, synthetic construct
  misc_feature 1..15: note = synthetic polymer
  source 1..15: mol_type = other DNA
ggcctagacaggctg

SEQ ID NO: 78 - DNA, 15 bp, synthetic construct
  misc_feature 1..15: note = synthetic polymer
  source 1..15: mol_type = other DNA
gaggcattcttatcg

SEQ ID NO: 79 - DNA, 15 bp, synthetic construct
  misc_feature 1..15: note = synthetic polymer
  source 1..15: mol_type = other DNA
gcctggaaacgttcc

SEQ ID NO: 80 - DNA, 14 bp, synthetic construct
  misc_feature 1..14: note = synthetic polymer
  source 1..14: mol_type = other DNA
gtgctctgacaata

SEQ ID NO: 81 - DNA, 14 bp, synthetic construct
  misc_feature 1..14: note = synthetic polymer
  source 1..14: mol_type = other DNA
gttttgcagcctcc

SEQ ID NO: 82 - DNA, 15 bp, synthetic construct
  misc_feature 1..15: note = synthetic polymer
  source 1..15: mol_type = other DNA
acagctgtggaacgt

SEQ ID NO: 83 - DNA, 15 bp, synthetic construct
  misc_feature 1..15: note = synthetic polymer
  source 1..15: mol_type = other DNA
ggctctcttcctcct

SEQ ID NO: 84 - DNA, 15 bp, synthetic construct
  misc_feature 1..15: note = synthetic polymer
  source 1..15: mol_type = other DNA
ctatcccaaaactct

SEQ ID NO: 85 - DNA, 14 bp, synthetic construct
  misc_feature 1..14: note = synthetic polymer
  source 1..14: mol_type = other DNA
gaaaaactatgtat

SEQ ID NO: 86 - DNA, 15 bp, synthetic construct
  misc_feature 1..15: note = synthetic polymer
  source 1..15: mol_type = other DNA
aggcaggctggttga

SEQ ID NO: 87 - DNA, 14 bp, synthetic construct
  misc_feature 1..14: note = synthetic polymer
  source 1..14: mol_type = other DNA
caatacaaccacgc

SEQ ID NO: 88 - DNA, 15 bp, synthetic construct
  misc_feature 1..15: note = synthetic polymer
  source 1..15: mol_type = other DNA
atgacggactcaact

SEQ ID NO: 89 - DNA, 14 bp, synthetic construct
  misc_feature 1..14: note = synthetic polymer
  source 1..14: mol_type = other DNA
cacaacatttgtaa

SEQ ID NO: 90 - DNA, 14 bp, synthetic construct
  misc_feature 1..14: note = synthetic polymer
  source 1..14: mol_type = other DNA
atttccagtgcaca

SEQ ID NO: 91 - DNA, 20 bp, synthetic construct
  misc_feature 1..20: note = synthetic polymer
  source 1..20: mol_type = other DNA
gtttagctcacccgtgagcc

SEQ ID NO: 92 - DNA, 20 bp, synthetic construct
  misc_feature 1..20: note = synthetic polymer
  source 1..20: mol_type = other DNA
cccaatattattgttctctg

SEQ ID NO: 93 - DNA, 20 bp, synthetic construct
  misc_feature 1..20: note = synthetic polymer
  source 1..20: mol_type = other DNA
ggggtgggataggggatacg

SEQ ID NO: 94 - DNA, 20 bp, synthetic construct
  misc_feature 1..20: note = synthetic polymer
  source 1..20: mol_type = other DNA
ggatccccctctacatttaa

SEQ ID NO: 95 - DNA, 20 bp, synthetic construct
  misc_feature 1..20: note = synthetic polymer
  source 1..20: mol_type = other DNA
gtgatcttgtacaaatcatt

SEQ ID NO: 96 - DNA, 20 bp, synthetic construct
  misc_feature 1..20: note = synthetic polymer
  source 1..20: mol_type = other DNA
ctacacagaatctgttagaa

SEQ ID NO: 97 - DNA, 20 bp, synthetic construct
  misc_feature 1..20: note = synthetic polymer
  source 1..20: mol_type = other DNA
taagctagagaatagatctc

SEQ ID NO: 98 - DNA, 20 bp, synthetic construct
  misc_feature 1..20: note = synthetic polymer
  source 1..20: mol_type = other DNA
tcaatacacttaatgattta

SEQ ID NO: 99 - DNA, 25 bp, synthetic construct
  misc_feature 1..25: note = synthetic polymer
  source 1..25: mol_type = other DNA
caccgggagccacgaaaacagatcc

SEQ ID NO: 100 - DNA, 25 bp, synthetic construct
  misc_feature 1..25: note = synthetic polymer
  source 1..25: mol_type = other DNA
caccgcgaaaacagatccagggaca

SEQ ID NO: 101 - DNA, 25 bp, synthetic construct
  misc_feature 1..25: note = synthetic polymer
  source 1..25: mol_type = other DNA
caccgagatccagggacacggtgct

SEQ ID NO: 102 - DNA, 25 bp, synthetic construct
  misc_feature 1..25: note = synthetic polymer
  source 1..25: mol_type = other DNA
caccggacacggtgctaggacagtg

SEQ ID NO: 103 - DNA, 25 bp, synthetic construct
  misc_feature 1..25: note = synthetic polymer
  source 1..25: mol_type = other DNA
caccggaaaatgacccaacagcctc

SEQ ID NO: 104 - DNA, 25 bp, synthetic construct
  misc_feature 1..25: note = synthetic polymer
  source 1..25: mol_type = other DNA
caccggcctggccggcctgaccact

SEQ ID NO: 105 - DNA, 25 bp, synthetic construct
  misc_feature 1..25: note = synthetic polymer
  source 1..25: mol_type = other DNA
caccgctgagcactgaaggcctggc

SEQ ID NO: 106 - DNA, 25 bp, synthetic construct
  misc_feature 1..25: note = synthetic polymer
  source 1..25: mol_type = other DNA
caccgtggtttccactgagcactga

SEQ ID NO: 107 - DNA, 25 bp, synthetic construct
  misc_feature 1..25: note = synthetic polymer
  source 1..25: mol_type = other DNA
caccggatagccaggagtcctttcg

SEQ ID NO: 108 - DNA, 25 bp, synthetic construct
  misc_feature 1..25: note = synthetic polymer
  source 1..25: mol_type = other DNA
caccggcgcttccagtgctcagact

SEQ ID NO: 109 - DNA, 25 bp, synthetic construct
  misc_feature 1..25: note = synthetic polymer
  source 1..25: mol_type = other DNA
caccgcagtgctcagactagggaag

SEQ ID NO: 110 - DNA, 25 bp, synthetic construct
  misc_feature 1..25: note = synthetic polymer
  source 1..25: mol_type = other DNA
caccggcccctcctccttcagagcc

SEQ ID NO: 111 - DNA, 25 bp, synthetic construct
  misc_feature 1..25: note = synthetic polymer
  source 1..25: mol_type = other DNA
caccgtccttcagagccaggagtcc

SEQ ID NO: 112 - DNA, 25 bp, synthetic construct
  misc_feature 1..25: note = synthetic polymer
  source 1..25: mol_type = other DNA
caccgtggtttccgagcttgaccct

SEQ ID NO: 113 - DNA, 25 bp, synthetic construct
  misc_feature 1..25: note = synthetic polymer
  source 1..25: mol_type = other DNA
caccgctgcagagtatctgctgggg

SEQ ID NO: 114 - DNA, 25 bp, synthetic construct
  misc_feature 1..25: note = synthetic polymer
  source 1..25: mol_type = other DNA
caccgcgttcctgcagagtatctgc

SEQ ID NO: 115 - DNA, 25 bp, synthetic construct
  misc_feature 1..25: note = synthetic polymer
  source 1..25: mol_type = other DNA
aaacggatctgttttcgtggctccc

SEQ ID NO: 116 - DNA, 25 bp, synthetic construct
  misc_feature 1..25: note = synthetic polymer
  source 1..25: mol_type = other DNA
aaactgtccctggatctgttttcgc

SEQ ID NO: 117 - DNA, 25 bp, synthetic construct
  misc_feature 1..25: note = synthetic polymer
  source 1..25: mol_type = other DNA
aaacagcaccgtgtccctggatctc

SEQ ID NO: 118 - DNA, 25 bp, synthetic construct
  misc_feature 1..25: note = synthetic polymer
  source 1..25: mol_type = other DNA
aaaccactgtcctagcaccgtgtcc

SEQ ID NO: 119 - DNA, 25 bp, synthetic construct
  misc_feature 1..25: note = synthetic polymer
  source 1..25: mol_type = other DNA
aaacgaggctgttgggtcattttcc

SEQ ID NO: 120 - DNA, 25 bp, synthetic construct
  misc_feature 1..25: note = synthetic polymer
  source 1..25: mol_type = other DNA
aaacagtggtcaggccggccaggcc

SEQ ID NO: 121 - DNA, 25 bp, synthetic construct
  misc_feature 1..25: note = synthetic polymer
  source 1..25: mol_type = other DNA
aaacgccaggccttcagtgctcagc

SEQ ID NO: 122 - DNA, 25 bp, synthetic construct
  misc_feature 1..25: note = synthetic polymer
  source 1..25: mol_type = other DNA
aaactcagtgctcagtggaaaccac

SEQ ID NO: 123 - DNA, 25 bp, synthetic construct
  misc_feature 1..25: note = synthetic polymer
  source 1..25: mol_type = other DNA
aaaccgaaaggactcctggctatcc

SEQ ID NO: 124 - DNA, 25 bp, synthetic construct
  misc_feature 1..25: note = synthetic polymer
  source 1..25: mol_type = other DNA
aaacagtctgagcactggaagcgcc

SEQ ID NO: 125 - DNA, 25 bp, synthetic construct
  misc_feature 1..25: note = synthetic polymer
  source 1..25: mol_type = other DNA
aaaccttccctagtctgagcactgc

SEQ ID NO: 126 - DNA, 25 bp, synthetic construct
  misc_feature 1..25: note = synthetic polymer
  source 1..25: mol_type = other DNA
aaacggctctgaaggaggaggggcc

SEQ ID NO: 127 - DNA, 25 bp, synthetic construct
  misc_feature 1..25: note = synthetic polymer
  source 1..25: mol_type = other DNA
aaacggactcctggctctgaaggac

SEQ ID NO: 128 - DNA, 25 bp, synthetic construct
  misc_feature 1..25: note = synthetic polymer
  source 1..25: mol_type = other DNA
aaacagggtcaagctcggaaaccac

SEQ ID NO: 129 - DNA, 25 bp, synthetic construct
  misc_feature 1..25: note = synthetic polymer
  source 1..25: mol_type = other DNA
aaacccccagcagatactctgcagc

SEQ ID NO: 130 - DNA, 25 bp, synthetic construct
  misc_feature 1..25: note = synthetic polymer
  source 1..25: mol_type = other DNA
aaacgcagatactctgcaggaacgc

SEQ ID NO: 131 - DNA, 20 bp, synthetic construct
  misc_feature 1..20: note = synthetic polymer
  source 1..20: mol_type = other DNA
tcccctcccagaaagacctg

SEQ ID NO: 132 - DNA, 20 bp, synthetic construct
  misc_feature 1..20: note = synthetic polymer
  source 1..20: mol_type = other DNA
tgggctccaagcaatcctgg

SEQ ID NO: 133 - DNA, 20 bp, synthetic construct
  misc_feature 1..20: note = synthetic polymer
  source 1..20: mol_type = other DNA
gtggctcaggaggtacctgg

SEQ ID NO: 134 - DNA, 20 bp, synthetic construct
  misc_feature 1..20: note = synthetic polymer
  source 1..20: mol_type = other DNA
gagccacgaaaacagatcca

SEQ ID NO: 135 - DNA, 20 bp, synthetic construct
  misc_feature 1..20: note = synthetic polymer
  source 1..20: mol_type = other DNA
aagtgaacggggaagggagg

SEQ ID NO: 136 - DNA, 20 bp, synthetic construct
  misc_feature 1..20: note = synthetic polymer
  source 1..20: mol_type = other DNA
gacaaaagccgaagtccagg

SEQ ID NO: 137 - DNA, 20 bp, synthetic construct
  misc_feature 1..20: note = synthetic polymer
  source 1..20: mol_type = other DNA
gtggttgataaacccacgtg

SEQ ID NO: 138 - DNA, 20 bp, synthetic construct
  misc_feature 1..20: note = synthetic polymer
  source 1..20: mol_type = other DNA
tgggaacagccacagcaggg

SEQ ID NO: 139 - DNA, 20 bp, synthetic construct
  misc_feature 1..20: note = synthetic polymer
  source 1..20: mol_type = other DNA
gcaggggaacggggatgcag

SEQ ID NO: 140 - DNA, 20 bp, synthetic construct
  misc_feature 1..20: note = synthetic polymer
  source 1..20: mol_type = other DNA
gagatggtggacgaggaagg

SEQ ID NO: 141 - DNA, 20 bp, synthetic construct
  misc_feature 1..20: note = synthetic polymer
  source 1..20: mol_type = other DNA
gagatggctccaggaaatgg

SEQ ID NO: 142 - DNA, 20 bp, synthetic construct
  misc_feature 1..20: note = synthetic polymer
  source 1..20: mol_type = other DNA
taaggaatctgcctaacagg

SEQ ID NO: 143 - DNA, 20 bp, synthetic construct
  misc_feature 1..20: note = synthetic polymer
  source 1..20: mol_type = other DNA
tcaggagactaggaaggagg

SEQ ID NO: 144 - DNA, 20 bp, synthetic construct
  misc_feature 1..20: note = synthetic polymer
  source 1..20: mol_type = other DNA
tataaggtggtcccagctcg

SEQ ID NO: 145 - DNA, 20 bp, synthetic construct
  misc_feature 1..20: note = synthetic polymer
  source 1..20: mol_type = other DNA
ctggaagatgccatgacagg

SEQ ID NO: 146 - DNA, 20 bp, synthetic construct
  misc_feature 1..20: note = synthetic polymer
  source 1..20: mol_type = other DNA
gcacagactagagaggtaag

SEQ ID NO: 147 - DNA, 20 bp, synthetic construct
  misc_feature 1..20: note = synthetic polymer
  source 1..20: mol_type = other DNA
acagactagagaggtaaggg

SEQ ID NO: 148 - DNA, 20 bp, synthetic construct
  misc_feature 1..20: note = synthetic polymer
  source 1..20: mol_type = other DNA
gagaggtgacccgaatccac

SEQ ID NO: 149 - DNA, 20 bp, synthetic construct
  misc_feature 1..20: note = synthetic polymer
  source 1..20: mol_type = other DNA
gcacaggccccagaaggaga

SEQ ID NO: 150 - DNA, 20 bp, synthetic construct
  misc_feature 1..20: note = synthetic polymer
  source 1..20: mol_type = other DNA
ccggagaggacccagacacg

SEQ ID NO: 151 - DNA, 20 bp, synthetic construct
  misc_feature 1..20: note = synthetic polymer
  source 1..20: mol_type = other DNA
gagaggacccagacacgggg

SEQ ID NO: 152 - DNA, 20 bp, synthetic construct
  misc_feature 1..20: note = synthetic polymer
  source 1..20: mol_type = other DNA
gcaacacagcagagagcaag

SEQ ID NO: 153 - DNA, 20 bp, synthetic construct
  misc_feature 1..20: note = synthetic polymer
  source 1..20: mol_type = other DNA
gaagagggagtggaggaaga

SEQ ID NO: 154 - DNA, 20 bp, synthetic construct
  misc_feature 1..20: note = synthetic polymer
  source 1..20: mol_type = other DNA
aagacggaacctgaaggagg

SEQ ID NO: 155 - DNA, 20 bp, synthetic construct
  misc_feature 1..20: note = synthetic polymer
  source 1..20: mol_type = other DNA
agaaagcggcacaggcccag

SEQ ID NO: 156 - DNA, 20 bp, synthetic construct
  misc_feature 1..20: note = synthetic polymer
  source 1..20: mol_type = other DNA
gggaaacagtgggccagagg

SEQ ID NO: 157 - DNA, 20 bp, synthetic construct
  misc_feature 1..20: note = synthetic polymer
  source 1..20: mol_type = other DNA
gtccggactcaggagagaga

SEQ ID NO: 158 - DNA, 20 bp, synthetic construct
  misc_feature 1..20: note = synthetic polymer
  source 1..20: mol_type = other DNA
ggcacagcaagggcactcgg

SEQ ID NO: 159 - DNA, 20 bp, synthetic construct
  misc_feature 1..20: note = synthetic polymer
  source 1..20: mol_type = other DNA
gaagaggggaagtcgaggga

SEQ ID NO: 160 - DNA, 20 bp, synthetic construct
  misc_feature 1..20: note = synthetic polymer
  source 1..20: mol_type = other DNA
gggaatggtaaggaggcctg

SEQ ID NO: 161 - DNA, 20 bp, synthetic construct
  misc_feature 1..20: note = synthetic polymer
  source 1..20: mol_type = other DNA
gcagagtggtcagcacagag

SEQ ID NO: 162 - DNA, 20 bp, synthetic construct
  misc_feature 1..20: note = synthetic polymer
  source 1..20: mol_type = other DNA
gcacagagtggctaagccca

SEQ ID NO: 163 - DNA, 20 bp, synthetic construct
  misc_feature 1..20: note = synthetic polymer
  source 1..20: mol_type = other DNA
gacggggtgtcagcataggg

SEQ ID NO: 164 - DNA, 20 bp, synthetic construct
  misc_feature 1..20: note = synthetic polymer
  source 1..20: mol_type = other DNA
gcccagggccaggaacgacg

SEQ ID NO: 165 - DNA, 20 bp, synthetic construct
  misc_feature 1..20: note = synthetic polymer
  source 1..20: mol_type = other DNA
ggtggagtccagcacggcgc

SEQ ID NO: 166 - DNA, 20 bp, synthetic construct
  misc_feature 1..20: note = synthetic polymer
  source 1..20: mol_type = other DNA
acaggccgccaggaactcgg

SEQ ID NO: 167 - DNA, 20 bp, synthetic construct
  misc_feature 1..20: note = synthetic polymer
  source 1..20: mol_type = other DNA
actaggaagtgtgtagcacc

SEQ ID NO: 168 - DNA, 20 bp, synthetic construct
  misc_feature 1..20: note = synthetic polymer
  source 1..20: mol_type = other DNA
atgaatagcagactgccccg

SEQ ID NO: 169 - DNA, 20 bp, synthetic construct
  misc_feature 1..20: note = synthetic polymer
  source 1..20: mol_type = other DNA
acacccctaaaagcacagtg

SEQ ID NO: 170 - DNA, 20 bp, synthetic construct
  misc_feature 1..20: note = synthetic polymer
  source 1..20: mol_type = other DNA
caaggagttccagcaggtgg

SEQ ID NO: 171 - DNA, 20 bp, synthetic construct
  misc_feature 1..20: note = synthetic polymer
  source 1..20: mol_type = other DNA
aaggagttccagcaggtggg

SEQ ID NO: 172 - DNA, 20 bp, synthetic construct
  misc_feature 1..20: note = synthetic polymer
  source 1..20: mol_type = other DNA
tggaaagaggagggaagagg

SEQ ID NO: 173 - DNA, 20 bp, synthetic construct
  misc_feature 1..20: note = synthetic polymer
  source 1..20: mol_type = other DNA
tcgaattcctaactgccccg

SEQ ID NO: 174 - DNA, 20 bp, synthetic construct
  misc_feature 1..20: note = synthetic polymer
  source 1..20: mol_type = other DNA
gacctgcccagcacaccctg

SEQ ID NO: 175 - DNA, 20 bp, synthetic construct
  misc_feature 1..20: note = synthetic polymer
  source 1..20: mol_type = other DNA
ggagcagctgcggcagtggg

SEQ ID NO: 176 - DNA, 20 bp, synthetic construct
  misc_feature 1..20: note = synthetic polymer
  source 1..20: mol_type = other DNA
gggagggagagcttggcagg

SEQ ID NO: 177 - DNA, 20 bp, synthetic construct
  misc_feature 1..20: note = synthetic polymer
  source 1..20: mol_type = other DNA
gttacgtggccaagaagcag

SEQ ID NO: 178 - DNA, 20 bp, synthetic construct
  misc_feature 1..20: note = synthetic polymer
  source 1..20: mol_type = other DNA
gctgaacagagaagagctgg

SEQ ID NO: 179 - DNA, 20 bp, synthetic construct
  misc_feature 1..20: note = synthetic polymer
  source 1..20: mol_type = other DNA
tctgagggtggagggactgg

SEQ ID NO: 180 - DNA, 20 bp, synthetic construct
  misc_feature 1..20: note = synthetic polymer
  source 1..20: mol_type = other DNA
ggagaggtgagggacttggg

SEQ ID NO: 181 - DNA, 20 bp, synthetic construct
  misc_feature 1..20: note = synthetic polymer
  source 1..20: mol_type = other DNA
gtgaaccaggcagacaacga

SEQ ID NO: 182 - DNA, 20 bp, synthetic construct
  misc_feature 1..20: note = synthetic polymer
  source 1..20: mol_type = other DNA
caggtacctcctgagccacg

SEQ ID NO: 183 - DNA, 20 bp, synthetic construct
  misc_feature 1..20: note = synthetic polymer
  source 1..20: mol_type = other DNA
gggggagtaggggcatgcag

SEQ ID NO: 184 - DNA, 20 bp, synthetic construct
  misc_feature 1..20: note = synthetic polymer
  source 1..20: mol_type = other DNA
gcaaatggccagcaagggtg

SEQ ID NO: 185 - DNA, 25 bp, synthetic construct
  misc_feature 1..25: note = synthetic polymer
  source 1..25: mol_type = other DNA
caccgaatcgagaagcgactcgaca

SEQ ID NO: 186 - DNA, 25 bp, synthetic construct
  misc_feature 1..25: note = synthetic polymer
  source 1..25: mol_type = other DNA
caccggtccctgggcgttgccctgc

SEQ ID NO: 187 - DNA, 25 bp, synthetic construct
  misc_feature 1..25: note = synthetic polymer
  source 1..25: mol_type = other DNA
caccgccctgggcgttgccctgcag

SEQ ID NO: 188 - DNA, 25 bp, synthetic construct
  misc_feature 1..25: note = synthetic polymer
  source 1..25: mol_type = other DNA
caccgccgtgggaagataaactaat

SEQ ID NO: 189 - DNA, 25 bp, synthetic construct
  misc_feature 1..25: note = synthetic polymer
  source 1..25: mol_type = other DNA
caccgtcccctgcagggcaacgccc

SEQ ID NO: 190 - DNA, 25 bp, synthetic construct
  misc_feature 1..25: note = synthetic polymer
  source 1..25: mol_type = other DNA
caccggtcgagtcgcttctcgatta

SEQ ID NO: 191 - DNA, 25 bp, synthetic construct
  misc_feature 1..25: note = synthetic polymer
  source 1..25: mol_type = other DNA
caccgctgctgcctcccgtcttgta

SEQ ID NO: 192 - DNA, 25 bp, synthetic construct
  misc_feature 1..25: note = synthetic polymer
  source 1..25: mol_type = other DNA
caccggagtgccgcaatacctttat

SEQ ID NO: 193 - DNA, 25 bp, synthetic construct
  misc_feature 1..25: note = synthetic polymer
  source 1..25: mol_type = other DNA
caccgacactttggtggtgcagcaa

SEQ ID NO: 194 - DNA, 25 bp, synthetic construct
  misc_feature 1..25: note = synthetic polymer
  source 1..25: mol_type = other DNA
caccgtctcaaatggtataaaactc

SEQ ID NO: 195 - DNA, 25 bp, synthetic construct
  misc_feature 1..25: note = synthetic polymer
  source 1..25: mol_type = other DNA
caccgaatcccgcccataatcgaga

SEQ ID NO: 196 - DNA, 25 bp, synthetic construct
  misc_feature 1..25: note = synthetic polymer
  source 1..25: mol_type = other DNA
caccgtcccgcccataatcgagaag

SEQ ID NO: 197 - DNA, 25 bp, synthetic construct
  misc_feature 1..25: note = synthetic polymer
  source 1..25: mol_type = other DNA
caccgcccataatcgagaagcgact

SEQ ID NO: 198 - DNA, 25 bp, synthetic construct
  misc_feature 1..25: note = synthetic polymer
  source 1..25: mol_type = other DNA
caccggagaagcgactcgacatgga

SEQ ID NO: 199 - DNA, 25 bp, synthetic construct
  misc_feature 1..25: note = synthetic polymer
  source 1..25: mol_type = other DNA
caccggaagcgactcgacatggagg

SEQ ID NO: 200 - DNA, 25 bp, synthetic construct
  misc_feature 1..25: note = synthetic polymer
  source 1..25: mol_type = other DNA
caccggcgactcgacatggaggcga

SEQ ID NO: 201 - DNA, 25 bp, synthetic construct
  misc_feature 1..25: note = synthetic polymer
  source 1..25: mol_type = other DNA
aaactgtcgagtcgcttctcgattc

SEQ ID NO: 202 - DNA, 25 bp, synthetic construct
  misc_feature 1..25: note = synthetic polymer
  source 1..25: mol_type = other DNA
aaacgcagggcaacgcccagggacc

SEQ ID NO: 203 - DNA, 25 bp, synthetic construct
  misc_feature 1..25: note = synthetic polymer
  source 1..25: mol_type = other DNA
aaacctgcagggcaacgcccagggc

SEQ ID NO: 204 - DNA, 25 bp, synthetic construct
  misc_feature 1..25: note = synthetic polymer
  source 1..25: mol_type = other DNA
aaacattagtttatcttcccacggc

SEQ ID NO: 205 - DNA, 25 bp, synthetic construct
  misc_feature 1..25: note = synthetic polymer
  source 1..25: mol_type = other DNA
aaacgggcgttgccctgcaggggac

SEQ ID NO: 206 - DNA, 25 bp, synthetic construct
  misc_feature 1..25: note = synthetic polymer
  source 1..25: mol_type = other DNA
aaactaatcgagaagcgactcgacc

SEQ ID NO: 207 - DNA, 25 bp, synthetic construct
  misc_feature 1..25: note = synthetic polymer
  source 1..25: mol_type = other DNA
aaactacaagacgggaggcagcagc

SEQ ID NO: 208 - DNA, 25 bp, synthetic construct
  misc_feature 1..25: note = synthetic polymer
  source 1..25: mol_type = other DNA
aaacataaaggtattgcggcactcc

SEQ ID NO: 209 - DNA, 25 bp, synthetic construct
  misc_feature 1..25: note = synthetic polymer
  source 1..25: mol_type = other DNA
aaacttgctgcaccaccaaagtgtc

SEQ ID NO: 210 - DNA, 25 bp, synthetic construct
  misc_feature 1..25: note = synthetic polymer
  source 1..25: mol_type = other DNA
aaacgagttttataccatttgagac

SEQ ID NO: 211 - DNA, 25 bp, synthetic construct
  misc_feature 1..25: note = synthetic polymer
  source 1..25: mol_type = other DNA
aaactctcgattatgggcgggattc

SEQ ID NO: 212 - DNA, 25 bp, synthetic construct
  misc_feature 1..25: note = synthetic polymer
  source 1..25: mol_type = other DNA
aaaccttctcgattatgggcgggac

SEQ ID NO: 213 - DNA, 25 bp, synthetic construct
  misc_feature 1..25: note = synthetic polymer
  source 1..25: mol_type = other DNA
aaacagtcgcttctcgattatgggc

SEQ ID NO: 214 - DNA, 25 bp, synthetic construct
  misc_feature 1..25: note = synthetic polymer
  source 1..25: mol_type = other DNA
aaactccatgtcgagtcgcttctcc

SEQ ID NO: 215 - DNA, 25 bp, synthetic construct
  misc_feature 1..25: note = synthetic polymer
  source 1..25: mol_type = other DNA
aaaccctccatgtcgagtcgcttcc

SEQ ID NO: 216 - DNA, 25 bp, synthetic construct
  misc_feature 1..25: note = synthetic polymer
  source 1..25: mol_type = other DNA
aaactcgcctccatgtcgagtcgcc

SEQ ID NO: 217 - DNA, 25 bp, synthetic construct
  misc_feature 1..25: note = synthetic polymer
  source 1..25: mol_type = other DNA
caccgacagggttaatgtgaagtcc

SEQ ID NO: 218 - DNA, 25 bp, synthetic construct
  misc_feature 1..25: note = synthetic polymer
  source 1..25: mol_type = other DNA
caccgtccccctctacatttaaagt

SEQ ID NO: 219 - DNA, 25 bp, synthetic construct
  misc_feature 1..25: note = synthetic polymer
  source 1..25: mol_type = other DNA
caccgcatttaaagttggtttaagt

SEQ ID NO: 220 - DNA, 25 bp, synthetic construct
  misc_feature 1..25: note = synthetic polymer
  source 1..25: mol_type = other DNA
caccgttagaaaatataaagaataa

SEQ ID NO: 221 - DNA, 25 bp, synthetic construct
  misc_feature 1..25: note = synthetic polymer
  source 1..25: mol_type = other DNA
caccgtaaatgcttactggtttgaa

SEQ ID NO: 222 - DNA, 25 bp, synthetic construct
  misc_feature 1..25: note = synthetic polymer
  source 1..25: mol_type = other DNA
caccgtcctgggtccagaaaaagat

SEQ ID NO: 223 - DNA, 25 bp, synthetic construct
  misc_feature 1..25: note = synthetic polymer
  source 1..25: mol_type = other DNA
caccgttgggtggtgagcatctgtg

SEQ ID NO: 224 - DNA, 25 bp, synthetic construct
  misc_feature 1..25: note = synthetic polymer
  source 1..25: mol_type = other DNA
caccgcggggagagtggagaaaaag

SEQ ID NO: 225 - DNA, 25 bp, synthetic construct
  misc_feature 1..25: note = synthetic polymer
  source 1..25: mol_type = other DNA
caccggttaaaactctttagacaac

SEQ ID NO: 226 - DNA, 25 bp, synthetic construct
  misc_feature 1..25: note = synthetic polymer
  source 1..25: mol_type = other DNA
caccggaaaatccccactaagatcc

SEQ ID NO: 227 - DNA, 25 bp, synthetic construct
  misc_feature 1..25: note = synthetic polymer
  source 1..25: mol_type = other DNA
aaacggacttcacattaaccctgtc

SEQ ID NO: 228 - DNA, 25 bp, synthetic construct
  misc_feature 1..25: note = synthetic polymer
  source 1..25: mol_type = other DNA
aaacactttaaatgtagagggggac

SEQ ID NO: 229 - DNA, 25 bp, synthetic construct
  misc_feature 1..25: note = synthetic polymer
  source 1..25: mol_type = other DNA
aaacacttaaaccaactttaaatgc

SEQ ID NO: 230 - DNA, 25 bp, synthetic construct
  misc_feature 1..25: note = synthetic polymer
  source 1..25: mol_type = other DNA
aaacttattctttatattttctaac

SEQ ID NO: 231 - DNA, 25 bp, synthetic construct
  misc_feature 1..25: note = synthetic polymer
  source 1..25: mol_type = other DNA
aaacttcaaaccagtaagcatttac

SEQ ID NO: 232 - DNA, 25 bp, synthetic construct
  misc_feature 1..25: note = synthetic polymer
  source 1..25: mol_type = other DNA
aaacatctttttctggacccaggac

SEQ ID NO: 233 - DNA, 25 bp, synthetic construct
  misc_feature 1..25: note = synthetic polymer
  source 1..25: mol_type = other DNA
aaaccacagatgctcaccacccaac

SEQ ID NO: 234 - DNA, 25 bp, synthetic construct
  misc_feature 1..25: note = synthetic polymer
  source 1..25: mol_type = other DNA
aaacctttttctccactctccccgc

SEQ ID NO: 235 - DNA, 25 bp, synthetic construct
  misc_feature 1..25: note = synthetic polymer
  source 1..25: mol_type = other DNA
aaacgttgtctaaagagttttaacc

SEQ ID NO: 236 - DNA, 25 bp, synthetic construct
  misc_feature 1..25: note = synthetic polymer
  source 1..25: mol_type = other DNA
aaacggatcttagtggggattttcc

SEQ ID NO: 237 - DNA, 20 bp, synthetic construct
  misc_feature 1..20: note = synthetic polymer
  source 1..20: mol_type = other DNA
agtagcagtaatgaagctgg

SEQ ID NO: 238 - DNA, 20 bp, synthetic construct
  misc_feature 1..20: note = synthetic polymer
  source 1..20: mol_type = other DNA
atacccagacgagaaagctg

SEQ ID NO: 239 - DNA, 20 bp, synthetic construct
  misc_feature 1..20: note = synthetic polymer
  source 1..20: mol_type = other DNA
tacccagacgagaaagctga

SEQ ID NO: 240 - DNA, 20 bp, synthetic construct
  misc_feature 1..20: note = synthetic polymer
  source 1..20: mol_type = other DNA
ggtggtgagcatctgtgtgg

SEQ ID NO: 241 - DNA, 20 bp, synthetic construct
  misc_feature 1..20: note = synthetic polymer
  source 1..20: mol_type = other DNA
aaatgagaagaagaggcaca

SEQ ID NO: 242 - DNA, 20 bp, synthetic construct
  misc_feature 1..20: note = synthetic polymer
  source 1..20: mol_type = other DNA
cttgtggcctgggagagctg

SEQ ID NO: 243 - DNA, 20 bp, synthetic construct
  misc_feature 1..20: note = synthetic polymer
  source 1..20: mol_type = other DNA
gctgtagaaggagacagagc

SEQ ID NO: 244 - DNA, 20 bp, synthetic construct
  misc_feature 1..20: note = synthetic polymer
  source 1..20: mol_type = other DNA
gagctggttgggaagacatg

SEQ ID NO: 245 - DNA, 20 bp, synthetic construct
  misc_feature 1..20: note = synthetic polymer
  source 1..20: mol_type = other DNA
ctggttgggaagacatgggg

SEQ ID NO: 246 - DNA, 20 bp, synthetic construct
  misc_feature 1..20: note = synthetic polymer
  source 1..20: mol_type = other DNA
cgtgaggatgggaaggaggg

SEQ ID NO: 247 - DNA, 20 bp, synthetic construct
  misc_feature 1..20: note = synthetic polymer
  source 1..20: mol_type = other DNA
atgcagagtcagcagaactg

SEQ ID NO: 248 - DNA, 20 bp, synthetic construct
  misc_feature 1..20: note = synthetic polymer
  source 1..20: mol_type = other DNA
aagacatcaagcacagaagg

SEQ ID NO: 249 - DNA, 20 bp, synthetic construct
  misc_feature 1..20: note = synthetic polymer
  source 1..20: mol_type = other DNA
tcaagcacagaaggaggagg

SEQ ID NO: 250 - DNA, 20 bp, synthetic construct
  misc_feature 1..20: note = synthetic polymer
  source 1..20: mol_type = other DNA
aaccgtcaataggcaaaggg

SEQ ID NO: 251 - DNA, 20 bp, synthetic construct
  misc_feature 1..20: note = synthetic polymer
  source 1..20: mol_type = other DNA
ccgtatttcagactgaatgg

SEQ ID NO: 252 - DNA, 20 bp, synthetic construct
  misc_feature 1..20: note = synthetic polymer
  source 1..20: mol_type = other DNA
gagaggacaggtgctacagg

SEQ ID NO: 253 - DNA, 20 bp, synthetic construct
  misc_feature 1..20: note = synthetic polymer
  source 1..20: mol_type = other DNA
aaccaaggaagggcaggagg

SEQ ID NO: 254 - DNA, 20 bp, synthetic construct
  misc_feature 1..20: note = synthetic polymer
  source 1..20: mol_type = other DNA
gacctctgggtggagacaga

SEQ ID NO: 255 - DNA, 20 bp, synthetic construct
  misc_feature 1..20: note = synthetic polymer
  source 1..20: mol_type = other DNA
cagatgaccatgacaagcag

SEQ ID NO: 256 - DNA, 20 bp, synthetic construct
  misc_feature 1..20: note = synthetic polymer
  source 1..20: mol_type = other DNA
aacaccagtgagtagagcgg

SEQ ID NO: 257 - DNA, 20 bp, synthetic construct
  misc_feature 1..20: note = synthetic polymer
  source 1..20: mol_type = other DNA
aggaccttgaagcacagaga

SEQ ID NO: 258 - DNA, 20 bp, synthetic construct
  misc_feature 1..20: note = synthetic polymer
  source 1..20: mol_type = other DNA
tacagaggcagactaaccca

SEQ ID NO: 259 - DNA, 20 bp, synthetic construct
  misc_feature 1..20: note = synthetic polymer
  source 1..20: mol_type = other DNA
acagaggcagactaacccag

SEQ ID NO: 260 - DNA, 20 bp, synthetic construct
  misc_feature 1..20: note = synthetic polymer
  source 1..20: mol_type = other DNA
taaatgacgtgctagacctg

SEQ ID NO: 261 - DNA, 20 bp, synthetic construct
  misc_feature 1..20: note = synthetic polymer
  source 1..20: mol_type = other DNA
agtaaccactcaggacaggg

SEQ ID NO: 262 - DNA, 20 bp, synthetic construct
  misc_feature 1..20: note = synthetic polymer
  source 1..20: mol_type = other DNA
accacaaaacagaaacacca

SEQ ID NO: 263 - DNA, 20 bp, synthetic construct
  misc_feature 1..20: note = synthetic polymer
  source 1..20: mol_type = other DNA
gtttgaagacaagcctgagg

SEQ ID NO: 264 - DNA, 20 bp, synthetic construct
  misc_feature 1..20: note = synthetic polymer
  source 1..20: mol_type = other DNA
gctgaaccccaaaagacagg

SEQ ID NO: 265 - DNA, 20 bp, synthetic construct
  misc_feature 1..20: note = synthetic polymer
  source 1..20: mol_type = other DNA
gcagctgagacacacaccag

SEQ ID NO: 266 - DNA, 20 bp, synthetic construct
  misc_feature 1..20: note = synthetic polymer
  source 1..20: mol_type = other DNA
aggacaccccaaagaagctg

SEQ ID NO: 267 - DNA, 20 bp, synthetic construct
  misc_feature 1..20: note = synthetic polymer
  source 1..20: mol_type = other DNA
ggacaccccaaagaagctga

SEQ ID NO: 268 - DNA, 20 bp, synthetic construct
  misc_feature 1..20: note = synthetic polymer
  source 1..20: mol_type = other DNA
ccagtgcaatggacagaaga

SEQ ID NO: 269 - DNA, 20 bp, synthetic construct
  misc_feature 1..20: note = synthetic polymer
  source 1..20: mol_type = other DNA
agaagagggagcctgcaagt

SEQ ID NO: 270 - DNA, 20 bp, synthetic construct
  misc_feature 1..20: note = synthetic polymer
  source 1..20: mol_type = other DNA
gtgtttgggccctagagcga

SEQ ID NO: 271 - DNA, 20 bp, synthetic construct
  misc_feature 1..20: note = synthetic polymer
  source 1..20: mol_type = other DNA
catgtgcctggtgcaatgca

SEQ ID NO: 272 - DNA, 20 bp, synthetic construct
  misc_feature 1..20: note = synthetic polymer
  source 1..20: mol_type = other DNA
tacaaagaggaagataagtg

SEQ ID NO: 273 - DNA, 20 bp, synthetic construct
  misc_feature 1..20: note = synthetic polymer
  source 1..20: mol_type = other DNA
gtcacagaatacaccactag

SEQ ID NO: 274 - DNA, 20 bp, synthetic construct
  misc_feature 1..20: note = synthetic polymer
  source 1..20: mol_type = other DNA
gggttaccctggacatggaa

SEQ ID NO: 275 - DNA, 20 bp, synthetic construct
  misc_feature 1..20: note = synthetic polymer
  source 1..20: mol_type = other DNA
catggaagggtattcactcg

SEQ ID NO: 276 - DNA, 20 bp, synthetic construct
  misc_feature 1..20: note = synthetic polymer
  source 1..20: mol_type = other DNA
agagtggcctagacaggctg

SEQ ID NO: 277 - DNA, 20 bp, synthetic construct
  misc_feature 1..20: note = synthetic polymer
  source 1..20: mol_type = other DNA
catgctggacagctcggcag

SEQ ID NO: 278 - DNA, 20 bp, synthetic construct
  misc_feature 1..20: note = synthetic polymer
  source 1..20: mol_type = other DNA
agtgaaagaagagaaaattc

SEQ ID NO: 279 - DNA, 20 bp, synthetic construct
  misc_feature 1..20: note = synthetic polymer
  source 1..20: mol_type = other DNA
tggtaagtctaagaaaccta

SEQ ID NO: 280 - DNA, 20 bp, synthetic construct
  misc_feature 1..20: note = synthetic polymer
  source 1..20: mol_type = other DNA
cccacagcctaaccacccta

SEQ ID NO: 281 - DNA, 20 bp, synthetic construct
  misc_feature 1..20: note = synthetic polymer
  source 1..20: mol_type = other DNA
aatatttcaaagccctaggg

SEQ ID NO: 282 - DNA, 20 bp, synthetic construct
  misc_feature 1..20: note = synthetic polymer
  source 1..20: mol_type = other DNA
gcactcggaacagggtctgg

SEQ ID NO: 283 - DNA, 20 bp, synthetic construct
  misc_feature 1..20: note = synthetic polymer
  source 1..20: mol_type = other DNA
agataggagctccaacagtg

SEQ ID NO: 284 - DNA, 20 bp, synthetic construct
  misc_feature 1..20: note = synthetic polymer
  source 1..20: mol_type = other DNA
aagttagagcagccaggaaa

SEQ ID NO: 285 - DNA, 20 bp, synthetic construct
  misc_feature 1..20: note = synthetic polymer
  source 1..20: mol_type = other DNA
tagagcagccaggaaaggga

SEQ ID NO: 286 - DNA, 20 bp, synthetic construct
  misc_feature 1..20: note = synthetic polymer
  source 1..20: mol_type = other DNA
tgaatacccttccatgtcca

SEQ ID NO: 287 - DNA, 20 bp, synthetic construct
  misc_feature 1..20: note = synthetic polymer
  source 1..20: mol_type = other DNA
cctgcattgcaccaggcaca

SEQ ID NO: 288 - DNA, 20 bp, synthetic construct
  misc_feature 1..20: note = synthetic polymer
  source 1..20: mol_type = other DNA
tctagggcccaaacacacct

SEQ ID NO: 289 - DNA, 20 bp, synthetic construct
  misc_feature 1..20: note = synthetic polymer
  source 1..20: mol_type = other DNA
tccctccatctatcaaaagg

SEQ ID NO: 290 - DNA, 20 bp, synthetic construct
  misc_feature 1..20: note = synthetic polymer
  source 1..20: mol_type = other DNA
agccctgagacagaagcagg

SEQ ID NO: 291 - DNA, 20 bp, synthetic construct
  misc_feature 1..20: note = synthetic polymer
  source 1..20: mol_type = other DNA
gccctgagacagaagcaggt

SEQ ID NO: 292 - DNA, 20 bp, synthetic construct
  misc_feature 1..20: note = synthetic polymer
  source 1..20: mol_type = other DNA
aggagatgcagtgatacgca

SEQ ID NO: 293 - DNA, 20 bp, synthetic construct
  misc_feature 1..20: note = synthetic polymer
  source 1..20: mol_type = other DNA
acaataccaagggtatccgg

SEQ ID NO: 294 - DNA, 20 bp, synthetic construct
  misc_feature 1..20: note = synthetic polymer
  source 1..20: mol_type = other DNA
tgataaagaaaacaaagtga

SEQ ID NO: 295 - DNA, 20 bp, synthetic construct
  misc_feature 1..20: note = synthetic polymer
  source 1..20: mol_type = other DNA
aaagaaaacaaagtgaggga

SEQ ID NO: 296 - DNA, 20 bp, synthetic construct
  misc_feature 1..20: note = synthetic polymer
  source 1..20: mol_type = other DNA
gtggcaagtggagaaattga

SEQ ID NO: 297 - DNA, 20 bp, synthetic construct
  misc_feature 1..20: note = synthetic polymer
  source 1..20: mol_type = other DNA
caagtggagaaattgaggga

SEQ ID NO: 298 - DNA, 20 bp, synthetic construct
  misc_feature 1..20: note = synthetic polymer
  source 1..20: mol_type = other DNA
gtggtgatgattgcagctgg

SEQ ID NO: 299 - DNA, 20 bp, synthetic construct
  misc_feature 1..20: note = synthetic polymer
  source 1..20: mol_type = other DNA
ctatgtgcctgacacacagg

SEQ ID NO: 300 - DNA, 20 bp, synthetic construct
  misc_feature 1..20: note = synthetic polymer
  source 1..20: mol_type = other DNA
gggttggaccaggaaagagg

SEQ ID NO: 301 - DNA, 20 bp, synthetic construct
  misc_feature 1..20: note = synthetic polymer
  source 1..20: mol_type = other DNA
gatgcctggaaaaggaaaga

SEQ ID NO: 302 - DNA, 20 bp, synthetic construct
  misc_feature 1..20: note = synthetic polymer
  source 1..20: mol_type = other DNA
tagtatgcacctgcaagagg

SEQ ID NO: 303 - DNA, 20 bp, synthetic construct
  misc_feature 1..20: note = synthetic polymer
  source 1..20: mol_type = other DNA
tatgcacctgcaagaggcgg

SEQ ID NO: 304 - DNA, 20 bp, synthetic construct
  misc_feature 1..20: note = synthetic polymer
  source 1..20: mol_type = other DNA
aggggaagaagagaagcaga

SEQ ID NO: 305 - DNA, 20 bp, synthetic construct
  misc_feature 1..20: note = synthetic polymer
  source 1..20: mol_type = other DNA
gctgaatcaagagacaagcg

SEQ ID NO: 306 - DNA, 20 bp, synthetic construct
  misc_feature 1..20: note = synthetic polymer
  source 1..20: mol_type = other DNA
aagcaaataaatctcctggg

SEQ ID NO: 307 - DNA, 20 bp, synthetic construct
  misc_feature 1..20: note = synthetic polymer
  source 1..20: mol_type = other DNA
agatgagtgctagagactgg

SEQ ID NO: 308 - DNA, 20 bp, synthetic construct
  misc_feature 1..20: note = synthetic polymer
  source 1..20: mol_type = other DNA
ctgatggttgagcacagcag

SEQ ID NO: 309 - DNA, 20 bp, synthetic construct
  misc_feature 1..20: note = synthetic polymer
  source 1..20: mol_type = other DNA
caaatggccagcaagggtgg

SEQ ID NO: 310 - DNA, 20 bp, synthetic construct
  misc_feature 1..20: note = synthetic polymer
  source 1..20: mol_type = other DNA
gcagaacctgaggatatgga

SEQ ID NO: 311 - DNA, 20 bp, synthetic construct
  misc_feature 1..20: note = synthetic polymer
  source 1..20: mol_type = other DNA
aatacacagaatgaaaatag

SEQ ID NO: 312 - DNA, 20 bp, synthetic construct
  misc_feature 1..20: note = synthetic polymer
  source 1..20: mol_type = other DNA
ctggtgactagaataggcag

SEQ ID NO: 313 - DNA, 20 bp, synthetic construct
  misc_feature 1..20: note = synthetic polymer
  source 1..20: mol_type = other DNA
tggtgactagaataggcagt

SEQ ID NO: 314 - DNA, 20 bp, synthetic construct
  misc_feature 1..20: note = synthetic polymer
  source 1..20: mol_type = other DNA
taaaagaatgtgaaaagatg

SEQ ID NO: 315 - DNA, 20 bp, synthetic construct
  misc_feature 1..20: note = synthetic polymer
  source 1..20: mol_type = other DNA
tcaggagttcaagaccaccc

SEQ ID NO: 316 - DNA, 20 bp, synthetic construct
  misc_feature 1..20: note = synthetic polymer
  source 1..20: mol_type = other DNA
tgtagtcccagttatgcagg

SEQ ID NO: 317 - DNA, 20 bp, synthetic construct
  misc_feature 1..20: note = synthetic polymer
  source 1..20: mol_type = other DNA
gggttcacaccacaaatgca

SEQ ID NO: 318 - DNA, 20 bp, synthetic construct
  misc_feature 1..20: note = synthetic polymer
  source 1..20: mol_type = other DNA
ggcaaatggccagcaagggt

SEQ ID NO: 319 - DNA, 20 bp, synthetic construct
  misc_feature 1..20: note = synthetic polymer
  source 1..20: mol_type = other DNA
agaaaccaatcccaaagcaa

SEQ ID NO: 320 - DNA, 20 bp, synthetic construct
  misc_feature 1..20: note = synthetic polymer
  source 1..20: mol_type = other DNA
gccaaggacaccaaaaccca

SEQ ID NO: 321 - DNA, 20 bp, synthetic construct
  misc_feature 1..20: note = synthetic polymer
  source 1..20: mol_type = other DNA
agtggtgataaggcaacagt

SEQ ID NO: 322 - DNA, 20 bp, synthetic construct
  misc_feature 1..20: note = synthetic polymer
  source 1..20: mol_type = other DNA
cctgagacagaagtattaag

SEQ ID NO: 323 - DNA, 20 bp, synthetic construct
  misc_feature 1..20: note = synthetic polymer
  source 1..20: mol_type = other DNA
aaggtcacacaatgaatagg

SEQ ID NO: 324 - DNA, 20 bp, synthetic construct
  misc_feature 1..20: note = synthetic polymer
  source 1..20: mol_type = other DNA
caccatactagggaagaaga

SEQ ID NO: 325 - DNA, 20 bp, synthetic construct
  misc_feature 1..20: note = synthetic polymer
  source 1..20: mol_type = other DNA
aataccctgcccttagtggg

SEQ ID NO: 326 - DNA, 20 bp, synthetic construct
  misc_feature 1..20: note = synthetic polymer
  source 1..20: mol_type = other DNA
ttagtggggggtggagtggg

SEQ ID NO: 327 - DNA, 20 bp, synthetic construct
  misc_feature 1..20: note = synthetic polymer
  source 1..20: mol_type = other DNA
caataccctgcccttagtgg

SEQ ID NO: 328 - DNA, 20 bp, synthetic construct
  misc_feature 1..20: note = synthetic polymer
  source 1..20: mol_type = other DNA
gtggggggtggagtgggggg

SEQ ID NO: 329 - DNA, 20 bp, synthetic construct
  misc_feature 1..20: note = synthetic polymer
  source 1..20: mol_type = other DNA
ggggggtggagtggggggtg

SEQ ID NO: 330 - DNA, 20 bp, synthetic construct
  misc_feature 1..20: note = synthetic polymer
  source 1..20: mol_type = other DNA
ggggtggagtggggggtggg

SEQ ID NO: 331 - DNA, 20 bp, synthetic construct
  misc_feature 1..20: note = synthetic polymer
  source 1..20: mol_type = other DNA
gggtggagtggggggtgggg

SEQ ID NO: 332 - DNA, 20 bp, synthetic construct
  misc_feature 1..20: note = synthetic polymer
  source 1..20: mol_type = other DNA
gggggtggggaaagacatcg

SEQ ID NO: 333 - DNA, 20 bp, synthetic construct
  misc_feature 1..20: note = synthetic polymer
  source 1..20: mol_type = other DNA
gcagctgtgaattctgatag

SEQ ID NO: 334 - DNA, 20 bp, synthetic construct
  misc_feature 1..20: note = synthetic polymer
  source 1..20: mol_type = other DNA
gagatcagagaaaccagatg

SEQ ID NO: 335 - DNA, 20 bp, synthetic construct
  misc_feature 1..20: note = synthetic polymer
  source 1..20: mol_type = other DNA
tctatactgattgcagccag

SEQ ID NO: 336-347 - skipped (12 entries)

SEQ ID NO: 348 - protein, 6 aa, synthetic construct
  REGION 1..6: note = synthetic polymer
  SITE 4: note = misc_feature - Xaa can be any naturally occurring amino acid
KKRXKR

SEQ ID NO: 349 - protein, 19 aa, synthetic construct
  REGION 1..19: note = synthetic polymer
  SITE 1: note = K may be replaced with R
  SITE 2: note = K may be replaced with R
  SITE 13: note = position may be deleted
  SITE 14: note = position may be deleted
  REGION 15..19: note = at least 3 of the 5 amino acids are K or R
KKXXXXXXXXXXXXKKKKK

SEQ ID NO: 350 - protein, 9 aa, synthetic construct
  REGION 1..9: note = synthetic polymer
PAAKRVKLD

SEQ ID NO: 351 - protein, 16 aa, synthetic construct
  REGION 1..16: note = synthetic polymer
KRPAATKKAGQAKKKK

SEQ ID NO: 352 - protein, 7 aa, synthetic construct
  REGION 1..7: note = synthetic polymer
PKKKRKV

SEQ ID NO: 353 - protein, 24 aa, synthetic construct
  REGION 1..24: note = synthetic polymer
DPKKKRKVDPKKKRKVDPKKKRKV

SEQ ID NO: 354-422 - skipped (69 entries)

SEQ ID NO: 423 - DNA, 333 bp, synthetic construct
  misc_feature 1..333: note = synthetic polymer
  source 1..333: mol_type = other DNA
ggcggatctggcggtagtgctgagtattgtctgagttacgaaacggaaatactcacggttgagtatgggcttcttccaattggcaaaatcgttgaaaagcgcatagagtgtacggtgtattccgtcgataacaacggtaatatctacacccagccggtagctcagtggcacgaccgaggcgaacaggaagtgttcgagtattgcttggaagatggctcccttatccgcgccactaaagaccataagtttatgacggttgacgggcagatgctgcctatagacgaaatatttgagagagagctggacttgatgagagtcgataatctgccaaat

SEQ ID NO: 424 - DNA, 180 bp, synthetic construct
  misc_feature 1..180: note = synthetic polymer
  source 1..180: mol_type = other DNA
ggcggatctggcggtagtgggggttccggatccataaagatagctactaggaaatatcttggcaaacaaaacgtctatgacataggagttgagcgagatcacaattttgctttgaagaatgggttcatcgcgtctaattgcttcaacgctagcggcgggtcaggaggctctggtggaagc

SEQ ID NO: 425 - DNA, 20 bp, synthetic construct
  misc_feature 1..20: note = synthetic polymer
  source 1..20: mol_type = other DNA
aatcgagaagcgactcgaca

SEQ ID NO: 426 - DNA, 20 bp, synthetic construct
  misc_feature 1..20: note = synthetic polymer
  source 1..20: mol_type = other DNA
tgccctgcaggggagtgagc

SEQ ID NO: 427 - DNA, 20 bp, synthetic construct
  misc_feature 1..20: note = synthetic polymer
  source 1..20: mol_type = other DNA
gaagcgactcgacatggagg

SEQ ID NO: 428 - DNA, 20 bp, synthetic construct
  misc_feature 1..20: note = synthetic polymer
  source 1..20: mol_type = other DNA
cctgcaggggagtgagcagc

SEQ ID NO: 429-500 - skipped (72 entries)

SEQ ID NO: 501 - DNA, 1716 bp, synthetic construct
  misc_feature 1..1716: note = synthetic polymer
  source 1..1716: mol_type = other DNA
atggcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 502 - DNA, 398 bp, synthetic construct; identical to SEQ ID NO: 2
  misc_feature 1..398: note = synthetic polymer
  misc_feature 14: note = n is a, c, g, or t
  misc_feature 21: note = n is a, c, g, or t
  misc_feature 59: note = n is a, c, g, or t
  misc_feature 65: note = n is a, c, g, or t
  misc_feature 70..71: note = n is a, c, g, or t
  misc_feature 81: note = n is a, c, g, or t
  misc_feature 85..86: note = n is a, c, g, or t
  misc_feature 88..89: note = n is a, c, g, or t
  misc_feature 137..139: note = n is a, c, g, or t
  misc_feature 143: note = n is a, c, g, or t
  misc_feature 156: note = n is a, c, g, or t
  misc_feature 171: note = n is a, c, g, or t
  misc_feature 188: note = n is a, c, g, or t
  misc_feature 211: note = n is a, c, g, or t
  misc_feature 214: note = n is a, c, g, or t
  misc_feature 218: note = n is a, c, g, or t
  misc_feature 223: note = n is a, c, g, or t
  misc_feature 231: note = n is a, c, g, or t
  misc_feature 245: note = n is a, c, g, or t
  misc_feature 261: note = n is a, c, g, or t
  misc_feature 271: note = n is a, c, g, or t
  misc_feature 275: note = n is a, c, g, or t
  misc_feature 304: note = n is a, c, g, or t
  misc_feature 307: note = n is a, c, g, or t
  misc_feature 366: note = n is a, c, g, or t
  misc_feature 386: note = n is a, c, g, or t
  misc_feature 397: note = n is a, c, g, or t
  source 1..398: mol_type = other DNA

SEQ ID NO: 503 - DNA, 1614 bp, synthetic construct
  misc_feature 1..1614: note = synthetic polymer
  source 1..1614: mol_type = other DNA
atgagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 504 - DNA, 372 bp, synthetic construct
  misc_feature 1..372: note = synthetic polymer
  misc_feature 33: note = n is a, c, g, or t
  misc_feature 39: note = n is a, c, g, or t
  misc_feature 44..45: note = n is a, c, g, or t
  misc_feature 55: note = n is a, c, g, or t
  misc_feature 59..60: note = n is a, c, g, or t
  misc_feature 62..63: note = n is a, c, g, or t
  misc_feature 111..113: note = n is a, c, g, or t
  misc_feature 117: note = n is a, c, g, or t
  misc_feature 130: note = n is a, c, g, or t
  misc_feature 145: note = n is a, c, g, or t
  misc_feature 162: note = n is a, c, g, or t
  misc_feature 185: note = n is a, c, g, or t
  misc_feature 188: note = n is a, c, g, or t
  misc_feature 192: note = n is a, c, g, or t
  misc_feature 197: note = n is a, c, g, or t
  misc_feature 205: note = n is a, c, g, or t
  misc_feature 219: note = n is a, c, g, or t
  misc_feature 235: note = n is a, c, g, or t
  misc_feature 245: note = n is a, c, g, or t
  misc_feature 249: note = n is a, c, g, or t
  misc_feature 278: note = n is a, c, g, or t
  misc_feature 281: note = n is a, c, g, or t
  misc_feature 340: note = n is a, c, g, or t
  misc_feature 360: note = n is a, c, g, or t
  misc_feature 371: note = n is a, c, g, or t
  source 1..372: mol_type = other DNA
mssddvmvrrtrrrrsssssdssdggrwshvdnvdghgntdavnndavkgddvsnryynnrnnkskkskwkdtmkkgvmgvrkdrrddywttwttygktmtrdrrwkawhnnnadvnsdrckvrvdyvknykhsdgvwrgrrvynagkvkygvrcsdtgycnmycggkrttvvsytdswyhymdnyynsvancamknkrcgtrknrgkdtskkgtkrkndvwskkvysshsamsndrtskkkvknadynkhmkgvdradysyysrrtvkwtkramymncansyavyksvrrkmgkmktahwtdddmdvdvstsgmraktsdcrsmdmrkhtavgsgkkknrrcrvcsvhkrstrymckcnhkgackyhtkny

SEQ ID NO: 505 - DNA, 1584 bp, synthetic construct
  misc_feature 1..1584: note = synthetic polymer
  source 1..1584: mol_type = other DNA
atgagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 506 - DNA, 364 bp, synthetic construct
  misc_feature 1..364: note = synthetic polymer
  misc_feature 25: note = n is a, c, g, or t
  misc_feature 31: note = n is a, c, g, or t
  misc_feature 36..37: note = n is a, c, g, or t
  misc_feature 47: note = n is a, c, g, or t
  misc_feature 51..52: note = n is a, c, g, or t
  misc_feature 54..55: note = n is a, c, g, or t
  misc_feature 103..105: note = n is a, c, g, or t
  misc_feature 109: note = n is a, c, g, or t
  misc_feature 122: note = n is a, c, g, or t
  misc_feature 137: note = n is a, c, g, or t
  misc_feature 154: note = n is a, c, g, or t
  misc_feature 177: note = n is a, c, g, or t
  misc_feature 180: note = n is a, c, g, or t
  misc_feature 184: note = n is a, c, g, or t
  misc_feature 189: note = n is a, c, g, or t
  misc_feature 197: note = n is a, c, g, or t
  misc_feature 211: note = n is a, c, g, or t
  misc_feature 227: note = n is a, c, g, or t
  misc_feature 237: note = n is a, c, g, or t
  misc_feature 241: note = n is a, c, g, or t
  misc_feature 270: note = n is a, c, g, or t
  misc_feature 273: note = n is a, c, g, or t
  misc_feature 332: note = n is a, c, g, or t
  misc_feature 352: note = n is a, c, g, or t
  misc_feature 363: note = n is a, c, g, or t
  source 1..364: mol_type = other DNA
mrtrrrrsssssdssdggrwshvdnvdghgntdavnndavkgddvsnryynnrnnkskkskwkdtmkkgvmgvrkdrrddywttwttygktmtrdrrwkawhnnnadvnsdrckvrvdyvknykhsdgvwrgrrvynagkvkygvrcsdtgycnmycggkrttvvsytdswyhymdnyynsvancamknkrcgtrknrgkdtskkgtkrkndvwskkvysshsamsndrtskkkvknadynkhmkgvdradysyysrrtvkwtkramymncansyavyksvrrkmgkmktahwtdddmdvdvstsgmraktsdcrsmdmrkhtavgsgkkknrrcrvcsvhkrstrymckcnhkgackyhtkny

SEQ ID NO: 507 - DNA, 1515 bp, synthetic construct
  misc_feature 1..1515: note = synthetic polymer
  source 1..1515: mol_type = other DNA
atggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 508 - DNA, 346 bp, synthetic construct
  misc_feature 1..346: note = synthetic polymer
  misc_feature 7: note = n is a, c, g, or t
  misc_feature 13: note = n is a, c, g, or t
  misc_feature 18..19: note = n is a, c, g, or t
  misc_feature 29: note = n is a, c, g, or t
  misc_feature 33..34: note = n is a, c, g, or t
  misc_feature 36..37: note = n is a, c, g, or t
  misc_feature 85..87: note = n is a, c, g, or t
  misc_feature 91: note = n is a, c, g, or t
  misc_feature 104: note = n is a, c, g, or t
  misc_feature 119: note = n is a, c, g, or t
  misc_feature 136: note = n is a, c, g, or t
  misc_feature 159: note = n is a, c, g, or t
  misc_feature 162: note = n is a, c, g, or t
  misc_feature 166: note = n is a, c, g, or t
  misc_feature 171: note = n is a, c, g, or t
  misc_feature 179: note = n is a, c, g, or t
  misc_feature 193: note = n is a, c, g, or t
  misc_feature 209: note = n is a, c, g, or t
  misc_feature 219: note = n is a, c, g, or t
  misc_feature 223: note = n is a, c, g, or t
  misc_feature 252: note = n is a, c, g, or t
  misc_feature 255: note = n is a, c, g, or t
  misc_feature 314: note = n is a, c, g, or t
  misc_feature 334: note = n is a, c, g, or t
  misc_feature 345: note = n is a, c, g, or t
  source 1..346: mol_type = other DNA
mwshvdnvdghgntdavnndavkgddvsnryynnrnnkskkskwkdtmkkgvmgvrkdrrddywttwttygktmtrdrrwkawhnnnadvnsdrckvrvdyvknykhsdgvwrgrrvynagkvkygvrcsdtgycnmycggkrttvvsytdswyhymdnyynsvancamknkrcgtrknrgkdtskkgtkrkndvwskkvysshsamsndrtskkkvknadynkhmkgvdradysyysrrtvkwtkramymncansyavyksvrrkmgkmktahwtdddmdvdvstsgmraktsdcrsmdmrkhtavgsgkkknrrcrvcsvhkrstrymckcnhkgackyhtkny

SEQ ID NO: 509 - DNA, 1452 bp, synthetic construct
  misc_feature 1..1452: note = synthetic polymer
  source 1..1452: mol_type = other DNA
atgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 510 - DNA, 335 bp, synthetic construct
  misc_feature 1..335: note = synthetic polymer
  misc_feature 2: note = n is a, c, g, or t
  misc_feature 7..8: note = n is a, c, g, or t
  misc_feature 18: note = n is a, c, g, or t
  misc_feature 22..23: note = n is a, c, g, or t
  misc_feature 25..26: note = n is a, c, g, or t
  misc_feature 74..76: note = n is a, c, g, or t
  misc_feature 80: note = n is a, c, g, or t
  misc_feature 93: note = n is a, c, g, or t
  misc_feature 108: note = n is a, c, g, or t
  misc_feature 125: note = n is a, c, g, or t
  misc_feature 148: note = n is a, c, g, or t
  misc_feature 151: note = n is a, c, g, or t
  misc_feature 155: note = n is a, c, g, or t
  misc_feature 160: note = n is a, c, g, or t
  misc_feature 168: note = n is a, c, g, or t
  misc_feature 182: note = n is a, c, g, or t
  misc_feature 198: note = n is a, c, g, or t
  misc_feature 208: note = n is a, c, g, or t
  misc_feature 212: note = n is a, c, g, or t
  misc_feature 241: note = n is a, c, g, or t
  misc_feature 244: note = n is a, c, g, or t
  misc_feature 303: note = n is a, c, g, or t
  misc_feature 323: note = n is a, c, g, or t
  misc_feature 334: note = n is a, c, g, or t
  source 1..335: mol_type = other DNA
mntdavnndavkgddvsnryynnrnnkskkskwkdtmkkgvmgvrkdrrddywttwttygktmtrdrrwkawhnnnadvnsdrckvrvdyvknykhsdgvwrgrrvynagkvkygvrcsdtgycnmycggkrttvvsytdswyhymdnyynsvancamknkrcgtrknrgkdtskkgtkrkndvwskkvysshsamsndrtskkkvknadynkhmkgvdradysyysrrtvkwtkramymncansyavyksvrrkmgkmktahwtdddmdvdvstsgmraktsdcrsmdmrkhtavgsgkkknrrcrvcsvhkrstrymckcnhkgackyhtkny

SEQ ID NO: 511 - DNA, 1662 bp, synthetic construct
  misc_feature 1..1662: note = synthetic polymer
  source 1..1662: mol_type = other DNA
atggcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaac

SEQ ID NO: 512 - DNA, 386 bp, synthetic construct
  misc_feature 1..386: note = synthetic polymer
  misc_feature 14: note = n is a, c, g, or t
  misc_feature 21: note = n is a, c, g, or t
  misc_feature 59: note = n is a, c, g, or t
  misc_feature 65: note = n is a, c, g, or t
  misc_feature 70..71: note = n is a, c, g, or t
  misc_feature 81: note = n is a, c, g, or t
  misc_feature 85..86: note = n is a, c, g, or t
  misc_feature 88..89: note = n is a, c, g, or t
  misc_feature 137..139: note = n is a, c, g, or t
  misc_feature 143: note = n is a, c, g, or t
  misc_feature 156: note = n is a, c, g, or t
  misc_feature 171: note = n is a, c, g, or t
  misc_feature 188: note = n is a, c, g, or t
  misc_feature 211: note = n is a, c, g, or t
  misc_feature 214: note = n is a, c, g, or t
  misc_feature 218: note = n is a, c, g, or t
  misc_feature 223: note = n is a, c, g, or t
  misc_feature 231: note = n is a, c, g, or t
  misc_feature 245: note = n is a, c, g, or t
  misc_feature 261: note = n is a, c, g, or t
  misc_feature 271: note = n is a, c, g, or t
  misc_feature 275: note = n is a, c, g, or t
  misc_feature 304: note = n is a, c, g, or t
  misc_feature 307: note = n is a, c, g, or t
  misc_feature 366: note = n is a, c, g, or t
  misc_feature 386: note = n is a, c, g, or t
  source 1..386: mol_type = other DNA
mahsdyddradksnyscdsdnastsddssddvmvrrtrrrrsssssdssdggrwshvdnvdghgntdavnndavkgddvsnryynnrnnkskkskwkdtmkkgvmgvrkdrrddywttwttygktmtrdrrwkawhnnnadvnsdrckvrvdyvknykhsdgvwrgrrvynagkvkygvrcsdtgycnmycggkrttvvsytdswyhymdnyynsvancamknkrcgtrknrgkdtskkgtkrkndvwskkvysshsamsndrtskkkvknadynkhmkgvdradysyysrrtvkwtkramymncansyavyksvrrkmgkmktahwtdddmdvdvstsgmraktsdcrsmdmrkhtavgsgkkknrrcrvcsvhkrstrymckcn

SEQ ID NO: 513 - DNA, 1587 bp, synthetic construct
  misc_feature 1..1587: note = synthetic polymer
  source 1..1587: mol_type = other DNA
atggcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaac

SEQ ID NO: 514 - DNA, 366 bp, synthetic construct
  misc_feature 1..366: note = synthetic polymer
  misc_feature 14: note = n is a, c, g, or t
  misc_feature 21: note = n is a, c, g, or t
  misc_feature 59: note = n is a, c, g, or t
  misc_feature 65: note = n is a, c, g, or t
  misc_feature 70..71: note = n is a, c, g, or t
  misc_feature 81: note = n is a, c, g, or t
  misc_feature 85..86: note = n is a, c, g, or t
  misc_feature 88..89: note = n is a, c, g, or t
  misc_feature 137..139: note = n is a, c, g, or t
  misc_feature 143: note = n is a, c, g, or t
  misc_feature 156: note = n is a, c, g, or t
  misc_feature 171: note = n is a, c, g, or t
  misc_feature 188: note = n is a, c, g, or t
  misc_feature 211: note = n is a, c, g, or t
  misc_feature 214: note = n is a, c, g, or t
  misc_feature 218: note = n is a, c, g, or t
  misc_feature 223: note = n is a, c, g, or t
  misc_feature 231: note = n is a, c, g, or t
  misc_feature 245: note = n is a, c, g, or t
  misc_feature 261: note = n is a, c, g, or t
  misc_feature 271: note = n is a, c, g, or t
  misc_feature 275: note = n is a, c, g, or t
  misc_feature 304: note = n is a, c, g, or t
  misc_feature 307: note = n is a, c, g, or t
  misc_feature 366: note = n is a, c, g, or t
  source 1..366: mol_type = other DNA
mahsdyddradksnyscdsdnastsddssddvmvrrtrrrrsssssdssdggrwshvdnvdghgntdavnndavkgddvsnryynnrnnkskkskwkdtmkkgvmgvrkdrrddywttwttygktmtrdrrwkawhnnnadvnsdrckvrvdyvknykhsdgvwrgrrvynagkvkygvrcsdtgycnmycggkrttvvsytdswyhymdnyynsvancamknkrcgtrknrgkdtskkgtkrkndvwskkvysshsamsndrtskkkvknadynkhmkgvdradysyysrrtvkwtkramymncansyavyksvrrkmgkmktahwtdddmdvdvstsgmraktsdcrsmdmrkhtavgsgkkkn

SEQ ID NO: 515-799 - skipped (285 entries)

SEQ ID NO: 800 - DNA, 20 bp, synthetic construct
  misc_feature 1..20: note = synthetic polymer
  source 1..20: mol_type = other DNA
ggagccacgaaaacagatcc

SEQ ID NO: 801 - DNA, 20 bp, synthetic construct
  misc_feature 1..20: note = synthetic polymer
  source 1..20: mol_type = other DNA
cgaaaacagatccagggaca

SEQ ID NO: 802 - DNA, 20 bp, synthetic construct
  misc_feature 1..20: note = synthetic polymer
  source 1..20: mol_type = other DNA
agatccagggacacggtgct

SEQ ID NO: 803 - DNA, 20 bp, synthetic construct
  misc_feature 1..20: note = synthetic polymer
  source 1..20: mol_type = other DNA
gacacggtgctaggacagtg

SEQ ID NO: 804 - DNA, 20 bp, synthetic construct
  misc_feature 1..20: note = synthetic polymer
  source 1..20: mol_type = other DNA
gaaaatgacccaacagcctc

SEQ ID NO: 805 - DNA, 20 bp, synthetic construct
  misc_feature 1..20: note = synthetic polymer
  source 1..20: mol_type = other DNA
gcctggccggcctgaccact

SEQ ID NO: 806 - DNA, 20 bp, synthetic construct
  misc_feature 1..20: note = synthetic polymer
  source 1..20: mol_type = other DNA
ctgagcactgaaggcctggc

SEQ ID NO: 807 - DNA, 20 bp, synthetic construct
  misc_feature 1..20: note = synthetic polymer
  source 1..20: mol_type = other DNA
tggtttccactgagcactga

SEQ ID NO: 808 - DNA, 20 bp, synthetic construct
  misc_feature 1..20: note = synthetic polymer
  source 1..20: mol_type = other DNA
gatagccaggagtcctttcg

SEQ ID NO: 809 - DNA, 20 bp, synthetic construct
  misc_feature 1..20: note = synthetic polymer
  source 1..20: mol_type = other DNA
gcgcttccagtgctcagact

SEQ ID NO: 810 - DNA, 20 bp, synthetic construct
  misc_feature 1..20: note = synthetic polymer
  source 1..20: mol_type = other DNA
cagtgctcagactagggaag

SEQ ID NO: 811 - DNA, 20 bp, synthetic construct
  misc_feature 1..20: note = synthetic polymer
  source 1..20: mol_type = other DNA
gcccctcctccttcagagcc

SEQ ID NO: 812 - DNA, 20 bp, synthetic construct
  misc_feature 1..20: note = synthetic polymer
  source 1..20: mol_type = other DNA
tccttcagagccaggagtcc

SEQ ID NO: 813 - DNA, 20 bp, synthetic construct
  misc_feature 1..20: note = synthetic polymer
  source 1..20: mol_type = other DNA
tggtttccgagcttgaccct

SEQ ID NO: 814 - DNA, 20 bp, synthetic construct
  misc_feature 1..20: note = synthetic polymer
  source 1..20: mol_type = other DNA
ctgcagagtatctgctgggg

SEQ ID NO: 815 - DNA, 20 bp, synthetic construct
  misc_feature 1..20: note = synthetic polymer
  source 1..20: mol_type = other DNA
cgttcctgcagagtatctgc

SEQ ID NO: 816 - DNA, 56 bp, synthetic construct
  misc_feature 1..56: note = synthetic polymer
  source 1..56: mol_type = other DNA
ctccactgtggggtgccgcagtggagatgacttgtttgttaacggcactttaacag

(19) United States
(12) Patent Application Publication
(10) Pub. No.: US 2025/0002876 A1
(43) Pub. Date: Jan. 2, 2025

(54) MOBILE ELEMENTS AND CHIMERIC CONSTRUCTS THEREOF

(71) Applicant: Saliogen Therapeutics, Inc., Lexington, MA (US)

(72) Inventors: Joseph J. HIGGINS, Lexington, MA (US); Ray TABIBIAZAR, Lexington, MA (US); Nancy CRAIG, Lexington, MA (US)

(21) Appl. No.: 18/706,118

(22) PCT Filed: Nov. 4, 2022
(86) PCT No.: PCT/US22/79292

§ 371 Date: Apr. 30, 2024

Related U.S. Application Data
(60) Provisional application No. 63/408,186, filed on Sep. 20, 2022.
(60) Provisional application No. 63/350,775, filed on Jun. 9, 2022.
(60) Provisional application No. 63/331,433, filed on Apr. 15, 2022.
(60) Provisional application No. 63/275,778, filed on Nov. 4, 2021.

(51) Int. Cl. C12N 9/12; C12N 9/22; C12N 15/90

(52) U.S. Cl. CPC C12N 9/1241; C12N 9/22; C12N 15/907; C12N 2310/20; C12N 2800/90

(57) ABSTRACT

Gene therapy compositions and methods related to transposition are provided.

### PRIORITY

This application claims priority to and the benefit of U.S. Provisional Patent Application No. 63/275,778, filed on Nov. 4, 2021, U.S. Provisional Patent Application No. 63/331,433, filed on Apr. 15, 2022, U.S. Provisional Patent Application No. 63/350,775, filed on Jun. 9, 2022, and U.S. Provisional Patent Application No. 63/408,186 filed on Sep. 20, 2022, the entire content of which are hereby incorporated herein by reference in its entirety.

### FIELD

The present disclosure relates to recombinant mobile element systems and uses thereof.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: “Sequence_Listing_SAL-012PC_126933-5012.xml”; date recorded: Nov. 4, 2022; file size: 970,752 bytes).

### BACKGROUND

Mobile elements are genetic sequences that are found, with small exceptions, in all living organisms. These elements have deep evolutionary origins and diversification and have an astonishing variety of forms and shapes. See Bourque, G., Burns, K. H., Gehring, M., Gorbunova, V., Seluanov, A., Hammell, M., . . . Feschotte, C. (2018). Ten things you should know about transposable elements. Genome Biol, 19(1), 199.

A nucleic acid movement to a new location in the human genome is performed by the action of a helper enzyme that binds to an “end sequence” and inserts a donor DNA sequence at a specific DNA sequence by a “cut and paste” mechanism. The donor DNA is flanked by end sequences in living organisms such as insects (e.g., Trichnoplusia ni). Genomic DNA is excised by double strand cleavage at the hosts' donor site and the donor DNA is integrated or inserted into a specific DNA sequence. Mobilization of the DNA sequences permits the intervening nucleic acid, or a transgene, to be inserted at the specific nucleotide sequence (i.e., TTAA) without a DNA footprint.

Two eukaryotic mobile elements have been widely used as a means for gene delivery in a variety of applications. See Kang, et al. (2009). For example, piggyBac (pB) is an integrating non-viral gene transfer vector that enhances the efficiency of gene-directed enzyme prodrug therapy (GDEPT). Cell Biol Int, 33(4), 509-515; Lacoste, et al. (2009). An efficient and reversible mobile element system for gene delivery and lineage-specific differentiation in human embryonic stem cells. Cell Stem Cell, 5(3), 332-342; Saridey, et al. (2009). PB-based inducible gene expression in vivo after somatic cell gene transfer. Mol Ther, 17(12), 2115-2120; Wang, et al. (2009). A pB-based genome-wide library of insertionally mutated Blm-deficient murine ES cells. Genome Res, 19(4), 667-673; Woltjen, et al. (2009). PB reprograms fibroblasts to induced pluripotent stem cells. Nature, 458(7239), 766-770; Wu, et al. (2006). piggyBac is a flexible and highly active mobile element as compared to sleeping beauty, Tol2, and Mos1 in mammalian cells. Proc Natl Acad Sci USA, 103(41), 15008-15013; Ivics, et al. (1997). Molecular reconstruction of Sleeping Beauty, a Tc1-like mobile element from fish, and its transposition in human cells. Cell, 91(4), 501-510; Ivics, et al. (2009). Mobile element-mediated genome manipulation in vertebrates. Nat Methods, 6(6), 415-422; Ding, et al. (2005). Efficient transposition of pB in mammalian cells and mice. Cell, 122(3), 473-483; Yusa, et al. (2011). A hyperactive pB mobile element for mammalian applications. Proc Natl Acad Sci USA, 108(4), 1531-1536. These mobile element systems, among others, have been shown to efficiently deliver transgenes in vitro and in vivo. See Ding, et al. (2005). Efficient transposition of the pB in mammalian cells and mice. Cell, 122(3), 473-483; Ivics, et al. (1997). Molecular reconstruction of Sleeping Beauty, a Tc1-like mobile element from fish, and its transposition in human cells. Cell, 91(4), 501-510; Montini, et al. (2002). In vivo correction of murine tyrosinemia type I by DNA-mediated transposition. Mol Ther, 6(6), 759-769; Wu, et al. (2006). PB is a flexible and highly active donor as compared to sleeping beauty, Tol2, and Mos1 in mammalian cells. Proc Natl Acad Sci USA, 103(41), 15008-15013; Yusa, et al. (2011). A hyperactive pB mobile element for mammalian applications. Proc Natl Acad Sci USA, 108(4), 1531-1536. Notably, these helper enzymes are able to integrate large gene cassettes of more than 100 kb. See Li, et al. (2011). Mobilization of giant pB mobile elements in the mouse genome. Nucleic Acids Res, 39(22), e148. Because both these mobile elements, carryout direct insertion into many genomic sites, issues related to safety and the risk of insertional mutagenesis are raised.

There is a need for safer helpers if this technology is to find use in medicine.

### SUMMARY

Accordingly, this disclosure describes, in part, a helper RNA that encodes for an excision competent/integration defective (Exc+Int−) helper enzyme that is optionally engineered to target a single human genomic locus by introducing DNA binding proteins at its N-terminus. The present disclosure provides a composition comprising a recombinant mobile element enzyme that has bioengineered enhanced gene cleavage [Excision (Exc+)] and/or integration deficient (Int−) and/or integration efficient (Int+) gene activity, and DNA binders (e.g., without limitation, dCas9, TALEs, and ZnF) that guide donor insertion to specific genomic sites.

In aspects there is provided a composition comprising (a) a helper enzyme or a nucleic acid encoding the helper enzyme and (b) a targeting element or a nucleic acid encoding the targeting element and (c) a linker connecting the helper enzyme and the targeting element, wherein: the helper enzyme comprises an amino acid sequence having at least about 80% sequence identity to SEQ ID NO: 9 and has a non-polar aliphatic amino acid at position 2 of SEQ ID NO: 9 or a position corresponding thereto and one or more of S8X₁ of SEQ ID NO: 9 or a position corresponding thereto, wherein X₁ is selected from alanine (A), glycine (G), valine (V), leucine (L), isoleucine (I), and proline (P); C13X₂ of SEQ ID NO: 9 or a position corresponding thereto, wherein X₂ is selected from lysine (K), arginine (R), and histidine (H); and N125X₃ of SEQ ID NO: 9 or a position corresponding thereto, wherein X₃ is selected from is selected from lysine (K), arginine (R), and histidine (H); the targeting element is or comprises one or more of a Cas enzyme, which is optionally catalytically inactive and which is optionally associated with a guide RNA (gRNA), a transcription activator-like effector (TALE) DNA binding domain (DBD), a Zinc finger (ZF), a catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof; and the linker comprises less than about 25 amino acids or 75 nucleotides.

In aspects there is provided a composition comprising (a) a helper enzyme or a nucleic acid encoding the helper enzyme and (b) a targeting element or a nucleic acid encoding the targeting element, wherein: the helper enzyme comprises an amino acid sequence having at least about 80% sequence identity to SEQ ID NO: 9 and has a non-polar aliphatic amino acid at position 2 of SEQ ID NO: 9 or a position corresponding thereto and one or more of S8X₁ of SEQ ID NO: 9 or a position corresponding thereto, wherein X₁ is selected from alanine (A), glycine (G), valine (V), leucine (L), isoleucine (I), and proline (P); C13X₂ of SEQ ID NO: 9 or a position corresponding thereto, wherein X₂ is selected from lysine (K), arginine (R), and histidine (H); and N125X₃ of SEQ ID NO: 9 or a position corresponding thereto, wherein X₃ is selected from is selected from lysine (K), arginine (R), and histidine (H); the targeting element is or comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof; and wherein the targeting element directs the helper enzyme to one or more nucleic acids sites that are upstream and/or downstream of the TTAA integration sites and within about 5 to about 30 base pairs of the TTAA integration sites or within about 15 to about 19 base pairs of the TTAA integration sites and optionally a linker connecting the helper enzyme and the targeting element, the linker comprises less than about 25 amino acids or 75 nucleotides.

In embodiments, the non-polar aliphatic amino acid is selected from alanine (A), glycine (G), valine (V), leucine (L), isoleucine (I), and proline (P).

In embodiments, the linker comprises about 10 amino acids to about 20 amino acids or about 12 amino acids to about 15 amino acids, or about 30 nucleotides to about 60 nucleotides or about 36 nucleotides to about 45 nucleotides. In embodiments, the er is substantially comprised of glycine (G) and serine (S) residues. In embodiments, the linker is or comprises (GSS)₄ or in the case of insertion of a DNA binder (TALE, ZnF) in an intrinsic DNA binding loop, the linker is (GS)1 on either side of the DNA binder (TALE, ZnF). In embodiments, the linker connects the targeting element to the N-terminus of the helper enzyme or connects the targeting element within the helper enzyme.

In embodiments, the helper enzyme is suitable of inserting a donor nucleic acid comprising a transgene in a genomic safe harbor site (GSHS) and/or wherein the targeting element is suitable for directing the helper enzyme to a GSHS. In embodiments, the GSHS is in an open chromatin location in a chromosome. In embodiments, the GSHS is selected from adeno-associated virus site 1 (AAVS1), chemokine (C-C motif) receptor 5 (CCR5) gene, HIV-1 coreceptor, and human Rosa26 locus. In embodiments, the GSHS comprises one or more TTAA integration sites. In embodiments, the targeting element directs the helper enzyme to one or more nucleic acid sites that are upstream and/or downstream of the TTAA integration sites. In embodiments, the targeting element directs the helper enzyme to either one or more nucleic acid sites that are upstream and/or downstream of the TTAA integration sites or to the TTAA integration sites and within about 5 to about 30 base pairs of the TTAA integration sites or within about 15 to about 19 base pairs of the TTAA integration sites. In embodiments, the targeting element directs the helper enzyme to two nucleic acid sites of the TTAA integration sites, wherein a first site is upstream of TTAA and within about 5 to about 30 base pairs or about 15 to about 19 base pairs of the TTAA and a second site is downstream of TTAA and within about 5 to about 30 base pairs or about 15 to about 19 base pairs of the TTAA.

In embodiments, the helper enzyme comprises an amino acid sequence having at least about 90% sequence identity to SEQ ID NO: 9. In embodiments, the helper enzyme comprises an amino acid sequence having at least about 95% sequence identity to SEQ ID NO: 9. In embodiments, the helper enzyme comprises an amino acid sequence having at least about 98% sequence identity to SEQ ID NO: 9.

In embodiments, a donor DNA and a helper RNA are transfected at a donor DNA to helper RNA ratio of about 1 to about 4, or about 1 to about 2, or about 1 to about 1.

In embodiments, the helper enzyme comprises an N- or C-terminal deletion, optionally at positions 1-35, or 1-45, or 1-55, or 1-65, or 1-75, or 1-85, or 1-95, or 1-105 or positions corresponding thereto, wherein the positions are relative to SEQ ID NO: 9. In embodiments, the helper enzyme comprises an N-terminal deletion, optionally at positions 1-34, or 1-45, or 1-68, or 1-89 or positions corresponding thereto, wherein the positions are relative to SEQ ID NO: 9. In embodiments, the helper enzyme comprises a C-terminal deletion, optionally at positions 555-573 or 530-573 or positions corresponding thereto, wherein the positions are relative to SEQ ID NO: 9. In embodiments, the N- or C-terminal deletion yields reduced or ablated off-target effects of the helper enzyme compared to the helper enzyme without the N- or C-terminal deletion. In embodiments, the helper enzyme comprising the N-terminal deletion is or comprises an amino acid sequence of SEQ ID NO: 506, or a sequence having at least about 80%, or at least about 90%, or at least about 95%, or at least about 98% identity thereto. In embodiments, the helper enzyme comprises at least one substitution at position D416, or a position corresponding thereto relative to SEQ ID NO: 9. In embodiments, the substitution at position D416 or a position corresponding thereto relative to SEQ ID NO: 9 is a polar and positively charged hydrophilic residue optionally selected from arginine (R) and lysine (K), a polar and neutral of charge hydrophilic residue selected from asparagine (N), glutamine (Q), serine (S), threonine (T), proline (P), and cysteine (C). In embodiments, the substitution at position D416 or a position corresponding thereto relative to SEQ ID NO: 9 is asparagine (N). In embodiments, the helper enzyme comprises at least one substitution at selected from the mutations of FIG. 8, FIG. 20, TABLE 1, and/or TABLE 2.

In embodiments, the composition is a nucleic acid, optionally an RNA. In embodiments, the composition further comprises a donor nucleic acid or is suitable for insertion of a donor nucleic acid, optionally wherein the donor nucleic acid is a transposon.

In embodiments, there is provided a method for inserting a gene into the genome of a cell, comprising contacting a cell with the composition described herein. In embodiments, there is provided a method for treating a disease or disorder ex vivo, comprising contacting a cell with the composition described herein and administering the cell to a subject in need thereof. In embodiments, there is provided a method for treating a disease or disorder in vivo, comprising administering the composition of described herein to a subject in need thereof.

In embodiments, the helper enzyme is an engineered form of an enzyme reconstructed from Myotis lucifugus. In embodiments, the helper enzyme includes but is not limited to an engineered version that is a monomer, dimer, tetramer (or another multimer), hyperactive (Exc+), and/or has a reduced interaction with non-TTAA recognitions sites (Int−), of a helper enzyme reconstructed from Myotis lucifugus or a predecessor thereof.

In some embodiments, the helper enzyme, having gene cleavage (Exc) and/or gene integration (Int) activity, has at least about 90% identity to the nucleotide sequence of SEQ ID NO: 1 or the amino acid sequence SEQ ID NO: 2. In some embodiments, the helper enzyme has at least about 95%, or at least about 96%, at least about 97%, at least about 98%, at least about 99% identity to the amino acid sequence variants or combination thereof shown in TABLE 1 and TABLE 2 or positions corresponding thereto, which correspond positions of SEQ ID NO: 9, or a nucleotide sequence encoding the same.

In embodiments, the helper enzyme has one or more mutations which confer hyperactivity and Exc+/Int−. In some embodiments, the helper enzyme has an amino acid sequence having mutations at positions which correspond to at least one of S8P and C13R, or both, mutations relative to the amino acid sequence of SEQ ID NO: 9 or a functional equivalent thereof.

In embodiments, the helper enzyme has deletions which confer hyperactivity and Exc+/Int−. In some embodiments, the helper enzyme has an amino acid sequence having deletions at N-terminus positions, e.g., 1-89, or C-terminus positions, e.g., 555-572, (FIG. 9) relative to the amino acid sequence of SEQ ID NO: 2 or SEQ ID NO: 9 and optionally fused to the amino acid sequence of SEQ ID NO: 6 (dCas9), or a functional equivalent thereof.

In embodiments, the helper enzyme has deletions which confer hyperactivity and Exc+/Int−. In some embodiments, the helper enzyme has an amino acid sequence having deletions at C-terminus, e.g., position 555-572, (FIG. 9) relative to the amino acid sequence of SEQ ID NO: 2 or SEQ ID NO: 9 and optionally fused to a protein binder on one monomer and its ligand on the other monomer to induce dimerization (FIG. 6E), or a functional equivalent thereof. In some embodiments, the helper enzyme has an extrinsic DNA binding domain inserted in a natural DNA binding loop (Y281-P339) which confers Exc+/Int− (FIG. 6F).

In embodiments, the helper enzyme of the present disclosure comprises a deletion at positions about 1-35, or about 1-45, or about 1-55, or about 1-65, or about 1-75, or about 1-85, or about 1-95, or about 1-105 or positions corresponding thereto, wherein the positions are relative to SEQ ID NO: 9 or SEQ ID NO: 502. In embodiments, the helper enzyme is an MLT. In embodiments, the deletion comprises an N or C terminal deletion. In embodiments, the N or C terminal deletion yields reduced or ablated off-target effects of the helper enzyme compared to the helper enzyme without the N or C terminal deletion. In embodiments, the helper enzyme comprising the N terminal deletion is N2. In embodiments, the helper enzyme comprising the N terminal deletion is or comprises SEQ ID NO: 506. In embodiments, the mutant with an N or C terminal deletion is further fused to a DNA binder. In embodiments, the DNA binder comprises TALEs, ZnF, and/or both.

In some embodiments, the composition comprises a gene transfer construct. The gene transfer donor DNA construct can be or can comprise a vector comprising a mobile element comprising one or more end sequences recognized by the helper enzyme. In some embodiments, the end sequences are left and right end sequences that are recombinant or synthetic sequences. In embodiments, the end sequences are selected from Myositis lucifugus, or end sequences with similarity to piggyBac-like mobile elements and exhibit duplications of their presumed TTAA target sites. In some embodiments, the end sequences are selected from nucleotide sequences of SEQ ID NO: 3, and SEQ ID NO: 4, or a nucleotide sequence having at least about 90% identity thereto or end sequences with 80 bp deletions at the 3′end of SEQ. ID NO: 3 or the 5′-end of SEQ ID NO: 4.

In some embodiments, the end sequences include at least one repeat from a nucleotide sequence having at least about 90% identity to the nucleotide sequence of SEQ ID NO: 3, and wherein the at least one repeat from the nucleotide sequence having at least about 90% identity to the nucleotide sequence of SEQ ID NO: 3 is positioned at the 5′ end of the donor. The end sequences can further include at least one repeat from a nucleotide sequence having at least about 90% identity to the nucleotide sequence of SEQ ID NO: 4, and wherein the at least one repeat from the nucleotide sequence having at least about 90% identity to the nucleotide sequence of SEQ ID NO: 4 is positioned at the 3′ end of the donor. The end sequences, which can be, e.g., Myotis lucifugus, are optionally flanked by a TTAA sequence.

In some embodiments, the helper enzyme is included in the gene transfer construct. In some embodiments, the composition comprises a nucleic acid binding component of a gene-editing system. In some embodiments, the gene-editing system is included in the gene transfer construct.

In some embodiments, the gene-editing system comprises a CRISPR/Cas enzyme (class I, class II), or their six subtypes (type I-VI) (e.g., Cas9, Cas12a, Cas12j, Cas12k), or a variant thereof. In some embodiments, the gene-editing system comprises a nuclease-deficient a CRISPR/Cas enzyme (class I, class II), or their six subtypes (type I-VI) (e.g., dCas9, dCas12a, dCas12j, dCas12k). In some embodiments, the gene-editing system comprises Cas9, Cas12a, Cas12j, or Cas12k, or a variant thereof. For example, the gene-editing system comprises a nuclease-deficient dCas9, dCas12a, dCas12j, or dCas12k.

In some embodiments, the composition has the helper enzyme and the nucleic acid binding component of the gene-editing system.

In some embodiments, the composition comprises a chimeric mobile element construct comprising the helper enzyme and the nucleic acid binding component of the gene-editing system fused or linked thereto. The helper enzyme and the nucleic acid binding component of the gene-editing system can be fused or linked to one another via a linker, which can be a flexible linker. The flexible linker can be substantially comprised of glycine and serine residues, optionally wherein the flexible linker comprises (Gly₄Ser)ₙ, where n is from about 1 to about 12. In some embodiments, the flexible linker is of or about 50, or about 100, or about 150, or about 200 amino acid residues. In some embodiments, the flexible linker comprises at least about 150 nucleotides (nt), or at least about 200 nt, or at least about 250 nt, or at least about 300 nt, or at least about 350 nt, or at least about 400 nt, or at least about 450 nt, or at least about 500 nt, or at least about 500 nt, or at least about 600 nt. In some embodiments, the flexible linker comprises from about 450 nt to about 500 nt. In some embodiments, the helper enzyme is capable of inserting a donor at a TA dinucleotide site or a TTAA tetranucleotide site in a genomic safe harbor site (GSHS) of a nucleic acid molecule.

In some embodiments, the donor comprises a gene encoding a complete polypeptide. In some embodiments, the donor comprises a gene which is defective or substantially absent in a disease state.

In some aspects, a composition is provided comprising (a) a nucleic acid binding component of a gene-editing system, and (b) a recombinant mammalian helper enzyme, the helper enzyme having at least about 90% identity to the amino acid sequence of SEQ ID NO: 2 or SEQ ID NO: 9, or a nucleotide sequence encoding the same. In some embodiments, the helper enzyme has at least about 95%, or at least about 96%, at least about 97%, at least about 98%, at least about 99% identity to the amino acid sequence of SEQ ID NO: 2 or SEQ ID NO: 9, or a nucleotide sequence encoding the same.

In some embodiments, a mobile element construct comprises a helper enzyme (both herein called “helper”) constructed as a DNA vector or RNA vector (FIG. 6A) fused or linked to a DNA binding domain (DBD), or TALE (FIG. 6B), zinc finger (ZnF) (FIG. 6C), inactive Cas protein (dCas9, dCas12a, dCas12j, or dCas12k) programmed by a guide RNA (gRNA) (FIG. 6D), a construct with an intein or dimerization enhancer such as SH3, biotin, avidin, or rapamycin binders (FIG. 6E), or a construct with an extrinsic DNA binding domain (TALE, ZnF) that interrupts the helper enzymes natural DNA binding loop (Y281-P339).

A composition comprising a recombinant mammalian helper enzyme in accordance with embodiments of the present disclosure can include one or more non-viral vectors. Also, the recombinant mammalian helper enzyme can be disposed on the same (cis) or different vector (trans) than a donor with a transgene. Accordingly, in some embodiments, the recombinant mammalian helper enzyme and the donor encompassing a transgene are in cis configuration such that they are included in the same vector. In some embodiments, the recombinant mammalian helper enzyme and the donor encompassing a transgene are in trans configuration such that they are included in different vectors. The vector is any non-viral vector in accordance with the present disclosure.

In some aspects, a nucleic acid encoding a recombinant mammalian helper enzyme in accordance with embodiments of the present disclosure is provided. The nucleic acid can be DNA or RNA. In some embodiments, the nucleic acid is DNA. In some embodiments, the nucleic acid is RNA that has a 5′-m7G cap (cap 0, cap1, or cap2) with pseudouridine substitution or N-methyl-pseudouridine substitution, and a poly-A tail of or about 30, or about 50, or about 100, of about 150 nucleotides in length. In some embodiments, the recombinant mammalian helper enzyme is incorporated into a vector. In some embodiments, the vector is a non-viral vector.

In some aspects, a host cell comprising the nucleic acid in accordance with embodiments of the present disclosure is provided.

In some embodiments, a composition or a nucleic acid in accordance with embodiments of the present disclosure is provided wherein the composition is in the form of a lipid nanoparticle (LNP). The composition can comprise one or more lipids selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy(polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC) and/or comprising of one or more molecules selected from polyethylenimine (PEI) and poly(lactic-co-glycolic acid) (PLGA), and N-Acetylgalactosamine (GalNAc).

In some embodiments, an LNP can be as described, e.g., in Patel et al., J Control Release 2019; 303:91-100. The LNP can comprise one or more of a structural lipid (e.g., DSPC), a PEG-conjugated lipid (CDM-PEG), a cationic lipid (MC3), cholesterol, and a targeting ligand (e.g., GalNAc).

In some aspects, a method for inserting a gene into the genome of a cell is provided that comprises contacting a cell with a recombinant mammalian helper enzyme in accordance with embodiments of the present disclosure. The method can be in vivo or ex vivo method.

In some embodiments, the cell is contacted with a nucleic acid encoding the helper enzyme. In some embodiments, the nucleic acid further comprises a donor having a gene. In some embodiments, the cell is contacted with a construct comprising a donor having a gene.

In some embodiments, the cell is contacted with an RNA encoding the helper enzyme.

In some embodiments, the cell is contacted with a DNA encoding the helper enzyme. In some embodiments, the donor is flanked by one or more end sequences, such as left and right end sequences. In some embodiments, the donor can be under control of a tissue-specific promoter. In some embodiments, the donor is an ATP Binding Cassette Subfamily A Member 4 gene (ABC) transporter gene (ABCA4), or functional fragment thereof. As another example, in some embodiments, the donor is a very low-density lipoprotein receptor gene (VLDLR) or a low-density lipoprotein receptor gene (LDLR), or a functional fragment thereof.

In some embodiments, the donor is a gene encoding a complete polypeptide. In some embodiments, the donor is a gene which is defective or substantially absent in a disease state.

In some embodiments, a kit is provided that comprises a recombinant mammalian helper enzyme and/or or a nucleic acid according to any embodiments, or combination thereof, of the present disclosure, and instructions for introducing DNA into a cell using the recombinant mammalian helper.

In embodiments, the present method, which makes use of a recombinant mammalian helper identified in accordance with embodiments of the present disclosure, provides reduced insertional mutagenesis or oncogenesis as compared to a method with a non-chimeric helper and as compared to non-mammalian helpers. Because the recombinant helper enzyme is from a mammalian genome, the mammalian helper enzyme is safer and more efficient than helpers from plants, insects, and bats.

In embodiments, the method is used to treat an inherited or acquired disease in a patient in need thereof.

For example, in some embodiments, the method is used for treating and/or mitigating a class of Inherited Macular Degeneration (IMDs) (also referred to as Macular dystrophies (MDs), including Stargardt disease (STGD), Best disease, X-linked retinoschisis, pattern dystrophy, Sorsby fundus dystrophy and autosomal dominant drusen. The STGD can be STGD Type 1 (STGD1). In some embodiments, the STGD can be STGD Type 3 (STGD3) or STGD Type 4 (STGD4) disease. The IMD can be characterized by one or more mutations in one or more of ABCA4, ELOVL4, PROM1, BEST1, and PRPH2. The gene therapy can be performed using donor-based vector systems, with the assistance by chimeric helpers in accordance with the present disclosure, which are provided on the same vector as the gene to be transferred (cis) or on a different vector (trans) or as RNA. The donor can comprise an ATP binding cassette subfamily A member 4 (ABCA4), or functional fragment thereof, and the donor-based vector systems can operate under the control of a retina-specific promoter.

In some embodiments, the method is used for treating and/or mitigating familial hypercholesterolemia (FH), such as homozygous FH (HoFH) or heterozygous FH (HeFH) or disorders associated with elevated levels of low-density lipoprotein cholesterol (LDL-C). The gene therapy can be performed using donor-based vector systems, with the assistance by chimeric helpers in accordance with the present disclosure, which are provided on the same vector (cis) as the gene to be transferred or on a different vector (trans). The donor can comprise a very low-density lipoprotein receptor gene (VLDLR) or a low-density lipoprotein receptor gene (LDLR), or a functional fragment thereof. The donor-based vector systems can operate under control of a liver-specific promoter. In some embodiments, the liver-specific promoter is an LP1 promoter. The LP1 promoter can be a human LP1 promoter, which can be constructed as described, e.g., in Nathwani et al. Blood vol. 107(7) (2006):2653-61.

In some embodiments, the promoter is a cytomegalovirus (CMV) or cytomegalovirus (CMV) enhancer fused to the chicken β-actin (CAG) promoter. See Alexopoulou et al., BMC Cell Biol. 2008; 9:2. Published 2008 Jan. 11.

It should be appreciated that any other inherited or acquired diseases can be treated and/or mitigated using the method in accordance with the present disclosure.

In aspects there is provided a method for identifying site-specific targeting to a nucleic acid by a helper enzyme and a targeting element, comprising: (a) transfecting a cell with a donor plasmid, the helper enzyme and a targeting element, and a reporter plasmid, wherein: the donor plasmid comprises a first fragment of a reporter gene under the control of a promoter and a splice-donor site (SD); the reporter plasmid comprises a landing pad for the targeting element comprising site specific DNA binding recognition sites flanking a TTAA followed by a splice acceptor site (SA) and a second fragment of a reporter gene; and (b) splicing and integrating into the landing pad, to permit the reconstitution of the reporter gene from the fragments thereof and thereby causing a reporter redout. In embodiments, the method further comprises (c) amplifying the donor plasmid to identify targeting. In embodiments, the method further comprises (d) sequencing the amplified product to analyze integration in specific sequence regions.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF DRAWINGS

FIG. 1A-FIG. 1C depict illustrative non-limiting concepts of bioengineering the MLT transposase protein for site-specific targeting and hetrodimerizarion. In FIG. 1A, the unengineered MLT transposase dimer binds the target DNA TTAA and flanking non-TTAA (nnnn) phosphodiester backbone (sequence independent). In FIG. 1B, the recruitment to a site-specific TTAA is directed by fusing (i.e., linking) protein sequence-specific DNA binding domains (e.g., TALE, ZnF, Cas) that recognize target DNA sequences flanking the TTAA. In FIG. 1C, mutations (X) in the intrinsic DNA binding domains decrease MLT transposase interactions with target DNA non-TTAA which flank the TTAA but leave excision and TTAA use intact (Exc+, Int−).

FIG. 2A-FIG. 2B depict the non-limiting types of covalent and non-covalent linkers that are used to directly fuse (i.e., link) protein sequence-specific DNA binding domains (e.g., TALE, ZnF, Cas) that recognize target DNA sequences flanking the TTAA. In FIG. 2A, the arrow shows covalent linker that fuses DNA binders to the N-terminus of MLT transposase. The linkers are strings of amino acids of varying lengths and flexibility. In FIG. 2B, the arrows show non-covalent linkers that an antipeptide antibody (Ab) fused to a DNA binder and a peptide tag fused to the N-terminus of MLT transposase. These components can be changed where the antipeptide Ab is fused to MLT transposase and the peptide tag is fused to the DNA binder.

FIG. 3 depicts an illustrative 5-step plasmid landing pad assay in HEK293 cells to identify site-specific targeting using MLT transposase or other mobile elements (e.g., recombinases, integrases, transposases). Step 1 involves transfection of HEK293 cells using a donor DNA with CMV driving the 5-half (left) of GFP followed by a splice-donor (SD) site, MLT transposase fusion helpers with various linkers and DNA binding fusions linked to the N-terminus of MLT transposase, and a plasmid landing pad (reporter plasmid) with site specific DNA binding recognition sites flanking a TTAA followed by a splice acceptor site (SA) and the 3-half (right) half of GFP. Step 2 shows the mechanism of splicing and integration into the landing pad after transfection. In Step 3, the left and right halves of GFP are joined and the SA and SD are spliced out thus turning on GFP (GFP readout). Step 4 is the PCR amplification step to identify targeting. Step 5 uses Amplicon-Seq to analyze integration in specific sequence regions.

FIG. 4A-FIG. 4B depict PCR amplification to identify targeting Step 4 in FIG. 3. In FIG. 4A, a landing pad with no DNA binding recognition sites (zinc fingers (ZnF) in this case, but could be TALE, Cas, etc.) is used as a negative control. Landing pads with DNA binding recognition sites (ZnF in this case, but could be TALE, Cas, etc.) on one or both sides of the target TTAA are analyzed for targeting. In FIG. 4B, a 2% agarose gel shows the PCR products using both covalent (Cov) and non-covalent (NC) linkers (shown in FIG. 2A and FIG. 2B) and landing pads with a single, double or no ZnF recognition sites. There are no unique PCR products when unengineered MLT transposase (labeled as “Sal” in the figure) or landing pads without DNA binding recognition sites are used. Targeted PCR products are seen using MLT transposase fusion proteins using both Cov and NC linkers. The highest targeted insertions are seen using covalently linked MLT transposase fusions when there are two flanking DNA binding recognition sites.

FIG. 5A-FIG. 5B depict Step 5 Amplicon-Seq results showing sequence-specific targeting at 15 base pairs (also occurs at 19 bp, data not shown) from the DNA binding recognition site (SEQ ID NO: 816). FIG. 5A depicts Next Generation sequencing results show on-target insertion (boxed) at 15 base pairs from the targeted TTAA with few off-targets within 350 bp on either side of the TTAA. FIG. 5B depicts a bar graph showing that covalent linker and a landing pad with flanking DNA binding recognition sites has about a 42% targeting efficiency (42% of total reads) compared to a single site landing pad (24%). Non-covalent linkers with a landing pad with flanking DNA binding recognition sites had a 29% efficiency with the least with a single DNA binding recognition site (12%).

FIG. 6A-FIG. 6F depict six illustrative bioengineered RNA helper constructs that are contained in a replication backbone (e.g., plasmid, miniplasmid, nanoplasmid, doggybone, or close-ended linear DNA) with a T7 promoter (cap dependent), beta-globin 5′-UTR, and a helper enzyme with 2 or more mutations in the Myotis lucifugus helper (SEQ ID NO: 1, SEQ ID NO: 2) followed by a beta-globin 3′-UTR, and a poly-alanine tail (FIG. 6A). TALEs (FIG. 6B, TABLE 8-TABLE 12), ZnF (FIG. 6C, TABLE 13-TABLE 17), or a dead Cas9 (dCas9) binding protein (FIG. 6D, SEQ ID NO: 5, SEQ ID NO: 6) with guide RNAs (TABLE 3-TABLE 7) were joined by a linker to the N-terminus to target the specific TTAA sites at hROSA 26, AAVS1, chromosome 4, chromosome 22, and chromosome X loci. FIG. 6E depicts a construct with a dimerization enhancer to assure activation of the two monomers. FIG. 6F depicts a construct with a DNA binder (TALE, ZnF) that interrupts an intrinsic DNA binding loop (Y281-P339) and renders the helper enzyme as Exc+/Int−. The extrinsic DNA binder (TALE, ZnF) then binds to specific genomic sequences and targets a specific TTAA target in the genome.

FIG. 7A depicts an illustrative core donor construct that is contained in a replication backbone (e.g., plasmid, miniplasmid, nanoplasmid, doggybone, or close-ended linear DNA) with a promoter driving a gene of interest (GOI) with a polyA tail flanked by two insulators and ITRs. The inverted terminal repeat (ITR) recognition sequences are included at the 5′-(SEQ ID NO: 3) and 3-ends (SEQ ID NO: 4). This construct is used for targeting genomic safe harbor sites (GSHS) or other loci.

FIG. 7B depicts an illustrative core donor construct that is contained in a replication backbone (e.g., plasmid, miniplasmid, nanoplasmid, doggybone, or close-ended linear DNA) with a splice acceptor site for exon 2 and other exons of a gene of interest (GOI) followed by a polyA tail and flanked by ITRs. The inverted terminal repeat (ITR) recognition sequences are included at the 5′-(SEQ ID NO: 3) and 3-ends (SEQ ID NO: 4). This construct is used for targeting endogenous genes in the first intron (or other introns) to repair downstream mutations.

FIG. 7C depicts an illustrative core donor construct that is contained in a replication backbone (e.g., plasmid, miniplasmid, nanoplasmid, doggybone, or close-ended linear DNA) with tandem promoters to affect expression in different tissues (e.g., without limitation, liver specific promoter, cardiac specific promoter) and a gene(s) of interest (GOI) followed by a polyA tail and flanked by ITRs. The inverted terminal repeat (ITR) recognition sequences are included at the 5′-(SEQ ID NO: 3) and 3-ends (SEQ ID NO: 4). This construct is used to differentially promote expression of genes in different organs, tissues or cell types.

FIG. 7D depicts an illustrative core donor construct that is contained in a replication backbone (e.g., plasmid or miniplasmid) with two or more genes of interest (GOI) linked by P2A “self-cleaving” peptides and followed by WPRE and a polyA tail. The construct is flanked by ITRs. The inverted terminal repeat (ITR) recognition sequences are included at the 5′-(SEQ ID NO: 3) and 3-ends (SEQ ID NO: 4). This construct is used for delivering multiple genes or genetic factors.

FIG. 7E depicts an illustrative core donor construct that is contained in a replication backbone (e.g., plasmid, miniplasmid, nanoplasmid, doggybone, or close-ended linear DNA) with a promoter(s) driving the expression of two or more genes as in FIG. 7D and linked to a sequence consisting of a 5′-miRNA, a sense and antisense miRNA pair, and completed with the 3′-miRNA. The construct is followed by WPRE and flanked by ITRs. The inverted terminal repeat (ITR) recognition sequences are included at the 5′-(SEQ ID NO: 3) and 3′-ends (SEQ ID NO: 4). This construct combines protein replacement and miRNA to inhibit the expression of other related proteins.

FIG. 8 depicts the results of integration and excision assays on mutants by amino acid residue. Number denotes the position of the amino acid residue relative to SEQ ID NO: 2.

FIG. 9 depicts the integration and excision activity of deletion mutants. Number denotes the position of the amino acid residue relative to SEQ ID NO: 2.

FIG. 10 depicts the integration and excision activity of fusion proteins mutants. Number denotes the position of the amino acid residue relative to SEQ ID NO: 2.

FIG. 11 depicts the TTAA site in hROSA26 (hg38 chr3:9,396,133-9,396,305) that is targeted by guideRNAs (TABLE 3), TALES (TABLE 8), and ZnF (TABLE 13).

FIG. 12 depicts two TTAA sites in AAVS1 (hg38 chr19:55,112,851-55,113,324) that are targeted by guideRNAs (TABLE 4) or TALES (TABLE 9), and ZnF (TABLE 14).

FIG. 13 depicts two TTAA sites in Chromosome 4 (hg38 chr4:30,793,534-30,875,476) that are targeted by guideRNAs (TABLE 5) or TALES (TABLE 10), and ZnF (TABLE 15).

FIG. 14 depicts two TTAA sites in Chromosome 22 (hg38 chr22:35,370,000-35,380,000) that are targeted by guideRNAs (TABLE 6) or TALES (TABLE 11), and ZnF (TABLE 16).

FIG. 15 depicts two TTAA sites in Chromosome X (hg38 chrX:134,419,661-134,541,172) that are targeted by guideRNAs (TABLE 7) or TALES (TABLE 12), and ZnF (TABLE 17).

FIG. 16 depicts the results of excision and integration assays on MLT helper that contains different deletions at the N- and C-termini. Bars represent % GFP cells measured by flow cytometry. MLT NO was used as a positive control known for high excision activity. Stuffer DNA (MLT Neg) that did not show expression served as negative controls. Abbreviations of test conditions are found in TABLE 18. For each sample, the left histogram is excision, and the right is integration.

FIG. 17 depicts the effects of fusing ZFs on the N-terminus of MLT. Abbreviations of test conditions are found in TABLE 18. For each sample, the left histogram is excision, and the right is integration.

FIGS. 18A-18C show comparison of integration pattern between full length MLT and N-terminal deleted [2-45aa] MLT (“N2”). FIG. 18A depicts a reduction in the number of integration sites in N-terminus deletions (N2). FIG. 18B shows the differences in the epigenetic profile in the MLT N2 mutant compared to hyperactive piggyBac (pB) and MLT. The heat map shows a shift from a strong association with promoters, transcription start sites to (H3K4me3 and H3K4me1), enhancers (H3K27ac) and gene bodies (H3K9me3 and H3K36me3) for pB and MLT compared to a weak signal for such sites with the N2 mutant. FIG. 18C depicts that the TTAA integration site is the main sequence for integration by the MLT N-terminus deletion mutant, N2.

FIG. 19 depicts the alignment of mammalian and amphibian transposases. The arrows show the positions of the MLT N-terminus deletions and their alignment to other transposases.

FIG. 20 depicts that the addition of MLT transposase D416N mutants to MLT transposase containing 2 or more mutants increases excision by ˜5-fold. Dark bars are excision, whereas light bars are integration.

### DETAILED DESCRIPTION

In aspects there is provided a composition comprising (a) a helper enzyme or a nucleic acid encoding the helper enzyme and (b) a targeting element or a nucleic acid encoding the targeting element and (c) a linker connecting the helper enzyme and the targeting element, wherein: the helper enzyme comprises an amino acid sequence having at least about 80% sequence identity to SEQ ID NO: 9 and has a non-polar aliphatic amino acid at position 2 of SEQ ID NO: 9 or a position corresponding thereto and one or more of S8X₁ of SEQ ID NO: 9 or a position corresponding thereto, wherein X₁ is selected from alanine (A), glycine (G), valine (V), leucine (L), isoleucine (I), and proline (P); C13X₂ of SEQ ID NO: 9 or a position corresponding thereto, wherein X₂ is selected from lysine (K), arginine (R), and histidine (H); and N125X₃ of SEQ ID NO: 9 or a position corresponding thereto, wherein X₃ is selected from is selected from lysine (K), arginine (R), and histidine (H); the targeting element is or comprises one or more of a Cas enzyme, which is optionally catalytically inactive and which is optionally associated with a guide RNA (gRNA), a transcription activator-like effector (TALE) DNA binding domain (DBD), a Zinc finger (ZF), a catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof; and the linker comprises less than about 25 amino acids or 75 nucleotides.

In aspects there is provided a composition comprising (a) a helper enzyme or a nucleic acid encoding the helper enzyme and (b) a targeting element or a nucleic acid encoding the targeting element, wherein: the helper enzyme comprises an amino acid sequence having at least about 80% sequence identity to SEQ ID NO: 9 and has a non-polar aliphatic amino acid at position 2 of SEQ ID NO: 9 or a position corresponding thereto and one or more of S8X₁ of SEQ ID NO: 9 or a position corresponding thereto, wherein X₁ is selected from alanine (A), glycine (G), valine (V), leucine (L), isoleucine (I), and proline (P); C13X₂ of SEQ ID NO: 9 or a position corresponding thereto, wherein X₂ is selected from lysine (K), arginine (R), and histidine (H); and N125X₃ of SEQ ID NO: 9 or a position corresponding thereto, wherein X₃ is selected from is selected from lysine (K), arginine (R), and histidine (H); the targeting element is or comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof; and wherein the targeting element directs the helper enzyme to one or more nucleic acids sites that are upstream and/or downstream of the TTAA integration sites and within about 5 to about 30 base pairs of the TTAA integration sites or within about 15 to about 19 base pairs of the TTAA integration sites and optionally a linker connecting the helper enzyme and the targeting element, the linker comprises less than about 25 amino acids or 75 nucleotides.

In embodiments, the non-polar aliphatic amino acid is selected from alanine (A), glycine (G), valine (V), leucine (L), isoleucine (I), and proline (P).

In embodiments, the linker comprises about 10 amino acids to about 20 amino acids or about 12 amino acids to about 15 amino acids, or about 30 nucleotides to about 60 nucleotides or about 36 nucleotides to about 45 nucleotides. In embodiments, the er is substantially comprised of glycine (G) and serine (S) residues. In embodiments, the linker is or comprises (GSS)₄ or in the case of insertion of a DNA binder (TALE, ZnF) in an intrinsic DNA binding loop, the linker is (GS)1 on either side of the DNA binder (TALE, ZnF). In embodiments, the linker connects the targeting element to the N-terminus of the helper enzyme or connects the targeting element within the helper enzyme.

In embodiments, the helper enzyme is suitable of inserting a donor nucleic acid comprising a transgene in a genomic safe harbor site (GSHS) and/or wherein the targeting element is suitable for directing the helper enzyme to a GSHS. In embodiments, the GSHS is in an open chromatin location in a chromosome. In embodiments, the GSHS is selected from adeno-associated virus site 1 (AAVS1), chemokine (C-C motif) receptor 5 (CCR5) gene, HIV-1 coreceptor, and human Rosa26 locus. In embodiments, the GSHS comprises one or more TTAA integration sites. In embodiments, the targeting element directs the helper enzyme to either one or more nucleic acid sites that are upstream and/or downstream of the TTAA integration sites or to the TTAA integration sites. In embodiments, the targeting element directs the helper enzyme to one or more nucleic acid sites that are upstream and/or downstream of the TTAA integration sites and within about 5 to about 30 base pairs of the TTAA integration sites or within about 15 to about 19 base pairs of the TTAA integration sites. In embodiments, the targeting element directs the helper enzyme to two nucleic acid sites of the TTAA integration sites, wherein a first site is upstream of TTAA and within about 5 to about 30 base pairs or about 15 to about 19 base pairs of the TTAA and a second site is downstream of TTAA and within about 5 to about 30 base pairs or about 15 to about 19 base pairs of the TTAA.

In embodiments, the helper enzyme comprises an amino acid sequence having at least about 90% sequence identity to SEQ ID NO: 9. In embodiments, the helper enzyme comprises an amino acid sequence having at least about 95% sequence identity to SEQ ID NO: 9. In embodiments, the helper enzyme comprises an amino acid sequence having at least about 98% sequence identity to SEQ ID NO: 9.

In embodiments, a donor DNA and a helper RNA are transfected at a donor DNA to helper RNA ratio of about 1 to about 4, or about 1 to about 2, or about 1 to about 1.

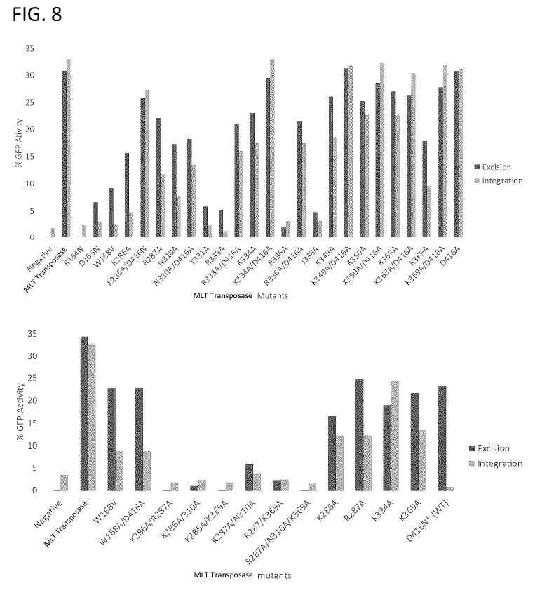

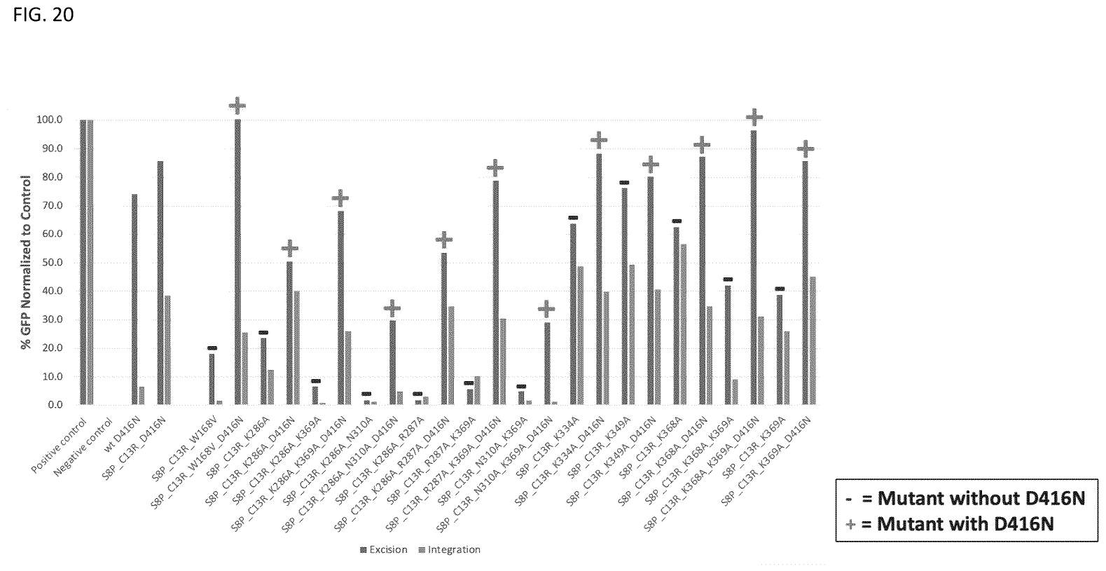

In embodiments, the helper enzyme comprises a an N- or C-terminal deletion, optionally at positions 1-35, or 1-45, or 1-55, or 1-65, or 1-75, or 1-85, or 1-95, or 1-105 or positions corresponding thereto, wherein the positions are relative to SEQ ID NO: 9. In embodiments, the helper enzyme comprises an N-terminal deletion, optionally at positions 1-34, or 1-45, or 1-68, or 1-89 or positions corresponding thereto, wherein the positions are relative to SEQ ID NO: 9. In embodiments, the helper enzyme comprises a C-terminal deletion, optionally at positions 555-573 or 530-573 or positions corresponding thereto, wherein the positions are relative to SEQ ID NO: 9. In embodiments, the N- or C-terminal deletion yields reduced or ablated off-target effects of the helper enzyme compared to the helper enzyme without the N- or C-terminal deletion. In embodiments, the helper enzyme comprising the N-terminal deletion is or comprises an amino acid sequence of SEQ ID NO: 506, or a sequence having at least about 80%, or at least about 90%, or at least about 95%, or at least about 98% identity thereto. In embodiments, the helper enzyme comprises at least one substitution at position D416, or a position corresponding thereto relative to SEQ ID NO: 9. In embodiments, the substitution at position D416 or a position corresponding thereto relative to SEQ ID NO: 9 is a polar and positively charged hydrophilic residue optionally selected from arginine (R) and lysine (K), a polar and neutral of charge hydrophilic residue selected from asparagine (N), glutamine (Q), serine (S), threonine (T), proline (P), and cysteine (C). In embodiments, the substitution at position D416 or a position corresponding thereto relative to SEQ ID NO: 9 is asparagine (N). In embodiments, the helper enzyme comprises at least one substitution at selected from the mutations of FIG. 8, FIG. 20, TABLE 1, and/or TABLE 2.

In embodiments, the composition is a nucleic acid, optionally an RNA. In embodiments, the composition further comprises a donor nucleic acid or is suitable for insertion of a donor nucleic acid, optionally wherein the donor nucleic acid is a transposon.

In embodiments, there is provided a method for inserting a gene into the genome of a cell, comprising contacting a cell with the composition described herein. In embodiments, there is provided a method for treating a disease or disorder ex vivo, comprising contacting a cell with the composition described herein and administering the cell to a subject in need thereof. In embodiments, there is provided a method for treating a disease or disorder in vivo, comprising administering the composition of described herein to a subject in need thereof.

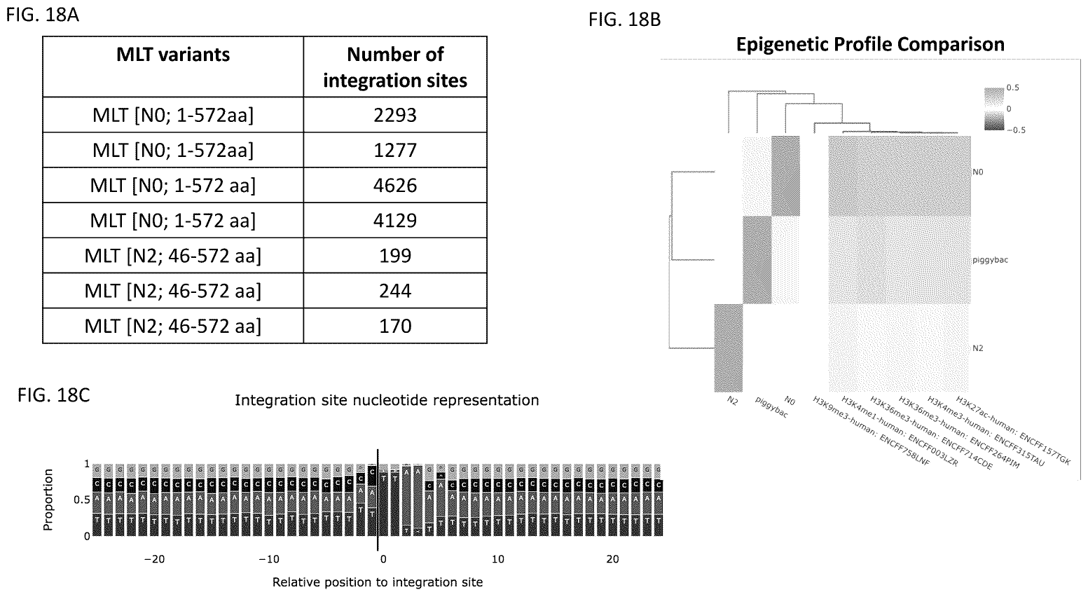

The present disclosure is based, in part, on the discovery of DNA binding proteins (e.g., without limitations, ZnF, TALE, Cas9), linkers, and fusion sites that target specific TTAA integration sites. In embodiments, the present disclosure provides a developed landing pad assay that can show site- and sequence-specific targeting. In embodiments, the landing pad assay enables Amplicon-seq to show high efficiency targeting using covalent linkers and flanking DNA binding recognition sites. In embodiments, the high efficiency targeting is up to about 10%, or up to about 20%, or up to about 30%, or up to about 40%, or up to about 50%, or up to about 60%, or up to about 70%, or up to about 80%, or up to about 90%, or up to about 100%. In embodiments, the flanking DNA binding recognition sites are within about 5 to about 30 base pairs of the target TTAA integration sites. In embodiments the flanking DNA binding recognition sites are within about 15 to about 19 base pairs of the target TTAA integration sites. In embodiments, the present disclosure provides MLT transposase N-terminus deletion mutants (FIG. 18, N2). In embodiments the MLT transposase N-terminus deletion mutants show favorable integration or epigenetic profile and promotes recruitment to intergenic target TTAA.

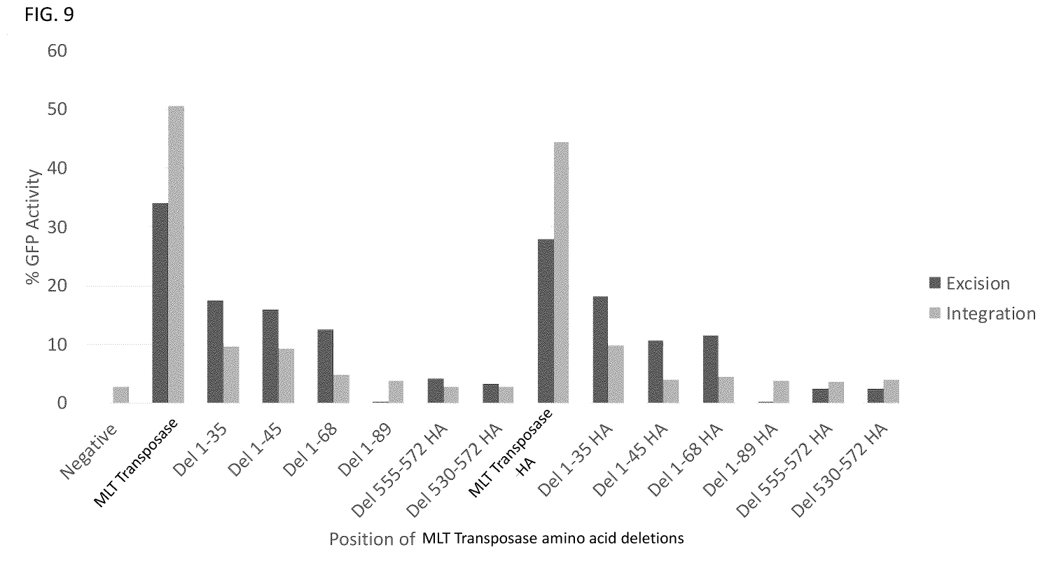

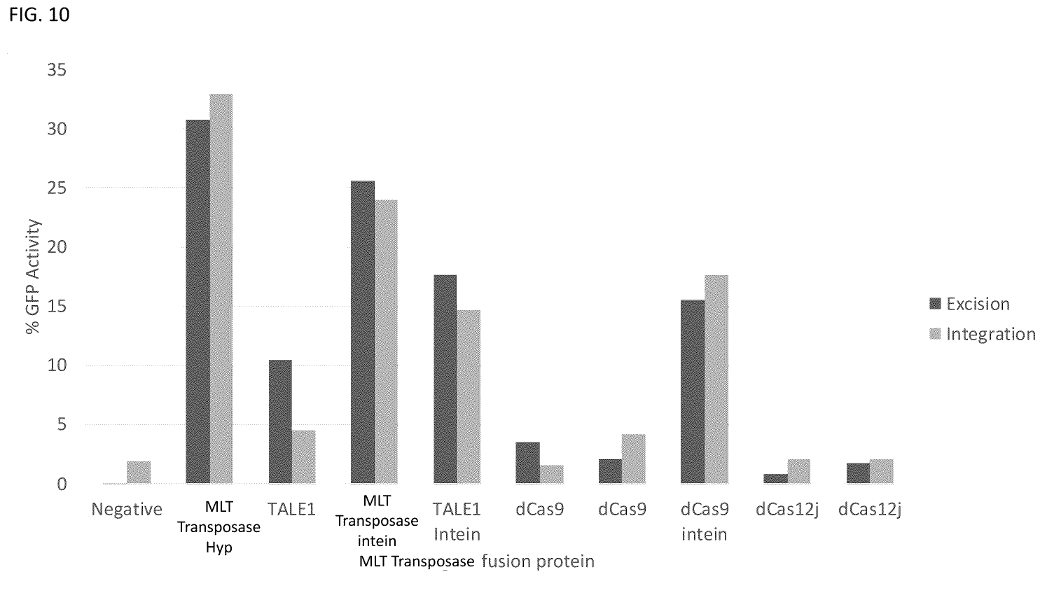

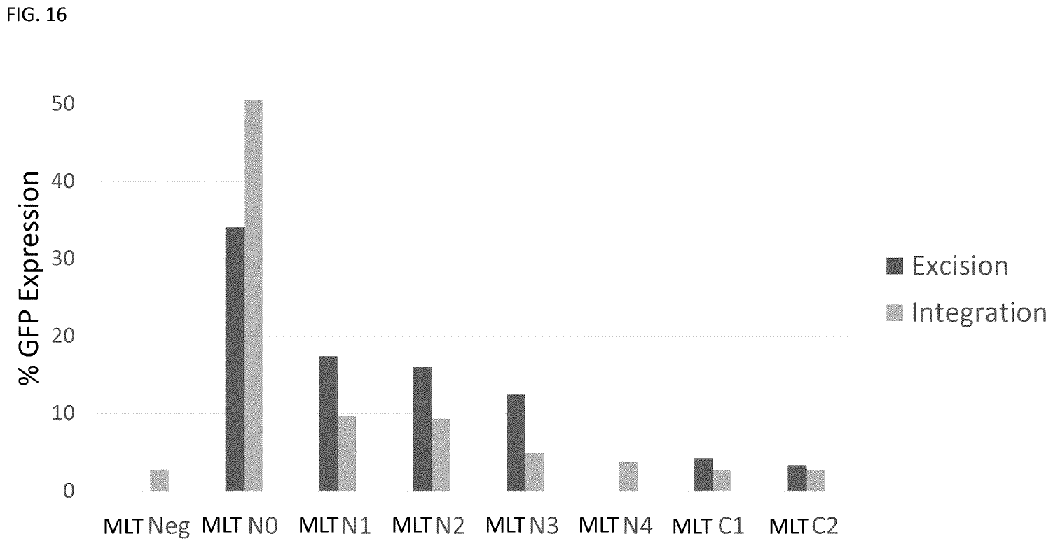

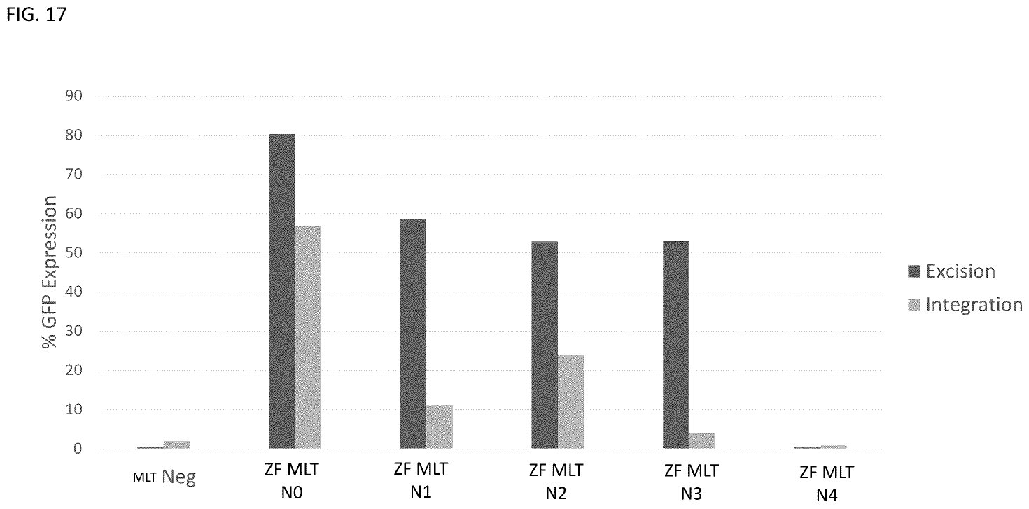

The present invention is based, in part, on the discovery of an engineered helper enzyme capable of gene insertion that finds uses in multiple applications, including, without limitation, in gene therapy. In aspects, there is provided an engineered enzyme, e.g., having an amino acid sequence of SEQ ID NO: 2 or SEQ ID NO: 9 or a variant thereof, inclusive of all variants disclosed herein (e.g., TABLE 1, TABLE 2, FIG. 8, FIG. 9, FIG. 10, FIG. 16, FIG. 17, FIG. 18A, and/or FIG. 20) (occasionally referred to as “engineered”, “the present MLT”, or “hyperactive helper”) or variants thereof. “MLT”, as used herein, refers to Myotis lucifugus helper, as engineered herein.

In embodiments, the illustrative bioengineered RNA helper constructs that are contained in a replication backbone (e.g., plasmid, miniplasmid, nanoplasmid, doggybone, or close-ended linear DNA) with a T7 promoter (cap dependent), beta-globin 5′-UTR, and a helper enzyme with 2 mutations in the Myotis lucifugus helper (SEQ ID NO: 1, SEQ ID NO: 2) followed by a beta-globin 3′-UTR, and a poly-alanine tail. In embodiments, doggybone DNA (dbDNA) is a novel, synthetic DNA vector and enzymatic DNA manufacturing process enabling rapid DNA production.

The present invention is based, in part, on the discovery that an enzyme capable of targeted genomic integration by transposition (e.g., a recombinase, an integrase, or a helper enzyme), as a monomer or a dimer, can be fused with a transcription activator-like effector proteins (TALE) DNA binding domain (DBD), a dCas9/gRNA, or a zinc finger sequence to thereby create a chimeric enzyme capable of a site- or locus-specific transposition. For instance, in the case of a fusion to a TALD DBD, the enzyme (e.g., without limitation, a chimeric helper) utilizes the specificity of TALE DBD to certain sites within a host genome, which allows using DBDs to target any desired location in the genome. In this way, the chimeric helper in accordance with the present disclosure allows achieving targeted integration of a transgene.

In embodiments, the helper has one or more mutations that confer hyperactivity. In embodiments, the helper is a mammal-derived helper, optionally a helper RNA helper. Thus, the present compositions and methods for gene transfer utilize a dual donor/helper system. Transposable elements are non-viral gene delivery vehicles found ubiquitously in nature. Donor-based vectors have the capacity of stable genomic integration and long-lasting expression of transgene constructs in cells. Generally, dual donor and helper systems work via a cut-and-paste mechanism whereby donor DNA containing a transgene(s) of interest is integrated into chromosomal DNA by a helper enzyme at a repetitive sequence site. Dual donor/helper (or “donor/helper”) plasmid systems insert a transgene flanked by inverted terminal ends (“ends”), such as TTAA (SEQ ID NO: 440) tetranucleotide sites, without leaving a DNA footprint in the human genome. The helper enzyme is transiently expressed (on the same or a different vector from a vector encoding the donor) and it catalyzes the insertion events from the donor plasmid to the host genome. Genomic insertions primarily target introns but may target other TTAA (SEQ ID NO: 440) sites and integrate into approximately 50% of human genes.

This disclosure describes a DNA integration system, which is highly active in mammals, and is derived from a mammalian mobile DNA element. This mammal-derived mobile genetic element is engineered to insert donor DNA at specific TTAA insertion “hotspots” that are frequently favored insertion sites for the un-engineered enzyme. This technology exploits a helper RNA encoding enzyme with engineered DNA binding proteins and a donor DNA contained between the ends of a mobile element of the gene to be inserted into the genome. The mammal-derived enzyme can be fused to a protein domain at its N-terminus without loss of activity and “engineered” by fusing DNA binding domains (DBD) that can target almost any location in the genome. Excision competent/target binding defective enzymes (Exc+/Int) mutants are described, that when combined with programmable, synthetic DBDs only insert at a TTAAs at a single target site. This enzyme described in this disclosure displays several highly desirable features that are of great advantage for transgene integration. In embodiments, no DNA double strand breaks are introduced into the target genome. Furthermore, upon enzyme-mediated excision containing a gene of interest from its donor DNA, the flanking donor backbone ends are very efficiently rejoined, leaving no double strand break in the donor DNA to signal DNA damage. The helper enzyme inserts the excised element at high frequency selectively into a TTAA target site. Notably, because excision from the donor site results in the covalent linkage of a TTAA segment to each 5′ donor end, the joining of the 3′ donor ends to staggered positions on the top and bottom strands of the DNA flanking the target TTAA, a simple ligation restores intact duplex DNA, and no DNA synthesis is required for repair. Finally, the helper enzyme delivers a large cargo size as compared to other mobile genetic elements or integrating viral systems to date. See Liang, et al. (2009). Chromosomal mobilization and reintegration of Sleeping Beauty and PiggyBac donors. Genesis, 47(6), 404-408; Mitra, et al. (2013). Functional characterization of piggyBat from the bat Myotis lucifugus unveils an active mammalian DNA donor. Proc Natl Acad Sci USA, 110(1), 234-239; Ray, et al. (2008). Multiple waves of recent DNA donor activity in the bat, Myotis lucifugus. Genome Res, 18(5), 717-728.

In embodiments, the helper enzyme is delivered as an RNA instead of as a DNA. Other mobile genetic elements including helpers such as hyperactive piggyBac (pB) and SB100X, when delivered as RNA, have significantly less activity when compared to DNA. See Bire, et al. (2013). Exogenous mRNA delivery and bioavailability in gene transfer mediated by piggyBac transposition. BMC Biotechnol, 13, 75; Bire, et al. (2013). Optimization of the piggyBac donor using mRNA and insulators: toward a more reliable gene delivery system. PLoS One, 8(12), e82559; Wilber, et al. (2006). RNA as a source of helper for Sleeping Beauty-mediated gene insertion and expression in somatic cells and tissues. Mol Ther, 13(3), 625-630. The helper enzyme described herein has the same or better activity when delivered as RNA. The use of helper RNA offers several advantages over delivery of a DNA molecule. Wilber, et al. (2006). RNA as a source of helper for Sleeping Beauty-mediated gene insertion and expression in somatic cells and tissues. Mol Ther, 13(3), 625-630. For instance, without wishing to be bound by theory, there is improved control with respect to the duration of helper enzyme expression, minimizing persistence in the tissue, and there is potential for transgene re-mobilization and re-insertion following the initial transposition event. Furthermore, in embodiments, the helper-encoding RNA sequence is incapable of integrating into the host genome, thereby eliminating concerns about long-term helper expression and destabilizing effects with respect to the gene of interest. This safety feature, in embodiments, prevents the integration of the helper enzyme gene into the human genome and circumvents potential oncogenic and mutagenic effects.

In embodiments, the present disclosure provides a dual DNA donor and RNA helper system. The donor DNA plasmid contains helper-specific inverted terminal repeats (ITRs) flanking the transgene while the helper-RNA transiently expresses a synthetic helper enzyme that catalyzes the insertion events from the donor plasmid to the host genome. This two component DNA/RNA system is, in embodiments, co-encapsulated in a single lipid nanoparticle using microfluidic technology and the lipid nanoparticles protect the RNA from extracellular degradation by in vivo injection.

In embodiments, the helper enzyme described herein is amenable to be fused to protein domain at the N-terminus without loss of activity. Deletions of the C-terminus, in embodiments, cause a loss of helper enzyme excision and integration activity that may be restored when fused to binding ligands (e.g., rapamycin-induced FRB-FKBP fusion, SH3 plus high affinity ligand). This feature permits, inter alia, the synthesis of an “engineered” helper enzyme that target specific genomic regions of interest by fusing to the helper enzyme particular DNA binding domains that can target almost any location in the genome.

### Helper Enzyme

In embodiments, the present disclosure provides a composition comprising a helper enzyme or a nucleic acid encoding the helper enzyme, wherein the helper enzyme comprises an amino acid sequence having at least about 80% sequence identity to SEQ ID NO: 9 and has an alanine residue at position 2 of SEQ ID NO: 9 or a position corresponding thereto.

SEQ ID NO: 9: amino acid sequence of a variant of
the hyperactive helper with S at position 8 and C
at position 13 (572 amino acids)

| | |
|---|---|
| 1 | MAQHSDYSDD EFCADKLSNY SCDSDLENAS TSDEDSSDDE |
| | VMVRPRTLRR RRISSSSSDS |
| 61 | ESDIEGGREE WSHVDNPPVL EDFLGHQGLN TDAVINNIED |
| | AVKLFIGDDF FEFLVEESNR |
| 121 | YYNQNRNNFK LSKKSLKWKD ITPQEMKKFL GLIVLMGQVR |
| | KDRRDDYWTT EPWTETPYFG |
| 181 | KTMTRDRFRQ IWKAWHFNNN ADIVNESDRL CKVRPVLDYF |
| | VPKFINIYKP HQQLSLDEGI |
| 241 | VPWRGRLFFR VYNAGKIVKY GILVRLLCES DTGYICNMEI |
| | YCGEGKRLLE TIQTVVSPYT |
| 301 | DSWYHIYMDN YYNSVANCEA LMKNKFRICG TIRKNRGIPK |
| | DFQTISLKKG ETKFIRKNDI |
| 361 | LLQVWQSKKP VYLISSIHSA EMEESQNIDR TSKKKIVKPN |
| | ALIDYNKHMK GVDRADQYLS |
| 421 | YYSILRRTVK WTKRLAMYMI NCALFNSYAV YKSVRQRKMG |
| | FKMFLKQTAI HWLTDDIPED |
| 481 | MDIVPDLQPV PSTSGMRAKP PTSDPPCRLS MDMRKHTLQA |
| | IVGSGKKKNI LRRCRVCSVH |
| 541 | KLRSETRYMC KFCNIPLHKG ACFEKYHTLK NY |

In embodiments, the helper enzyme comprises an amino acid sequence of at least about 90% identity to SEQ ID NO: 9. In embodiments, the helper enzyme comprises an amino acid sequence of at least about 93% identity to SEQ ID NO: 9. In embodiments, the helper enzyme comprises an amino acid sequence of at least about 95% identity to SEQ ID NO: 9. In embodiments, the helper enzyme comprises an amino acid sequence of at least about 98% identity to SEQ ID NO: 9. In embodiments, the helper enzyme comprises an amino acid sequence of at least about 99% identity to SEQ ID NO: 9.

In embodiments, the helper enzyme has one or more mutations which confer hyperactivity.

In embodiments, the helper enzyme has one or more amino acid substitutions selected from S8X₁ and/or C13X₂ or substitutions at positions corresponding thereto. In embodiments, the helper enzyme has 8X₁ and C13X₂ substitutions or substitutions at positions corresponding thereto. In embodiments, the X₁ is selected from G, A, V, L, I and P and X₂ is selected from K, R, and H. In embodiments, the X₁ is P and X₂ is R.

In embodiments, the helper enzyme comprises an amino acid sequence of SEQ ID NO: 2.

SEQ ID NO: 2: amino acid sequence of hyperactive
helper (572 amino acids)

| | |
|---|---|
| 1 | MAQHSDYPDD EFRADKLSNY SCDSDLENAS TSDEDSSDDE |
| | VMVRPRTLRR RRISSSSSDS |
| 61 | ESDIEGGREE WSHVDNPPVL EDFLGHQGLN TDAVINNIED |
| | AVKLFIGDDF FEFLVEESNR |
| 121 | YYNQNRNNFK LSKKSLKWKD ITPQEMKKFL GLIVLMGQVR |
| | KDRRDDYWTT EPWTETPYFG |
| 181 | KTMTRDRFRQ IWKAWHFNNN ADIVNESDRL CKVRPVLDYF |
| | VPKFINIYKP HQQLSLDEGI |
| 241 | VPWRGRLFFR VYNAGKIVKY GILVRLLCES DTGYICNMEI |
| | YCGEGKRLLE TIQTVVSPYT |
| 301 | DSWYHIYMDN YYNSVANCEA LMKNKFRICG TIRKNRGIPK |
| | DFQTISLKKG ETKFIRKNDI |
| 361 | LLQVWQSKKP VYLISSIHSA EMEESQNIDR TSKKKIVKPN |
| | ALIDYNKHMK GVDRADQYLS |
| 421 | YYSILRRTVK WTKRLAMYMI NCALFNSYAV YKSVRQRKMG |
| | FKMFLKQTAI HWLTDDIPED |
| 481 | MDIVPDLQPV PSTSGMRAKP PTSDPPCRLS MDMRKHTLQA |
| | IVGSGKKKNI LRRCRVCSVH |
| 541 | KLRSETRYMC KFCNIPLHKG ACFEKYHTLK NY |

In embodiments, the nucleic acid that encodes the helper enzyme has a nucleotide sequence of SEQ ID NO: 11 or a codon-optimized form thereof.

SEQ ID NO: 11: nucleotide sequence encoding the
hyperactive helper (1719 nt)

| | |
|---|---|
| 1 | ATGGCCCAGC ACAGCGACTA CCCCGACGAC GAGTTCAGAG |
| | CCGATAAGCT GAGTAACTAC |
| 61 | AGCTGCGACA GCGACCTGGA AAACGCCAGC ACATCCGACG |
| | AGGACAGCTC TGACGACGAG |
| 121 | GTGATGGTGC GGCCCAGAAC CCTGAGACGG AGAAGAATCA |
| | GCAGCTCTAG CAGCGACTCT |
| 181 | GAATCCGACA TCGAGGGGGG CCGGGAAGAG TGGAGCCACG |
| | TGGACAACCC TCCTGTTCTG |
| 241 | GAAGATTTTC TGGGCCATCA GGGCCTGAAC ACCGACGCCG |
| | TGATCAACAA CATCGAGGAT |
| 301 | GCCGTGAAGC TGTTCATAGG AGATGATTTC TTTGAGTTCC |
| | TGGTCGAGGA ATCCAACCGC |
| 361 | TATTACAACC AGAATAGAAA CAACTTCAAG CTGAGCAAGA |
| | AAAGCCTGAA GTGGAAGGAC |
| 421 | ATCACCCCTC AGGAGATGAA AAAGTTCCTG GGACTGATCG |
| | TTCTGATGGG ACAGGTGCGG |
| 481 | AAGGACAGAA GGGATGATTA CTGGACAACC GAACCTTGGA |
| | CCGAGACCCC TTACTTTGGC |
| 541 | AAGACCATGA CCAGAGACAG ATTCAGACAG ATCTGGAAAG |
| | CCTGGCACTT CAACAACAAT |
| 601 | GCTGATATCG TGAACGAGTC TGATAGACTG TGTAAAGTGC |
| | GGCCAGTGTT GGATTACTTC |
| 661 | GTGCCTAAGT TCATCAACAT CTATAAGCCT CACCAGCAGC |
| | TGAGCCTGGA TGAAGGCATC |
| 721 | GTGCCCTGGC GGGGCAGACT GTTCTTCAGA GTGTACAATG |
| | CTGGCAAGAT CGTCAAATAC |
| 781 | GGCATCCTGG TGCGCCTTCT GTGCGAGAGC GATACAGGCT |
| | ACATCTGTAA TATGGAAATC |
| 841 | TACTGCGGCG AGGGCAAAAG ACTGCTGGAA ACCATCCAGA |
| | CCGTCGTTTC CCCTTATACC |
| 901 | GACAGCTGGT ACCACATCTA CATGGACAAC TACTACAATT |
| | CTGTGGCCAA CTGCGAGGCC |
| 961 | CTGATGAAGA ACAAGTTTAG AATCTGCGGC ACAATCAGAA |
| | AAAACAGAGG CATCCCTAAG |
| 1021 | GACTTCCAGA CCATCTCTCT GAAGAAGGGC GAAACCAAGT |
| | TCATCAGAAA GAACGACATC |
| 1081 | CTGCTCCAAG TGTGGCAGTC CAAGAAACCC GTGTACCTGA |
| | TCAGCAGCAT CCATAGCGCC |
| 1141 | GAGATGGAAG AAAGCCAGAA CATCGACAGA ACAAGCAAGA |
| | AGAAGATCGT GAAGCCCAAT |
| 1201 | GCTCTGATCG ACTACAACAA GCACATGAAA GGCGTGGACC |
| | GGGCCGACCA GTACCTGTCT |
| 1261 | TATTACTCTA TCCTGAGAAG AACAGTGAAA TGGACCAAGA |
| | GACTGGCCAT GTACATGATC |
| 1321 | AATTGCGCCC TGTTCAACAG CTACGCCGTG TACAAGTCCG |
| | TGCGACAAAG AAAAATGGGA |
| 1381 | TTCAAGATGT TCCTGAAGCA GACAGCCATC CACTGGCTGA |
| | CAGACGACAT TCCTGAGGAC |
| 1441 | ATGGACATTG TGCCAGATCT GCAACCTGTG CCCAGCACCT |
| | CTGGTATGAG AGCTAAGCCT |
| 1501 | CCCACCAGCG ATCCTCCATG TAGACTGAGC ATGGACATGC |
| | GGAAGCACAC CCTGCAGGCC |
| 1561 | ATCGTCGGCA GCGGCAAGAA GAAGAACATC CTTAGACGGT |
| | GCAGGGTGTG CAGCGTGCAC |
| 1621 | AAGCTGCGGA GCGAGACTCG GTACATGTGC AAGTTTTGCA |
| | ACATTCCCCT GCACAAGGGA |
| 1681 | GCCTGCTTCG AGAAGTACCA CACCCTGAAG AATTACTAG |

In embodiments, the helper enzyme comprises at least one substitution at positions selected from TABLE 1 and/or TABLE 2 or positions corresponding thereto, which correspond positions of SEQ ID NO: 9.

In embodiments, the helper enzyme comprises at least one substitution at positions selected from TABLE 1 and/or TABLE 2 or positions corresponding thereto, which correspond positions of SEQ ID NO: 2.

In embodiments, the helper enzyme comprises at least one substitution at positions selected from: 164, 165, 168, 286, 287, 310, 331, 333, 334, 336, 338, 349, 350, 368, 369, 416, or positions corresponding thereto relative to SEQ ID NO: 9. In embodiments, the helper enzyme comprises at least one substitution at positions selected from: R164N, D165N, W168V, W168A, K286A, R287A, N310A, T331A, R333A, K334A, R336A, I338A, K349A, K350A, K368A, K369A, D416A, D416N, or positions corresponding thereto relative to SEQ ID NO: 9. In embodiments, the helper enzyme comprises at least one substitution at position corresponding to: 331, 333, and/or 416 or positions corresponding thereto relative to SEQ ID NO: 9. In embodiments, the substitution is selected from G, A, V, N, and Q. In embodiments, the helper enzyme comprises at least one substitution at selected from: T331A, R333A, and/or D416N or positions corresponding thereto relative to SEQ ID NO: 9.

In embodiments, the helper enzyme comprises a deletion of about 30, or about 40, or about 50, or about 60, or about 70, or about 80, or about 90, or about 100 amino acids from an N-terminus of the polypeptide having an amino acid sequence of SEQ ID NO: 9. In embodiments, the helper enzyme comprises a deletion at positions about 1-35, or about 1-45, or about 1-55, or about 1-65, or about 1-75, or about 1-85, or about 1-95, or about 1-105 or positions corresponding thereto, wherein the positions are relative to SEQ ID NO: 9. In embodiments, the helper enzyme has increased activity relative to an en-zyme comprising an amino acid sequence of SEQ ID NO: 9 or functional equivalent thereof.

In embodiments, the helper enzyme is excision positive. In embodiments, the helper enzyme is integration deficient. In embodiments, the helper enzyme has decreased integration activity relative to a helper enzyme comprising an amino acid sequence of SEQ ID NO: 9 or functional equivalent thereof. In embodiments, the helper enzyme has increased excision activity relative to a helper enzyme comprising an amino acid sequence of SEQ ID NO: 9 or functional equivalent thereof.

In embodiments, the helper enzyme of the present disclosure comprises a deletion at positions about 1-35, or about 1-45, or about 1-55, or about 1-65, or about 1-75, or about 1-85, or about 1-95, or about 1-105 or positions corresponding thereto, wherein the positions are relative to SEQ ID NO: 9 or SEQ ID NO: 502. In embodiments, the enzyme is an MLT. In embodiments, the deletion comprises an N or C terminal deletion. In embodiments, the N or C terminal deletion yields reduced or ablated off-target effects of the helper enzyme compared to the helper enzyme without the N or C terminal deletion. In embodiments, the helper enzyme comprising the N terminal deletion is N2. In embodiments, the helper enzyme comprising the N terminal deletion is or comprises SEQ ID NO: 506. In embodiments, the mutant with an N or C terminal deletion is further fused to a DNA binder. In embodiments, the DNA binder comprises TALEs, ZnF, and/or both. In embodiments, the helper enzyme comprises a targeting element. In embodiments, the helper enzyme is capable of inserting a donor comprising a transgene in a genomic safe harbor site (GSHS). In embodiments, the binding of a GSHS of a nucleic acid molecule in a mammalian cell is with high target specificity, relative to a control. In embodiments, the control is a composition comprising a helper enzyme comprising an amino acid sequence of SEQ ID NO: 9 or a nucleic acid comprising a nucleotide sequence of SEQ ID NO: 10 or a codon-optimized form thereof.

SEQ ID NO: 10: nucleotide sequence encoding SEQ ID
NO: 9 (1719 nt)

| | |
|---|---|
| 1 | ATGGCCCAGC ACAGCGACTA CCCCGACGAC GAGTTCAGAG |
| | CCGATAAGCT GAGTAACTAC |
| 61 | AGCTGCGACA GCGACCTGGA AAACGCCAGC ACATCCGACG |
| | AGGACAGCTC TGACGACGAG |
| 121 | GTGATGGTGC GGCCCAGAAC CCTGAGACGG AGAAGAATCA |
| | GCAGCTCTAG CAGCGACTCT |
| 181 | GAATCCGACA TCGAGGGCGG CCGGGAAGAG TGGAGCCACG |
| | TGGACAACCC TCCTGTTCTG |
| 241 | GAAGATTTTC TGGGCCATCA GGGCCTGAAC ACCGACGCCG |
| | TGATCAACAA CATCGAGGAT |
| 301 | GCCGTGAAGC TGTTCATAGG AGATGATTTC TTTGAGTTCC |
| | TGGTCGAGGA ATCCAACCGC |
| 361 | TATTACAACC AGAATAGAAA CAACTTCAAG CTGAGCAAGA |
| | AAAGCCTGAA GTGGAAGGAC |
| 421 | ATCACCCCTC AGGAGATGAA AAAGTTCCTG GGACTGATCG |
| | TTCTGATGGG ACAGGTGCGG |
| 481 | AAGGACAGAA GGGATGATTA CTGGACAACC GAACCTTGGA |
| | CCGAGACCCC TTACTTTGGC |
| 541 | AAGACCATGA CCAGAGACAG ATTCAGACAG ATCTGGAAAG |
| | CCTGGCACTT CAACAACAAT |
| 601 | GCTGATATCG TGAACGAGTC TGATAGACTG TGTAAAGTGC |
| | GGCCAGTGTT GGATTACTTC |
| 661 | GTGCCTAAGT TCATCAACAT CTATAAGCCT CACCAGCAGC |
| | TGAGCCTGGA TGAAGGCATC |
| 721 | GTGCCCTGGC GGGGCAGACT GTTCTTCAGA GTGTACAATG |
| | CTGGCAAGAT CGTCAAATAC |
| 781 | GGCATCCTGG TGCGCCTTCT GTGCGAGAGC GATACAGGCT |
| | ACATCTGTAA TATGGAAATC |
| 841 | TACTGCGGCG AGGGCAAAAG ACTGCTGGAA ACCATCCAGA |
| | CCGTCGTTTC CCCTTATACC |
| 901 | GACAGCTGGT ACCACATCTA CATGGACAAC TACTACAATT |
| | CTGTGGCCAA CTGCGAGGCC |
| 961 | CTGATGAAGA ACAAGTTTAG AATCTGCGGC ACAATCAGAA |
| | AAAACAGAGG CATCCCTAAG |
| 1021 | GACTTCCAGA CCATCTCTCT GAAGAAGGGC GAAACCAAGT |
| | TCATCAGAAA GAACGACATC |
| 1081 | CTGCTCCAAG TGTGGCAGTC CAAGAAACCC GTGTACCTGA |
| | TCAGCAGCAT CCATAGCGCC |
| 1141 | GAGATGGAAG AAAGCCAGAA CATCGACAGA ACAAGCAAGA |
| | AGAAGATCGT GAAGCCCAAT |
| 1201 | GCTCTGATCG ACTACAACAA GCACATGAAA GGCGTGGACC |
| | GGGCCGACCA GTACCTGTCT |
| 1261 | TATTACTCTA TCCTGAGAAG AACAGTGAAA TGGACCAAGA |
| | GACTGGCCAT GTACATGATC |
| 1321 | AATTGCGCCC TGTTCAACAG CTACGCCGTG TACAAGTCCG |
| | TGCGACAAAG AAAAATGGGA |
| 1381 | TTCAAGATGT TCCTGAAGCA GACAGCCATC CACTGGCTGA |
| | CAGACGACAT TCCTGAGGAC |
| 1441 | ATGGACATTG TGCCAGATCT GCAACCTGTG CCCAGCACCT |
| | CTGGTATGAG AGCTAAGCCT |
| 1501 | CCCACCAGCG ATCCTCCATG TAGACTGAGC ATGGACATGC |
| | GGAAGCACAC CCTGCAGGCC |
| 1561 | ATCGTCGGCA GCGGCAAGAA GAAGAACATC CTTAGACGGT |
| | GCAGGGTGTG CAGCGTGCAC |
| 1621 | AAGCTGCGGA GCGAGACTCG GTACATGTGC AAGTTTTGCA |
| | ACATTCCCCT GCACAAGGGA |
| 1681 | GCCTGCTTCG AGAAGTACCA CACCCTGAAG AATTACTAG |

In embodiments, the control is a composition comprising a helper enzyme comprising an amino acid sequence of SEQ ID NO: 2 or a nucleic acid comprising a nucleotide sequence of SEQ ID NO: 11 or a codon-optimized form thereof.

SEQ ID NO: 11: nucleotide sequence encoding
hyperactive helper (1719 nt)

| | |
|---|---|
| 1 | ATGGCCCAGC ACAGCGACTA CCCCGACGAC GAGTTCAGAG |
| | CCGATAAGCT GAGTAACTAC |
| 61 | AGCTGCGACA GCGACCTGGA AAACGCCAGC ACATCCGACG |
| | AGGACAGCTC TGACGACGAG |
| 121 | GTGATGGTGC GGCCCAGAAC CCTGAGACGG AGAAGAATCA |
| | GCAGCTCTAG CAGCGACTCT |
| 181 | GAATCCGACA TCGAGGGCGG CCGGGAAGAG TGGAGCCACG |
| | TGGACAACCC TCCTGTTCTG |
| 241 | GAAGATTTTC TGGGCCATCA GGGCCTGAAC ACCGACGCCG |
| | TGATCAACAA CATCGAGGAT |
| 301 | GCCGTGAAGC TGTTCATAGG AGATGATTTC TTTGAGTTCC |
| | TGGTCGAGGA ATCCAACCGC |
| 361 | TATTACAACC AGAATAGAAA CAACTTCAAG CTGAGCAAGA |
| | AAAGCCTGAA GTGGAAGGAC |
| 421 | ATCACCCCTC AGGAGATGAA AAAGTTCCTG GGACTGATCG |
| | TTCTGATGGG ACAGGTGCGG |
| 481 | AAGGACAGAA GGGATGATTA CTGGACAACC GAACCTTGGA |
| | CCGAGACCCC TTACTTTGGC |
| 541 | AAGACCATGA CCAGAGACAG ATTCAGACAG ATCTGGAAAG |
| | CCTGGCACTT CAACAACAAT |
| 601 | GCTGATATCG TGAACGAGTC TGATAGACTG TGTAAAGTGG |
| | GGCCAGTGTT GGATTACTTC |
| 661 | GTGCCTAAGT TCATCAACAT CTATAAGCCT CACCAGCAGC |
| | TGAGCCTGGA TGAAGGCATC |
| 721 | GTGCCCTGGC GGGGCAGACT GTTCTTCAGA GTGTACAATG |
| | CTGGCAAGAT CGTCAAATAC |
| 781 | GGCATCCTGG TGCGCCTTCT GTGCGAGAGC GATACAGGCT |
| | ACATCTGTAA TATGGAAATC |
| 841 | TACTGCGGCG AGGGCAAAAG ACTGCTGGAA ACCATCCAGA |
| | CCGTCGTTTC CCCTTATACC |
| 901 | GACAGCTGGT ACCACATCTA CATGGACAAC TACTACAATT |
| | CTGTGGCCAA CTGCGAGGCC |
| 961 | CTGATGAAGA ACAAGTTTAG AATCTGCGGC ACAATCAGAA |
| | AAAACAGAGG CATCCCTAAG |
| 1021 | GACTTCCAGA CCATCTCTCT GAAGAAGGGC GAAACCAAGT |
| | TCATCAGAAA GAACGACATC |
| 1081 | CTGCTCCAAG TGTGGCAGTC CAAGAAACCC GTGTACCTGA |
| | TCAGCAGCAT CCATAGCGCC |
| 1141 | GAGATGGAAG AAAGCCAGAA CATCGACAGA ACAAGCAAGA |
| | AGAAGATCGT GAAGCCCAAT |
| 1201 | GCTCTGATCG ACTACAACAA GCACATGAAA GGCGTGGACC |
| | GGGCCGACCA GTACCTGTCT |
| 1261 | TATTACTCTA TCCTGAGAAG AACAGTGAAA TGGACCAAGA |
| | GACTGGCCAT GTACATGATC |
| 1321 | AATTGCGCCC TGTTCAACAG CTACGCCGTG TACAAGTCCG |
| | TGCGACAAAG AAAAATGGGA |
| 1381 | TTCAAGATGT TCCTGAAGCA GACAGCCATC CACTGGCTGA |
| | CAGACGACAT TCCTGAGGAC |
| 1441 | ATGGACATTG TGCCAGATCT GCAACCTGTG CCCAGCACCT |
| | CTGGTATGAG AGCTAAGCCT |
| 1501 | CCCACCAGCG ATCCTCCATG TAGACTGAGC ATGGACATGC |
| | GGAAGCACAC CCTGCAGGCC |
| 1561 | ATCGTCGGCA GCGGCAAGAA GAAGAACATC CTTAGACGGT |
| | GCAGGGTGTG CAGCGTGCAC |
| 1621 | AAGCTGCGGA GCGAGACTCG GTACATGTGC AAGTTTTGCA |
| | ACATTCCCCT GCACAAGGGA |
| 1681 | GCCTGCTTCG AGAAGTACCA CACCCTGAAG AATTACTAG |

In embodiments, the targeting element is able to direct a transposition machinery to the GSHS of a nucleic acid molecule in a mammalian cell. In embodiments, the GSHS is in an open chromatin location in a chromosome. In embodiments, the GSHS is selected from adeno-associated virus site 1 (AAVS1), chemokine (C-C motif) receptor 5 (CCR5) gene, HIV-1 coreceptor, and human Rosa26 locus. In embodiments, the GSHS is an adeno-associated virus site 1 (AAVS1). In embodiments, the GSHS is a human Rosa26 locus. In embodiments, the GSHS is located on human chromosome 2, 4, 6, 10, 11, 17, 22, or X.

In embodiments, the GSHS is selected from TABLES 3-17. In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TA-LER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the targeting element is or comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof. In embodiments, the targeting element comprises a TALE DBD. In embodiments, the TALE DBD comprises one or more repeat sequences. In embodiments, the TALE DBD comprises about 14, or about 15, or about, 16, or about 17, or about 18, or about 18.5 repeat sequences. In embodiments, the repeat sequences each independently comprises about 33 or 34 amino acids. In embodiments, the repeat sequences each independently comprises a repeat variable di-residue (RVD) at residue 12 or 13 of the 33 or 34 amino acids, respectively. In embodiments, the RVD recognizes one base pair in a target nucleic acid sequence. In embodiments, the RVD recognizes a C residue in the target nucleic acid sequence and is selected from HD, N(gap), HA, ND, and HI. In embodiments, the RVD recognizes a G residue in the target nucleic acid sequence and is selected from NN, NH, NK, HN, and NA. In embodiments, the RVD recognizes an A residue in the target nucleic acid sequence and is selected from NI and NS. In embodiments, the RVD recognizes a T residue in the target nucleic acid sequence and is selected from NG, HG, H(gap), and IG.

In embodiments, the TALE DBD targets one or more of GSHS sites selected from TABLES 8-12 and TABLE 20.

In embodiments, the TALE DBD comprises one or more of RVD se-lected from TABLES 8-12 and TABLE 20, or variants thereof comprising about 1, about 2, about 3, about 4, about 5, about 6, about 7, about 8, about 9, about 10, about 11, about 12, about 13, about 14, about 15, about 16, about 17, about 18, about 19, or about 20 mutations.

In embodiments, the targeting element comprises a Cas9 enzyme associated with a gRNA. In embodiments, the Cas9 enzyme associated with a gRNA comprises a catalytically inactive dCas9 associated with a gRNA.

In embodiments, the catalytically inactive dCas9 comprises at least about 90%, or at least about 95%, or at least about 96%, or at least about 97%, or at least about 98%, or at least about 99% identity to an amino acid sequence of SEQ ID NO: 6 or a nucleic acid comprising a nucleotide sequence of SEQ ID NO: 5 or a codon-optimized form thereof.

| SEQ ID NO: 5: nucleotide sequence of dead Cas9 DNA BINDING protein (5004 bp) | |
|---|---|
| 1 | ATGGACAAGA AGTACTCCAT TGGGCTCGCT ATCGGCACAA ACAGCGTCGG CTGGGCCGTC |
| 61 | ATTACGGACG AGTACAAGGT GCCGAGCAAA AAATTCAAAG TTCTGGGCAA TACCGATCGC |
| 121 | CACAGCATAA AGAAGAACCT CATTGGCGCC CTCCTGTTCG ACTCCGGGGA GACGGCCGAA |
| 181 | GCCACGCGGC TCAAAAGAAC AGCACGGCGC AGATATACCC GCAGAAAGAA TCGGATCTGC |
| 241 | TACCTGCAGG AGATCTTTAG TAATGAGATG GCTAAGGTGG ATGACTCTTT CTTCCATAGG |
| 301 | CTGGAGGAGT CCTTTTTGGT GGAGGAGGAT AAAAAGCACG AGCGCCACCC AATCTTTGGC |
| 361 | AATATCGTGG ACGAGGTGGC GTACCATGAA AAGTACCCAA CCATATATCA TCTGAGGAAG |
| 421 | AAGCTTGTAG ACAGTACTGA TAAGGCTGAC TTGCGGTTGA TCTATCTCGC GCTGGCGCAT |
| 481 | ATGATCAAAT TTCGGGGACA CTTCCTCATC GAGGGGGACC TGAACCCAGA CAACAGCGAT |
| 541 | GTCGACAAAC TCTTTATCCA ACTGGTTCAG ACTTACAATC AGCTTTTCGA AGAGAACCCG |
| 601 | ATCAACGCAT CCGGAGTTGA CGCCAAAGCA ATCCTGAGCG CTAGGCTGTC CAAATCCCGG |
| 661 | CGGCTCGAAA ACCTCATCGC ACAGCTCCCT GGGGAGAAGA AGAACGGCCT GTTTGGTAAT |
| 721 | CTTATCGCCC TGTCACTCGG GCTGACCCCC AACTTTAAAT CTAACTTCGA CCTGGCCGAA |
| 781 | GATGCCAAGC TTCAACTGAG CAAAGACACC TACGATGATG ATCTCGACAA TCTGCTGGCC |
| 841 | CAGATCGGCG ACCAGTACGC AGACCTTTTT TTGGCGGCAA AGAACCTGTC AGACGCCATT |
| 901 | CTGCTGAGTG ATATTCTGCG AGTGAACACG GAGATCACCA AAGCTCCGCT GAGCGCTAGT |
| 961 | ATGATCAAGC GCTATGATGA GCACCACCAA GACTTGACTT TGCTGAAGGC CCTTGTCAGA |
| 1021 | CAGCAACTGC CTGAGAAGTA CAAGGAAATT TTCTTCGATC AGTCTAAAAA TGGCTACGCC |
| 1081 | GGATACATTG ACGGCGGAGC AAGCCAGGAG GAATTTTACA AATTTATTAA GCCCATCTTG |
| 1141 | GAAAAAATGG ACGGCACCGA GGAGCTGCTG GTAAAGCTTA ACAGAGAAGA TCTGTTGCGC |
| 1201 | AAACAGCGCA CTTTCGACAA TGGAAGCATC CCCCACCAGA TTCACCTGGG CGAACTGCAC |
| 1261 | GCTATCCTCA GGCGGCAAGA GGATTTCTAC CCCTTTTTGA AAGATAACAG GGAAAAGATT |
| 1321 | GAGAAAATCC TCACATTTCG GATACCCTAC TATGTAGGCC CCCTCGCCCG GGGAAATTCC |
| 1381 | AGATTCGCGT GGATGACTCG CAAATCAGAA GAGACCATCA CTCCCTGGAA CTTCGAGGAA |
| 1441 | GTCGTGGATA AGGGGGCCTC TGCCCAGTCC TTCATCGAAA GGATGACTAA CTTTGATAAA |
| 1501 | AATCTGCCTA ACGAAAAGGT GCTTCCTAAA CACTCTCTGC TGTACGAGTA CTTCACAGTT |
| 1561 | TATAACGAGC TCACCAAGGT CAAATACGTC ACAGAAGGGA TGAGAAAGCC AGCATTCCTG |
| 1621 | TCTGGAGAGC AGAAGAAAGC TATCGTGGAC CTCCTCTTCA AGACGAACCG GAAAGTTACC |
| 1681 | GTGAAACAGC TCAAAGAAGA CTATTTCAAA AAGATTGAAT GTTTCGACTC TGTTGAAATC |
| 1741 | AGCGGAGTGG AGGATCGCTT CAACGCATCC CTGGGAACGT ATCACGATCT CCTGAAAATC |
| 1801 | ATTAAAGACA AGGACTTCCT GGACAATGAG GAGAACGAGG ACATTCTTGA GGACATTGTC |
| 1861 | CTCACCCTTA CGTTGTTTGA AGATAGGGAG ATGATTGAAG AACGCTTGAA AACTTACGCT |
| 1921 | CATCTCTTCG ACGACAAAGT CATGAAACAG CTCAAGAGGC GCCGATATAC AGGATGGGGG |
| 1981 | CGGCTGTCAA GAAAACTGAT CAATGGGATC CGAGACAAGC AGAGTGGAAA GACAATCCTG |
| 2041 | GATTTTCTTA AGTCCGATGG ATTTGCCAAC CGGAACTTCA TGCAGTTGAT CCATGATGAC |
| 2101 | TCTCTCACCT TTAAGGAGGA CATCCAGAAA GCACAAGTTT CTGGCCAGGG GGACAGTCTT |
| 2161 | CACGAGCACA TCGCTAATCT TGCAGGTAGC CCAGCTATCA AAAAGGGAAT ACTGCAGACC |
| 2221 | GTTAAGGTCG TGGATGAACT CGTCAAAGTA ATGGGAAGGC ATAAGCCCGA GAATATCGTT |
| 2281 | ATCGAGATGG CCCGAGAGAA CCAAACTACC CAGAAGGGAC AGAAGAACAG TAGGGAAAGG |
| 2341 | ATGAAGAGGA TTGAAGAGGG TATAAAAGAA CTGGGGTCCC AAATCCTTAA GGAACACCCA |
| 2401 | GTTGAAAACA CCCAGCTTCA GAATGAGAAG CTCTACCTGT ACTACCTGCA GAACGGCAGG |
| 2461 | GACATGTACG TGGATCAGGA ACTGGACATC AATCGGCTCT CCGACTACGA CGTGGCTGCT |
| 2521 | ATCGTGCCCC AGTCTTTTCT CAAAGATGAT TCTATTGATA ATAAAGTGTT GACAAGATCC |
| 2581 | GATAAAGCTA GAGGGAAGAG TGATAACGTC CCCTCAGAAG AAGTTGTCAA GAAAATGAAA |
| 2641 | AATTATTGGC GGCAGCTGCT GAACGCCAAA CTGATCACAC AACGGAAGTT CGATAATCTG |
| 2701 | ACTAAGGCTG AACGAGGTGG CCTGTCTGAG TTGGATAAAG CCGGCTTCAT CAAAAGGCAG |
| 2761 | CTTGTTGAGA CACGCCAGAT CACCAAGCAC GTGGCCCAAA TTCTCGATTC ACGCATGAAC |
| 2821 | ACCAAGTACG ATGAAAATGA CAAACTGATT CGAGAGGTGA AAGTTATTAC TCTGAAGTCT |
| 2881 | AAGCTGGTCT CAGATTTCAG AAAGGACTTT CAGTTTTATA AGGTGAGAGA GATCAACAAT |
| 2941 | TACCACCATG CGCATGATGC CTACCTGAAT GCAGTGGTAG GCACTGCACT TATCAAAAAA |
| 3001 | TATCCCAAGC TTGAATCTGA ATTTGTTTAC GGAGACTATA AAGTGTACGA TGTTAGGAAA |
| 3061 | ATGATCGCAA AGTCTGAGCA GGAAATAGGC AAGGCCACCG CTAAGTACTT CTTTTACAGC |
| 3121 | AATATTATGA ATTTTTTCAA GACCGAGATT ACACTGGCCA ATGGAGAGAT TCGGAAGCGA |
| 3181 | CCACTTATCG AAACAAACGG AGAAACAGGA GAAATCGTGT GGGACAAGGG TAGGGATTTC |
| 3241 | GCGACAGTCC GGAAGGTCCT GTCCATGCCG CAGGTGAACA TCGTTAAAAA GACCGAAGTA |
| 3301 | CAGACCGGAG GCTTCTCCAA GGAAAGTATC CTCCCGAAAA GGAACAGCGA CAAGCTGATC |
| 3361 | GCACGCAAAA AAGATTGGGA CCCCAAGAAA TACGGCGGAT TCGATTCTCC TACAGTCGCT |
| 3421 | TACAGTGTAC TGGTTGTGGC CAAAGTGGAG AAAGGGAAGT CTAAAAAACT CAAAAGCGTC |
| 3481 | AAGGAACTGC TGGGCATCAC AATCATGGAG CGATCAAGCT TCGAAAAAAA CCCCATCGAC |
| 3541 | TTTCTGGAGG CGAAAGGATA TAAAGAGGTC AAAAAAGACC TCATCATTAA GCTTCCCAAG |
| 3601 | TACTCTCTCT TTGAGCTTGA AAACGGCCGG AAACGAATGC TCGCTAGTGC GGGCGAGCTG |
| 3661 | CAGAAAGGTA ACGAGCTGGC ACTGCCCTCT AAATACGTTA ATTTCTTGTA TCTGGCCAGC |
| 3721 | CACTATGAAA AGCTCAAAGG GTCTCCCGAA GATAATGAGC AGAAGCAGCT GTTCGTGGAA |
| 3781 | CAACACAAAC ACTACCTTGA TGAGATCATC GAGCAAATAA GCGAATTCTC CAAAAGAGTG |
| 3841 | ATCCTCGCCG ACGCTAACCT CGATAAGGTG CTTTCTGCTT ACAATAAGCA CAGGGATAAG |
| 3901 | CCCATCAGGG AGCAGGCAGA AAACATTATC CACTTGTTTA CTCTGACCAA CTTGGGCGCG |
| 3961 | CCTGCAGCCT TCAAGTACTT CGACACCACC ATAGACAGAA AGCGGTACAC CTCTACAAAG |
| 4021 | GAGGTCCTGG ACGCCACACT GATTCATCAG TCAATTACGG GGCTCTATGA AACAAGAATC |
| 4081 | GACCTCTCTC AGCTCGGTGG AGAC |
| SEQ ID NO: 6: amino acid sequence of dead Cas9 DNA BINDING protein (1368 | |
| amino acids) | |
| 1 | MDKKYSIGLA IGTNSVGWAV ITDEYKVPSK KFKVLGNTDR HSIKKNLIGA LLFDSGETAE |
| 61 | ATRLKRTARR RYTRRKNRIC YLQEIFSNEM AKVDDSFFHR LEESFLVEED KKHERHPIFG |
| 121 | NIVDEVAYHE KYPTIYHLRK KLVDSTDKAD LRLIYLALAH MIKFRGHFLI EGDLNPDNSD |
| 181 | VDKLFIQLVQ TYNQLFEENP INASGVDAKA ILSARLSKSR RLENLIAQLP GEKKNGLFGN |
| 241 | LIALSLGLTP NFKSNFDLAE DAKLQLSKDT YDDDLDNLLA QIGDQYADLF LAAKNLSDAI |
| 301 | LLSDILRVNT EITKAPLSAS MIKRYDEHHQ DLTLLKALVR QQLPEKYKEI FFDQSKNGYA |
| 361 | GYIDGGASQE EFYKFIKPIL EKMDGTEELL VKLNREDLLR KQRTFDNGSI PHQIHLGELH |
| 421 | AILRRQEDFY PFLKDNREKI EKILTFRIPY YVGPLARGNS RFAWMTRKSE ETITPWNFEE |
| 481 | VVDKGASAQS FIERMTNFDK NLPNEKVLPK HSLLYEYFTV YNELTKVKYV TEGMRKPAFL |
| 541 | SGEQKKAIVD LLFKTNRKVT VKQLKEDYFK KIECFDSVEI SGVEDRFNAS LGTYHDLLKI |
| 601 | IKDKDFLDNE ENEDILEDIV LTLTLFEDRE MIEERLKTYA HLFDDKVMKQ LKRRRYTGWG |
| 661 | RLSRKLINGI RDKQSGKTIL DFLKSDGFAN RNFMQLIHDD SLTFKEDIQK AQVSGOGDSL |
| 721 | HEHIANLAGS PAIKKGILQT VKVVDELVKV MGRHKPENIV IEMARENQTT QKGQKNSRER |
| 781 | MKRIEEGIKE LGSQILKEHP VENTQLQNEK LYLYYLQNGR DMYVDQELDI NRLSDYDVAA |
| 841 | IVPQSFLKDD SIDNKVLTRS DKARGKSDNV PSEEVVKKMK NYWRQLLNAK LITQRKFDNL |
| 901 | TKAERGGLSE LDKAGFIKRQ LVETRQITKH VAQILDSRMN TKYDENDKLI REVKVITLKS |
| 961 | KLVSDFRKDF QFYKVREINN YHHAHDAYLN AVVGTALIKK YPKLESEFVY GDYKVYDVRK |
| 1021 | MIAKSEQEIG KATAKYFFYS NIMNFFKTEI TLANGEIRKR PLIETNGETG EIVWDKGRDF |
| 1081 | ATVRKVLSMP QVNIVKKTEV QTGGFSKESI LPKRNSDKLI ARKKDWDPKK YGGFDSPTVA |
| 1141 | YSVLVVAKVE KGKSKKLKSV KELLGITIME RSSFEKNPID FLEAKGYKEV KKDLIIKLPK |
| 1201 | YSLFELENGR KRMLASAGEL QKGNELALPS KYVNFLYLAS HYEKLKGSPE DNEQKQLFVE |
| 1261 | QHKHYLDEII EQISEFSKRV ILADANLDKV LSAYNKHRDK PIREQAENII HLFTLTNLGA |
| 1321 | PAAFKYFDTT IDRKRYTSTK EVLDATLIHQ SITGLYETRI DLSQLGGD |

In embodiments, the targeting element comprises a Cas12 enzyme associated with a gRNA. In embodiments, the targeting element comprises a catalytically inactive Cas12 associated with a gRNA, optionally wherein the catalytically inactive Cas12 is dCas12j or dCas12a. In embodiments, the targeting element comprises a TnsC, TnsB, TnsA, TniQ, Cas6, Cas7, Cas8 enzyme associated with a gRNA.

In embodiments, the targeting element comprises a TnsD.

In embodiments, the guide RNA is selected from TABLES 3-7 and TABLE 19, or variants thereof comprising about 1, about 2, about 3, about 4, about 5, about 6, about 7, about 8, about 9, about 10, about 11, about 12, about 13, about 14, about 15, about 16, about 17, about 18, about 19, or about 20 mutations. In embodiments, the guide RNA targets one or more sites selected from TABLES 3-7 and TABLE 19. In embodiments, the zinc finger comprises one of the sequences selected from TABLES 13-17, or variants thereof comprising about 99, about 98, about 97, about 95, about 94, about 93, about 92, about 91, about 90, about 89, about 88, about 87, about 86, about 85, about 84, about 83, about 82, about 81, about 80 percent identity to the sequence. In embodiments, the zinc finger targets one or more sites selected from TABLES 13-17.

In embodiments, the targeting element comprises a nucleic acid binding component of a gene-editing system. In embodiments, the helper enzyme or variant thereof and the targeting element are connected. In embodiments, the helper enzyme and the targeting element are fused to one another or linked via a linker to one another. In embodiments, the linker is a flexible linker. In embodiments, the flexible linker is substantially comprised of glycine and serine residues, optionally wherein the flexible linker comprises (Gly₄Ser)ₙ, where n is an integer from 1-12. In embodiments, the flexible linker is of about 20, or about 30, or about 40, or about 50, or about 60 amino acid residues. In embodiments, the helper enzyme is directly fused to the N-terminus of the targeting element and, optionally, wherein the targeting element is or comprises dCas9 enzyme.

In embodiments, the TnsD comprises a nucleic acid binding component of a gene-editing system. In embodiments, the enzyme or variant thereof (optionally, wherein the enzyme is a helper enzyme, optionally, wherein the helper enzyme is reconstructed from Myotis lucifugus) and the TnsD are connected. In embodiments, the helper enzyme and the TnsD are fused to one another or linked via a linker to one another. In embodiments, the linker is a flexible linker. In embodiments, the flexible linker is substantially comprised of glycine and serine residues, optionally wherein the flexible linker comprises (Gly₄Ser)ₙ, where n is an integer from 1-12. In embodiments, the flexible linker is of about 20, or about 30, or about 40, or about 50, or about 60 amino acid residues. In embodiments, the helper enzyme is directly fused to the N-terminus of the TnsD.

In embodiments, the E. coli TnsD comprises at least about 90%, or at least about 95%, or at least about 96%, or at least about 97%, or at least about 98%, or at least about 99% identity to an amino acid sequence of SEQ ID NO: 12. In embodiments, the TnsD comprises a truncated TnsD. In embodiments, the TnsD is truncated at its C-terminus. In embodiments, the TnsD is truncated at its N-terminus. In embodiments, the TnsD or variant thereof comprises a zinc finger motif. In embodiments, the zinc finger motif comprises a C3H-type motif (e.g., CCCH).

SEQ ID NO: 12: amino acid sequence of
E. coli TnsD (508 amino acids)

| | |
|---|---|
| 1 | MRNFPVPYSN ELIYSTIARA GVYQGIVSPK |
| | QLLDEVYGNR KVVATLGLPS HLGVIARHLH |
| 61 | QTGRYAVQQL IYEHTLFPLY APFVGKERRD |
| | EAIRLMEYQA QGAVHLMLGV AASRVKSDNR |
| 121 | FRYCPDCVAL QLNRYGEAFW QRDWYLPALP |
| | YCPKHGALVF FDRAVDDHRH QFWALGHTEL |
| 181 | LSDYPKDSLS QLTALAAYIA PLLDAPRAQE |
| | LSPSLEQWTL FYQRLAQDLG LTKSKHIRHD |
| 241 | LVAERVRQTF SDEALEKLDL KLAENKDTCW |
| | LKSIFRKHRK AFSYLQHSIV WQALLPKLTV |
| 301 | IEALQQASAL TEHSITTRPV SQSVQPNSED |
| | LSVKHKDWQQ LVHKYQGIKA ARQSLEGGVL |
| 361 | YAWLYRHDRD WLVHWNQQHQ QERLAPAPRV |
| | DWNQRDRIAV RQLLRIIKRL DSSLDHPRAT |
| 421 | SSWLLKQTPN GTSLAKNLQK LPLVALCLKR |
| | YSESVEDYQI RRISQAFIKL KQEDVELRRW |
| 481 | RLLRSATLSK ERITEEAQRF LEMVYGEE |

In embodiments, the TnsD binds at or near an attTn7 attachment site. In embodiments, the TnsD binds at or near a region downstream of the glmS gene. GlmS (L-glucosamine-fructose-6-phosphate aminotransferase) is highly conserved and found in a wide variety of organisms from bacteria to humans. In embodiments, the TnsD binding region of glmS encodes the active site region of GlmS. In embodiments, TnsD binds at or near the human homologs of glmS, e.g., gfpt-1 and gfpt-2. In embodiments, TnsD binds the human glmS homologs gfpt-1 and gfpt-2. In embodiments, the transgene is inserted into attTn7.

In embodiments, the helper enzyme or variant thereof is able to directly or indirectly cause transposition of a target gene. In embodiments, the helper enzyme or variant thereof is able to directly or indirectly interact and/or form a complex with one or more proteins or nucleic acids.

### Construct

In some embodiments, the composition (e.g., without limitation, a hyperactive helper of the present disclosure), system, or method further comprising a nucleic acid encoding a donor comprising a transgene to be integrated. In some embodiments, the transgene is defective or substantially absent in a disease state. In some embodiments, the transgene comprises a cargo nucleic acid sequence and a first and a second donor end sequences. In some embodiments, the cargo nucleic acid sequence is flanked by the first and the second donor end sequences.

In some embodiments, the donor end sequences are selected from nucleotide sequences of SEQ ID NO: 3 and/or SEQ ID NO: 4, or a nucleotide sequence having at least about 90% identity thereto.

```
SEQ ID NO: 3:
hyperactive helper Left ITR (157 bp)
The left ITR retains recognition activity when
the underlined nucleotides are deleted (80 bp).
    1 ttaacacttg gattgcggga aacgagttaa
        gtcggctcgc gtgaattgcg cgtactccgc
  61 gggagccgtc ttaactcggt tcatatagat
        ttgcggtgga gtgcgggaaa cgtgtaaact
121 cgggccgatt gtaactgcgt attaccaaat
        atttgtt
SEQ ID NO: 4:
hyperactive helper Right ITR (212 bp)
The right ITR retains recognition activity when
the underlined nucleotides are deleted (80 bp).
    1 aattatttat gtactgaata gataaaaaaa
        tgtctgtgat tgaataaatt ttcatttttt
  61 acacaagaaa ccgaaaattt catttcaatc
        gaacccatac ttcaaaagat ataggcattt
121 taaactaact ctgattttgc gcgggaaacc
        taaataattg cccgcgccat cttatatttt
181 ggcgggaaat tcacccgaca ccgtagtgtt
        aa
```

In some embodiments, the end sequences include at least one repeat from a nucleotide sequence having at least about 90% identity to the nucleotide sequence of SEQ ID NO: 3. In some embodiments, the at least one repeat from the nucleotide sequence having at least about 90% identity to the nucleotide sequence of SEQ ID NO: 3 is positioned at the 5′ end of the donor. In some embodiments, the end sequences can further include at least one repeat from a nucleotide sequence having at least about 90% identity to the nucleotide sequence of SEQ ID NO: 4. In some embodiments, the at least one repeat from the nucleotide sequence having at least about 90% identity to the nucleotide sequence of SEQ ID NO: 4 is positioned at the 3′ end of the donor.

In some embodiments, the helper enzyme or variant thereof is incorporated into a vector or a vector-like particle. In some embodiments, the vector or a vector-like particle comprises one or more expression cassettes. In some embodiments, the vector or a vector-like particle comprises one expression cassette. In some embodiments, the expression cassette further comprises the helper enzyme or variant thereof, the transgene, the donor end sequences, or a combination thereof.

In some embodiments, the helper enzyme or variant thereof, the transgene, the donor end sequences, or a combination thereof are incorporated into one or more vectors or vector-like particles. In some embodiments, the helper enzyme or variant thereof, the transgene, the donor end sequences, or combination thereof are incorporated into a same vector or vector-like particle. In some embodiments, the helper enzyme or variant thereof, the transgene, the donor end sequences, or combination thereof is incorporated into different vectors vector-like particles. In some embodiments, the vector or vector-like particle is nonviral. In some embodiments, the composition comprises DNA, RNA, or both. In some embodiments, the helper enzyme or variant thereof is in the form of RNA.

In embodiments, the donor is under the control of at least one tissue-specific promoter. In embodiments, the at least one tissue-specific promoter is a single promoter. In embodiments, the at least one tissue-specific promoter is under the control of a dual promoter or a tandem promoter.

In embodiments, the transgene to be integrated comprises at least one gene of interest. In embodiments, the transgene to be integrated comprises one gene of interest. In embodiments, the transgene to be integrated comprises two genes of interest.

In embodiments, the at least one gene of interest comprises peptides for linking genes of interest. In embodiments, the peptides are 2A self-cleaving peptides, or functional variants thereof, wherein the 2A self-cleaving peptide is optionally selected from P2A, E2A, F2A, and T2A, or derivative thereof.

In embodiments, the at least one gene of interest is linked to polynucleotide comprising a sequence comprising a 5′-miRNA, a sense and antisense miRNA pair, and/or a 3-miRNA.

In embodiments, the donor is used in combination with a gene silencing construct. In embodiments, there is provided a method of gene therapy in a cell comprising contacting the cell with a construct comprising the helper enzyme and/or donor or transgene described herein and/or a gene silencing construct. In embodiments, there is provided a method of gene replacement and silencing comprising contacting the cell with a construct comprising the helper enzyme and/or donor or transgene described herein and/or a gene silencing construct. In embodiments, there is provided a method of gene therapy in a subject comprising administering a construct comprising the helper enzyme and/or donor or transgene described herein and/or a gene silencing construct. In embodiments, there is provided a method of gene replacement and silencing in a subject comprising administering a construct comprising the helper enzyme and/or donor or transgene described herein and/or a gene silencing construct. In embodiments, the donor or transgene described herein and the gene silencing construct are separate constructs. In embodiments, the donor or transgene described herein and the gene silencing construct are separate DNA constructs.

In embodiments, the donor is dual gene construct. In embodiments, the donor is dual gene construct which comprises DNA. In embodiments, the donor is a bicistronic construct. In embodiments, the donor is a multicistrionic construct. In embodiments, the bicistronic construct allows for the contemporaneous expression of two proteins, e.g., separately from the same RNA transcript. In embodiments, the multicistrionic construct allows for the contemporaneous expression of multiple proteins, e.g., separately from the same RNA transcript.

In embodiments, the bicistronic and/or multicistronic construct comprises a gene of interest and a genetic silencing element. In embodiments, the genetic silencing element provides regulation of gene expression in a cell to prevent, reduce, or ablate the expression of a certain gene. In embodiments, the gene silencing element is capable of silencing during either transcription or translation. In embodiments, the gene silencing element is capable of gene knockdown or knockout. Accordingly, in embodiments, the donor is suitable for contemporaneous “knocking in” and “knocking out” of two or more genes. For example, in embodiments, a gene of interest is provided to a cell to have a beneficial effect and a deleterious gene is knocked out of a cell to reduce or eliminate a deleterious effect.

In embodiments, the gene silencing element is or comprises an RNA-based gene inhibitor or silencer. In embodiments, the gene silencing element is or comprises a short interfering RNA (siRNA), a microRNA (miRNA) and/or a short hairpin RNA (shRNA). embodiments, the donor is a bicistronic and/or multicistronic construct comprising one or more genes of interest, e.g., a transgene to be integrated, optionally wherein the transgene is defective or substantially absent in a disease state and one or more gene silencing element, e.g., one or more siRNA, miRNA, and shRNA. In embodiments, the donor is a bicistronic and/or multicistronic construct comprising one or more genes of interest, e.g., a transgene to be integrated, optionally wherein the transgene is defective or substantially absent in a disease state and one or more gene silencing element, e.g., one or more siRNA, miRNA, and shRNA and the donor is flanked by a first and a second donor end sequences.

In embodiments, the present compositions and methods provide for the helper enzyme or variant thereof excising and/or integrating both one or more one or more genes of interest, e.g., a transgene to be integrated, and one or more gene silencing element, e.g., one or more siRNA, miRNA, and shRNA. In embodiments, the present compositions and methods provide for gene replacement and silencing via a signal donor construct.

### N or C Terminal Deletion Variants

In aspects, the present disclosure further provides a hyperactive helper enzyme with a deletion of various amino acids at either the N or C terminus. In embodiments, the hyperactive helper enzyme comprises a deletion in the N-terminus. In embodiments, the hyperactive helper enzyme comprises a deletion in the C-terminus. In embodiments, the deletion in the N or C termini begins at various positions. In embodiments, the deletion in the N or C termini comprises various lengths.

In embodiments, the helper enzyme of the present disclosure comprises a deletion at positions about 1-35, or about 1-45, or about 1-55, or about 1-65, or about 1-75, or about 1-85, or about 1-95, or about 1-105 or positions corresponding thereto, wherein the positions are relative to SEQ ID NO: 502. In embodiments, the helper enzyme comprises an N-terminal deletion, optionally at positions about 1-34, or about 1-45, or about 1-68, or about 1-89 or positions corresponding thereto, wherein the positions are relative to SEQ ID NO: 9 or SEQ ID NO: 502. In embodiments, the helper enzyme comprises a C-terminal deletion, optionally at positions about 555-573 or about 530-573 or positions corresponding thereto, wherein the positions are relative to SEQ ID NO: 9 or SEQ ID NO: 502. In embodiments, the helper enzyme is an MLT. In embodiments, the deletion comprises an N or C terminal deletion. In embodiments, the N or C terminal deletion yields reduced or ablated off-target effects of the helper enzyme compared to the helper enzyme without the N or C terminal deletion. In embodiments, the helper enzyme comprising the N terminal deletion is N2. In embodiments, the helper enzyme comprising the N terminal deletion is or comprises SEQ ID NO: 506. In embodiments, the mutant with an N or C terminal deletion is further fused to a DNA binder. In embodiments, the DNA binder comprises TALEs, ZnF, and/or both.

In embodiments, the hyperactive helper enzyme comprises a deletion from an N- or C-terminus of the polypeptide having an amino acid sequence of SEQ ID NO: 502.

```
SEQ ID NO: 501:
Myositis lucifugus (hyperactive helper)
nucleotide sequence(NO). 1716 bp
      1 ATGGCCCAGC ACAGCGACTA CCCCGACGAC
          GAGTTCAGAG CCGATAAGCT GAGTAACTAC
    61 AGCTGCGACA GCGACCTGGA AAACGCCAGC
          ACATCCGACG AGGACAGCTC TGACGACGAG
  121 GTGATGGTGC GGCCCAGAAC CCTGAGACGG
          AGAAGAATCA GCAGCTCTAG CAGCGACTCT
  181 GAATCCGACA TCGAGGGCGG CCGGGAAGAG
          TGGAGCCACG TGGACAACCC TCCTGTTCTG
  241 GAAGATTTTC TGGGCCATCA GGGCCTGAAC
          ACCGACGCCG TGATCAACAA CATCGAGGAT
  301 GCCGTGAAGC TGTTCATAGG AGATGATTTC
          TTTGAGTTCC TGGTCGAGGA ATCCAACCGC
  361 TATTACAACC AGAATAGAAA CAACTTCAAG
          CTGAGCAAGA AAAGCCTGAA GTGGAAGGAC
  421 ATCACCCCTC AGGAGATGAA AAAGTTCCTG
          GGACTGATCG TTCTGATGGG ACAGGTGCGG
  481 AAGGACAGAA GGGATGATTA CTGGACAACC
          GAACCTTGGA CCGAGACCCC TTACTTTGGC
  541 AAGACCATGA CCAGAGACAG ATTCAGACAG
          ATCTGGAAAG CCTGGCACTT CAACAACAAT
  601 GCTGATATCG TGAACGAGTC TGATAGACTG
          TGTAAAGTGC GGCCAGTGTT GGATTACTTC
  661 GTGCCTAAGT TCATCAACAT CTATAAGCCT
          CACCAGCAGC TGAGCCTGGA TGAAGGCATC
  721 GTGCCCTGGC GGGGCAGACT GTTCTTCAGA
          GTGTACAATG CTGGCAAGAT CGTCAAATAC
  781 GGCATCCTGG TGCGCCTTCT GTGCGAGAGC
          GATACAGGCT ACATCTGTAA TATGGAAATC
  841 TACTGCGGCG AGGGCAAAAG ACTGCTGGAA
          ACCATCCAGA CCGTCGTTTC CCCTTATACC
  901 GACAGCTGGT ACCACATCTA CATGGACAAC
          TACTACAATT CTGTGGCCAA CTGCGAGGCC
  961 CTGATGAAGA ACAAGTTTAG AATCTGCGGC
          ACAATCAGAA AAAACAGAGG CATCCCTAAG
1021 GACTTCCAGA CCATCTCTCT GAAGAAGGGC
          GAAACCAAGT TCATCAGAAA GAACGACATC
1081 CTGCTCCAAG TGTGGCAGTC CAAGAAACCC
          GTGTACCTGA TCAGCAGCAT CCATAGCGCC
1141 GAGATGGAAG AAAGCCAGAA CATCGACAGA
          ACAAGCAAGA AGAAGATCGT GAAGCCCAAT
1201 GCTCTGATCG ACTACAACAA GCACATGAAA
          GGCGTGGACC GGGCCGACCA GTACCTGTCT
1261 TATTACTCTA TCCTGAGAAG AACAGTGAAA
          TGGACCAAGA GACTGGCCAT GTACATGATC
1321 AATTGCGCCC TGTTCAACAG CTACGCCGTG
          TACAAGTCCG TGCGACAAAG AAAAATGGGA
1381 TTCAAGATGT TCCTGAAGCA GACAGCCATC
          CACTGGCTGA CAGACGACAT TCCTGAGGAC
1441 ATGGACATTG TGCCAGATCT GCAACCTGTG
          CCCAGCACCT CTGGTATGAG AGCTAAGCCT
1501 CCCACCAGCG ATCCTCCATG TAGACTGAGC
          ATGGACATGC GGAAGCACAC CCTGCAGGCC
1561 ATCGTCGGCA GCGGCAAGAA GAAGAACATC
          CTTAGACGGT GCAGGGTGTG CAGCGTGCAC
1621 AAGCTGCGGA GCGAGACTCG GTACATGTGC
          AAGTTTTGCA ACATTCCCCT GCACAAGGGA
1681 GCCTGCTTCG AGAAGTACCA CACCCTGAAG
          AATTAC
SEQ ID NO: 502:
Myositis lucifugus (hyperactive helper)
amino acid sequence(NO). 572 aa
      1 MAQHSDYPDD EFRADKLSNY SCDSDLENAS
          TSDEDSSDDE VMVRPRTLRR RRISSSSSDS
    61 ESDIEGGREE WSHVDNPPVL EDFLGHQGLN
          TDAVINNIED AVKLFIGDDF FEFLVEESNR
  121 YYNQNRNNFK LSKKSLKWKD ITPQEMKKFL
          GLIVLMGQVR KDRRDDYWTT EPWTETPYFG
  181 KTMTRDRFRQ IWKAWHENNN ADIVNESDRL
          CKVRPVLDYF VPKFINIYKP HQQLSLDEGI
  241 VPWRGRLFFR VYNAGKIVKY GILVRLLCES
          DTGYICNMEI YCGEGKRLLE TIQTVVSPYT
  301 DSWYHIYMDN YYNSVANCEA LMKNKFRICG
          TIRKNRGIPK DFQTISLKKG ETKFIRKNDI
  361 LLQVWQSKKP VYLISSIHSA EMEESQNIDR
          TSKKKIVKPN ALIDYNKHMK GVDRADQYLS
  421 YYSILRRTVK WTKRLAMYMI NCALFNSYAV
          YKSVRQRKMG FKMFLKQTAI HWLTDDIPED
  481 MDIVPDLQPV PSTSGMRAKP PTSDPPCRLS
          MDMRKHTLQA IVGSGKKKNI LRRCRVCSVH
  541 KLRSETRYMC KFCNIPLHKG ACFEKYHTLK
          NY
```

In embodiments, the hyperactive helper enzyme comprises a deletion of about 5, or about 10, or about 20, or about 30, or about 40, or about 50, or about 60, or about 70, or about 80, or about 90, or about 100, or about 110, or about 120, or about 130, or about 140, or about 150, or about 160 amino acids from an N-terminus of the polypeptide having an amino acid sequence of SEQ ID NO: 502, or a sequence having at least about 90% identity thereto.

In embodiments, the hyperactive helper enzyme with deletion from the N-terminus comprises SEQ ID NO: 504, SEQ ID NO: 506, SEQ ID NO: 508, or SEQ ID NO: 510, or a sequence having at least about 90% identity thereto.

```
SEQ ID NO: 503: N-terminal deletion Myositis lucifugus
(hyperactive helper) nucleotide sequence (N1; nucleotide
4-105 deletion). 1614 bp
      1 ATGAGCTCTG ACGACGAGGT GATGGTGCGG CCCAGAACCC TGAGACGGAG AAGAATCAGC
    61 AGCTCTAGCA GCGACTCTGA ATCCGACATC GAGGGCGGCC GGGAAGAGTG GAGCCACGTG
  121 GACAACCCTC CTGTTCTGGA AGATTTTCTG GGCCATCAGG GCCTGAACAC CGACGCCGTG
  181 ATCAACAACA TCGAGGATGC CGTGAAGCTG TTCATAGGAG ATGATTTCTT TGAGTTCCTG
  241 GTCGAGGAAT CCAACCGCTA TTACAACCAG AATAGAAACA ACTTCAAGCT GAGCAAGAAA
  301 AGCCTGAAGT GGAAGGACAT CACCCCTCAG GAGATGAAAA AGTTCCTGGG ACTGATCGTT
  361 CTGATGGGAC AGGTGCGGAA GGACAGAAGG GATGATTACT GGACAACCGA ACCTTGGACC
  421 GAGACCCCTT ACTTTGGCAA GACCATGACC AGAGACAGAT TCAGACAGAT CTGGAAAGCC
  481 TGGCACTTCA ACAACAATGC TGATATCGTG AACGAGTCTG ATAGACTGTG TAAAGTGCGG
  541 CCAGTGTTGG ATTACTTCGT GCCTAAGTTC ATCAACATCT ATAAGCCTCA CCAGCAGCTG
  601 AGCCTGGATG AAGGCATCGT GCCCTGGCGG GGCAGACTGT TCTTCAGAGT GTACAATGCT
  661 GGCAAGATCG TCAAATACGG CATCCTGGTG CGCCTTCTGT GCGAGAGCGA TACAGGCTAC
  721 ATCTGTAATA TGGAAATCTA CTGCGGCGAG GGCAAAAGAC TGCTGGAAAC CATCCAGACC
  781 GTCGTTTCCC CTTATACCGA CAGCTGGTAC CACATCTACA TGGACAACTA CTACAATTCT
  841 GTGGCCAACT GCGAGGCCCT GATGAAGAAC AAGTTTAGAA TCTGCGGCAC AATCAGAAAA
  901 AACAGAGGCA TCCCTAAGGA CTTCCAGACC ATCTCTCTGA AGAAGGGCGA AACCAAGTTC
  961 ATCAGAAAGA ACGACATCCT GCTCCAAGTG TGGCAGTCCA AGAAACCCGT GTACCTGATC
1021 AGCAGCATCC ATAGCGCCGA GATGGAAGAA AGCCAGAACA TCGACAGAAC AAGCAAGAAG
1081 AAGATCGTGA AGCCCAATGC TCTGATCGAC TACAACAAGC ACATGAAAGG CGTGGACCGG
1141 GCCGACCAGT ACCTGTCTTA TTACTCTATC CTGAGAAGAA CAGTGAAATG GACCAAGAGA
1201 CTGGCCATGT ACATGATCAA TTGCGCCCTG TTCAACAGCT ACGCCGTGTA CAAGTCCGTG
1261 CGACAAAGAA AAATGGGATT CAAGATGTTC CTGAAGCAGA CAGCCATCCA CTGGCTGACA
1321 GACGACATTC CTGAGGACAT GGACATTGTG CCAGATCTGC AACCTGTGCC CAGCACCTCT
1381 GGTATGAGAG CTAAGCCTCC CACCAGCGAT CCTCCATGTA GACTGAGCAT GGACATGCGG
1441 AAGCACACCC TGCAGGCCAT CGTCGGCAGC GGCAAGAAGA AGAACATCCT TAGACGGTGC
1501 AGGGTGTGCA GCGTGCACAA GCTGCGGAGC GAGACTCGGT ACATGTGCAA GTTTTGCAAC
1561 ATTCCCCTGC ACAAGGGAGC CTGCTTCGAG AAGTACCACA CCCTGAAGAA TTAC
SEQ ID NO: 504: Myositis lucifugus (hyperactive helper)
amino acid sequence (N1, amino acid 2-35 deletion).
538 aa
      1 MSSDDEVMVR PRTLRRRRIS SSSSDSESDI EGGREEWSHV DNPPVLEDFL GHQGLNTDAV
    61 INNIEDAVKL FIGDDFFEFL VEESNRYYNQ NRNNFKLSKK SLKWKDITPQ EMKKFLGLIV
  121 LMGQVRKDRR DDYWTTEPWT ETPYFGKTMT RDRFRQIWKA WHENNNADIV NESDRLCKVR
  181 PVLDYFVPKF INIYKPHQQL SLDEGIVPWR GRLFFRVYNA GKIVKYGILV RLLCESDTGY
  241 ICNMEIYCGE GKRLLETIQT VVSPYTDSWY HIYMDNYYNS VANCEALMKN KFRICGTIRK
  301 NRGIPKDFQT ISLKKGETKF IRKNDILLQV WQSKKPVYLI SSIHSAEMEE SQNIDRTSKK
  361 KIVKPNALID YNKHMKGVDR ADQYLSYYSI LRRTVKWTKR LAMYMINCAL FNSYAVYKSV
  421 RQRKMGFKMF LKQTAIHWLT DDIPEDMDIV PDLQPVPSTS GMRAKPPTSD PPCRLSMDMR
  481 KHTLQAIVGS GKKKNILRRC RVCSVHKLRS ETRYMCKFCN IPLHKGACFE KYHTLKNY
SEQ ID NO: 505: N-terminal deletion Myositis lucifugus
(hyperactive helper) nucleotide sequence (N2; nucleotide
4-135 deletion). 1584 bp
      1 ATGAGAACCC TGAGACGGAG AAGAATCAGC AGCTCTAGCA GCGACTCTGA ATCCGACATC
    61 GAGGGCGGCC GGGAAGAGTG GAGCCACGTG GACAACCCTC CTGTTCTGGA AGATTTTCTG
  121 GGCCATCAGG GCCTGAACAC CGACGCCGTG ATCAACAACA TCGAGGATGC CGTGAAGCTG
  181 TTCATAGGAG ATGATTTCTT TGAGTTCCTG GTCGAGGAAT CCAACCGCTA TTACAACCAG
  241 AATAGAAACA ACTTCAAGCT GAGCAAGAAA AGCCTGAAGT GGAAGGACAT CACCCCTCAG
  301 GAGATGAAAA AGTTCCTGGG ACTGATCGTT CTGATGGGAC AGGTGCGGAA GGACAGAAGG
  361 GATGATTACT GGACAACCGA ACCTTGGACC GAGACCCCTT ACTTTGGCAA GACCATGACC
  421 AGAGACAGAT TCAGACAGAT CTGGAAAGCC TGGCACTTCA ACAACAATGC TGATATCGTG
  481 AACGAGTCTG ATAGACTGTG TAAAGTGCGG CCAGTGTTGG ATTACTTCGT GCCTAAGTTC
  541 ATCAACATCT ATAAGCCTCA CCAGCAGCTG AGCCTGGATG AAGGCATCGT GCCCTGGCGG
  601 GGCAGACTGT TCTTCAGAGT GTACAATGCT GGCAAGATCG TCAAATACGG CATCCTGGTG
  661 CGCCTTCTGT GCGAGAGCGA TACAGGCTAC ATCTGTAATA TGGAAATCTA CTGCGGCGAG
  721 GGCAAAAGAC TGCTGGAAAC CATCCAGACC GTCGTTTCCC CTTATACCGA CAGCTGGTAC
  781 CACATCTACA TGGACAACTA CTACAATTCT GTGGCCAACT GCGAGGCCCT GATGAAGAAC
  841 AAGTTTAGAA TCTGCGGCAC AATCAGAAAA AACAGAGGCA TCCCTAAGGA CTTCCAGACC
  901 ATCTCTCTGA AGAAGGGCGA AACCAAGTTC ATCAGAAAGA ACGACATCCT GCTCCAAGTG
  961 TGGCAGTCCA AGAAACCCGT GTACCTGATC AGCAGCATCC ATAGCGCCGA GATGGAAGAA
1021 AGCCAGAACA TCGACAGAAC AAGCAAGAAG AAGATCGTGA AGCCCAATGC TCTGATCGAC
1081 TACAACAAGC ACATGAAAGG CGTGGACCGG GCCGACCAGT ACCTGTCTTA TTACTCTATC
1141 CTGAGAAGAA CAGTGAAATG GACCAAGAGA CTGGCCATGT ACATGATCAA TTGCGCCCTG
1201 TTCAACAGCT ACGCCGTGTA CAAGTCCGTG CGACAAAGAA AAATGGGATT CAAGATGTTC
1261 CTGAAGCAGA CAGCCATCCA CTGGCTGACA GACGACATTC CTGAGGACAT GGACATTGTG
1321 CCAGATCTGC AACCTGTGCC CAGCACCTCT GGTATGAGAG CTAAGCCTCC CACCAGCGAT
1381 CCTCCATGTA GACTGAGCAT GGACATGCGG AAGCACACCC TGCAGGCCAT CGTCGGCAGC
1441 GGCAAGAAGA AGAACATCCT TAGACGGTGC AGGGTGTGCA GCGTGCACAA GCTGCGGAGC
1501 GAGACTCGGT ACATGTGCAA GTTTTGCAAC ATTCCCCTGC ACAAGGGAGC CTGCTTCGAG
1561 AAGTACCACA CCCTGAAGAA TTAC
SEQ ID NO: 506: Myositis lucifugus (hyperactive helper)
amino acid sequence (N2, amino acid 2-45 deletion).
528 aa
      1 MRTLRRRRIS SSSSDSESDI EGGREEWSHV DNPPVLEDFL GHQGLNTDAV INNIEDAVKL
    61 FIGDDFFEFL VEESNRYYNQ NRNNFKLSKK SLKWKDITPQ EMKKFLGLIV LMGQVRKDRR
  121 DDYWTTEPWT ETPYFGKTMT RDRFRQIWKA WHENNNADIV NESDRLCKVR PVLDYFVPKF
  181 INIYKPHQQL SLDEGIVPWR GRLFFRVYNA GKIVKYGILV RLLCESDTGY ICNMEIYCGE
  241 GKRLLETIQT VVSPYTDSWY HIYMDNYYNS VANCEALMKN KFRICGTIRK NRGIPKDFQT
  301 ISLKKGETKF IRKNDILLQV WQSKKPVYLI SSIHSAEMEE SQNIDRTSKK KIVKPNALID
  361 YNKHMKGVDR ADQYLSYYSI LRRTVKWTKR LAMYMINCAL FNSYAVYKSV RQRKMGFKMF
  421 LKQTAIHWLT DDIPEDMDIV PDLQPVPSTS GMRAKPPTSD PPCRLSMDMR KHTLQAIVGS
  481 GKKKNILRRC RVCSVHKLRS ETRYMCKFCN IPLHKGACFE KYHTLKNY
SEQ ID NO: 507: N-terminal deletion Myositis lucifugus
(hyperactive helper) nucleotide sequence (N3; nucleotide
4-204 deletion). 1515 bp
      1 ATGGAAGAGT GGAGCCACGT GGACAACCCT CCTGTTCTGG AAGATTTTCT GGGCCATCAG
    61 GGCCTGAACA CCGACGCCGT GATCAACAAC ATCGAGGATG CCGTGAAGCT GTTCATAGGA
  121 GATGATTTCT TTGAGTTCCT GGTCGAGGAA TCCAACCGCT ATTACAACCA GAATAGAAAC
  181 AACTTCAAGC TGAGCAAGAA AAGCCTGAAG TGGAAGGACA TCACCCCTCA GGAGATGAAA
  241 AAGTTCCTGG GACTGATCGT TCTGATGGGA CAGGTGCGGA AGGACAGAAG GGATGATTAC
  301 TGGACAACCG AACCTTGGAC CGAGACCCCT TACTTTGGCA AGACCATGAC CAGAGACAGA
  361 TTCAGACAGA TCTGGAAAGC CTGGCACTTC AACAACAATG CTGATATCGT GAACGAGTCT
  421 GATAGACTGT GTAAAGTGCG GCCAGTGTTG GATTACTTCG TGCCTAAGTT CATCAACATC
  481 TATAAGCCTC ACCAGCAGCT GAGCCTGGAT GAAGGCATCG TGCCCTGGCG GGGCAGACTG
  541 TTCTTCAGAG TGTACAATGC TGGCAAGATC GTCAAATACG GCATCCTGGT GCGCCTTCTG
  601 TGCGAGAGCG ATACAGGCTA CATCTGTAAT ATGGAAATCT ACTGCGGCGA GGGCAAAAGA
  661 CTGCTGGAAA CCATCCAGAC CGTCGTTTCC CCTTATACCG ACAGCTGGTA CCACATCTAC
  721 ATGGACAACT ACTACAATTC TGTGGCCAAC TGCGAGGCCC TGATGAAGAA CAAGTTTAGA
  781 ATCTGCGGCA CAATCAGAAA AAACAGAGGC ATCCCTAAGG ACTTCCAGAC CATCTCTCTG
  841 AAGAAGGGCG AAACCAAGTT CATCAGAAAG AACGACATCC TGCTCCAAGT GTGGCAGTCC
  901 AAGAAACCCG TGTACCTGAT CAGCAGCATC CATAGCGCCG AGATGGAAGA AAGCCAGAAC
  961 ATCGACAGAA CAAGCAAGAA GAAGATCGTG AAGCCCAATG CTCTGATCGA CTACAACAAG
1021 CACATGAAAG GCGTGGACCG GGCCGACCAG TACCTGTCTT ATTACTCTAT CCTGAGAAGA
1081 ACAGTGAAAT GGACCAAGAG ACTGGCCATG TACATGATCA ATTGCGCCCT GTTCAACAGC
1141 TACGCCGTGT ACAAGTCCGT GCGACAAAGA AAAATGGGAT TCAAGATGTT CCTGAAGCAG
1201 ACAGCCATCC ACTGGCTGAC AGACGACATT CCTGAGGACA TGGACATTGT GCCAGATCTG
1261 CAACCTGTGC CCAGCACCTC TGGTATGAGA GCTAAGCCTC CCACCAGCGA TCCTCCATGT
1321 AGACTGAGCA TGGACATGCG GAAGCACACC CTGCAGGCCA TCGTCGGCAG CGGCAAGAAG
1381 AAGAACATCC TTAGACGGTG CAGGGTGTGC AGCGTGCACA AGCTGCGGAG CGAGACTCGG
1441 TACATGTGCA AGTTTTGCAA CATTCCCCTG CACAAGGGAG CCTGCTTCGA GAAGTACCAC
1501 ACCCTGAAGA ATTAC
SEQ ID NO: 508: Myositis lucifugus (hyperactive helper) amino acid
sequence (N3, amino acid 2-68 deletion) 505 aa
      1 MEEWSHVDNP PVLEDFLGHQ GLNTDAVINN IEDAVKLFIG DDFFEFLVEE SNRYYNQNRN
    61 NFKLSKKSLK WKDITPQEMK KFLGLIVLMG QVRKDRRDDY WTTEPWTETP YFGKTMTRDR
  121 FRQIWKAWHF NNNADIVNES DRLCKVRPVL DYFVPKFINI YKPHQQLSLD EGIVPWRGRL
  181 FFRVYNAGKI VKYGILVRLL CESDTGYICN MEIYCGEGKR LLETIQTVVS PYTDSWYHIY
  241 MDNYYNSVAN CEALMKNKFR ICGTIRKNRG IPKDFQTISL KKGETKFIRK NDILLQVWQS
  301 KKPVYLISSI HSAEMEESQN IDRTSKKKIV KPNALIDYNK HMKGVDRADQ YLSYYSILRR
  361 TVKWTKRLAM YMINCALFNS YAVYKSVRQR KMGFKMFLKQ TAIHWLTDDI PEDMDIVPDL
  421 QPVPSTSGMR AKPPTSDPPC RLSMDMRKHT LQAIVGSGKK KNILRRCRVC SVHKLRSETR
  481 YMCKFCNIPL HKGACFEKYH TLKNY
SEQ ID NO: 509: N-terminal deletion Myositis lucifugus
(hyperactive helper) nucleotide sequence (N4; nucleotide
4-267 deletion). 1452 bp
      1 ATGAACACCG ACGCCGTGAT CAACAACATC GAGGATGCCG TGAAGCTGTT CATAGGAGAT
    61 GATTTCTTTG AGTTCCTGGT CGAGGAATCC AACCGCTATT ACAACCAGAA TAGAAACAAC
  121 TTCAAGCTGA GCAAGAAAAG CCTGAAGTGG AAGGACATCA CCCCTCAGGA GATGAAAAAG
  181 TTCCTGGGAC TGATCGTTCT GATGGGACAG GTGCGGAAGG ACAGAAGGGA TGATTACTGG
  241 ACAACCGAAC CTTGGACCGA GACCCCTTAC TTTGGCAAGA CCATGACCAG AGACAGATTC
  301 AGACAGATCT GGAAAGCCTG GCACTTCAAC AACAATGCTG ATATCGTGAA CGAGTCTGAT
  361 AGACTGTGTA AAGTGCGGCC AGTGTTGGAT TACTTCGTGC CTAAGTTCAT CAACATCTAT
  421 AAGCCTCACC AGCAGCTGAG CCTGGATGAA GGCATCGTGC CCTGGCGGGG CAGACTGTTC
  481 TTCAGAGTGT ACAATGCTGG CAAGATCGTC AAATACGGCA TCCTGGTGCG CCTTCTGTGC
  541 GAGAGCGATA CAGGCTACAT CTGTAATATG GAAATCTACT GCGGCGAGGG CAAAAGACTG
  601 CTGGAAACCA TCCAGACCGT CGTTTCCCCT TATACCGACA GCTGGTACCA CATCTACATG
  661 GACAACTACT ACAATTCTGT GGCCAACTGC GAGGCCCTGA TGAAGAACAA GTTTAGAATC
  721 TGCGGCACAA TCAGAAAAAA CAGAGGCATC CCTAAGGACT TCCAGACCAT CTCTCTGAAG
  781 AAGGGCGAAA CCAAGTTCAT CAGAAAGAAC GACATCCTGC TCCAAGTGTG GCAGTCCAAG
  841 AAACCCGTGT ACCTGATCAG CAGCATCCAT AGCGCCGAGA TGGAAGAAAG CCAGAACATC
  901 GACAGAACAA GCAAGAAGAA GATCGTGAAG CCCAATGCTC TGATCGACTA CAACAAGCAC
  961 ATGAAAGGCG TGGACCGGGC CGACCAGTAC CTGTCTTATT ACTCTATCCT GAGAAGAACA
1021 GTGAAATGGA CCAAGAGACT GGCCATGTAC ATGATCAATT GCGCCCTGTT CAACAGCTAC
1081 GCCGTGTACA AGTCCGTGCG ACAAAGAAAA ATGGGATTCA AGATGTTCCT GAAGCAGACA
1141 GCCATCCACT GGCTGACAGA CGACATTCCT GAGGACATGG ACATTGTGCC AGATCTGCAA
1201 CCTGTGCCCA GCACCTCTGG TATGAGAGCT AAGCCTCCCA CCAGCGATCC TCCATGTAGA
1261 CTGAGCATGG ACATGCGGAA GCACACCCTG CAGGCCATCG TCGGCAGCGG CAAGAAGAAG
1321 AACATCCTTA GACGGTGCAG GGTGTGCAGC GTGCACAAGC TGCGGAGCGA GACTCGGTAC
1381 ATGTGCAAGT TTTGCAACAT TCCCCTGCAC AAGGGAGCCT GCTTCGAGAA GTACCACACC
1441 CTGAAGAATT AC
SEQ ID NO: 510: Myositis lucifugus (hyperactive helper)
amino acid sequence (N4, amino acid 2-89 deletion).
484 aa
      1 MNTDAVINNI EDAVKLFIGD DFFEFLVEES NRYYNQNRNN FKLSKKSLKW KDITPQEMKK
    61 FLGLIVLMGQ VRKDRRDDYW TTEPWTETPY FGKTMTRDRF RQIWKAWHEN NNADIVNESD
  121 RLCKVRPVLD YFVPKFINIY KPHQQLSLDE GIVPWRGRLF FRVYNAGKIV KYGILVRLLC
  181 ESDTGYICNM EIYCGEGKRL LETIQTVVSP YTDSWYHIYM DNYYNSVANC EALMKNKFRI
  241 CGTIRKNRGI PKDFQTISLK KGETKFIRKN DILLQVWQSK KPVYLISSIH SAEMEESQNI
  301 DRTSKKKIVK PNALIDYNKH MKGVDRADQY LSYYSILRRT VKWTKRLAMY MINCALFNSY
  361 AVYKSVRQRK MGFKMFLKQT AIHWLTDDIP EDMDIVPDLQ PVPSTSGMRA KPPTSDPPCR
  421 LSMDMRKHTL QAIVGSGKKK NILRRCRVCS VHKLRSETRY MCKFCNIPLH KGACFEKYHT
  481 LKNY
```

In embodiments, the hyperactive helper enzyme comprises a deletion of about 5, or about 10, or about 20, or about 30, or about 40, or about 50, or about 60, or about 70, or about 80, or about 90, or about 100, or about 110, or about 120, or about 130, or about 140, or about 150, or about 160 amino acids from an C-terminus of the polypeptide having an amino acid sequence of SEQ ID NO: 502.

In embodiments, the hyperactive helper enzyme with deletion from the C-terminus comprises SEQ ID NO: 512 or SEQ ID NO: 514.

```
SEQ ID NO: 511: C-terminal deletion Myositis lucifugus
(hyperactive helper) nucleotide sequence
(C1; nucleotide 1663-1716 deletion). 1662 bp
      1 ATGGCCCAGC ACAGCGACTA CCCCGACGAC GAGTTCAGAG CCGATAAGCT GAGTAACTAC
    61 AGCTGCGACA GCGACCTGGA AAACGCCAGC ACATCCGACG AGGACAGCTC TGACGACGAG
  121 GTGATGGTGC GGCCCAGAAC CCTGAGACGG AGAAGAATCA GCAGCTCTAG CAGCGACTCT
  181 GAATCCGACA TCGAGGGCGG CCGGGAAGAG TGGAGCCACG TGGACAACCC TCCTGTTCTG
  241 GAAGATTTTC TGGGCCATCA GGGCCTGAAC ACCGACGCCG TGATCAACAA CATCGAGGAT
  301 GCCGTGAAGC TGTTCATAGG AGATGATTTC TTTGAGTTCC TGGTCGAGGA ATCCAACCGC
  361 TATTACAACC AGAATAGAAA CAACTTCAAG CTGAGCAAGA AAAGCCTGAA GTGGAAGGAC
  421 ATCACCCCTC AGGAGATGAA AAAGTTCCTG GGACTGATCG TTCTGATGGG ACAGGTGCGG
  481 AAGGACAGAA GGGATGATTA CTGGACAACC GAACCTTGGA CCGAGACCCC TTACTTTGGC
  541 AAGACCATGA CCAGAGACAG ATTCAGACAG ATCTGGAAAG CCTGGCACTT CAACAACAAT
  601 GCTGATATCG TGAACGAGTC TGATAGACTG TGTAAAGTGC GGCCAGTGTT GGATTACTTC
  661 GTGCCTAAGT TCATCAACAT CTATAAGCCT CACCAGCAGC TGAGCCTGGA TGAAGGCATC
  721 GTGCCCTGGC GGGGCAGACT GTTCTTCAGA GTGTACAATG CTGGCAAGAT CGTCAAATAC
  781 GGCATCCTGG TGCGCCTTCT GTGCGAGAGC GATACAGGCT ACATCTGTAA TATGGAAATC
  841 TACTGCGGCG AGGGCAAAAG ACTGCTGGAA ACCATCCAGA CCGTCGTTTC CCCTTATACC
  901 GACAGCTGGT ACCACATCTA CATGGACAAC TACTACAATT CTGTGGCCAA CTGCGAGGCC
  961 CTGATGAAGA ACAAGTTTAG AATCTGCGGC ACAATCAGAA AAAACAGAGG CATCCCTAAG
1021 GACTTCCAGA CCATCTCTCT GAAGAAGGGC GAAACCAAGT TCATCAGAAA GAACGACATC
1081 CTGCTCCAAG TGTGGCAGTC CAAGAAACCC GTGTACCTGA TCAGCAGCAT CCATAGCGCC
1141 GAGATGGAAG AAAGCCAGAA CATCGACAGA ACAAGCAAGA AGAAGATCGT GAAGCCCAAT
1201 GCTCTGATCG ACTACAACAA GCACATGAAA GGCGTGGACC GGGCCGACCA GTACCTGTCT
1261 TATTACTCTA TCCTGAGAAG AACAGTGAAA TGGACCAAGA GACTGGCCAT GTACATGATC
1321 AATTGCGCCC TGTTCAACAG CTACGCCGTG TACAAGTCCG TGCGACAAAG AAAAATGGGA
1381 TTCAAGATGT TCCTGAAGCA GACAGCCATC CACTGGCTGA CAGACGACAT TCCTGAGGAC
1441 ATGGACATTG TGCCAGATCT GCAACCTGTG CCCAGCACCT CTGGTATGAG AGCTAAGCCT
1501 CCCACCAGCG ATCCTCCATG TAGACTGAGC ATGGACATGC GGAAGCACAC CCTGCAGGCC
1561 ATCGTCGGCA GCGGCAAGAA GAAGAACATC CTTAGACGGT GCAGGGTGTG CAGCGTGCAC
1621 AAGCTGCGGA GCGAGACTCG GTACATGTGC AAGTTTTGCA AC
SEQ ID NO: 512:
Myositis lucifugus (hyperactive helper) amino acid
sequence (C1, amino acid 555-572 deletion).
554 aa
      1 MAQHSDYPDD EFRADKLSNY SCDSDLENAS TSDEDSSDDE VMVRPRTLRR RRISSSSSDS
    61 ESDIEGGREE WSHVDNPPVL EDFLGHQGLN TDAVINNIED AVKLFIGDDF FEFLVEESNR
  121 YYNQNRNNFK LSKKSLKWKD ITPQEMKKFL GLIVLMGQVR KDRRDDYWTT EPWTETPYFG
  181 KTMTRDRFRQ IWKAWHFNNN ADIVNESDRL CKVRPVLDYF VPKFINIYKP HQQLSLDEGI
  241 VPWRGRLFFR VYNAGKIVKY GILVRLLCES DTGYICNMEI YCGEGKRLLE TIQTVVSPYT
  301 DSWYHIYMDN YYNSVANCEA LMKNKFRICG TIRKNRGIPK DFOTISLKKG ETKFIRKNDI
  361 LLQVWQSKKP VYLISSIHSA EMEESQNIDR TSKKKIVKPN ALIDYNKHMK GVDRADQYLS
  421 YYSILRRTVK WTKRLAMYMI NCALFNSYAV YKSVRORKMG FKMFLKQTAI HWLTDDIPED
  481 MDIVPDLQPV PSTSGMRAKP PTSDPPCRLS MDMRKHTLQA IVGSGKKKNI LRRCRVCSVH
  541 KLRSETRYMC KFCN
SEQ ID NO: 513: C-terminal deletion Myositis lucifugus
(hyperactive helper) nucleotide sequence (C2; nucleotide
1588-1716 deletion). 1587 bp
      1 ATGGCCCAGC ACAGCGACTA CCCCGACGAC GAGTTCAGAG CCGATAAGCT GAGTAACTAC
    61 AGCTGCGACA GCGACCTGGA AAACGCCAGC ACATCCGACG AGGACAGCTC TGACGACGAG
  121 GTGATGGTGC GGCCCAGAAC CCTGAGACGG AGAAGAATCA GCAGCTCTAG CAGCGACTCT
  181 GAATCCGACA TCGAGGGCGG CCGGGAAGAG TGGAGCCACG TGGACAACCC TCCTGTTCTG
  241 GAAGATTTTC TGGGCCATCA GGGCCTGAAC ACCGACGCCG TGATCAACAA CATCGAGGAT
  301 GCCGTGAAGC TGTTCATAGG AGATGATTTC TTTGAGTTCC TGGTCGAGGA ATCCAACCGC
  361 TATTACAACC AGAATAGAAA CAACTTCAAG CTGAGCAAGA AAAGCCTGAA GTGGAAGGAC
  421 ATCACCCCTC AGGAGATGAA AAAGTTCCTG GGACTGATCG TTCTGATGGG ACAGGTGCGG
  481 AAGGACAGAA GGGATGATTA CTGGACAACC GAACCTTGGA CCGAGACCCC TTACTTTGGC
  541 AAGACCATGA CCAGAGACAG ATTCAGACAG ATCTGGAAAG CCTGGCACTT CAACAACAAT
  601 GCTGATATCG TGAACGAGTC TGATAGACTG TGTAAAGTGC GGCCAGTGTT GGATTACTTC
  661 GTGCCTAAGT TCATCAACAT CTATAAGCCT CACCAGCAGC TGAGCCTGGA TGAAGGCATC
  721 GTGCCCTGGC GGGGCAGACT GTTCTTCAGA GTGTACAATG CTGGCAAGAT CGTCAAATAC
  781 GGCATCCTGG TGCGCCTTCT GTGCGAGAGC GATACAGGCT ACATCTGTAA TATGGAAATC
  841 TACTGCGGCG AGGGCAAAAG ACTGCTGGAA ACCATCCAGA CCGTCGTTTC CCCTTATACC
  901 GACAGCTGGT ACCACATCTA CATGGACAAC TACTACAATT CTGTGGCCAA CTGCGAGGCC
  961 CTGATGAAGA ACAAGTTTAG AATCTGCGGC ACAATCAGAA AAAACAGAGG CATCCCTAAG
1021 GACTTCCAGA CCATCTCTCT GAAGAAGGGC GAAACCAAGT TCATCAGAAA GAACGACATC
1081 CTGCTCCAAG TGTGGCAGTC CAAGAAACCC GTGTACCTGA TCAGCAGCAT CCATAGCGCC
1141 GAGATGGAAG AAAGCCAGAA CATCGACAGA ACAAGCAAGA AGAAGATCGT GAAGCCCAAT
1201 GCTCTGATCG ACTACAACAA GCACATGAAA GGCGTGGACC GGGCCGACCA GTACCTGTCT
1261 TATTACTCTA TCCTGAGAAG AACAGTGAAA TGGACCAAGA GACTGGCCAT GTACATGATC
1321 AATTGCGCCC TGTTCAACAG CTACGCCGTG TACAAGTCCG TGCGACAAAG AAAAATGGGA
1381 TTCAAGATGT TCCTGAAGCA GACAGCCATC CACTGGCTGA CAGACGACAT TCCTGAGGAC
1441 ATGGACATTG TGCCAGATCT GCAACCTGTG CCCAGCACCT CTGGTATGAG AGCTAAGCCT
1501 CCCACCAGCG ATCCTCCATG TAGACTGAGC ATGGACATGC GGAAGCACAC CCTGCAGGCC
1561 ATCGTCGGCA GCGGCAAGAA GAAGAAC
SEQ ID NO: 514:
Myositis lucifugus (hyperactive helper) amino acid sequence
(C2, amino acid 530-572 deletion). 529 aa
      1 MAQHSDYPDD EFRADKLSNY SCDSDLENAS TSDEDSSDDE VMVRPRTLRR RRISSSSSDS
    61 ESDIEGGREE WSHVDNPPVL EDFLGHQGLN TDAVINNIED AVKLFIGDDF FEFLVEESNR
  121 YYNQNRNNFK LSKKSLKWKD ITPQEMKKFL GLIVLMGQVR KDRRDDYWTT EPWTETPYFG
  181 KTMTRDRFRQ IWKAWHENNN ADIVNESDRL CKVRPVLDYF VPKFINIYKP HOOLSLDEGI
  241 VPWRGRLFFR VYNAGKIVKY GILVRLLCES DTGYICNMEI YCGEGKRLLE TIQTVVSPYT
  301 DSWYHIYMDN YYNSVANCEA LMKNKFRICG TIRKNRGIPK DFQTISLKKG ETKFIRKNDI
  361 LLQVWQSKKP VYLISSIHSA EMEESQNIDR TSKKKIVKPN ALIDYNKHMK GVDRADQYLS
  421 YYSILRRTVK WTKRLAMYMI NCALFNSYAV YKSVRQRKMG FKMFLKQTAI HWLTDDIPED
  481 MDIVPDLQPV PSTSGMRAKP PTSDPPCRLS MDMRKHTLQA IVGSGKKKN
```

In embodiments, the hyperactive helper enzyme comprises a deletion at positions about 1-5, or about 1-15, or about 1-25, or about 1-35, or about 1-45, or about 1-55, or about 1-65, or about 1-75, or about 1-85, or about 1-95, or about 1-105, or about 1-115, or about 1-125, or about 1-135, or about 1-145, or about 1-155 or positions corresponding thereto, wherein the positions are relative to SEQ ID NO: 502.

In aspects, the N terminal deletion variant is further fused one or more DNA binders. In embodiments, the DNA binder comprises, without limitation, dCas9, dCas12j, TALEs, and ZnF. In embodiments, the DNA binder guides donor insertion to specific genomic sites. In embodiments, the C terminal deletion variant is further fused one or more DNA binders. In embodiments, the N terminal deletion variant is further fused one or more DNA binders at the N-terminus. In embodiments, the N terminal deletion variant is further fused one or more DNA binders at the C-terminus. In embodiments, the C terminal deletion variant is further fused one or more DNA binders at the N-terminus. In embodiments, the C terminal deletion variant is further fused one or more DNA binders at the C-terminus.

In embodiments, the hyperactive helper mutant exhibits improved excision frequencies compared to those without the terminal deletions and/or DNA binders. In embodiments, the hyperactive helper mutant exhibits improved integration frequencies compared to those without the terminal deletions and/or DNA binders. In embodiments, the hyperactive helper mutant exhibits improved excision and integration frequencies compared to those without the terminal deletions and/or DNA binders.

In embodiments, the N or C terminal mutant exhibit different Exc+/Int− frequencies. In embodiments, deletion of either N or C termini can result in MLT mutants with higher excision activity. In embodiments, N-terminal deletion yields a mutant with decreased integration compared to mutant without N-terminal deletion. In embodiments, C-terminal deletion yields a mutant with reduced excision and no integration.

In embodiments, the N or C terminal deletion yields reduced or ablated off-target effects of the helper enzyme compared to the helper enzyme without the N or C terminal deletion.

### Host Cell

In some aspects, the present disclosure further provides a host cell comprising the composition in accordance with embodiments of the present disclosure.

### Methods

In certain embodiments, the present disclosure provides a method for inserting a gene into the genome of a cell, comprising contacting a cell with the composition of the present disclosure or host cell of the present disclosure. In some embodiments, the method further comprises contacting the cell with a polynucleotide encoding a donor.

In some embodiments, the donor comprises a gene encoding a complete polypeptide.

In some embodiments, the donor comprises a gene which is defective or substantially absent in a disease state.

In certain embodiments, the present disclosure provides a method for treating a disease or disorder ex vivo, comprising contacting a cell with the composition of the present disclosure or host cell of the present disclosure and administering the cell to a subject in need thereof.

In certain embodiments, the present disclosure provides a method for treating a disease or disorder in vivo, comprising administering the composition of the present disclosure or host cell of the present disclosure to a subject in need thereof.

### Transgene

In embodiments, the transgene is an exogenous wild-type gene that, e.g., corrects a defective function of one or more mutations in a recipient. For instance, in embodiments, the recipient may have a mutation that provides a disease phenotype (e.g., a defective or absent gene product). In embodiments, the donor system or method of the present disclosure provides a correction that restores the gene product and diminishes the disease phenotype.

In embodiments, the transgene is a gene that replaces, inactivates, or provides suicide or helper functions.

In embodiments, the transgene and/or disease to be treated is one or more of:
beta-thalassemia: BCL11a or β-globin or βA-T87Q-globin,
LCA: RPE65,
LHON: ND4,
Achromatopsia: CNGA3 or CNGA3/CNGB3,
Choroideremia: REP1,
PKD: RPK (Red cell PK),
Hemophilia: F8,
ADA-SCID: ADA,
Fabry disease: GLA,
MPS type I: IDUA, and
MPS type II: IDS.

In embodiments, the donor comprises a gene encoding a complete polypeptide. In embodiments, the donor comprises a gene which is defective or substantially absent in a disease state.

In embodiments, the transfecting of the cell is carried out using electroporation or calcium phosphate precipitation.

In embodiments, the transfecting of the cell is carried out using a lipid vehicle, optionally N-[1-(2,3-dioleoyloxy)propyl]-N,N,N-trimethylammonium chloride (DOTMA), 1,2-bis(oleoyloxy)-3-3-(trimethylammonia) propane (DOTAP), or 1,2-dioleoyl-3-dimethylammonium-propane (DODAP), dioleoylphosphatidylethanolamine (DOPE), cholesterol, LIPOFECTIN (cationic liposome formulation), LIPOFECTAMINE (cationic liposome formulation), LIPOFECTAMINE 2000 (cationic liposome formulation), LIPOFECTAMINE 3000 (cationic liposome formulation), TRANSFECTAM (cationic liposome formulation), a lipid nanoparticle, or a liposome and combinations thereof.

In embodiments, the transfecting of the cell is carried out using a lipid selected from one or more of the following categories: cationic lipids; anionic lipids; neutral lipids; multi-valent charged lipids; and zwitterionic lipids. In embodiments, a cationic lipid may be used to facilitate a charge-charge interaction with nucleic acids. In embodiments, the lipid is a neutral lipid. In embodiments, the neutral lipid is dioleoylphosphatidylethanolamine (DOPE), 1,2-Dioleoyl-sn-glycero-3-phosphocholine (DOPC), or cholesterol. In embodiments, cholesterol is derived from plant sources. In other embodiments, cholesterol is derived from animal, fungal, bacterial, or archaeal sources. In embodiments, the lipid is a cationic lipid. In embodiments, the cationic lipid is N-[1-(2,3-dioleoyloxy)propyl]-N,N,N-trimethylammonium chloride (DOTMA), 1,2-bis(oleoyloxy)-3-3-(trimethylammonia) propane (DOTAP), or 1,2-dioleoyl-3-dimethylammonium-propane (DODAP). In embodiments, one or more of the phospholipids 18:0 PC, 18:1 PC, 18:2 PC, DMPC, DSPE, DOPE, 18:2 PE, DMPE, or a combination thereof are used as lipids. In embodiments, the lipid is DOTMA and DOPE, optionally in a ratio of about 1:1. In embodiments, the lipid is DHDOS and DOPE, optionally in a ratio of about 1:1. In embodiments, the lipid is a commercially available product (e.g., LIPOFECTIN (cationic liposome formulation), LIPOFECTAMINE (cationic liposome formulation), LIPOFECTAMINE 2000 (cationic liposome formulation), LIPOFECTAMINE 3000 (cationic liposome formulation) (Life Technologies)).

In embodiments, the transfecting of the cell is carried out using a cationic vehicle, optionally LIPOFECTIN or TRANSFECTAM.

In embodiments, the transfecting of the cell is carried out using a lipid nanoparticle or a liposome.

In embodiments, the method is helper virus-free.

Epigenetic regulatory elements can be used to protect a transgene from unwanted epigenetic effects when placed near the transgene on a vector, including the transgene. See Ley et al., PloS One vol. 8,4 e62784. 30 Apr. 2013, doi:10.1371/journal.pone.0062784. For example, MARs were shown to increase genomic integration and integration of a transgene while preventing heterochromatin silencing, as exemplified by the human MAR 1-68. See id.; see also Grandjean et al., Nucleic Acids Res. 2011 August; 39(15):e104. MARs can also act as insulators and thereby prevent the activation of neighboring cellular genes. Gaussin et al., Gene Ther. 2012 January; 19(1):15-24. It has been shown that a piggyBac donor containing human MARs in CHO cells mediated efficient and sustained expression from a few transgene copies, using cell populations generated without an antibiotic selection procedure. See Ley et al. (2013).

In embodiments, the cell is further transfected with a third nucleic acid having at least one chromatin element, wherein the at least one chromatin element is optionally a Matrix Attachment Region (MAR) element. MARs are expression-enhancing, epigenetic regulator elements which are used to enhance and/or facilitate transgene expression, as described, for example, in PCT/IB2010/002337 (WO2011033375), which is incorporated by reference herein in its entirety. A MAR element can be located in cis or trans to the transgene.

In embodiments, the transgene has a size of 100,000 bases or less, e.g., about 100,000 bases, or about 50,000 bases, or about 30,000 bases, or about 10,000 bases, or about 5,000 bases, or about 10,000 to about 100,000 bases, or about 30,000 to about 100,000 bases, or about 50,000 to about 100,000 bases, or about 10,000 to about 50,000 bases, or about 10,000 to about 30,000 bases, or about 30,000 to about 50,000 bases.

In embodiments, the transgene has a size of about 200,000 bases or less, e.g., about 200,000 bases, or about 10,000 to about 200,000 bases, or about 30,000 to about 200,000 bases, or about 50,000 to about 200,000 bases, or about 100,000 to about 200,000 bases, or about 150,000 to about 200,000 bases.

### Targeting Chimeric Constructs

In aspects, the present disclosure provides for a donor system, e.g., in embodiments, a helper enzyme comprises a targeting element.

In embodiments, the helper enzyme associated with the targeting element, is capable of inserting the donor comprising a transgene, optionally at a TA dinucleotide site or a TTAA (SEQ ID NO: 440) tetranucleotide site in a genomic safe harbor site (GSHS).

In embodiments, the helper enzyme associated with the targeting element has one or more mutations which confer hyperactivity.

In embodiments, the helper enzyme associated with the targeting element has gene cleavage (Exc) and/or gene integration (Int+) activity.

In embodiments, the helper enzyme associated with the targeting element has gene cleavage (Exc) and/or a lack of gene integration (Int−) activity.

In embodiments, the targeting element comprises one or more proteins or nucleic acids that are capable of binding to a nucleic acid.

In embodiments, the targeting element comprises one or more of a of a gRNA, optionally associated with a Cas enzyme, which is optionally catalytically inactive, transcription activator-like effector (TALE), Zinc finger, catalytically inactive transcription factor, nickase, a transcriptional activator, a transcriptional repressor, a recombinase, a DNA methyltransferase, a histone methyltransferase, and paternally expressed gene 10 (PEG10).

In embodiments, the targeting element comprises a transcription activator-like effector (TALE) DNA binding domain (DBD).

In embodiments, the TALE DBD comprises one or more repeat sequences. In embodiments, the TALE DBD comprises about 14, or about 15, or about, 16, or about 17, or about 18, or about 18.5 repeat sequences. In embodiments, the TALE DBD repeat sequences comprise 33 or 34 amino acids. In embodiments, the TALE DBD repeat sequences comprise a repeat variable di-residue (RVD) at residue 12 or 13 of the 33 or 34 amino acids. In embodiments, the RVD recognizes one base pair in the nucleic acid molecule. In embodiments, the RVD recognizes a C residue in the nucleic acid molecule and is selected from HD, N(gap), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from NI and NS. In embodiments, the RVD recognizes a T residue in the nucleic acid molecule and is selected from NG, HG, H(gap), and IG. In embodiments, the GSHS is in an open chromatin location in a chromosome. In embodiments, the GSHS is selected from adeno-associated virus site 1 (AAVS1), chemokine (C-C motif) receptor 5 (CCR5) gene, HIV-1 coreceptor, and human Rosa26 locus. In embodiments, the GSHS is located on human chromosome 2, 4, 6, 10, 11, or 17. In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the targeting element comprises a Cas9 enzyme guide RNA complex. In embodiments, the Cas9 enzyme guide RNA complex comprises a nuclease-deficient dCas9 guide RNA complex. In embodiments, the targeting element comprises a Cas12 enzyme guide RNA complex. In embodiments, the targeting element comprises a nuclease-deficient dCas12 guide RNA complex, optionally dCas12j guide RNA complex or dCas12a guide RNA complex. In embodiments, the targeting element comprises a Cas12k enzyme guide RNA complex. In embodiments, the targeting element comprises a nuclease-deficient dCas12 guide RNA complex, optionally dCas12k guide RNA complex.

In embodiments, a targeting chimeric system or construct, having a DBD fused to the helper enzyme directs binding of the helper to a specific sequence (e.g., transcription activator-like effector proteins (TALE) repeat variable di-residues (RVD) or gRNA) near a helper enzyme recognition site. The helper enzyme is thus prevented from binding to random recognition sites. In embodiments, the targeting chimeric construct binds to human GSHS. In embodiments, dCas9 (i.e., deficient for nuclease activity) is programmed with gRNAs directed to bind at a desired sequence of DNA in GSHS.

In embodiments, TALEs described herein can physically sequester the helper enzyme to GSHS and promote transposition to nearby TTAA (SEQ ID NO: 440) sequences in close proximity to the RVD TALE nucleotide sequences. GSHS in open chromatin sites are specifically targeted based on the predilection for helpers to insert into open chromatin.

In embodiments, the helper enzyme is capable of targeted genomic integration by transposition is linked to or fused with a TALE DNA binding domain (DBD) or a Cas-based gene-editing system, such as, e.g., Cas9 or a variant thereof.

In embodiments, the targeting element targets the helper enzyme to a locus of interest. In embodiments, the targeting element comprises CRISPR (Clustered Regularly Interspaced Short Palindromic Repeat) associated protein 9 (Cas9), or a variant thereof. A CRISPR/Cas9 tool only requires Cas9 nuclease for DNA cleavage and a single-guide RNA (sgRNA) for target specificity. See Jinek et al. (2012) Science 337, 816-821; Chylinski et al. (2014) Nucleic Acids Res 42, 6091-6105. The inactivated form of Cas9, which is a nuclease-deficient (or inactive, or “catalytically dead” Cas9, is typically denoted as “dCas9,” has no substantial nuclease activity. Qi, L. S. et al. (2013). Cell 152, 1173-1183. CRISPR/dCas9 binds precisely to specific genomic sequences through targeting of guide RNA (gRNA) sequences. See Dominguez et al., Nat Rev Mol Cell Biol. 2016; 17:5-15; Wang et al., Annu Rev Biochem. 2016; 85:227-64. dCas9 is utilized to edit gene expression when applied to the transcription binding site of a desired site and/or locus in a genome. When the dCas9 protein is coupled to guide RNA (gRNA) to create dCas9 guide RNA complex, dCas9 prevents the proliferation of repeating codons and DNA sequences that might be harmful to an organism's genome. Essentially, when multiple repeat codons are produced, it elicits a response, or recruits an abundance of dCas9 to combat the overproduction of those codons and results in the shut-down of transcription. Thus, dCas9 works synergistically with gRNA and directly affects the DNA polymerase II from continuing transcription.

In embodiments, the targeting element comprises a nuclease-deficient Cas enzyme guide RNA complex. In embodiments, the targeting element comprises a nuclease-deficient (or inactive, or “catalytically dead” Cas, e.g., Cas9, typically denoted as “dCas” or “dCas9”) guide RNA complex.

In embodiments, the dCas9/gRNA complex comprises a guide RNA selected from: GTTTAGCTCACCCGTGAGCC (SEQ ID NO: 91), CCCAATATTATTGTTCTCTG (SEQ ID NO: 92), GGGGTGGGATAGGGGATACG (SEQ ID NO: 93), GGATCCCCCTCTACATTTAA (SEQ ID NO: 94), GTGATCTTGTACAAATCATT (SEQ ID NO: 95), CTACACAGAATCTGTTAGAA (SEQ ID NO: 96), TAAGCTAGAGAATAGATCTC (SEQ ID NO: 97), and TCAATACACTTAATGATTTA (SEQ ID NO: 98), wherein the guide RNA directs the helper enzyme to a chemokine (C-C motif) receptor 5 (CCR5) gene.

In embodiments, the dCas9/gRNA complex comprises a guide RNA selected from:

(SEQ ID NO: 99)
CACCGGGAGCCACGAAAACAGATCC;
(SEQ ID NO: 100)
CACCGCGAAAACAGATCCAGGGACA;
(SEQ ID NO: 101)
CACCGAGATCCAGGGACACGGTGCT;
(SEQ ID NO: 102)
CACCGGACACGGTGCTAGGACAGTG;
(SEQ ID NO: 103)
CACCGGAAAATGACCCAACAGCCTC;
(SEQ ID NO: 104)
CACCGGCCTGGCCGGCCTGACCACT;
(SEQ ID NO: 105)
CACCGCTGAGCACTGAAGGCCTGGC;
(SEQ ID NO: 106)
CACCGTGGTTTCCACTGAGCACTGA;
(SEQ ID NO: 107)
CACCGGATAGCCAGGAGTCCTTTCG;
(SEQ ID NO: 108)
CACCGGCGCTTCCAGTGCTCAGACT;
(SEQ ID NO: 109)
CACCGCAGTGCTCAGACTAGGGAAG;
(SEQ ID NO: 110)
CACCGGCCCCTCCTCCTTCAGAGCC;
(SEQ ID NO: 111)
CACCGTCCTTCAGAGCCAGGAGTCC;
(SEQ ID NO: 112)
CACCGTGGTTTCCGAGCTTGACCCT;
(SEQ ID NO: 113)
CACCGCTGCAGAGTATCTGCTGGGG;
(SEQ ID NO: 114)
CACCGCGTTCCTGCAGAGTATCTGC;
(SEQ ID NO: 115)
AAACGGATCTGTTTTCGTGGCTCCC;
(SEQ ID NO: 116)
AAACTGTCCCTGGATCTGTTTTCGC;
(SEQ ID NO: 117)
AAACAGCACCGTGTCCCTGGATCTC;
(SEQ ID NO: 118)
AAACCACTGTCCTAGCACCGTGTCC;
(SEQ ID NO: 119)
AAACGAGGCTGTTGGGTCATTTTCC;
(SEQ ID NO: 120)
AAACAGTGGTCAGGCCGGCCAGGCC;
(SEQ ID NO: 121)
AAACGCCAGGCCTTCAGTGCTCAGC;
(SEQ ID NO: 122)
AAACTCAGTGCTCAGTGGAAACCAC;
(SEQ ID NO: 123)
AAACCGAAAGGACTCCTGGCTATCC;
(SEQ ID NO: 124)
AAACAGTCTGAGCACTGGAAGCGCC;
(SEQ ID NO: 125)
AAACCTTCCCTAGTCTGAGCACTGC;
(SEQ ID NO: 126)
AAACGGCTCTGAAGGAGGAGGGGCC;
(SEQ ID NO: 127)
AAACGGACTCCTGGCTCTGAAGGAC;
(SEQ ID NO: 128)
AAACAGGGTCAAGCTCGGAAACCAC;
(SEQ ID NO: 129)
AAACCCCCAGCAGATACTCTGCAGC;
(SEQ ID NO: 130)
AAACGCAGATACTCTGCAGGAACGC;
(SEQ ID NO: 131)
TCCCCTCCCAGAAAGACCTG;
(SEQ ID NO: 132)
TGGGCTCCAAGCAATCCTGG;
(SEQ ID NO: 133)
GTGGCTCAGGAGGTACCTGG;
(SEQ ID NO: 134)
GAGCCACGAAAACAGATCCA;
(SEQ ID NO: 135)
AAGTGAACGGGGAAGGGAGG;
(SEQ ID NO: 136)
GACAAAAGCCGAAGTCCAGG;
(SEQ ID NO: 137)
GTGGTTGATAAACCCACGTG;
(SEQ ID NO: 138)
TGGGAACAGCCACAGCAGGG;
(SEQ ID NO: 139)
GCAGGGGAACGGGGATGCAG;
(SEQ ID NO: 140)
GAGATGGTGGACGAGGAAGG;
(SEQ ID NO: 141)
GAGATGGCTCCAGGAAATGG;
(SEQ ID NO: 142)
TAAGGAATCTGCCTAACAGG;
(SEQ ID NO: 143)
TCAGGAGACTAGGAAGGAGG;
(SEQ ID NO: 144)
TATAAGGTGGTCCCAGCTCG;
(SEQ ID NO: 145)
CTGGAAGATGCCATGACAGG;
(SEQ ID NO: 146)
GCACAGACTAGAGAGGTAAG;
(SEQ ID NO: 147)
ACAGACTAGAGAGGTAAGGG;
(SEQ ID NO: 148)
GAGAGGTGACCCGAATCCAC;
(SEQ ID NO: 149)
GCACAGGCCCCAGAAGGAGA;
(SEQ ID NO: 150)
CCGGAGAGGACCCAGACACG;
(SEQ ID NO: 151)
GAGAGGACCCAGACACGGGG;
(SEQ ID NO: 152)
GCAACACAGCAGAGAGCAAG;
(SEQ ID NO: 153)
GAAGAGGGAGTGGAGGAAGA;
(SEQ ID NO: 154)
AAGACGGAACCTGAAGGAGG;
(SEQ ID NO: 155)
AGAAAGCGGCACAGGCCCAG;
(SEQ ID NO: 156)
GGGAAACAGTGGGCCAGAGG;
(SEQ ID NO: 157)
GTCCGGACTCAGGAGAGAGA;
(SEQ ID NO: 158)
GGCACAGCAAGGGCACTCGG;
(SEQ ID NO: 159)
GAAGAGGGGAAGTCGAGGGA;
(SEQ ID NO: 160)
GGGAATGGTAAGGAGGCCTG;
(SEQ ID NO: 161)
GCAGAGTGGTCAGCACAGAG;
(SEQ ID NO: 162)
GCACAGAGTGGCTAAGCCCA;
(SEQ ID NO: 163)
GACGGGGTGTCAGCATAGGG;
(SEQ ID NO: 164)
GCCCAGGGCCAGGAACGACG;
(SEQ ID NO: 165)
GGTGGAGTCCAGCACGGCGC;
(SEQ ID NO: 166)
ACAGGCCGCCAGGAACTCGG;
(SEQ ID NO: 167)
ACTAGGAAGTGTGTAGCACC;
(SEQ ID NO: 168)
ATGAATAGCAGACTGCCCCG;
(SEQ ID NO: 169)
ACACCCCTAAAAGCACAGTG;
(SEQ ID NO: 170)
CAAGGAGTTCCAGCAGGTGG;
(SEQ ID NO: 171)
AAGGAGTTCCAGCAGGTGGG;
(SEQ ID NO: 172)
TGGAAAGAGGAGGGAAGAGG;
(SEQ ID NO: 173)
TCGAATTCCTAACTGCCCCG;
(SEQ ID NO: 174)
GACCTGCCCAGCACACCCTG;
(SEQ ID NO: 175)
GGAGCAGCTGCGGCAGTGGG;
(SEQ ID NO: 176)
GGGAGGGAGAGCTTGGCAGG;
(SEQ ID NO: 177)
GTTACGTGGCCAAGAAGCAG;
(SEQ ID NO: 178)
GCTGAACAGAGAAGAGCTGG;
(SEQ ID NO: 179)
TCTGAGGGTGGAGGGACTGG;
(SEQ ID NO: 180)
GGAGAGGTGAGGGACTTGGG;
(SEQ ID NO: 181)
GTGAACCAGGCAGACAACGA;
(SEQ ID NO: 182)
CAGGTACCTCCTGAGCCACG;
(SEQ ID NO: 183)
GGGGGAGTAGGGGCATGCAG;
(SEQ ID NO: 184)
GCAAATGGCCAGCAAGGGTG;
(SEQ ID NO: 309)
CAAATGGCCAGCAAGGGTGG;
(SEQ ID NO: 310)
GCAGAACCTGAGGATATGGA;
(SEQ ID NO: 311)
AATACACAGAATGAAAATAG;
(SEQ ID NO: 312)
CTGGTGACTAGAATAGGCAG;
(SEQ ID NO: 313)
TGGTGACTAGAATAGGCAGT;
(SEQ ID NO: 314)
TAAAAGAATGTGAAAAGATG;
(SEQ ID NO: 315)
TCAGGAGTTCAAGACCACCC;
(SEQ ID NO: 316)
TGTAGTCCCAGTTATGCAGG;
(SEQ ID NO: 317)
GGGTTCACACCACAAATGCA;
(SEQ ID NO: 318)
GGCAAATGGCCAGCAAGGGT;
(SEQ ID NO: 319)
AGAAACCAATCCCAAAGCAA;
(SEQ ID NO: 320)
GCCAAGGACACCAAAACCCA;
(SEQ ID NO: 321)
AGTGGTGATAAGGCAACAGT;
(SEQ ID NO: 322)
CCTGAGACAGAAGTATTAAG;
(SEQ ID NO: 323)
AAGGTCACACAATGAATAGG;
(SEQ ID NO: 324)
CACCATACTAGGGAAGAAGA;
(SEQ ID NO: 327)
CAATACCCTGCCCTTAGTGG;
(SEQ ID NO: 325)
AATACCCTGCCCTTAGTGGG;
(SEQ ID NO: 326)
TTAGTGGGGGGTGGAGTGGG;
(SEQ ID NO: 328)
GTGGGGGGTGGAGTGGGGGG;
(SEQ ID NO: 329)
GGGGGGTGGAGTGGGGGGTG;
(SEQ ID NO: 330)
GGGGTGGAGTGGGGGGTGGG;
(SEQ ID NO: 331)
GGGTGGAGTGGGGGGTGGGG;
(SEQ ID NO: 332)
GGGGGGGGGAAAGACATCG;
(SEQ ID NO: 333)
GCAGCTGTGAATTCTGATAG;
(SEQ ID NO: 334)
GAGATCAGAGAAACCAGATG;
(SEQ ID NO: 335)
TCTATACTGATTGCAGCCAG;
(SEQ ID NO: 185)
CACCGAATCGAGAAGCGACTCGACA;
(SEQ ID NO: 186)
CACCGGTCCCTGGGCGTTGCCCTGC;
(SEQ ID NO: 187)
CACCGCCCTGGGCGTTGCCCTGCAG;
(SEQ ID NO: 188)
CACCGCCGTGGGAAGATAAACTAAT;
(SEQ ID NO: 189)
CACCGTCCCCTGCAGGGCAACGCCC;
(SEQ ID NO: 190)
CACCGGTCGAGTCGCTTCTCGATTA;
(SEQ ID NO: 191)
CACCGCTGCTGCCTCCCGTCTTGTA;
(SEQ ID NO: 192)
CACCGGAGTGCCGCAATACCTTTAT;
(SEQ ID NO: 193)
CACCGACACTTTGGTGGTGCAGCAA;
(SEQ ID NO: 194)
CACCGTCTCAAATGGTATAAAACTC;
(SEQ ID NO: 195)
CACCGAATCCCGCCCATAATCGAGA;
(SEQ ID NO: 196)
CACCGTCCCGCCCATAATCGAGAAG;
(SEQ ID NO: 197)
CACCGCCCATAATCGAGAAGCGACT;
(SEQ ID NO: 198)
CACCGGAGAAGCGACTCGACATGGA;
(SEQ ID NO: 199)
CACCGGAAGCGACTCGACATGGAGG;
(SEQ ID NO: 200)
CACCGGCGACTCGACATGGAGGCGA;
(SEQ ID NO: 201)
AAACTGTCGAGTCGCTTCTCGATTC;
(SEQ ID NO: 202)
AAACGCAGGGCAACGCCCAGGGACC;
(SEQ ID NO: 203)
AAACCTGCAGGGCAACGCCCAGGGC;
(SEQ ID NO: 204)
AAACATTAGTTTATCTTCCCACGGC;
(SEQ ID NO: 205)
AAACGGGCGTTGCCCTGCAGGGGAC;
(SEQ ID NO: 206)
AAACTAATCGAGAAGCGACTCGACC;
(SEQ ID NO: 207)
AAACTACAAGACGGGAGGCAGCAGC;
(SEQ ID NO: 208)
AAACATAAAGGTATTGCGGCACTCC;
(SEQ ID NO: 209)
AAACTTGCTGCACCACCAAAGTGTC;
(SEQ ID NO: 210)
AAACGAGTTTTATACCATTTGAGAC;
(SEQ ID NO: 211)
AAACTCTCGATTATGGGCGGGATTC;
(SEQ ID NO: 212)
AAACCTTCTCGATTATGGGGGGGAC;
(SEQ ID NO: 213)
AAACAGTCGCTTCTCGATTATGGGC;
(SEQ ID NO: 214)
AAACTCCATGTCGAGTCGCTTCTCC;
(SEQ ID NO: 215)
AAACCCTCCATGTCGAGTCGCTTCC;
(SEQ ID NO: 216)
AAACTCGCCTCCATGTCGAGTCGCC;
(SEQ ID NO: 217)
CACCGACAGGGTTAATGTGAAGTCC;
(SEQ ID NO: 218)
CACCGTCCCCCTCTACATTTAAAGT;
(SEQ ID NO: 219)
CACCGCATTTAAAGTTGGTTTAAGT;
(SEQ ID NO: 220)
CACCGTTAGAAAATATAAAGAATAA;
(SEQ ID NO: 221)
CACCGTAAATGCTTACTGGTTTGAA;
(SEQ ID NO: 222)
CACCGTCCTGGGTCCAGAAAAAGAT;
(SEQ ID NO: 223)
CACCGTTGGGTGGTGAGCATCTGTG;
(SEQ ID NO: 224)
CACCGCGGGGAGAGTGGAGAAAAAG;
(SEQ ID NO: 225)
CACCGGTTAAAACTCTTTAGACAAC;
(SEQ ID NO: 226)
CACCGGAAAATCCCCACTAAGATCC;
(SEQ ID NO: 227)
AAACGGACTTCACATTAACCCTGTC;
(SEQ ID NO: 228)
AAACACTTTAAATGTAGAGGGGGAC;
(SEQ ID NO: 229)
AAACACTTAAACCAACTTTAAATGC;
(SEQ ID NO: 230)
AAACTTATTCTTTATATTTTCTAAC;
(SEQ ID NO: 231)
AAACTTCAAACCAGTAAGCATTTAC;
(SEQ ID NO: 232)
AAACATCTTTTTCTGGACCCAGGAC;
(SEQ ID NO: 233)
AAACCACAGATGCTCACCACCCAAC;
(SEQ ID NO: 234)
AAACCTTTTTCTCCACTCTCCCCGC;
(SEQ ID NO: 235)
AAACGTTGTCTAAAGAGTTTTAACC;
(SEQ ID NO: 236)
AAACGGATCTTAGTGGGGATTTTCC;
(SEQ ID NO: 237)
AGTAGCAGTAATGAAGCTGG;
(SEQ ID NO: 238)
ATACCCAGACGAGAAAGCTG;
(SEQ ID NO: 239)
TACCCAGACGAGAAAGCTGA;
(SEQ ID NO: 240)
GGTGGTGAGCATCTGTGTGG;
(SEQ ID NO: 241)
AAATGAGAAGAAGAGGCACA;
(SEQ ID NO: 242)
CTTGTGGCCTGGGAGAGCTG;
(SEQ ID NO: 243)
GCTGTAGAAGGAGACAGAGC;
(SEQ ID NO: 244)
GAGCTGGTTGGGAAGACATG;
(SEQ ID NO: 245)
CTGGTTGGGAAGACATGGGG;
(SEQ ID NO: 246)
CGTGAGGATGGGAAGGAGGG;
(SEQ ID NO: 247)
ATGCAGAGTCAGCAGAACTG;
(SEQ ID NO: 248)
AAGACATCAAGCACAGAAGG;
(SEQ ID NO: 249)
TCAAGCACAGAAGGAGGAGG;
(SEQ ID NO: 250)
AACCGTCAATAGGCAAAGGG;
(SEQ ID NO: 251)
CCGTATTCAGACTGAATGG;
(SEQ ID NO: 252)
GAGAGGACAGGTGCTACAGG;
(SEQ ID NO: 253)
AACCAAGGAAGGGCAGGAGG;
(SEQ ID NO: 254)
GACCTCTGGGTGGAGACAGA;
(SEQ ID NO: 255)
CAGATGACCATGACAAGCAG;
(SEQ ID NO: 256)
AACACCAGTGAGTAGAGCGG;
(SEQ ID NO: 257)
AGGACCTTGAAGCACAGAGA;
(SEQ ID NO: 258)
TACAGAGGCAGACTAACCCA;
(SEQ ID NO: 259)
ACAGAGGCAGACTAACCCAG;
(SEQ ID NO: 260)
TAAATGACGTGCTAGACCTG;
(SEQ ID NO: 261)
AGTAACCACTCAGGACAGGG;
(SEQ ID NO: 262)
ACCACAAAACAGAAACACCA;
(SEQ ID NO: 263)
GTTTGAAGACAAGCCTGAGG;
(SEQ ID NO: 264)
GCTGAACCCCAAAAGACAGG;
(SEQ ID NO: 265)
GCAGCTGAGACACACACCAG;
(SEQ ID NO: 266)
AGGACACCCCAAAGAAGCTG;
(SEQ ID NO: 267)
GGACACCCCAAAGAAGCTGA;
(SEQ ID NO: 268)
CCAGTGCAATGGACAGAAGA;
(SEQ ID NO: 269)
AGAAGAGGGAGCCTGCAAGT;
(SEQ ID NO: 270)
GTGTTTGGGCCCTAGAGCGA;
(SEQ ID NO: 271)
CATGTGCCTGGTGCAATGCA;
(SEQ ID NO: 272)
TACAAAGAGGAAGATAAGTG;
(SEQ ID NO: 273)
GTCACAGAATACACCACTAG;
(SEQ ID NO: 274)
GGGTTACCCTGGACATGGAA;
(SEQ ID NO: 275)
CATGGAAGGGTATTCACTCG;
(SEQ ID NO: 276)
AGAGTGGCCTAGACAGGCTG;
(SEQ ID NO: 277)
CATGCTGGACAGCTCGGCAG;
(SEQ ID NO: 278)
AGTGAAAGAAGAGAAAATTC;
(SEQ ID NO: 279)
TGGTAAGTCTAAGAAACCTA;
(SEQ ID NO: 280)
CCCACAGCCTAACCACCCTA;
(SEQ ID NO: 281)
AATATTTCAAAGCCCTAGGG;
(SEQ ID NO: 282)
GCACTCGGAACAGGGTCTGG;
(SEQ ID NO: 283)
AGATAGGAGCTCCAACAGTG;
(SEQ ID NO: 284)
AAGTTAGAGCAGCCAGGAAA;
(SEQ ID NO: 285)
TAGAGCAGCCAGGAAAGGGA;
(SEQ ID NO: 286)
TGAATACCCTTCCATGTCCA;
(SEQ ID NO: 287)
CCTGCATTGCACCAGGCACA;
(SEQ ID NO: 288)
TCTAGGGCCCAAACACACCT;
(SEQ ID NO: 289)
TCCCTCCATCTATCAAAAGG;
(SEQ ID NO: 290)
AGCCCTGAGACAGAAGCAGG;
(SEQ ID NO: 291)
GCCCTGAGACAGAAGCAGGT;
(SEQ ID NO: 292)
AGGAGATGCAGTGATACGCA;
(SEQ ID NO: 293)
ACAATACCAAGGGTATCCGG;
(SEQ ID NO: 294)
TGATAAAGAAAACAAAGTGA;
(SEQ ID NO: 295)
AAAGAAAACAAAGTGAGGGA;
(SEQ ID NO: 296)
GTGGCAAGTGGAGAAATTGA;
(SEQ ID NO: 297)
CAAGTGGAGAAATTGAGGGA;
(SEQ ID NO: 298)
GTGGTGATGATTGCAGCTGG;
(SEQ ID NO: 299)
CTATGTGCCTGACACACAGG;
(SEQ ID NO: 300)
GGGTTGGACCAGGAAAGAGG;
(SEQ ID NO: 301)
GATGCCTGGAAAAGGAAAGA;
(SEQ ID NO: 302)
TAGTATGCACCTGCAAGAGG;
(SEQ ID NO: 303)
TATGCACCTGCAAGAGGGGG;
(SEQ ID NO: 304)
AGGGGAAGAAGAGAAGCAGA;
(SEQ ID NO: 305)
GCTGAATCAAGAGACAAGCG;
(SEQ ID NO: 306)
AAGCAAATAAATCTCCTGGG;
(SEQ ID NO: 307)
AGATGAGTGCTAGAGACTGG;
and
(SEQ ID NO: 308)
CTGATGGTTGAGCACAGCAG.

In embodiments, the guide RNAs are: AATCGAGAAGCGACTCGACA (SEQ ID NO: 425), and tgccctgcaggggagtgagc (SEQ ID NO: 426). In embodiments, the guide RNAs are gaagcgactcgacatggagg (SEQ ID NO: 427) and cctgcaggggagtgagcagc (SEQ ID NO: 428).

In embodiments, guide RNAs (gRNAs) for targeting human genomic safe harbor sites using any of the gRNA-based targeting elements, e.g., without limitation dCas, in areas of open chromatin are as shown in TABLE 19.

TABLE 19

| GSHS | Identifier | Sequence |
|---|---|---|
| AAVS1 | 14F | ggagccacgaaaacagatcc |
| | | (SEQ ID NO: 800) |
| AAVS1 | 15F | cgaaaacagatccagggaca |
| | | (SEQ ID NO: 801) |
| AAVS1 | 16F | agatccagggacacggtgct |
| | | (SEQ ID NO: 802) |
| AAVS1 | 17F | gacacggtgctaggacagtg |
| | | (SEQ ID NO: 803) |
| AAVS1 | 18F | gaaaatgacccaacagcctc |
| | | (SEQ ID NO: 804) |
| AAVS1 | 19F | gcctggccggcctgaccact |
| | | (SEQ ID NO: 805) |
| AAVS1 | 20F | ctgagcactgaaggcctggc |
| | | (SEQ ID NO: 806) |
| AAVS1 | 21F | tggtttccactgagcactga |
| | | (SEQ ID NO: 807) |
| AAVS1 | 22F | gatagccaggagtcctttcg |
| | | (SEQ ID NO: 808) |
| AAVS1 | 23F | gcgcttccagtgctcagact |
| | | (SEQ ID NO: 809) |
| AAVS1 | 24F | cagtgctcagactagggaag |
| | | (SEQ ID NO: 810) |
| AAVS1 | 25F | gcccctcctccttcagagcc |
| | | (SEQ ID NO: 811) |
| AAVS1 | 26F | tccttcagagccaggagtcc |
| | | (SEQ ID NO: 812) |
| AAVS1 | 27F | tggtttccgagcttgaccct |
| | | (SEQ ID NO: 813) |
| AAVS1 | 28F | ctgcagagtatctgctgggg |
| | | (SEQ ID NO: 814) |
| AAVS1 | 29F | cgttcctgcagagtatctgc |
| | | (SEQ ID NO: 815) |
| AAVS1 | AAVS1 | tcccctcccagaaagacctg |
| | | (SEQ ID NO: 131) |
| AAVS1 | gAAVS2 | tgggctccaagcaatcctgg |
| | | (SEQ ID NO: 132) |
| AAVS1 | gAAVS3 | gtggctcaggaggtacctgg |
| | | (SEQ ID NO: 133) |
| AAVS1 | gAAVS4 | gagccacgaaaacagatcca |
| | | (SEQ ID NO: 134) |
| AAVS1 | gAAVS5 | aagtgaacggggaagggagg |
| | | (SEQ ID NO: 135) |
| AAVS1 | gAAVS6 | gacaaaagccgaagtccagg |
| | | (SEQ ID NO: 136) |
| AAVS1 | gAAVS7 | gtggttgataaacccacgtg |
| | | (SEQ ID NO: 137) |
| AAVS1 | gAAVS8 | tgggaacagccacagcaggg |
| | | (SEQ ID NO: 138) |
| AAVS1 | gAAVS9 | gcaggggaacggggatgcag |
| | | (SEQ ID NO: 139) |
| AAVS1 | gAAVS10 | gagatggtggacgaggaagg |
| | | (SEQ ID NO: 140) |
| AAVS1 | gAAVS11 | gagatggctccaggaaatgg |
| | | (SEQ ID NO: 141) |
| AAVS1 | gAAVS12 | taaggaatctgcctaacagg |
| | | (SEQ ID NO: 142) |
| AAVS1 | gAAVS13 | tcaggagactaggaaggagg |
| | | (SEQ ID NO: 143) |
| AAVS1 | gAAVS14 | tataaggtggtcccagctcg |
| | | (SEQ ID NO: 144) |
| AAVS1 | gAAVS15 | ctggaagatgccatgacagg |
| | | (SEQ ID NO: 145) |
| AAVS1 | gAAVS16 | gcacagactagagaggtaag |
| | | (SEQ ID NO: 146) |
| AAVS1 | gAAVS17 | acagactagagaggtaaggg |
| | | (SEQ ID NO: 147) |
| AAVS1 | gAAVS18 | gagaggtgacccgaatccac |
| | | (SEQ ID NO: 148) |
| AAVS1 | gAAVS19 | gcacaggccccagaaggaga |
| | | (SEQ ID NO: 149) |
| AAVS1 | gAAVS20 | ccggagaggacccagacacg |
| | | (SEQ ID NO: 150) |
| AAVS1 | gAAVS21 | gagaggacccagacacgggg |
| | | (SEQ ID NO: 151) |
| AAVS1 | gAAVS22 | gcaacacagcagagagcaag |
| | | (SEQ ID NO: 152) |
| AAVS1 | gAAVS23 | gaagagggagtggaggaaga |
| | | (SEQ ID NO: 153) |
| AAVS1 | gAAVS24 | aagacggaacctgaaggagg |
| | | (SEQ ID NO: 154) |
| AAVS1 | gAAVS25 | agaaagcggcacaggcccag |
| | | (SEQ ID NO: 155) |
| AAVS1 | gAAVS26 | gggaaacagtgggccagagg |
| | | (SEQ ID NO: 156) |
| AAVS1 | gAAVS27 | gtccggactcaggagagaga |
| | | (SEQ ID NO: 157) |
| AAVS1 | gAAVS28 | ggcacagcaagggcactcgg |
| | | (SEQ ID NO: 158) |
| AAVS1 | gAAVS29 | gaagaggggaagtcgaggga |
| | | (SEQ ID NO: 159) |
| AAVS1 | gAAVS30 | gggaatggtaaggaggcctg |
| | | (SEQ ID NO: 160) |
| AAVS1 | gAAVS31 | gcagagtggtcagcacagag |
| | | (SEQ ID NO: 161) |
| AAVS1 | gAAVS32 | gcacagagtggctaagccca |
| | | (SEQ ID NO: 162) |
| AAVS1 | gAAVS33 | gacggggtgtcagcataggg |
| | | (SEQ ID NO: 163) |
| AAVS1 | gAAVS34 | gcccagggccaggaacgacg |
| | | (SEQ ID NO: 164) |
| AAVS1 | gAAVS35 | ggtggagtccagcacggcgc |
| | | (SEQ ID NO: 165) |
| AAVS1 | gAAVS36 | acaggccgccaggaactcgg |
| | | (SEQ ID NO: 166) |
| AAVS1 | gAAVS37 | actaggaagtgtgtagcacc |
| | | (SEQ ID NO: 167) |
| AAVS1 | gAAVS38 | atgaatagcagactgccccg |
| | | (SEQ ID NO: 168) |
| AAVS1 | gAAVS39 | acacccctaaaagcacagtg |
| | | (SEQ ID NO: 169) |
| AAVS1 | gAAVS40 | caaggagttccagcaggtgg |
| | | (SEQ ID NO: 170) |
| AAVS1 | gAAVS41 | aaggagttccagcaggtggg |
| | | (SEQ ID NO: 171) |
| AAVS1 | gAAVS42 | tggaaagaggagggaagagg |
| | | (SEQ ID NO: 172) |
| AAVS1 | gAAVS43 | tcgaattcctaactgccccg |
| | | (SEQ ID NO: 173) |
| AAVS1 | gAAVS44 | gacctgcccagcacaccctg |
| | | (SEQ ID NO: 174) |
| AAVS1 | gAAVS45 | ggagcagctgcggcagtggg |
| | | (SEQ ID NO: 175) |
| AAVS1 | gAAVS46 | gggagggagagcttggcagg |
| | | (SEQ ID NO: 176) |
| AAVS1 | gAAVS47 | gttacgtggccaagaagcag |
| | | (SEQ ID NO: 177) |
| AAVS1 | gAAVS48 | gctgaacagagaagagctgg |
| | | (SEQ ID NO: 178) |
| AAVS1 | gAAVS49 | tctgagggtggagggactgg |
| | | (SEQ ID NO: 179) |
| AAVS1 | gAAVS50 | ggagaggtgagggacttggg |
| | | (SEQ ID NO: 180) |
| AAVS1 | gAAVS51 | gtgaaccaggcagacaacga |
| | | (SEQ ID NO: 181) |
| AAVS1 | gAAVS52 | caggtacctcctgagccacg |
| | | (SEQ ID NO: 182) |
| AAVS1 | gAAVS53 | gggggagtaggggcatgcag |
| | | (SEQ ID NO: 183) |
| hROSA26 | gHROSA26-1 | gcaaatggccagcaagggtg |
| | | (SEQ ID NO: 184) |
| hROSA26 | gHROSA26-2 | caaatggccagcaagggtgg |
| | | (SEQ ID NO: 309) |
| hROSA26 | gHROSA26-3 | gcagaacctgaggatatgga |
| | | (SEQ ID NO: 310) |
| hROSA26 | gHROSA26-3 | aatacacagaatgaaaatag |
| | | (SEQ ID NO: 311) |
| hROSA26 | gHROSA26-4 | ctggtgactagaataggcag |
| | | (SEQ ID NO: 312) |
| hROSA26 | gHROSA26-5 | tggtgactagaataggcagt |
| | | (SEQ ID NO: 313) |
| hROSA26 | gHROSA26-6 | taaaagaatgtgaaaagatg |
| | | (SEQ ID NO: 314) |
| hROSA26 | gHROSA26-7 | tcaggagttcaagaccaccc |
| | | (SEQ ID NO: 315) |
| hROSA26 | gHROSA26-8 | tgtagtcccagttatgcagg |
| | | (SEQ ID NO: 316) |
| hROSA26 | gHROSA26-9 | gggttcacaccacaaatgca |
| | | (SEQ ID NO: 317) |
| hROSA26 | gHROSA26-10 | ggcaaatggccagcaagggt |
| | | (SEQ ID NO: 318) |
| hROSA26 | gHROSA26-11 | agaaaccaatcccaaagcaa |
| | | (SEQ ID NO: 319) |
| hROSA26 | gHROSA26-12 | gccaaggacaccaaaaccca |
| | | (SEQ ID NO: 320) |
| hROSA26 | gHROSA26-13 | agtggtgataaggcaacagt |
| | | (SEQ ID NO: 321) |
| hROSA26 | gHROSA26-14 | cctgagacagaagtattaag |
| | | (SEQ ID NO: 322) |
| hROSA26 | gHROSA26-15 | aaggtcacacaatgaatagg |
| | | (SEQ ID NO: 323) |
| hROSA26 | gHROSA26-16 | caccatactagggaagaaga |
| | | (SEQ ID NO: 324) |
| hROSA26 | gHROSA26-17 | caataccctgcccttagtgg |
| | | (SEQ ID NO: 327) |
| hROSA26 | gHROSA26-18 | aataccctgcccttagtggg |
| | | (SEQ ID NO: 325) |
| hROSA26 | gHROSA26-19 | ttagtggggggggagtggg |
| | | (SEQ ID NO: 326) |
| hROSA26 | gHROSA26-20 | gtggggggggagtgggggg |
| | | (SEQ ID NO: 328) |
| hROSA26 | gHROSA26-21 | ggggggtggagtggggggtg |
| | | (SEQ ID NO: 329) |
| hROSA26 | gHROSA26-22 | ggggtggagtggggggtggg |
| | | (SEQ ID NO: 330) |
| hROSA26 | gHROSA26-23 | gggtggagtggggggtgggg |
| | | (SEQ ID NO: 331) |
| hROSA26 | gHROSA26-24 | gggggggggaaagacatcg |
| | | (SEQ ID NO: 332) |
| hROSA26 | gHROSA26-25 | gcaaatggccagcaagggtg |
| | | (SEQ ID NO: 184) |
| hROSA26 | gHROSA26-26 | caaatggccagcaagggtgg |
| | | (SEQ ID NO: 309) |
| hROSA26 | gHROSA26-27 | gcagaacctgaggatatgga |
| | | (SEQ ID NO: 310) |
| hROSA26 | gHROSA26-28 | aatacacagaatgaaaatag |
| | | (SEQ ID NO: 311) |
| hROSA26 | gHROSA26-29 | ctggtgactagaataggcag |
| | | (SEQ ID NO: 312) |
| hROSA26 | gHROSA26-30 | tggtgactagaataggcagt |
| | | (SEQ ID NO: 313) |
| hROSA26 | gHROSA26-31 | taaaagaatgtgaaaagatg |
| | | (SEQ ID NO: 314) |
| hROSA26 | gHROSA26-32 | tcaggagttcaagaccaccc |
| | | (SEQ ID NO: 315) |
| hROSA26 | gHROSA26-33 | tgtagtcccagttatgcagg |
| | | (SEQ ID NO: 316) |
| hROSA26 | gHROSA26-34 | gggttcacaccacaaatgca |
| | | (SEQ ID NO: 317) |
| hROSA26 | gHROSA26-35 | ggcaaatggccagcaagggt |
| | | (SEQ ID NO: 318) |
| hROSA26 | gHROSA26-36 | agaaaccaatcccaaagcaa |
| | | (SEQ ID NO: 319) |
| hROSA26 | gHROSA26-37 | gccaaggacaccaaaaccca |
| | | (SEQ ID NO: 320) |
| hROSA26 | gHROSA26-38 | agtggtgataaggcaacagt |
| | | (SEQ ID NO: 321) |
| hROSA26 | gHROSA26-39 | cctgagacagaagtattaag |
| | | (SEQ ID NO: 322) |
| hROSA26 | gHROSA26-40 | aaggtcacacaatgaatagg |
| | | (SEQ ID NO: 323) |
| hROSA26 | gHROSA26-41 | caccatactagggaagaaga |
| | | (SEQ ID NO: 324) |
| hROSA26 | gHROSA26-42 | caataccctgcccttagtgg |
| | | (SEQ ID NO: 327) |
| hROSA26 | gHROSA26-43 | aataccctgcccttagtggg |
| | | (SEQ ID NO: 325) |
| hROSA26 | gHROSA26-44 | ttagtggggggtggagtggg |
| | | (SEQ ID NO: 326) |
| hROSA26 | gHROSA26-45 | gtggggggtggagtgggggg |
| | | (SEQ ID NO: 328) |
| hROSA26 | gHROSA26-46 | ggggggggagtggggggtg |
| | | (SEQ ID NO: 329) |
| hROSA26 | gHROSA26-47 | ggggtggagtggggggtggg |
| | | (SEQ ID NO: 330) |
| hROSA26 | gHROSA26-48 | gggtggagtggggggtgggg |
| | | (SEQ ID NO: 331) |
| hROSA26 | gHROSA26-49 | gggggggggaaagacatcg |
| | | (SEQ ID NO: 332) |
| hROSA26 | gHROSA26-50 | gcagctgtgaattctgatag |
| | | (SEQ ID NO: 333) |
| hROSA26 | gHROSA26-51 | gagatcagagaaaccagatg |
| | | (SEQ ID NO: 334) |
| hROSA26 | gHROSA26-52 | tctatactgattgcagccag |
| | | (SEQ ID NO: 335) |
| hROSA26 | gHROSA26-1 | gcaaatggccagcaagggtg |
| | | (SEQ ID NO: 184) |
| hROSA26 | 44F | AATCGAGAAGCGACTCGACA |
| | | (SEQ ID NO: 185) |
| hROSA26 | 45F | GTCCCTGGGCGTTGCCCTGC |
| | | (SEQ ID NO: 186) |
| hROSA26 | 46F | CCCTGGGCGTTGCCCTGCAG |
| | | (SEQ ID NO: 187) |
| hROSA26 | 1nF | ccgtgggaagataaactaat |
| | | (SEQ ID NO: 188) |
| hROSA26 | 2nF | tcccctgcagggcaacgccc |
| | | (SEQ ID NO: 189) |
| hROSA26 | 3nF | gtcgagtcgcttctcgatta |
| | | (SEQ ID NO: 190) |
| hROSA26 | 4nF | ctgctgcctcccgtcttgta |
| | | (SEQ ID NO: 191) |
| hROSA26 | 5nF | gagtgccgcaatacctttat |
| | | (SEQ ID NO: 192) |
| hROSA26 | 6nF | ACACTTTGGTGGTGCAGCAA |
| | | (SEQ ID NO: 193) |
| hROSA26 | 7nF | TCTCAAATGGTATAAAACTC |
| | | (SEQ ID NO: 194) |
| hROSA26 | 8nF | ccgtgggaagataaactaat |
| | | (SEQ ID NO: 188) |
| hROSA26 | 9F | aatcccgcccataatcgaga |
| | | (SEQ ID NO: 195) |
| hROSA26 | 10F | tcccgcccataatcgagaag |
| | | (SEQ ID NO: 196) |
| hROSA26 | 11F | cccataatcgagaagcgact |
| | | (SEQ ID NO: 197) |
| hROSA26 | 12F | gagaagcgactcgacatgga |
| | | (SEQ ID NO: 198) |
| hROSA26 | 13F | gaagcgactcgacatggagg |
| | | (SEQ ID NO: 199) |
| hROSA26 | 14F | gcgactcgacatggaggcga |
| | | (SEQ ID NO: 200) |
| hROSA26 | 44F | aaacTGTCGAGTCGCTTCTCGATTc |
| | | (SEQ ID NO: 201) |
| hROSA26 | 45F | aaacGCAGGGCAACGCCCAGGGACc |
| | | (SEQ ID NO: 202) |
| hROSA26 | 46F | aaacCTGCAGGGCAACGCCCAGGGc |
| | | (SEQ ID NO: 203) |
| CCR5 | 1F | acagggttaatgtgaagtcc |
| | | (SEQ ID NO: 217) |
| CCR5 | 2F | tccccctctacatttaaagt |
| | | (SEQ ID NO: 218) |
| CCR5 | 3F | catttaaagttggtttaagt |
| | | (SEQ ID NO: 219) |
| CCR5 | 4F | ttagaaaatataaagaataa |
| | | (SEQ ID NO: 220) |
| CCR5 | 5 | TAAATGCTTACTGGTTTGAA |
| | | (SEQ ID NO: 221) |
| CCR5 | 6F | TCCTGGGTCCAGAAAAAGAT |
| | | (SEQ ID NO: 222) |
| CCR5 | 7F | TTGGGTGGTGAGCATCTGTG |
| | | (SEQ ID NO: 223) |
| CCR5 | 8F | CGGGGAGAGTGGAGAAAAAG |
| | | (SEQ ID NO: 224) |
| CCR5 | 9F | GTTAAAACTCTTTAGACAAC |
| | | (SEQ ID NO: 225) |
| CCR5 | 10F | GAAAATCCCCACTAAGATCC |
| | | (SEQ ID NO: 226) |
| CCR5 | gCCR5-1 | agtagcagtaatgaagctgg |
| | | (SEQ ID NO: 237) |
| CCR5 | gCCR5-2 | atacccagacgagaaagctg |
| | | (SEQ ID NO: 238) |
| CCR5 | gCCR5-3 | tacccagacgagaaagctga |
| | | (SEQ ID NO: 239) |
| CCR5 | gCCR5-4 | ggtggtgagcatctgtgtgg |
| | | (SEQ ID NO: 240) |
| CCR5 | gCCR5-5 | aaatgagaagaagaggcaca |
| | | (SEQ ID NO: 241) |
| CCR5 | gCCR5-6 | cttgtggcctgggagagctg |
| | | (SEQ ID NO: 242) |
| CCR5 | gCCR5-7 | gctgtagaaggagacagagc |
| | | (SEQ ID NO: 243) |
| CCR5 | gCCR5-8 | gagctggttgggaagacatg |
| | | (SEQ ID NO: 244) |
| CCR5 | gCCR5-9 | ctggttgggaagacatgggg |
| | | (SEQ ID NO: 245) |
| CCR5 | gCCR5-10 | cgtgaggatgggaaggaggg |
| | | (SEQ ID NO: 246) |
| CCR5 | gCCR5-11 | atgcagagtcagcagaactg |
| | | (SEQ ID NO: 247) |
| CCR5 | gCCR5-12 | aagacatcaagcacagaagg |
| | | (SEQ ID NO: 248) |
| CCR5 | gCCR5-13 | tcaagcacagaaggaggagg |
| | | (SEQ ID NO: 249) |
| CCR5 | gCCR5-14 | aaccgtcaataggcaaaggg |
| | | (SEQ ID NO: 250) |
| CCR5 | gCCR5-15 | ccgtatttcagactgaatgg |
| | | (SEQ ID NO: 251) |
| CCR5 | gCCR5-16 | gagaggacaggtgctacagg |
| | | (SEQ ID NO: 252) |
| CCR5 | gCCR5-17 | aaccaaggaagggcaggagg |
| | | (SEQ ID NO: 253) |
| CCR5 | gCCR5-18 | gacctctgggtggagacaga |
| | | (SEQ ID NO: 254) |
| CCR5 | gCCR5-19 | cagatgaccatgacaagcag |
| | | (SEQ ID NO: 255) |
| CCR5 | gCCR5-20 | aacaccagtgagtagagcgg |
| | | (SEQ ID NO: 256) |
| CCR5 | gCCR5-21 | aggaccttgaagcacagaga |
| | | (SEQ ID NO: 257) |
| CCR5 | gCCR5-22 | tacagaggcagactaaccca |
| | | (SEQ ID NO: 258) |
| CCR5 | gCCR5-23 | acagaggcagactaacccag |
| | | (SEQ ID NO: 259) |
| CCR5 | gCCR5-24 | taaatgacgtgctagacctg |
| | | (SEQ ID NO: 260) |
| CCR5 | gCCR5-25 | agtaaccactcaggacaggg |
| | | (SEQ ID NO: 261) |
| chr2 | gchr2-1 | accacaaaacagaaacacca |
| | | (SEQ ID NO: 262) |
| chr2 | gchr2-2 | gtttgaagacaagcctgagg |
| | | (SEQ ID NO: 263) |
| chr4 | gchr4-1 | gctgaaccccaaaagacagg |
| | | (SEQ ID NO: 264) |
| chr4 | gchr4-2 | gcagctgagacacacaccag |
| | | (SEQ ID NO: 265) |
| chr4 | gchr4-3 | aggacaccccaaagaagctg |
| | | (SEQ ID NO: 266) |
| chr4 | gchr4-4 | ggacaccccaaagaagctga |
| | | (SEQ ID NO: 267) |
| chr6 | gchr6-1 | ccagtgcaatggacagaaga |
| | | (SEQ ID NO: 268) |
| chr6 | gchr6-2 | agaagagggagcctgcaagt |
| | | (SEQ ID NO: 269) |
| chr6 | gchr6-3 | gtgtttgggccctagagcga |
| | | (SEQ ID NO: 270) |
| chr6 | gchr6-4 | catgtgcctggtgcaatgca |
| | | (SEQ ID NO: 271) |
| chr6 | gchr6-5 | tacaaagaggaagataagtg |
| | | (SEQ ID NO: 272) |
| chr6 | gchr6-6 | gtcacagaatacaccactag |
| | | (SEQ ID NO: 273) |
| chr6 | gchr6-7 | gggttaccctggacatggaa |
| | | (SEQ ID NO: 274) |
| chr6 | gchr6-8 | catggaagggtattcactcg |
| | | (SEQ ID NO: 275) |
| chr6 | gchr6-9 | agagtggcctagacaggctg |
| | | (SEQ ID NO: 276) |
| chr6 | gchr6-10 | catgctggacagctcggcag |
| | | (SEQ ID NO: 277) |
| chr6 | gchr6-11 | agtgaaagaagagaaaattc |
| | | (SEQ ID NO: 278) |
| chr6 | gchr6-12 | tggtaagtctaagaaaccta |
| | | (SEQ ID NO: 279) |
| chr6 | gchr6-13 | cccacagcctaaccacccta |
| | | (SEQ ID NO: 280) |
| chr6 | gchr6-14 | aatatttcaaagccctaggg |
| | | (SEQ ID NO: 281) |
| chr6 | gchr6-15 | gcactcggaacagggtctgg |
| | | (SEQ ID NO: 282) |
| chr6 | gchr6-16 | agataggagctccaacagtg |
| | | (SEQ ID NO: 283) |
| chr6 | gchr6-17 | aagttagagcagccaggaaa |
| | | (SEQ ID NO: 284) |
| chr6 | gchr6-18 | tagagcagccaggaaaggga |
| | | (SEQ ID NO: 285) |
| chr6 | gchr6-19 | tgaatacccttccatgtcca |
| | | (SEQ ID NO: 286) |
| chr6 | gchr6-20 | cctgcattgcaccaggcaca |
| | | (SEQ ID NO: 287) |
| chr6 | gchr6-21 | tctagggcccaaacacacct |
| | | (SEQ ID NO: 288) |
| chr6 | gchr6-22 | tccctccatctatcaaaagg |
| | | (SEQ ID NO: 289) |
| chr10 | gchr10-1 | agccctgagacagaagcagg |
| | | (SEQ ID NO: 290) |
| chr10 | gchr10-2 | gccctgagacagaagcaggt |
| | | (SEQ ID NO: 291) |
| chr10 | gchr10-3 | aggagatgcagtgatacgca |
| | | (SEQ ID NO: 292) |
| chr10 | gchr10-4 | acaataccaagggtatccgg |
| | | (SEQ ID NO: 293) |
| chr10 | gchr10-5 | tgataaagaaaacaaagtga |
| | | (SEQ ID NO: 294) |
| chr10 | gchr10-6 | aaagaaaacaaagtgaggga |
| | | (SEQ ID NO: 295) |
| chr10 | gchr10-7 | gtggcaagtggagaaattga |
| | | (SEQ ID NO: 296) |
| chr10 | gchr10-8 | caagtggagaaattgaggga |
| | | (SEQ ID NO: 297) |
| chr10 | gchr10-9 | gtggtgatgattgcagctgg |
| | | (SEQ ID NO: 298) |
| chr11 | gchr11-1 | ctatgtgcctgacacacagg |
| | | (SEQ ID NO: 299) |
| chr11 | gchr11-2 | gggttggaccaggaaagagg |
| | | (SEQ ID NO: 300) |
| chr17 | gchr17-1 | gatgcctggaaaaggaaaga |
| | | (SEQ ID NO: 301) |
| chr17 | gchr17-2 | tagtatgcacctgcaagagg |
| | | (SEQ ID NO: 302) |
| chr17 | gchr17-3 | tatgcacctgcaagaggcgg |
| | | (SEQ ID NO: 303) |
| chr17 | gchr17-4 | aggggaagaagagaagcaga |
| | | (SEQ ID NO: 304) |
| chr17 | gchr17-5 | gctgaatcaagagacaagcg |
| | | (SEQ ID NO: 305) |
| chr17 | gchr17-6 | aagcaaataaatctcctggg |
| | | (SEQ ID NO: 306) |
| chr17 | gchr17-7 | agatgagtgctagagactgg |
| | | (SEQ ID NO: 307) |
| chr17 | gchr17-8 | ctgatggttgagcacagcag |
| | | (SEQ ID NO: 308) |

In embodiments, gRNAs for targeting human genomic safe harbor sites using any of the gRNA-based targeting elements, e.g., without limitation, dCas, in areas of open chromatin are shown in TABLES 3-7.

In embodiments, the gRNA comprises one or more of the sequences outlined herein or a variant sequence having at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

In embodiments, a Cas-based targeting element comprises Cas2 or a variant thereof, e.g., without limitation, Cas12a (e.g., dCas2a), or Cas12j (e.g., dCas2j), or Cas12k (e.g., dCas2k). In embodiments, the targeting element comprises a Cas2 enzyme guide RNA complex. In embodiments, comprises a nuclease-deficient dCas2 guide RNA complex, optionally dCas12j guide RNA complex or dCas2a guide RNA complex.

In embodiments, the targeting element is selected from a zinc finger (e), transcription activator-like effector (TALE), meganuclease, and clustered regularly interspaced short palindromic repeat (CRISPR)-associated protein, any of which are, in embodiments, catalytically inactive. In embodiments, the CRISPR-associated protein is selected from Cas9, CasX, CasY, Cas12a (Cpf1), and gRNA complexes thereof. In embodiments, the CRISPR-associated protein is selected from Cas9, xCas9, Cas 6, Cas7, Cas8, Cas12a (Cpf1), Cas13a, Cas14, CasX, CasY, a Class 1 Cas protein, a Class 2 Cas protein, MAD7, MG1 nuclease, MG2 nuclease, MG3 nuclease, or catalytically inactive forms thereof, and gRNA complexes thereof.

In embodiments, the helper enzyme of the present disclosure is capable of inserting a donor DNA at a TA dinucleotide site or a TTAA tetranucleotide site in a genomic safe harbor site (GSHS) of a nucleic acid molecule. The helper enzyme of the present disclosure is suitable for causing insertion of the donor DNA in a GSHS when contacted with a biological cell.

In embodiments, the targeting element is suitable for directing the helper enzyme of the present disclosure to the GSHS sequence.

In embodiments, the targeting element comprises transcription activator-like effector (TALE) DNA binding domain (DBD). The TALE DBD comprises one or more repeat sequences. For example, in embodiments, the TALE DBD comprises about 14, or about 15, or about, 16, or about 17, or about 18, or about 18.5 repeat sequences. In embodiments, the TALE DBD repeat sequences comprise 33 or 34 amino acids.

In embodiments, the one or more of the TALE DBD repeat sequences comprise a repeat variable di-residue (RVD) at residue 12 or 13 of the 33 or 34 amino acids.

In embodiments, the targeting element (e.g., TALE or Cas (e.g., Cas9 or Cas12, or variants thereof) DBDs cause the the helper enzyme of the present disclosure to bind specifically to human GSHS. In embodiments, the TALEs or Cas DBDs sequester the helper to GSHS and promote transposition to nearby TA dinucleotide or a TTAA tetranucleotide sites which can be located in proximity to the repeat variable di-residues (RVD) TALE or gRNA nucleotide sequences. The GSHS regions are located in open chromatin sites that are susceptible to helper activity. Accordingly, the helper enzyme of the present disclosure does not only operate based on its ability to recognize TA or TTAA sites, but it also directs a donor DNA (having a transgene) to specific locations in proximity to a TALE or Cas DBD. The helper enzyme of the present disclosure in accordance with embodiments of the present disclosure has negligible risk of genotoxicity and exhibits superior features as compared to existing gene therapies.

In embodiments, the helper enzyme of the present disclosure is mutated to be characterized by reduced or inhibited binding of off-target sequences and consequently reliant on a DBD fused thereto, such as a TALE or Cas DBD, for transposition.

The described cells, compositions, and methods allow reducing vector and transgene insertions that increase a mutagenic risk. The described cells and methods make use of a gene transfer system that reduces genotoxicity compared to viral- and nuclease-mediated gene therapies.

In embodiments, TALE or Cas DBDs are customizable, such as a TALE or Cas DBDs is selected for targeting a specific genomic location. In embodiments, the genomic location is in proximity to a TA dinucleotide site or a TTAA (SEQ ID NO: 440) tetranucleotide site.

Embodiments of the present disclosure make use of the ability of TALE or Cas or dCas9/gRNA DBDs to target specific sites in a host genome. The DNA targeting ability of a TALE or Cas DBD or dCas9/gRNA DBD is provided by TALE repeat sequences (e.g., modular arrays) or gRNA which are linked together to recognize flanking DNA sequences. Each TALE or gRNA can recognize certain base pair(s) or residue(s).

TALE nucleases (TALENs) are a known tool for genome editing and introducing targeted double-stranded breaks. TALENs comprise endonucleases, such as FokI nuclease domain, fused to a customizable DBD. This DBD is composed of highly conserved repeats from TALEs, which are proteins secreted by Xanthomonas bacteria to alter transcription of genes in host plant cells. The DBD includes a repeated highly conserved 33-34 amino acid sequence with divergent 12th and 13th amino acids. These two positions, referred to as the RVD, are highly variable and show a strong correlation with specific base pair or nucleotide recognition. This straightforward relationship between amino acid sequence and DNA recognition has allowed for the engineering of specific DBDs by selecting a combination of repeat segments containing the appropriate RVDs. Boch et al. Nature Biotechnology. 2011; 29 (2): 135-6.

Accordingly, TALENs can be readily designed using a “protein-DNA code” that relates modular DNA-binding TALE repeat domains to individual bases in a target-binding site. See Joung et al. Nat Rev Mol Cell Biol. 2013; 14(1):49-55. doi:10.1038/nrm3486. The following table, for example, shows such code:

| RVD | Nucleotide | RVD | Nucleotide |
|---|---|---|---|
| HD | C | NI | A |
| NH | G | NN | G, A |
| NK | G | NS | G, C, A |
| NG | T, mC | | |

It has been demonstrated that TALENs can be used to target essentially any DNA sequence of interest in human cell. Miller et al. Nat Biotechnol. 2011; 29:143-148. Guidelines for selection of potential target sites and for use of particular TALE repeat domains (harboring NH residues at the hypervariable positions) for recognition of G bases have been proposed. See Streubel et al. Nat Biotechnol. 2012; 30:593-595.

Accordingly, in embodiments, the TALE DBD comprises one or more repeat sequences. In embodiments, the TALE DBD comprises about 15, or about, 16, or about 17, or about 18, or about 18.5 repeat sequences. In embodiments, the TALE DBD repeat sequences comprise 33 or 34 amino acids.

In embodiments, the one or more of the TALE DBD repeat sequences comprise an RVD at residue 12 or 13 of the 33 or 34 amino acids. The RVD can recognize certain base pair(s) or residue(s). In embodiments, the RVD recognizes one base pair in the nucleic acid molecule. In embodiments, the RVD recognizes a C residue in the nucleic acid molecule and is selected from HD, N(gap), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from NI and NS. In embodiments, the RVD recognizes a T residue in the nucleic acid molecule and is selected from NG, HG, H(gap), and IG.

In embodiments, the GSHS is in an open chromatin location in a chromosome. In embodiments, the GSHS is selected from adeno-associated virus site 1 (AAVS1), chemokine (C-C motif) receptor 5 (CCR5) gene, HIV-1 coreceptor; and human Rosa26 locus. In embodiments, the GSHS is located on human chromosome 2, 4, 6, 10, 11, or 17.

In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the GSHS comprises one or more of TGGCCGGCCTGACCACTGG (SEQ ID NO: 23), TGAAGGCCTGGCCGGCCTG (SEQ ID NO: 24), TGAGCACTGAAGGCCTGGC (SEQ ID NO: 25), TCCACTGAGCACTGAAGGC (SEQ ID NO: 26), TGGTTTCCACTGAGCACTG (SEQ ID NO: 27), TGGGGAAAATGACCCAACA (SEQ ID NO: 28), TAGGACAGTGGGGAAAATG (SEQ ID NO: 29), TCCAGGGACACGGTGCTAG (SEQ ID NO: 30), TCAGAGCCAGGAGTCCTGG (SEQ ID NO: 31), TCCTTCAGAGCCAGGAGTC (SEQ ID NO: 32), TCCTCCTTCAGAGCCAGGA (SEQ ID NO: 33), TCCAGCCCCTCCTCCTTCA (SEQ ID NO: 34), TCCGAGCTTGACCCTTGGA (SEQ ID NO: 35), TGGTTTCCGAGCTTGACCC (SEQ ID NO: 36), TGGGGTGGTTTCCGAGCTT (SEQ ID NO: 37), TCTGCTGGGGTGGTTTCCG (SEQ ID NO: 38), TGCAGAGTATCTGCTGGGG (SEQ ID NO: 39), CCAATCCCCTCAGT (SEQ ID NO: 40), CAGTGCTCAGTGGAA (SEQ ID NO: 41), GAAACATCCGGCGACTCA (SEQ ID NO: 42), TCGCCCCTCAAATCTTACA (SEQ ID NO: 43), TCAAATCTTACAGCTGCTC (SEQ ID NO: 44), TCTTACAGCTGCTCACTCC (SEQ ID NO: 45), TACAGCTGCTCACTCCCCT (SEQ ID NO: 46), TGCTCACTCCCCTGCAGGG (SEQ ID NO: 47), TCCCCTGCAGGGCAACGCC (SEQ ID NO: 48), TGCAGGGCAACGCCCAGGG (SEQ ID NO: 49), TCTCGATTATGGGCGGGAT (SEQ ID NO: 50), TCGCTTCTCGATTATGGGC (SEQ ID NO: 51), TGTCGAGTCGCTTCTCGAT (SEQ ID NO: 52), TCCATGTCGAGTCGCTTCT (SEQ ID NO: 53), TCGCCTCCATGTCGAGTCG (SEQ ID NO: 54), TCGTCATCGCCTCCATGTC (SEQ ID NO: 55), TGATCTCGTCATCGCCTCC (SEQ ID NO: 56), GCTTCAGCTTCCTA (SEQ ID NO: 57), CTGTGATCATGCCA (SEQ ID NO: 58), ACAGTGGTACACACCT (SEQ ID NO: 59), CCACCCCCCACTAAG (SEQ ID NO: 60), CATTGGCCGGGCAC (SEQ ID NO: 61), GCTTGAACCCAGGAGA (SEQ ID NO: 62), ACACCCGATCCACTGGG (SEQ ID NO: 63), GCTGCATCAACCCC (SEQ ID NO: 64), GCCACAAACAGAAATA (SEQ ID NO: 65), GGTGGCTCATGCCTG (SEQ ID NO: 66), GATTTGCACAGCTCAT (SEQ ID NO: 67), AAGCTCTGAGGAGCA (SEQ ID NO: 68), CCCTAGCTGTCCC (SEQ ID NO: 69), GCCTAGCATGCTAG (SEQ ID NO: 70), ATGGGCTTCACGGAT (SEQ ID NO: 71), GAAACTATGCCTGC (SEQ ID NO: 72), GCACCATTGCTCCC (SEQ ID NO: 73), GACATGCAACTCAG (SEQ ID NO: 74), ACACCACTAGGGGT (SEQ ID NO: 75), GTCTGCTAGACAGG (SEQ ID NO: 76), GGCCTAGACAGGCTG (SEQ ID NO: 77), GAGGCATTCTTATCG (SEQ ID NO: 78), GCCTGGAAACGTTCC (SEQ ID NO: 79), GTGCTCTGACAATA (SEQ ID NO: 80), GTTTTGCAGCCTCC (SEQ ID NO: 81), ACAGCTGTGGAACGT (SEQ ID NO: 82), GGCTCTCTTCCTCCT (SEQ ID NO: 83), CTATCCCAAAACTCT (SEQ ID NO: 84), GAAAAACTATGTAT (SEQ ID NO: 85), AGGCAGGCTGGTTGA (SEQ ID NO: 86), CAATACAACCACGC (SEQ ID NO: 87), ATGACGGACTCAACT (SEQ ID NO: 88), CACAACATTTGTAA (SEQ ID NO: 89), and ATTTCCAGTGCACA (SEQ ID NO: 90).

In embodiments, the TALE DBD binds to one of TGGCCGGCCTGACCACTGG (SEQ ID NO: 23), TGAAGGCCTGGCCGGCCTG (SEQ ID NO: 24), TGAGCACTGAAGGCCTGGC (SEQ ID NO: 25), TCCACTGAGCACTGAAGGC (SEQ ID NO: 26), TGGTTTCCACTGAGCACTG (SEQ ID NO: 27), TGGGGAAAATGACCCAACA (SEQ ID NO: 28), TAGGACAGTGGGGAAAATG (SEQ ID NO: 29), TCCAGGGACACGGTGCTAG (SEQ ID NO: 30), TCAGAGCCAGGAGTCCTGG (SEQ ID NO: 31), TCCTTCAGAGCCAGGAGTC (SEQ ID NO: 32), TCCTCCTTCAGAGCCAGGA (SEQ ID NO: 33), TCCAGCCCCTCCTCCTTCA (SEQ ID NO: 34), TCCGAGCTTGACCCTTGGA (SEQ ID NO: 35), TGGTTTCCGAGCTTGACCC (SEQ ID NO: 36), TGGGGTGGTTTCCGAGCTT (SEQ ID NO: 37), TCTGCTGGGGTGGTTTCCG (SEQ ID NO: 38), TGCAGAGTATCTGCTGGGG (SEQ ID NO: 39), CCAATCCCCTCAGT (SEQ ID NO: 40), CAGTGCTCAGTGGAA (SEQ ID NO: 41), GAAACATCCGGCGACTCA (SEQ ID NO: 42), TCGCCCCTCAAATCTTACA (SEQ ID NO: 43), TCAAATCTTACAGCTGCTC (SEQ ID NO: 44), TCTTACAGCTGCTCACTCC (SEQ ID NO: 45), TACAGCTGCTCACTCCCCT (SEQ ID NO: 46), TGCTCACTCCCCTGCAGGG (SEQ ID NO: 47), TCCCCTGCAGGGCAACGCC (SEQ ID NO: 48), TGCAGGGCAACGCCCAGGG (SEQ ID NO: 49), TCTCGATTATGGGCGGGAT (SEQ ID NO: 50), TCGCTTCTCGATTATGGGC (SEQ ID NO: 51), TGTCGAGTCGCTTCTCGAT (SEQ ID NO: 52), TCCATGTCGAGTCGCTTCT (SEQ ID NO: 53), TCGCCTCCATGTCGAGTCG (SEQ ID NO: 54), TCGTCATCGCCTCCATGTC (SEQ ID NO: 55), TGATCTCGTCATCGCCTCC (SEQ ID NO: 56), GCTTCAGCTTCCTA (SEQ ID NO: 57), CTGTGATCATGCCA (SEQ ID NO: 58), ACAGTGGTACACACCT (SEQ ID NO: 59), CCACCCCCCACTAAG (SEQ ID NO: 60), CATTGGCCGGGCAC (SEQ ID NO: 61), GCTTGAACCCAGGAGA (SEQ ID NO: 62), ACACCCGATCCACTGGG (SEQ ID NO: 63), GCTGCATCAACCCC (SEQ ID NO: 64), GCCACAAACAGAAATA (SEQ ID NO: 65), GGTGGCTCATGCCTG (SEQ ID NO: 66), GATTTGCACAGCTCAT (SEQ ID NO: 67), AAGCTCTGAGGAGCA (SEQ ID NO: 68), CCCTAGCTGTCCC (SEQ ID NO: 69), GCCTAGCATGCTAG (SEQ ID NO: 70), ATGGGCTTCACGGAT (SEQ ID NO: 71), GAAACTATGCCTGC (SEQ ID NO: 72), GCACCATTGCTCCC (SEQ ID NO: 73), GACATGCAACTCAG (SEQ ID NO: 74), ACACCACTAGGGGT (SEQ ID NO: 75), GTCTGCTAGACAGG (SEQ ID NO: 76), GGCCTAGACAGGCTG (SEQ ID NO: 77), GAGGCATTCTTATCG (SEQ ID NO: 78), GCCTGGAAACGTTCC (SEQ ID NO: 79), GTGCTCTGACAATA (SEQ ID NO: 80), GTTTTGCAGCCTCC (SEQ ID NO: 81), ACAGCTGTGGAACGT (SEQ ID NO: 82), GGCTCTCTTCCTCCT (SEQ ID NO: 83), CTATCCCAAAACTCT (SEQ ID NO: 84), GAAAAACTATGTAT (SEQ ID NO: 85), AGGCAGGCTGGTTGA (SEQ ID NO: 86), CAATACAACCACGC (SEQ ID NO: 87), ATGACGGACTCAACT (SEQ ID NO: 88), CACAACATTTGTAA (SEQ ID NO: 89), and ATTTCCAGTGCACA (SEQ ID NO: 90).

In embodiments, the TALE DBD comprises one or more of NH NH HD HD NH NH HD HD NG NH NI HD HD NI HD NG NH NH, NH NI NI NH NH HD HD NG NH NH HD HD NH NH HD HD NG NH, NH NI NH HD NI HD NG NH NI NI NH NH HD HD NG NH NH HD, HD HD NI HD NG NH NI NH HD NI HD NG NH NI NI NH NH HD, NH NH NG NG NG HD HD NI HD NG NH NI NH HD NI HD NG NH, NH NH NH NH NI NI NI NI NG NH NI HD HD HD NI NI HD NI, NI NH NH NI HD NI NH NG NH NH NH NH NI NI NI NI NG NH, HD HD NI NH NH NH NI HD NI HD NH NH NG NH HD NG NI NH, HD NI NH NI NH HD HD NI NH NH NI NH NG HD HD NG NH NH, HD HD NG NG HD NI NH NI NH HD HD NI NH NH NI NH NG HD, HD HD NG HD HD NG NG HD NI NH NI NH HD HD NI NH NH NI, HD HD NI NH HD HD HD HD NG HD HD NG HD HD NG NG HD NI, HD HD NH NI NH HD NG NG NH NI HD HD HD NG NG NH NH NI, NH NH NG NG NG HD HD NH NI NH HD NG NG NH NI HD HD HD, NH NH NH NH NG NH NH NG NG NG HD HD NH NI NH HD NG NG, HD NG NH HD NG NH NH NH NH NG NH NH NG NG NG HD HD NH, NH HD NI NH NI NH NG NI NG HD NG NH HD NG NH NH NH NH, HD HD NI NI NG HD HD HD HD NG HD NI NH NG, HD NI NH NG NH HD NG HD NI NH NG NH NH NI NI, NH NI NI NI HD NI NG HD HD NH NH HD NH NI HD NG HD NI, HD NH HD HD HD HD NG HD NI NI NI NG HD NG NG NI HD NI, HD NI NI NI NG HD NG NG NI HD NI NH HD NG NH HD NG HD, HD NG NG NI HD NI NH HD NG NH HD NG HD NI HD NG HD HD, NI HD NI NH HD NG NH HD NG HD NI HD NG HD HD HD HD NG, NH HD NG HD NI HD NG HD HD HD HD NG NH HD NI NH NH NH, HD HD HD HD NG NH HD NI NH NH NH HD NI NI HD NH HD HD, NH HD NI NH NH NH HD NI NI HD NH HD HD HD NI NH NH NH, HD NG HD NH NI NG NG NI NG NH NH NH HD NH NH NH NI NG, HD NH HD NG NG HD NG HD NH NI NG NG NI NG NH NH NH HD, NH NG HD NH NI NH NG HD NH HD NG NG HD NG HD NH NI NG, HD HD NI NG NH NG HD NH NI NH NG HD NH HD NG NG HD NG, HD NH HD HD NG HD HD NI NG NH NG HD NH NI NH NG HD NH, HD NH NG HD NI NG HD NH HD HD NG HD HD NI NG NH NG HD, NH NI NG HD NG HD NH NG HD NI NG HD NH HD HD NG HD HD, NH HD NG NG HD NI NH HD NG NG HD HD NG NI, HD NG NK NG NH NI NG HD NI NG NH HD HD NI, NI HD NI NN NG NN NN NG NI HD NI HD NI HD HD NG, HD HD NI HD HD HD HD HD HD NI HD NG NI NI NN, HD NI NG NG NN NN HD HD NN NN NN HD NI HD, NN HD NG NG NN NI NI HD HD HD NI NN NN NI NN NI, NI HD NI HD HD HD NN NI NG HD HD NI HD NG NN NN NN, NN HD NG NN HD NI NG HD NI NI HD HD HD HD, NN NN HD NI HD NN NI NI NI HD NI HD HD HD NG HD HD, NN NN NG NN NN HD NG HD NI NG NN HD HD NG NN, NN NI NG NG NG NN HD NI HD NI NN HD NG HD NI NG, NI NI NH HD NG HD NG NH NI NH NH NI NH HD, HD HD HD NG NI NK HD NG NH NG HD HD HD HD, NH HD HD NG NI NH HD NI NG NH HD NG NI NH, NI NG NH NH NH HD NG NG HD NI HD NH NH NI NG, NH NI NI NI HD NG NI NG NH HD HD NG NH HD, NH HD NI HD HD NI NG NG NH HD NG HD HD HD, NH NI HD NI NG NH HD NI NI HD NG HD NI NH, NI HD NI HD HD NI HD NG NI NH NH NH NH NG, NH NG HD NG NH HD NG NI NH NI HD NI NH NH, NH NH HD HD NG NI NH NI HD NI NH NH HD NG NH, NH NI NH NH HD NI NG NG HD NG NG NI NG HD NH, NN HD HD NG NN NN NI NI NI HD NN NG NG HD HD, NN NG NN HD NG HD NG NN NI HD NI NI NG NI, NN NG NG NG NG NN HD NI NN HD HD NG HD HD, NI HD NI NN HD NG NN NG NN NN NI NI HD NN NG, HD NI NI NN NI HD HD NN NI NN HD NI HD NG NN HD NG NN, HD NG NI NG HD HD HD NI NI NI NI HD NG HD NG, NH NI NI NI NI NI HD NG NI NG NH NG NI NG, NI NH NH HD NI NH NH HD NG NH NH NG NG NH NI, HD NI NI NG NI HD NI NI HD HD NI HD NN HD, NI NG NN NI HD NN NN NI HD NG HD NI NI HD NG, HD NI HD NI NI HD NI NG NG NG NN NG NI NI, and NI NG NG NG HD HD NI NN NG NN HD NI HD NI.

In embodiments, the TALE DBD comprises one or more of the sequences outlined herein or a variant sequence having at least about 90%, or at least about 93%, or at least about 95%, or at least about 97%, or at least about 98%, or at least about 99% identity thereto, or at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

In embodiments, the GSHS and the TALE DBD sequences are selected from:

(SEQ ID NO: 23)
TGGCCGGCCTGACCACTGG
and
NH NH HD HD NH NH HD HD NG NH NI HD HD NI HD NG NH
NH;
(SEQ ID NO: 24)
TGAAGGCCTGGCCGGCCTG
and
NH NI NI NH NH HD HD NG NH NH HD HD NH NH HD HD NG
NH;
(SEQ ID NO: 25)
TGAGCACTGAAGGCCTGGC
and
NH NI NH HD NI HD NG NH NI NI NH NH HD HD NG NH NH
HD;
(SEQ ID NO: 26)
TCCACTGAGCACTGAAGGC
and
HD HD NI HD NG NH NI NH HD NI HD NG NH NI NI NH NH
HD;
(SEQ ID NO: 27)
TGGTTTCCACTGAGCACTG
and
NH NH NG NG NG HD HD NI HD NG NH NI NH HD NI HD NG
NH;
(SEQ ID NO: 28)
TGGGGAAAATGACCCAACA
and
NH NH NH NH NI NI NI NI NG NH NI HD HD HD NI NI HD
NI;
(SEQ ID NO: 29)
TAGGACAGTGGGGAAAATG
and
NI NH NH NI HD NI NH NG NH NH NH NH NI NI NI NI NG
NH;
(SEQ ID NO: 30)
TCCAGGGACACGGTGCTAG
and
HD HD NI NH NH NH NI HD NI HD NH NH NG NH HD NG NI
NH;
(SEQ ID NO: 31)
TCAGAGCCAGGAGTCCTGG
and
HD NI NH NI NH HD HD NI NH NH NI NH NG HD HD NG NH
NH;
(SEQ ID NO: 32)
TCCTTCAGAGCCAGGAGTC
and
HD HD NG NG HD NI NH NI NH HD HD NI NH NH NI NH NG
HD;
(SEQ ID NO: 33)
TCCTCCTTCAGAGCCAGGA
and
HD HD NG HD HD NG NG HD NI NH NI NH HD HD NI NH NH
NI;
(SEQ ID NO: 34)
TCCAGCCCCTCCTCCTTCA
and
HD HD NI NH HD HD HD HD NG HD HD NG HD HD NG NG HD
NI;
(SEQ ID NO: 35)
TCCGAGCTTGACCCTTGGA
and
HD HD NH NI NH HD NG NG NH NI HD HD HD NG NG NH NH
NI;
(SEQ ID NO: 36)
TGGTTTCCGAGCTTGACCC
and
NH NH NG NG NG HD HD NH NI NH HD NG NG NH NI HD HD
HD;
(SEQ ID NO: 37)
TGGGGTGGTTTCCGAGCTT
and
NH NH NH NH NG NH NH NG NG NG HD HD NH NI NH HD NG
NG;
(SEQ ID NO: 38)
TCTGCTGGGGTGGTTTCCG
and
HD NG NH HD NG NH NH NH NH NG NH NH NG NG NG HD
HD NH;
(SEQ ID NO: 39)
TGCAGAGTATCTGCTGGGG
and
NH HD NI NH NI NH NG NI NG HD NG NH HD NG NH NH NH
NH;
(SEQ ID NO: 40)
CCAATCCCCTCAGT
and
HD HD NI NI NG HD HD HD HD NG HD NI NH NG;
(SEQ ID NO: 41)
CAGTGCTCAGTGGAA
and
HD NI NH NG NH HD NG HD NI NH NG NH NH NI NI;
(SEQ ID NO: 42)
GAAACATCCGGCGACTCA
and
NH NI NI NI HD NI NG HD HD NH NH HD NH NI HD NG HD
NI;
(SEQ ID NO: 43)
TCGCCCCTCAAATCTTACA
and
HD NH HD HD HD HD NG HD NI NI NI NG HD NG NG NI HD
NI;
(SEQ ID NO: 44)
TCAAATCTTACAGCTGCTC
and
HD NI NI NI NG HD NG NG NI HD NI NH HD NG NH HD NG
HD;
(SEQ ID NO: 45)
TCTTACAGCTGCTCACTCC
and
HD NG NG NI HD NI NH HD NG NH HD NG HD NI HD NG HD
HD;
(SEQ ID NO: 46)
TACAGCTGCTCACTCCCCT
and
NI HD NI NH HD NG NH HD NG HD NI HD NG HD HD HD HD
NG;
(SEQ ID NO: 47)
TGCTCACTCCCCTGCAGGG
and
NH HD NG HD NI HD NG HD HD HD HD NG NH HD NI NH NH
NH;
(SEQ ID NO: 48)
TCCCCTGCAGGGCAACGCC
and
HD HD HD HD NG NH HD NI NH NH NH HD NI NI HD NH HD
HD;
(SEQ ID NO: 49)
TGCAGGGCAACGCCCAGGG
and
NH HD NI NH NH NH HD NI NI HD NH HD HD HD NI NH NH
NH;
(SEQ ID NO: 50)
TCTCGATTATGGGGGGGAT
and
HD NG HD NH NI NG NG NI NG NH NH NH HD NH NH NH NI
NG;
(SEQ ID NO: 51)
TCGCTTCTCGATTATGGGC
and
HD NH HD NG NG HD NG HD NH NI NG NG NI NG NH NH NH
HD;
(SEQ ID NO: 52)
TGTCGAGTCGCTTCTCGAT
and
NH NG HD NH NI NH NG HD NH HD NG NG HD NG HD NH NI
NG;
(SEQ ID NO: 53)
TCCATGTCGAGTCGCTTCT
and
HD HD NI NG NH NG HD NH NI NH NG HD NH HD NG NG HD
NG;
(SEQ ID NO: 54)
TCGCCTCCATGTCGAGTCG
and
HD NH HD HD NG HD HD NI NG NH NG HD NH NI NH NG HD
NH;
(SEQ ID NO: 55)
TCGTCATCGCCTCCATGTC
and
HD NH NG HD NI NG HD NH HD HD NG HD HD NI NG NH NG
HD;
(SEQ ID NO: 56)
TGATCTCGTCATCGCCTCC
and
NH NI NG HD NG HD NH NG HD NI NG HD NH HD HD NG HD
HD;
(SEQ ID NO: 57)
GCTTCAGCTTCCTA
and
NH HD NG NG HD NI NH HD NG NG HD HD NG NI;
(SEQ ID NO: 58)
CTGTGATCATGCCA
and
HD NG NK NG NH NI NG HD NI NG NH HD HD NI;
(SEQ ID NO: 59)
ACAGTGGTACACACCT
and
NI HD NI NN NG NN NN NG NI HD NI HD NI HD HD NG;
(SEQ ID NO: 60)
CCACCCCCCACTAAG
and
HD HD NI HD HD HD HD HD HD NI HD NG NI NI NN;
(SEQ ID NO: 61)
CATTGGCCGGGCAC
and
HD NI NG NG NN NN HD HD NN NN NN HD NI HD;
(SEQ ID NO: 62)
GCTTGAACCCAGGAGA
and
NN HD NG NG NN NI NI HD HD HD NI NN NN NI NN NI;
(SEQ ID NO: 63)
ACACCCGATCCACTGGG
and
NI HD NI HD HD HD NN NI NG HD HD NI HD NG NN NN
NN;
(SEQ ID NO: 64)
GCTGCATCAACCCC
and
NN HD NG NN HD NI NG HD NI NI HD HD HD HD;
(SEQ ID NO: 65)
GCCACAAACAGAAATA
and
NN NN HD NI HD NN NI NI NI HD NI HD HD HD NG HD
HD;
(SEQ ID NO: 66)
GGTGGCTCATGCCTG
and
NN NN NG NN NN HD NG HD NI NG NN HD HD NG NN;
(SEQ ID NO: 67)
GATTTGCACAGCTCAT
and
NN NI NG NG NG NN HD NI HD NI NN HD NG HD NI NG;
(SEQ ID NO: 68)
AAGCTCTGAGGAGCA
and
NI NI NH HD NG HD NG NH NI NH NH NI NH HD;
(SEQ ID NO: 69)
CCCTAGCTGTCCC
and
HD HD HD NG NI NK HD NG NH NG HD HD HD HD;
(SEQ ID NO: 70)
GCCTAGCATGCTAG
and
NH HD HD NG NI NH HD NI NG NH HD NG NI NH;
(SEQ ID NO: 71)
ATGGGCTTCACGGAT
and
NI NG NH NH NH HD NG NG HD NI HD NH NH NI NG;
(SEQ ID NO: 72)
GAAACTATGCCTGC
and
NH NI NI NI HD NG NI NG NH HD HD NG NH HD;
(SEQ ID NO: 73)
GCACCATTGCTCCC
and
NH HD NI HD HD NI NG NG NH HD NG HD HD HD;
(SEQ ID NO: 74)
GACATGCAACTCAG
and
NH NI HD NI NG NH HD NI NI HD NG HD NI NH;
(SEQ ID NO: 75)
ACACCACTAGGGGT
and
NI HD NI HD HD NI HD NG NI NH NH NH NH NG;
(SEQ ID NO: 76)
GTCTGCTAGACAGG
and
NH NG HD NG NH HD NG NI NH NI HD NI NH NH;
(SEQ ID NO: 77)
GGCCTAGACAGGCTG
and
NH NH HD HD NG NI NH NI HD NI NH NH HD NG NH;
(SEQ ID NO: 78)
GAGGCATTCTTATCG
and
NH NI NH NH HD NI NG NG HD NG NG NI NG HD NH;
(SEQ ID NO: 79)
GCCTGGAAACGTTCC
and
NN HD HD NG NN NN NI NI NI HD NN NG NG HD HD;
(SEQ ID NO: 80)
GTGCTCTGACAATA
and
NN NG NN HD NG HD NG NN NI HD NI NI NG NI;
(SEQ ID NO: 81)
GTTTTGCAGCCTCC
and
NN NG NG NG NG NN HD NI NN HD HD NG HD HD;
(SEQ ID NO: 82)
ACAGCTGTGGAACGT
and
NI HD NI NN HD NG NN NG NN NN NI NI HD NN NG;
(SEQ ID NO: 83)
GGCTCTCTTCCTCCT
and
HD NI NI NN NI HD HD NN NI NN HD NI HD NG NN HD NG
NN;
(SEQ ID NO: 84)
CTATCCCAAAACTCT
and
HD NG NI NG HD HD HD NI NI NI NI HD NG HD NG;
(SEQ ID NO: 85)
GAAAAACTATGTAT
and
NH NI NI NI NI NI HD NG NI NG NH NG NI NG;
(SEQ ID NO: 86)
AGGCAGGCTGGTTGA
and
NI NH NH HD NI NH NH HD NG NH NH NG NG NH NI;
(SEQ ID NO: 87)
CAATACAACCACGC
and
HD NI NI NG NI HD NI NI HD HD NI HD NN HD;
(SEQ ID NO: 88)
ATGACGGACTCAACT
and
NI NG NN NI HD NN NN NI HD NG HD NI NI HD NG;
and
(SEQ ID NO: 89)
CACAACATTTGTAA
and
HD NI HD NI NI HD NI NG NG NG NN NG NI NI.

In embodiments, the GSHS is within about 25, or about 50, or about 100, or about 150, or about 200, or about 300, or about 500 nucleotides of the TA dinucleotide site or TTAA (SEQ ID NO: 440) tetranucleotide site.

Illustrative DNA binding codes for targeting human genomic safe harbor in areas of open chromatin via TALES, encompassed by various embodiments are provided in TABLE 20.

TABLE 20

| GSHS | ID | Sequence | TALE (DNA binding code) |
|---|---|---|---|
| AAVS1 | 1 | tggccggcctgaccactgg (SEQ ID | NH NH HD HD NH NH HD HD NG NH NI |
| | | NO: 23) | HD HD NI HD NG NH NH |
| AAVS1 | 2 | tgaaggcctggccggcctg (SEQ ID | NH NI NI NH NH HD HD NG NH NH HD |
| | | NO: 24) | HD NH NH HD HD NG NH |
| AAVS1 | 3 | tgagcactgaaggcctggc (SEQ ID | NH NI NH HD NI HD NG NH NI NI NH NH |
| | | NO: 25) | HD HD NG NH NH HD |
| AAVS1 | 4 | tccactgagcactgaaggc (SEQ ID | HD HD NI HD NG NH NI NH HD NI HD |
| | | NO: 26) | NG NH NI NI NH NH HD |
| AAVS1 | 5 | tggtttccactgagcactg (SEQ ID | NH NH NG NG NG HD HD NI HD NG NH |
| | | NO: 27) | NI NH HD NI HD NG NH |
| AAVS1 | 6 | tggggaaaatgacccaaca (SEQ | NH NH NH NH NI NI NI NI NG NH NI HD |
| | | ID NO: 28) | HD HD NI NI HD NI |
| AAVS1 | 7 | taggacagtggggaaaatg (SEQ | NI NH NH NI HD NI NH NG NH NH NH |
| | | ID NO: 29) | NH NI NI NI NI NG NH |
| AAVS1 | 8 | tccagggacacggtgctag (SEQ ID | HD HD NI NH NH NH NI HD NI HD NH |
| | | NO: 30) | NH NG NH HD NG NI NH |
| AAVS1 | 9 | tcagagccaggagtcctgg (SEQ ID | HD NI NH NI NH HD HD NI NH NH NI NH |
| | | NO: 31) | NG HD HD NG NH NH |
| AAVS1 | 10 | tccttcagagccaggagtc (SEQ ID | HD HD NG NG HD NI NH NI NH HD HD |
| | | NO: 32) | NI NH NH NI NH NG HD |
| AAVS1 | 11 | tcctccttcagagccagga (SEQ ID | HD HD NG HD HD NG NG HD NI NH NI |
| | | NO: 33) | NH HD HD NI NH NH NI |
| AAVS1 | 12 | tccagcccctcctccttca (SEQ ID | HD HD NI NH HD HD HD HD NG HD HD |
| | | NO: 34) | NG HD HD NG NG HD NI |
| AAVS1 | 13 | tccgagcttgacccttgga (SEQ ID | HD HD NH NI NH HD NG NG NH NI HD |
| | | NO: 35) | HD HD NG NG NH NH NI |
| AAVS1 | 14 | tggtttccgagcttgaccc (SEQ ID | NH NH NG NG NG HD HD NH NI NH HD |
| | | NO: 36) | NG NG NH NI HD HD HD |
| AAVS1 | 15 | tggggtggtttccgagctt (SEQ ID | NH NH NH NH NG NH NH NG NG NG |
| | | NO: 37) | HD HD NH NI NH HD NG NG |
| AAVS1 | 16 | tctgctggggtggtttccg (SEQ ID | HD NG NH HD NG NH NH NH NH NG |
| | | NO: 38) | NH NH NG NG NG HD HD NH |
| AAVS1 | 17 | tgcagagtatctgctgggg (SEQ ID | NH HD NI NH NI NH NG NI NG HD NG |
| | | NO: 39) | NH HD NG NH NH NH NH |
| AAVS1 | AVS1 | CCAATCCCCTCAGT (SEQ | HD HD NI NI NG HD HD HD HD NG HD |
| | | ID NO: 40) | NI NH NG |
| AAVS1 | AVS2 | CAGTGCTCAGTGGAA (SEQ | HD NI NH NG NH HD NG HD NI NH NG |
| | | ID NO: 41) | NH NH NI NI |
| AAVS1 | AVS3 | GAAACATCCGGCGACTCA | NH NI NI NI HD NI NG HD HD NH NH HD |
| | | (SEQ ID NO: 42) | NH NI HD NG HD NI |
| hROSA26 | 1F | tcgcccctcaaatcttaca (SEQ ID | HD NH HD HD HD HD NG HD NI NI NI |
| | | NO: 43) | NG HD NG NG NI HD NI |
| hROSA26 | 2F | tcaaatcttacagctgctc (SEQ ID | HD NI NI NI NG HD NG NG NI HD NI NH |
| | | NO: 44) | HD NG NH HD NG HD |
| hROSA26 | 3F | tcttacagctgctcactcc (SEQ ID | HD NG NG NI HD NI NH HD NG NH HD |
| | | NO: 45) | NG HD NI HD NG HD HD |
| hROSA26 | 4F | tacagctgctcactcccct (SEQ ID | NI HD NI NH HD NG NH HD NG HD NI |
| | | NO: 46) | HD NG HD HD HD HD NG |
| hROSA26 | 5F | tgctcactcccctgcaggg (SEQ ID | NH HD NG HD NI HD NG HD HD HD HD |
| | | NO: 47) | NG NH HD NI NH NH NH |
| hROSA26 | 6F | tcccctgcagggcaacgcc (SEQ | HD HD HD HD NG NH HD NI NH NH NH |
| | | ID NO: 48) | HD NI NI HD NH HD HD |
| hROSA26 | 7F | tgcagggcaacgcccaggg (SEQ | NH HD NI NH NH NH HD NI NI HD NH |
| | | ID NO: 49) | HD HD HD NI NH NH NH |
| hROSA26 | 8R | tctcgattatggggggat (SEQ ID | HD NG HD NH NI NG NG NI NG NH NH |
| | | NO: 50) | NH HD NH NH NH NI NG |
| hROSA26 | 9R | tcgcttctcgattatgggc (SEQ ID | HD NH HD NG NG HD NG HD NH NI NG |
| | | NO: 51) | NG NI NG NH NH NH HD |
| hROSA26 | 10R | tgtcgagtcgcttctcgat (SEQ ID | NH NG HD NH NI NH NG HD NH HD NG |
| | | NO: 52) | NG HD NG HD NH NI NG |
| hROSA26 | 11R | tccatgtcgagtcgcttct (SEQ ID | HD HD NI NG NH NG HD NH NI NH NG |
| | | NO: 53) | HD NH HD NG NG HD NG |
| hROSA26 | 12R | tcgcctccatgtcgagtcg (SEQ ID | HD NH HD HD NG HD HD NI NG NH NG |
| | | NO: 54) | HD NH NI NH NG HD NH |
| hROSA26 | 13R | tcgtcatcgcctccatgtc (SEQ ID | HD NH NG HD NI NG HD NH HD HD NG |
| | | NO: 55) | HD HD NI NG NH NG HD |
| hROSA26 | 14R | tgatctcgtcatcgcctcc (SEQ ID | NH NI NG HD NG HD NH NG HD NI NG |
| | | NO: 56) | HD NH HD HD NG HD HD |
| hROSA26 | ROSA1 | GCTTCAGCTTCCTA (SEQ | NH HD NG NG HD NI NH HD NG NG HD |
| | | ID NO: 57) | HD NG NI |
| hROSA26 | ROSA2 | CTGTGATCATGCCA (SEQ | HD NG NK NG NH NI NG HD NI NG NH |
| | | ID NO: 58) | HD HD NI |
| hROSA26 | TALER2 | ACAGTGGTACACACCT | NI HD NI NN NG NN NN NG NI HD NI HD |
| | | (SEQ ID NO: 59) | NI HD HD NG |
| hROSA26 | TALER3 | CCACCCCCCACTAAG (SEQ | HD HD NI HD HD HD HD HD HD NI HD |
| | | ID NO: 60) | NG NI NI NN |
| hROSA26 | TALER4 | CATTGGCCGGGCAC (SEQ | HD NI NG NG NN NN HD HD NN NN NN |
| | | ID NO: 61) | HD NI HD |
| hROSA26 | TALER5 | GCTTGAACCCAGGAGA | NN HD NG NG NN NI NI HD HD HD NI |
| | | (SEQ ID NO: 62) | NN NN NI NN NI |
| CCR5 | TALC3 | ACACCCGATCCACTGGG | NI HD NI HD HD HD NN NI NG HD HD |
| | | (SEQ ID NO: 63) | NI HD NG NN NN NN |
| CCR5 | TALC4 | GCTGCATCAACCCC (SEQ | NN HD NG NN HD NI NG HD NI NI HD |
| | | ID NO: 64) | HD HD HD |
| CCR5 | TALC5 | GCCACAAACAGAAATA | NN NN HD NI HD NN NI NI NI HD NI HD |
| | | (SEQ ID NO: 65) | HD HD NG HD HD |
| CCR5 | TALC7 | GGTGGCTCATGCCTG | NN NN NG NN NN HD NG HD NI NG NN |
| | | (SEQ ID NO: 66) | HD HD NG NN |
| CCR5 | TALC8 | GATTTGCACAGCTCAT | NN NI NG NG NG NN HD NI HD NI NN |
| | | (SEQ ID NO: 67) | HD NG HD NI NG |
| Chr 2 | SHCHR2-1 | AAGCTCTGAGGAGCA (SEQ | NI NI NH HD NG HD NG NH NI NH NH |
| | | ID NO: 68) | NI NH HD |
| Chr 2 | SHCHR2-2 | CCCTAGCTGTCCC (SEQ ID | HD HD HD NG NI NK HD NG NH NG HD |
| | | NO: 69) | HD HD HD |
| Chr 2 | SHCHR2-3 | GCCTAGCATGCTAG (SEQ | NH HD HD NG NI NH HD NI NG NH HD |
| | | ID NO: 70) | NG NI NH |
| Chr 2 | SHCHR2-4 | ATGGGCTTCACGGAT (SEQ | NI NG NH NH NH HD NG NG HD NI HD |
| | | ID NO: 71) | NH NH NI NG |
| Chr 4 | SHCHR4-1 | GAAACTATGCCTGC (SEQ | NH NI NI NI HD NG NI NG NH HD HD NG |
| | | ID NO: 72) | NH HD |
| Chr 4 | SHCHR4-2 | GCACCATTGCTCCC (SEQ | NH HD NI HD HD NI NG NG NH HD NG |
| | | ID NO: 73) | HD HD HD |
| Chr 4 | SHCHR4-3 | GACATGCAACTCAG (SEQ | NH NI HD NI NG NH HD NI NI HD NG HD |
| | | ID NO: 74) | NI NH |
| Chr 6 | SHCHR6-1 | ACACCACTAGGGGT (SEQ | NI HD NI HD HD NI HD NG NI NH NH NH |
| | | ID NO: 75) | NH NG |
| Chr 6 | SHCHR6-2 | GTCTGCTAGACAGG (SEQ | NH NG HD NG NH HD NG NI NH NI HD |
| | | ID NO: 76) | NI NH NH |
| Chr 6 | SHCHR6-3 | GGCCTAGACAGGCTG | NH NH HD HD NG NI NH NI HD NI NH |
| | | (SEQ ID NO: 77) | NH HD NG NH |
| Chr 6 | SHCHR6-4 | GAGGCATTCTTATCG (SEQ | NH NI NH NH HD NI NG NG HD NG NG |
| | | ID NO: 78) | NI NG HD NH |
| Chr 10 | SHCHR10-1 | GCCTGGAAACGTTCC (SEQ | NN HD HD NG NN NN NI NI NI HD NN |
| | | ID NO: 79) | NG NG HD HD |
| Chr 10 | SHCHR10-2 | GTGCTCTGACAATA (SEQ | NN NG NN HD NG HD NG NN NI HD NI |
| | | ID NO: 80) | NI NG NI |
| Chr 10 | SHCHR10-3 | GTTTTGCAGCCTCC (SEQ | NN NG NG NG NG NN HD NI NN HD HD |
| | | ID NO: 81) | NG HD HD |
| Chr 10 | SHCHR10-4 | ACAGCTGTGGAACGT (SEQ | NI HD NI NN HD NG NN NG NN NN NI |
| | | ID NO: 82) | NI HD NN NG |
| Chr 10 | SHCHR10-5 | GGCTCTCTTCCTCCT (SEQ | HD NI NI NN NI HD HD NN NI NN HD NI |
| | | ID NO: 83) | HD NG NN HD NG NN |
| Chr 11 | SHCHR11-1 | CTATCCCAAAACTCT (SEQ | HD NG NI NG HD HD HD NI NI NI NI HD |
| | | ID NO: 84) | NG HD NG |
| Chr 11 | SHCHR11-2 | GAAAAACTATGTAT (SEQ ID | NH NI NI NI NI NI HD NG NI NG NH NG |
| | | NO: 85) | NI NG |
| Chr 11 | SHCHR11-3 | AGGCAGGCTGGTTGA | NI NH NH HD NI NH NH HD NG NH NH |
| | | (SEQ ID NO: 86) | NG NG NH NI |
| Chr 17 | SHCHR17-1 | CAATACAACCACGC (SEQ | HD NI NI NG NI HD NI NI HD HD NI HD |
| | | ID NO: 87) | NN HD |
| Chr 17 | SHCHR17-2 | ATGACGGACTCAACT (SEQ | NI NG NN NI HD NN NN NI HD NG HD |
| | | ID NO: 88) | NI NI HD NG |
| Chr 17 | SHCHR17-3 | CACAACATTTGTAA (SEQ ID | HD NI HD NI NI HD NI NG NG NG NN |
| | | NO: 89) | NG NI NI |
| Chr 17 | SHCHR17-4 | ATTTCCAGTGCACA (SEQ | NI NG NG NG HD HD NI NN NG NN HD |
| | | ID NO: 90) | NI HD NI |

Further illustrative DNA binding codes for targeting human genomic safe harbor in areas of open chromatin via TALES, encompassed by embodiments are provided in TABLES 8-12. In embodiments, the helper enzyme of the present disclosure is capable of inserting a donor DNA at a TA dinucleotide site. In embodiments, the helper enzyme of the present disclosure is capable of inserting a donor DNA at a TTAA (SEQ ID NO: 440) tetranucleotide site.

In embodiments, the present disclosure relates to a system having nucleic acids encoding the enzyme (e.g., without limitation, the helper enzyme) and the donor DNA, respectively.

### Linkers

In some embodiments, the targeting element comprises a nucleic acid binding component of a gene-editing system. In some embodiments, the helper enzyme the targeting element are connected. Without wishing to be bound by a particular theory, the targeting element may refer to a nucleic acid binding component of the gene-editing system. In some embodiments, the helper enzyme and the targeting element are connected. For example, in embodiments, the the helper enzyme and the targeting element are fused to one another or linked via a linker to one another.

In some embodiments, the linker is a flexible linker. In some embodiments, the flexible linker is substantially comprised of glycine and serine residues, optionally wherein the flexible linker comprises (Gly₄Ser)ₙ, where n is an integer from 1 to 12. In some embodiments, the flexible linker is of about 20, or about 30, or about 40, or about 50, or about 60 amino acid residues. In embodiments, the flexible linker is about 50, or about 100, or about 150, or about 200 amino acid residues in length. In embodiments, the flexible linker comprises at least about 150 nucleotides (nt), or at least about 200 nt, or at least about 250 nt, or at least about 300 nt, or at least about 350 nt, or at least about 400 nt, or at least about 450 nt, or at least about 500 nt, or at least about 500 nt, or at least about 600 nt. In embodiments, the flexible linker comprises from about 450 nt to about 500 nt.

### Inteins

Inteins (INTervening protEINS) are mobile genetic elements that are protein domains, found in nature, with the capability to carry out the process of protein splicing. See Sarmiento & Camarero (2019) Current protein & peptide science, 20(5), 408-424, which is incorporated by reference herein in its entirety. Protein spicing is a post-translation biochemical modification which results in the cleavage and formation of peptide bonds between precursor polypeptide segments flanking the intein. Id. Inteins apply standard enzymatic strategies to excise themselves post-translationally from a precursor protein via protein splicing. Nanda et al., Microorganisms vol. 8, 12 2004. 16 Dec. 2020, doi:10.3390/microorganisms8122004. An intein can splice its flanking N- and C-terminal domains to become a mature protein and excise itself from a sequence. For example, split inteins have been used to control the delivery of heterologous genes into transgenic organisms. See Wood & Camarero (2014) J Biol Chem. 289(21):14512-14519. This approach relies on splitting the target protein into two segments, which are then post-translationally reconstituted in vivo by protein trans-splicing (PTS). See Aboye & Camarero (2012) J. Biol. Chem. 287, 27026-27032. More recently, an intein-mediated split-Cas9 system has been developed to incorporate Cas9 into cells and reconstitute nuclease activity efficiently. Truong et al., Nucleic Acids Res. 2015, 43 (13), 6450-6458. The protein splicing excises the internal region of the precursor protein, which is then followed by the ligation of the N-extein and C-extein fragments, resulting in two polypeptides—the excised intein and the new polypeptide produced by joining the C- and N-exteins. Sarmiento & Camarero (2019).

In embodiments, intein-mediated incorporation of DNA binders such as, without limitation, dCas9, dCas12j, or TALEs, allows creation of a split-enzyme system such as, without limitation, split helper system, that permits reconstitution of the full-length enzyme, e.g., helper, from two smaller fragments. This allows avoiding the need to express DNA binders at the N- or C-terminus of an enzyme, e.g., helper. In this approach, the two portions of an enzyme, e.g., helper, are fused to the intein and, after co-expression, the intein allows producing a full-length enzyme, e.g., helper, by post-translation modification. Thus, in embodiments, a nucleic acid encoding the enzyme capable of targeted genomic integration by transposition comprises an intein. In embodiments, the nucleic acid encodes the helper enzyme in the form of first and second portions with the intein encoded between the first and second portions, such that the first and second portions are fused into a functional helper enzyme upon post-translational excision of the intein from the helper enzyme.

In embodiments, an intein is a suitable ligand-dependent intein, for example, an intein selected from those described in U.S. Pat. No. 9,200,045; Mootz et al., J. Am. Chem. Soc. 2002; 124, 9044-9045; Mootz et al., J. Am. Chem. Soc. 2003; 125, 10561-10569; Buskirk et al., Proc. Natl. Acad. Sci. USA. 2004; 101, 10505-10510; Skretas & Wood. Protein Sci. 2005; 14, 523-532; Schwartz, et al., Nat. Chem. Biol. 2007; 3, 50-54; Peck et al., Chem. Biol. 2011; 18 (5), 619-630; the entire contents of each of which are hereby incorporated by reference herein.

In embodiments the intein is NpuN (Intein-N) (SEQ ID NO: 423) and/or NpuC (Intein-C) (SEQ ID NO: 424), or a variant thereof, e.g., a sequence having at least about 90%, or at least about 93%, or at least about 95%, or at least about 97%, or at least about 98%, or at least about 99% identity thereto.

SEQ ID NO: 423: nucleotide sequence of NpuN
(Intein-N)
GGCGGATCTGGCGGTAGTGCTGAGTATTGTCTGAGTTACGAAACGGAAAT
ACTCACGGTTGAGTATGGGCTTCTTCCAATTGGCAAAATCGTTGAAAAGC
GCATAGAGTGTACGGTGTATTCCGTCGATAACAACGGTAATATCTACACC
CAGCCGGTAGCTCAGTGGCACGACCGAGGCGAACAGGAAGTGTTCGAGTA
TTGCTTGGAAGATGGCTCCCTTATCCGCGCCACTAAAGACCATAAGTTTA
TGACGGTTGACGGGCAGATGCTGCCTATAGACGAAATATTTGAGAGAGAG
CTGGACTTGATGAGAGTCGATAATCTGCCAAAT
SEQ ID NO: 424: nucleotide sequence of NpuC
(Intein-C)
GGCGGATCTGGCGGTAGTGGGGGTTCCGGATCCATAAAGATAGCTACTAG
GAAATATCTTGGCAAACAAAACGTCTATGACATAGGAGTTGAGCGAGATC
ACAATTTTGCTTTGAAGAATGGGTTCATCGCGTCTAATTGCTTCAACGCT
AGCGGCGGGTCAGGAGGCTCTGGTGGAAGC

### Dimerization Enhancers

In embodiments, a nucleic acid encoding the helper enzyme capable of targeted genomic integration by transposition comprises a dimerization enhancer. In embodiments, the nucleic acid encodes the helper enzyme in the form of first and second portions with the dimerization enhancer encoded between the first and second portions, such that the first and sec-ond portions are fused into a functional helper enzyme upon post-translational excision of the dimerization enhancer from the helper enzyme. In embodiments, the dimerization enhancer is suitable for linking the helper enzyme and the targeting element. In embodiments, the dimerization enhancer is selected from: a protein comprising a SH3 domain, biotin, avidin, or a rapamycin binder, optionally, wherein the rapamycin binder is FKBP12 or mTOR, or a variant thereof.

### Nucleic Acids of the Disclosure

In embodiments, a nucleic acid encoding the enzyme (e.g., without limitation, the helper enzyme) is RNA. In embodiments, a nucleic acid encoding the transgene is DNA.

In embodiments, the enzyme (e.g., without limitation, the helper enzyme) is encoded by a recombinant or synthetic nucleic acid. In embodiments, the nucleic acid is RNA, optionally a helper RNA. In embodiments, the nucleic acid is RNA that has a 5′-m7G cap (cap0, or cap1, or cap2), optionally with pseudouridine substitution (e.g., without limitation n-methyl-pseudouridine), and optionally a poly-A tail of about 30, or about 50, or about 100, of about 150 nucleotides in length. In embodiments, the poly-A tail is of about 30 nucleotides in length, optionally 34 nucleotides in length. In embodiments, a nuclear localization signal is placed before the enzyme start codon at the N-terminus, optionally at the C-terminus.

In embodiments, the nucleic acid that is RNA has a 5′-m7G cap (cap 0, or cap 1, or cap 2).

In embodiments, the nucleic acid comprises a 5′ cap structure, a 5′-UTR comprising a Kozak consensus sequence, a 5′-UTR comprising a sequence that increases RNA stability in vivo, a 3′-UTR comprising a sequence that increases RNA stability in vivo, and/or a 3′ poly(A) tail.

In embodiments, the enzyme (e.g., without limitation, a helper) is incorporated into a vector or a vector-like particle. In embodiments, the vector is a non-viral vector.

In embodiments, a nucleic acid encoding the helper enzyme in accordance with embodiments of the present disclosure, is DNA.

In various embodiments, a construct comprising a donor is any suitable genetic construct, such as a nucleic acid construct, a plasmid, or a vector. In various embodiments, the construct is DNA, which is referred to herein as a donor DNA. In embodiments, sequences of a nucleic acid encoding the donor is codon optimized to provide improved mRNA stability and protein expression in mammalian systems.

In embodiments, the helper enzyme and the donor are included in different vectors. In embodiments, the helper enzyme and the donor are included in the same vector.

In various embodiments, a nucleic acid encoding the helper enzyme capable of targeted genomic integration by transposition (e.g., without limitation, the helper enzyme) is RNA (e.g., helper RNA), and a nucleic acid encoding a donor is DNA.

As would be appreciated in the art, a donor often includes an open reading frame that encodes a transgene at the middle of donor and terminal repeat sequences at the 5′ and 3′ end of the donor. The translated helper (e.g., without limitation, the helper enzyme) binds to the 5′ and 3′ sequence of the donor and carries out the transposition function.

In embodiments, a donor is used interchangeably with transposable elements, which are used to refer to polynucleotides capable of inserting copies of themselves into other polynucleotides. The term donor is well known to those skilled in the art and includes classes of donors that can be distinguished on the basis of sequence organization, for example inverted terminal sequences at each end, and/or directly repeated long terminal repeats (LTRs) at the ends. In embodiments, the donor as described herein may be described as a piggyBac like element, e.g., a donor element that is characterized by its traceless excision, which recognizes TTAA (SEQ ID NO: 440) sequence and restores the sequence at the insert site back to the original TTAA (SEQ ID NO: 440) sequence after removal of the donor.

In embodiments, the donor is flanked by one or more end sequences or terminal ends. In embodiments, the donor is or comprises a gene encoding a complete polypeptide. In embodiments, the donor is or comprises a gene which is defective or substantially absent in a disease state.

In embodiments, a transgene is associated with various regulatory elements that are selected to ensure stable expression of a construct with the transgene. Thus, in embodiments, a transgene is encoded by a non-viral vector (e.g., without limitation, a DNA plasmid) that can comprise one or more insulator sequences that prevent or mitigate activation or inactivation of nearby genes. The insulators flank the donor (transgene cassette) to reduce transcriptional silencing and position effects imparted by chromosomal sequences. As an additional effect, the insulators can eliminate functional interactions of the transgene enhancer and promoter sequences with neighboring chromosomal sequences. In embodiments, the one or more insulator sequences comprise an HS4 insulator (1.2-kb 5′-HS4 chicken β-globin (cHS4) insulator element) and an D4Z4 insulator (tandem macrosatellite repeats linked to Facio-Scapulo-Humeral Dystrophy (FSHD). In embodiments, the sequences of the HS4 insulator and the D4Z4 insulator are as described in Rival-Gervier et al. Mol Ther. 2013 August; 21(8):1536-50, which is incorporated herein by reference in its entirety.

In embodiments, the transgene is inserted into a GSHS location in a host genome. GSHSs is defined as loci well-suited for gene transfer, as integrations within these sites are not associated with adverse effects such as proto-oncogene activation, tumor suppressor inactivation, or insertional mutagenesis. GSHSs can defined by the following criteria: (1) distance of at least 50 kb from the 5′ end of any gene, (2) distance of at least 300 kb from any cancer-related gene, (3) distance of at least 300 kb from any microRNA (miRNA), (4) location outside a transcription unit, and (5) location outside ultra-conserved regions (UCRs) of the human genome. See Papapetrou et al. Nat Biotechnol 2011; 29:73-8; Bejerano et al. Science 2004; 304:1321-5.

Furthermore, the use of GSHS locations can allow stable transgene expression across multiple cell types. One such site, chemokine C-C motif receptor 5 (CCR5) has been identified and used for integrative gene transfer. CCR5 is a member of the beta chemokine receptor family and is required for the entry of R5 tropic viral strains involved in primary infections. A homozygous 32 bp deletion in the CCR5 gene confers resistance to HIV-1 virus infections in humans. Disrupted CCR5 expression, naturally occurring in about 1% of the Caucasian population, does not appear to result in any reduction in immunity. Lobritz at al., Viruses 2010; 2:1069-105. A clinical trial has demonstrated safety and efficacy of disrupting CCR5 via targetable nucleases. Tebas at al., HIV. N Engl J Med 2014; 370:901-10.

In embodiments, the donor is under control of a tissue-specific promoter. The tissue-specific promoter is, e.g., without limitation, a liver-specific promoter. In embodiments, the liver-specific promoter is an LP1 promoter that, in embodiments, is a human LP1 promoter. The LP1 promoter is described, e.g., in Nathwani et al. Blood vol. 2006; 107(7):2653-61, and it is constructed, without limitation, as described in Nathawani et al.

It should be appreciated however that a variety of promoters can be used, including other tissue-specific promoters, inducible promoters, constitutive promoters, etc.

In embodiments, the present nucleic acids include polymeric form of nucleotides of any length, either ribonucleotides or deoxyribonucleotides, or analogs or derivatives thereof. In embodiments, there is provided double- and single-stranded DNA, as well as double- and single-stranded RNA, and RNA-DNA hybrids. In embodiments, transcriptionally-activated polynucleotides such as methylated or capped polynucleotides are provided. In embodiments, the present compositions are mRNA or DNA.

In embodiments, the present non-viral vectors are linear or circular DNA molecules that comprise a polynucleotide encoding a polypeptide and is operably linked to control sequences, wherein the control sequences provide for expression of the polynucleotide encoding the polypeptide. In embodiments, the non-viral vector comprises a promoter sequence, and transcriptional and translational stop signal sequences. Such vectors may include, among others, chromosomal and episomal vectors, e.g., vectors bacterial plasmids, from donors, from yeast episomes, from insertion elements, from yeast chromosomal elements, and vectors from combinations thereof. The present constructs may contain control regions that regulate as well as engender expression.

In embodiments, the construct comprising the helper enzyme and/or transgene is codon optimized. Transgene codon optimization is used to optimize therapeutic potential of the transgene and its expression in the host organism. Codon optimization is performed to match the codon usage in the transgene with the abundance of transfer RNA (tRNA) for each codon in a host organism or cell. Codon optimization methods are known in the art and described in, for example, WO 2007/142954, which is incorporated by reference herein in its entirety. Optimization strategies can include, for example, the modification of translation initiation regions, alteration of mRNA structural elements, and the use of different codon biases.

In embodiments, the construct comprising the helper enzyme and/or transgene includes several other regulatory elements that are selected to ensure stable expression of the construct. Thus, in embodiments, the non-viral vector is a DNA plasmid that can comprise one or more insulator sequences that prevent or mitigate activation or inactivation of nearby genes. In embodiments, the one or more insulator sequences comprise an HS4 insulator (1.2-kb 5′-HS4 chicken β-globin (cHS4) insulator element) and an D4Z4 insulator (tandem macrosatellite repeats linked to Facio-Scapulo-Humeral Dystrophy (FSHD). In embodiments, the sequences of the HS4 insulator and the D4Z4 insulator are as described in Rival-Gervier et al. Mol Ther. 2013 August; 21(8):1536-50, which is incorporated herein by reference in its entirety. In embodiments, the gene of the construct comprising the helper enzyme and/or transgene is capable of transposition in the presence of a helper. In embodiments, the non-viral vector in accordance with embodiments of the present disclosure comprises a nucleic acid construct encoding a helper. The helper (e.g., without limitation, the helper enzyme of the present disclosure) is an RNA helper plasmid. In embodiments, the non-viral vector further comprises a nucleic acid construct encoding a DNA helper plasmid. In embodiments, the helper is an in vitro-transcribed mRNA helper. The helper (e.g., without limitation, the helper enzyme of the present disclosure) is capable of excising and/or transposing the gene from the construct comprising the helper enzyme and/or transgene to site- or locus-specific genomic regions.

In embodiments, the enzyme (e.g., without limitation, the helper enzyme) and the donor are included in the same vector.

In embodiments, the helper enzyme is disposed on the same (cis) or different vector (trans) than a donor with a transgene. Accordingly, in embodiments, the helper enzyme and the donor encompassing a transgene are in cis configuration such that they are included in the same vector. In embodiments, the helper enzyme and the donor encompassing a transgene are in trans configuration such that they are included in different vectors. The vector is any non-viral vector in accordance with the present disclosure.

In some aspects, a nucleic acid encoding the donor system of the present disclosure capable of targeted genomic integration by transposition (e.g., a helper) in accordance with embodiments of the present disclosure is provided. The nucleic acid is or comprises DNA or RNA. In embodiments, the nucleic acid encoding the helper enzyme is DNA. In embodiments, the nucleic acid encoding the helper enzyme capable of targeted genomic integration by transposition (e.g., a helper of the present disclosure) is RNA such as, e.g., helper RNA. In embodiments, the helper is incorporated into a vector. In embodiments, the vector is a non-viral vector.

In embodiments, a nucleic acid encoding the transgene in accordance with embodiments of the present disclosure is provided. The nucleic acid is or comprises DNA or RNA. In embodiments, the nucleic acid encoding the transgene is DNA. In embodiments, the nucleic acid encoding the transgene is RNA such as, e.g., helper RNA. In embodiments, the transgene is incorporated into a vector. In embodiments, the vector is a non-viral vector.

In embodiments, the present helper enzyme can be in the form or an RNA or DNA and have one or two N-terminus nuclear localization signal (NLS) to shuttle the protein more efficiently into the nucleus. For example, in embodiments, the present helper enzyme further comprises one, two, three, four, five, or more NLSs. Examples of NLS are provided in Kosugi et al. (J. Biol. Chem. (2009) 284:478-485; incorporated by reference herein). In a particular embodiment, the NLS comprises the consensus sequence K(K/R)X(K/R) (SEQ ID NO: 348). In an embodiment, the NLS comprises the consensus sequence (K/R)(K/R)X₁₀₋₁₂(K/R)_{3/5}(SEQ ID NO: 349), where (K/R)_{3/5} represents at least three of the five amino acids is either lysine or arginine. In an embodiment, the NLS comprises the c-myc NLS. In a particular embodiment, the c-myc NLS comprises the sequence PAAKRVKLD (SEQ ID NO: 350). In a particular embodiment, the NLS is the nucleoplasmin NLS. In embodiments, the nucleoplasmin NLS comprises the sequence KRPAATKKAGQAKKKK (SEQ ID NO: 351). In embodiments, the NLS comprises the SV40 Large T-antigen NLS. In embodiments, the SV40 Large T-antigen NLS comprises the sequence PKKKRKV (SEQ ID NO: 352). In a particular embodiment, the NLS comprises three SV40 Large T-antigen NLSs (e.g., DPKKKRKVDPKKKRKVDPKKKRKV (SEQ ID NO: 353). In embodiments, the NLS may comprise mutations/variations in the above sequences such that they contain 1 or more substitutions, additions, or deletions (e.g., about 1, or about 2, or about 3, or about 4, or about 5, or about 10 substitutions, additions, or deletions).

In some aspects, a host cell comprising the nucleic acid in accordance with embodiments of the present disclosure is provided.

### Lipids and LNP Delivery

In embodiments, a composition or a nucleic acid in accordance with embodiments of the present disclosure is provided wherein the composition is in the form of a lipid nanoparticle (LNP). In embodiments, the composition is encapsulated in an LNP.

In embodiments, a nucleic acid encoding the helper enzyme and a nucleic acid encoding the transgene are contained within the same lipid nanoparticle (LNP). In embodiments, the nucleic acid encoding the helper enzyme and the nucleic acid encoding the donor are a mixture incorporated into or associated with the same LNP. In embodiments, the polynucleotide encoding the helper enzyme and the polynucleotide encoding the donor are in the form of the same LNP, optionally in a co-formulation.

In embodiments, the LNP is selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy(polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC) and/or comprising of one or more molecules selected from polyethylenimine (PEI) and poly(lactic-co-glycolic acid) (PLGA), and N-Acetylgalactosamine (GalNAc).

In embodiments, an LNP is as described, e.g., in Patel et al., J Control Release 2019; 303:91-100. The LNP can comprise one or more of a structural lipid (e.g., DSPC), a PEG-conjugated lipid (CDM-PEG), a cationic lipid (MC3), cholesterol, and a targeting ligand (e.g., GalNAc).

In embodiments, a nanoparticle is a particle having a diameter of less than about 1000 nm. In embodiments, nanoparticles of the present disclosure have a greatest dimension (e.g., diameter) of about 500 nm or less, or about 400 nm or less, or about 300 nm or less, or about 200 nm or less, or about 100 nm or less. In embodiments, nanoparticles of the present disclosure have a greatest dimension ranging between about 50 nm and about 150 nm, or between about 70 nm and about 130 nm, or between about 80 nm and about 120 nm, or between about 90 nm and about 110 nm. In embodiments, the nanoparticles of the present disclosure have a greatest dimension (e.g., a diameter) of about 100 nm.

In some aspects, the cell in accordance with the present disclosure is prepared via an in vivo genetic modification method. In embodiments, a genetic modification in accordance with the present disclosure is performed via an ex vivo method.

In some aspects, the cell in accordance with the present disclosure is prepared by contacting a cell with a helper enzyme capable of targeted genomic integration by transposition (e.g., without limitation, the helper enzyme) in vivo.

In embodiments, the cell is contacted with the helper enzyme ex vivo.

In embodiments, the present method provides high specific targeting as compared to a method that does not use the helper enzyme with a target selector.

### Therapeutic Applications

In embodiments, the transgene of interest in accordance with embodiments of the present disclosure can encode various genes.

In embodiments, the helper enzyme and the donor are included in the same pharmaceutical composition.

In embodiments, the helper enzyme and the donor are included in different pharmaceutical compositions.

In embodiments, the helper enzyme and the donor are co-transfected.

In embodiments the helper enzyme and the donor are transfected separately.

In embodiments, a transfected cell for gene therapy is provided, wherein the transfected cell is generated using the helper enzyme in accordance with embodiments of the present disclosure.

In embodiments, a method of delivering a cell therapy is provided, comprising administering to a patient in need thereof the transfected cell generated using the helper enzyme in accordance with embodiments of the present disclosure.

In embodiments, a method of treating a disease or condition using a cell therapy, comprising administering to a patient in need thereof the transfected cell generated using the helper enzyme in accordance with embodiments of the present disclosure.

In embodiments, the disease or condition may comprise cancer. In embodiments, the cancer is or comprises an adrenal cancer, a biliary track cancer, a bladder cancer, a bone/bone marrow cancer, a brain cancer, a breast cancer, a cervical cancer, a colorectal cancer, a cancer of the esophagus, a gastric cancer, a head/neck cancer, a hepatobiliary cancer, a kidney cancer, a liver cancer, a lung cancer, an ovarian cancer, a pancreatic cancer, a pelvis cancer, a pleura cancer, a prostate cancer, a renal cancer, a skin cancer, a stomach cancer, a testis cancer, a thymus cancer, a thyroid cancer, a uterine cancer, a lymphoma, a melanoma, a multiple myeloma, or a leukemia.

In embodiments, the cancer is selected from one or more of the basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; glioblastoma; hepatic carcinoma; hepatoma; intra-epithelial neoplasm; kidney or renal cancer; larynx cancer; leukemia; liver cancer; lung cancer; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; Hodgkin's lymphoma; non-Hodgkin's lymphoma; B-cell lymphoma; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's Macroglobulinemia; chronic lymphocytic leukemia (CLL); acute lymphoblastic leukemia (ALL); and Hairy cell leukemia.

In embodiments, the cancer is selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), and Meigs syndrome.

In embodiments, the disease or condition is or comprises an infectious disease. In embodiments, the infectious disease is a coronavirus infection, optionally selected from infection with SAR-CoV, MERS-CoV, and SARS-CoV-2, or variants thereof.

In embodiments, the infectious disease is or comprises a disease comprising a viral infection, a parasitic infection, or a bacterial infection. In embodiments, the viral infection is caused by a virus of family Flaviviridae, a virus of family Picornaviridae, a virus of family Orthomyxoviridae, a virus of family Coronaviridae, a virus of family Retroviridae, a virus of family Paramyxoviridae, a virus of family Bunyaviridae, or a virus of family Reoviridae.

In embodiments, the virus of family Coronaviridae comprises a betacoronavirus or an alphacoronavirus, optionally wherein the betacoronavirus is selected from SARS-CoV-2, SARS-CoV, MERS-CoV, HCoV-HKU1, and HCoV-OC43, or the alphacoronavirus is selected from a HCoV-NL63 and HCoV-229E. In embodiments, the infectious disease comprises a coronavirus infection 2019 (COVID-19).

In embodiments, the method requires a single administration. In embodiments, the method requires a plurality of administrations.

### Isolated Cell

In some aspects of the present disclosure, an isolated cell is provided that comprises the transfected cell in accordance with embodiments of the present disclosure.

In some aspects, the present disclosure provides an ex vivo gene therapy approach. Accordingly, in embodiments, the method that is used to treat an inherited or acquired disease in a patient in need thereof comprises (a) contacting a cell obtained from a patient (autologous) or another individual (allogeneic) with a transfected cell in accordance with embodiments of the present disclosure; and (b) administering the cell to a patient in need thereof.

One of the advantages of ex vivo gene therapy is the ability to “sample” the transduced cells before patient administration. This facilitates efficacy and allows performing safety checks before introducing the cell(s) to the patient. For example, the transduction efficiency and/or the clonality of integration can be assessed before infusion of the product. The present disclosure provides transfected cells and methods that can be effectively used for ex vivo gene modification.

In embodiments, a composition comprising transfected cells in accordance with the present disclosure comprises a pharmaceutically acceptable carrier, excipient, or diluent.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile, and the fluid should be easy to draw up by a syringe. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly[1,3-bis(carboxyphenoxy)propane-co-sebacic-acid] (PCPP-SA) matrix, fatty acid dimer-sebacic acid (FAD-SA) copolymer, poly(lactide-co-glycolide)), polyglycolic acid, collagen, polyorthoesters, polyethyleneglycol-coated liposomes, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006; 79(3-4): 141-152.

In embodiments, there is provided a method of transforming a cell using the construct comprising the helper enzyme and/or transgene described herein in the presence of a helper (e.g., without limitation, the helper enzyme) to produce a stably transfected cell which results from the stable integration of a gene of interest into the cell. In embodiments, the stable integration comprises an introduction of a polynucleotide into a chromosome or mini-chromosome of the cell and, therefore, becomes a relatively permanent part of the cellular genome.

In embodiments, there is provided a transgenic organism that may comprise cells which have been transformed by the methods of the present disclosure. In embodiments, the organism may be a mammal or an insect. When the organism is a mammal, the organism may include, but is not limited to, a mouse, a rat, a chimpanzee, an elephant, a dog, a rabbit, a raccoon, and the like. When the organism is an insect, the organism may include, but is not limited to, a fruit fly, an ant, a mosquito, a bollworm, and the like.

### Methods for Identifying Site-Specific Targeting to a Nucleic Acid

In aspects, there is provided a method for identifying site-specific targeting to a nucleic acid by a helper enzyme and a targeting element, comprising: (a) transfecting a cell with a donor plasmid, the helper enzyme and a targeting element, and a reporter plasmid, wherein: the donor plasmid comprises a first fragment of a reporter gene under the control of a promoter and a splice-donor site (SD); the reporter plasmid comprises a landing pad for the targeting element comprising site specific DNA binding recognition sites flanking a TTAA followed by a splice acceptor site (SA) and a second fragment of a reporter gene; and (b) splicing and integrating into the landing pad, to permit the reconstitution of the reporter gene from the fragments thereof and thereby causing a reporter readout. In embodiments, the method further comprises (c) amplifying the donor plasmid to identify targeting. In embodiments, the method further comprises (d) sequencing the amplified product to analyze integration in specific sequence regions. In embodiments, the SA and SD are spliced out of the donor plasmid in step (b).

In embodiments, the amplifying is via PCR. In embodiments, the sequencing is amplicon sequencing in embodiments, the fluorescent protein is or comprises a monomeric red fluorescent protein (mRFP). In embodiments, the mRFP is selected from mCherry, DsRed, mRFP1, mStrawberry, mOrange, and dTomato. In embodiments, the fluorescent protein is or comprises a green fluorescent protein (GFP). In embodiments, the reporter readout is fluorescence. In embodiments, the promoter is selected from cytomegalovirus (CMV), CMV enhancer fused to the chicken β-actin (CAG), chicken β-actin (CBA), simian vacuolating virus 40 (SV40), β glucuronidase (GUSB), polyubiquitin C gene (UBC), elongation-factor 1α subunit (EF-1α), and phosphoglycerate kinase (PGK).

In embodiments, the helper enzyme is a recombinase, integrase or a transposase. In embodiments, the helper enzyme is a mammal-derived transposase. In embodiments, the helper enzyme is derived from Bombyx mori, Xenopus tropicalis, Trichoplusia ni, Myotis lucifugus, Rhinolophus ferrumequinum, Rousettus aegyptiacus, Phyllostomus discolor, Myotis myotis, Pteropus vampyrus, Pipistrellus kuhlii, troglodytes, Molossus molossus, or Homo sapiens. In embodiments, the helper enzyme comprises an amino acid sequence having at least about 80% sequence identity to SEQ ID NO: 9 and has a non-polar aliphatic amino acid at position 2 of SEQ ID NO: 9 or a position corresponding thereto and one or more of S8X₁ of SEQ ID NO: 9 or a position corresponding thereto, wherein X₁ is selected from alanine (A), glycine (G), valine (V), leucine (L), isoleucine (I), and proline (P); C13X₂ of SEQ ID NO: 9 or a position corresponding thereto, wherein X₂ is selected from lysine (K), arginine (R), and histidine (H); and N125X₃ of SEQ ID NO: 9 or a position corresponding thereto, wherein X₃ is selected from is selected from lysine (K), arginine (R), and histidine (H).

In embodiments, the targeting element is or comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof.

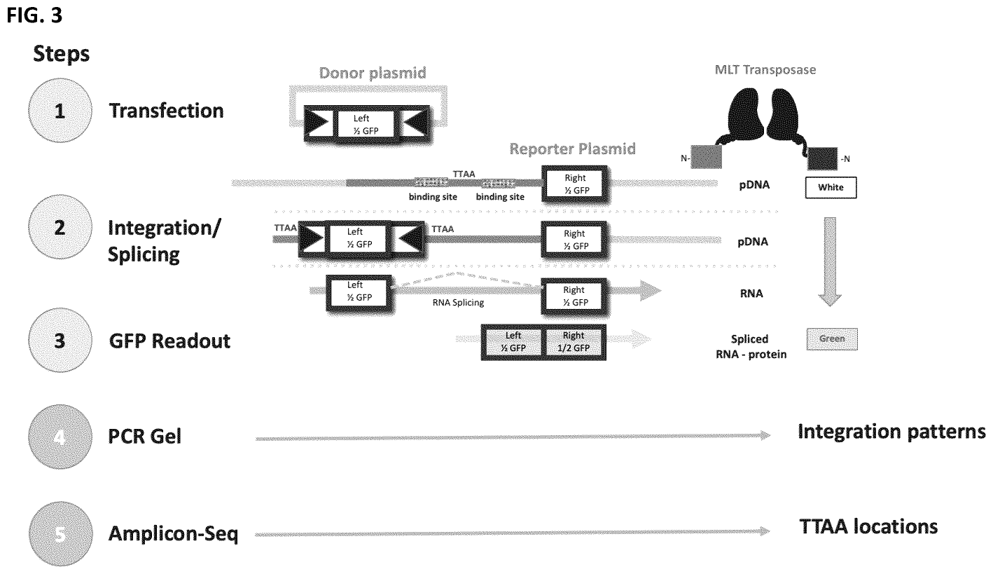

In embodiments, the method is substantially as in FIG. 3.

### Definitions

The following definitions are used in connection with the disclosure disclosed herein. Unless defined otherwise, all technical and scientific terms used herein have the same meaning as commonly understood to one of skill in the art to which this invention belongs.

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

The term “in vivo” refers to an event that takes place in a subject's body.

The term “ex vivo” refers to an event which involves treating or performing a procedure on a cell, tissue and/or organ which has been removed from a subject's body. Aptly, the cell, tissue and/or organ may be returned to the subject's body in a method of treatment or surgery.

As used herein, the term “variant” encompasses but is not limited to nucleic acids or proteins which comprise a nucleic acid or amino acid sequence which differs from the nucleic acid or amino acid sequence of a reference by way of one or more substitutions, deletions and/or additions at certain positions. The variant may comprise one or more conservative substitutions. Conservative substitutions may involve, e.g., the substitution of similarly charged or uncharged amino acids.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, lipid, or the like, which is nontoxic, and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD₅₀ (the dose lethal to about 50% of the population) and the ED₅₀ (the dose therapeutically effective in about 50% of the population). The dosage can vary depending upon the dosage form employed and the route of administration utilized. The dose ratio between toxic and therapeutic effects is the therapeutic index and can be expressed as the ratio LD₅₀/ED₅₀. In embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC₅₀ as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

As used herein, “methods of treatment” are equally applicable to use of a composition for treating the diseases or disorders described herein and/or compositions for use and/or uses in the manufacture of a medicaments for treating the diseases or disorders described herein.

### SELECTED SEQUENCES

In embodiments, the present disclosure provides for any of the sequence provided herein, including the below, and a variant sequence having at least about 90%, or at least about 93%, or at least about 95%, or at least about 97%, or at least about 98%, or at least about 99% identity thereto, or at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

| SEQ ID NO: 9: amino acid sequence of a variant of the hyperactive helper with S | |
|---|---|
| at position 8 and C at position 13 (572 amino acids) | |
| 1 | MAQHSDYSDD EFCADKLSNY SCDSDLENAS TSDEDSSDDE VMVRPRTLRR RRISSSSSDS |
| 61 | ESDIEGGREE WSHVDNPPVL EDFLGHQGLN TDAVINNIED AVKLFIGDDF FEFLVEESNR |
| 121 | YYNQNRNNFK LSKKSLKWKD ITPQEMKKFL GLIVLMGQVR KDRRDDYWTT EPWTETPYFG |
| 181 | KTMTRDRFRQ IWKAWHFNNN ADIVNESDRL CKVRPVLDYF VPKFINIYKP HQQLSLDEGI |
| 241 | VPWRGRLFFR VYNAGKIVKY GILVRLLCES DTGYICNMEI YCGEGKRLLE TIQTVVSPYT |
| 301 | DSWYHIYMDN YYNSVANCEA LMKNKFRICG TIRKNRGIPK DFQTISLKKG ETKFIRKNDI |
| 361 | LLQVWQSKKP VYLISSIHSA EMEESQNIDR TSKKKIVKPN ALIDYNKHMK GVDRADQYLS |
| 421 | YYSILRRTVK WTKRLAMYMI NCALFNSYAV YKSVRQRKMG FKMFLKQTAI HWLTDDIPED |
| 481 | MDIVPDLQPV PSTSGMRAKP PTSDPPCRLS MDMRKHTLQA IVGSGKKKNI LRRCRVCSVH |
| 541 | KLRSETRYMC KFCNIPLHKG ACFEKYHTLK NY |
| SEQ ID NO: 10: nucleotide sequence encoding SEQ ID NO: 9 (1719 nt) | |
| 1 | ATGGCCCAGC ACAGCGACTA CCCCGACGAC GAGTTCAGAG CCGATAAGCT GAGTAACTAC |
| 61 | AGCTGCGACA GCGACCTGGA AAACGCCAGC ACATCCGACG AGGACAGCTC TGACGACGAG |
| 121 | GTGATGGTGC GGCCCAGAAC CCTGAGACGG AGAAGAATCA GCAGCTCTAG CAGCGACTCT |
| 181 | GAATCCGACA TCGAGGGCGG CCGGGAAGAG TGGAGCCACG TGGACAACCC TCCTGTTCTG |
| 241 | GAAGATTTTC TGGGCCATCA GGGCCTGAAC ACCGACGCCG TGATCAACAA CATCGAGGAT |
| 301 | GCCGTGAAGC TGTTCATAGG AGATGATTTC TTTGAGTTCC TGGTCGAGGA ATCCAACCGC |
| 361 | TATTACAACC AGAATAGAAA CAACTTCAAG CTGAGCAAGA AAAGCCTGAA GTGGAAGGAC |
| 421 | ATCACCCCTC AGGAGATGAA AAAGTTCCTG GGACTGATCG TTCTGATGGG ACAGGTGCGG |
| 481 | AAGGACAGAA GGGATGATTA CTGGACAACC GAACCTTGGA CCGAGACCCC TTACTTTGGC |
| 541 | AAGACCATGA CCAGAGACAG ATTCAGACAG ATCTGGAAAG CCTGGCACTT CAACAACAAT |
| 601 | GCTGATATCG TGAACGAGTC TGATAGACTG TGTAAAGTGC GGCCAGTGTT GGATTACTTC |
| 661 | GTGCCTAAGT TCATCAACAT CTATAAGCCT CACCAGCAGC TGAGCCTGGA TGAAGGCATC |
| 721 | GTGCCCTGGC GGGGCAGACT GTTCTTCAGA GTGTACAATG CTGGCAAGAT CGTCAAATAC |
| 781 | GGCATCCTGG TGCGCCTTCT GTGCGAGAGC GATACAGGCT ACATCTGTAA TATGGAAATC |
| 841 | TACTGCGGCG AGGGCAAAAG ACTGCTGGAA ACCATCCAGA CCGTCGTTTC CCCTTATACC |
| 901 | GACAGCTGGT ACCACATCTA CATGGACAAC TACTACAATT CTGTGGCCAA CTGCGAGGCC |
| 961 | CTGATGAAGA ACAAGTTTAG AATCTGCGGC ACAATCAGAA AAAACAGAGG CATCCCTAAG |
| 1021 | GACTTCCAGA CCATCTCTCT GAAGAAGGGC GAAACCAAGT TCATCAGAAA GAACGACATC |
| 1081 | CTGCTCCAAG TGTGGCAGTC CAAGAAACCC GTGTACCTGA TCAGCAGCAT CCATAGCGCC |
| 1141 | GAGATGGAAG AAAGCCAGAA CATCGACAGA ACAAGCAAGA AGAAGATCGT GAAGCCCAAT |
| 1201 | GCTCTGATCG ACTACAACAA GCACATGAAA GGCGTGGACC GGGCCGACCA GTACCTGTCT |
| 1261 | TATTACTCTA TCCTGAGAAG AACAGTGAAA TGGACCAAGA GACTGGCCAT GTACATGATC |
| 1321 | AATTGCGCCC TGTTCAACAG CTACGCCGTG TACAAGTCCG TGCGACAAAG AAAAATGGGA |
| 1381 | TTCAAGATGT TCCTGAAGCA GACAGCCATC CACTGGCTGA CAGACGACAT TCCTGAGGAC |
| 1441 | ATGGACATTG TGCCAGATCT GCAACCTGTG CCCAGCACCT CTGGTATGAG AGCTAAGCCT |
| 1501 | CCCACCAGCG ATCCTCCATG TAGACTGAGC ATGGACATGC GGAAGCACAC CCTGCAGGCC |
| 1561 | ATCGTCGGCA GCGGCAAGAA GAAGAACATC CTTAGACGGT GCAGGGTGTG CAGCGTGCAC |
| 1621 | AAGCTGCGGA GCGAGACTCG GTACATGTGC AAGTTTTGCA ACATTCCCCT GCACAAGGGA |
| 1681 | GCCTGCTTCG AGAAGTACCA CACCCTGAAG AATTACTAG |
| SEQ ID NO: 1: nucleotide sequence of hyperactive helper mRNA helper construct | |
| (1956 bp)(Order of underlined sequences: T7 promoter, hyperactive helper, | |
| polyA tail; the 5′-globin and 3′-globin UTRs are in capital letters). | |
| 1 | taatacgact cactataagg aagCTTCTTG TTCTTTTTGC AGAAGCTCAG AATAAACGCT |
| 61 | CAACTTTGGc cgccaccatg gcccagcaca gcgactaccc cgacgacgag ttcagagccg |
| 121 | ataagctgag taactacagc tgcgacagcg acctggaaaa cgccagcaca tccgacgagg |
| 181 | acagctctga cgacgaggtg atggtgcggc ccagaaccct gagacggaga agaatcagca |
| 241 | gctctagcag cgactctgaa tccgacatcg agggcggccg ggaagagtgg agccacgtgg |
| 301 | acaaccctcc tgttctggaa gattttctgg gccatcaggg cctgaacacc gacgccgtga |
| 361 | tcaacaacat cgaggatgcc gtgaagctgt tcataggaga tgatttcttt gagttcctgg |
| 421 | tcgaggaatc caaccgctat tacaaccaga atagaaacaa cttcaagctg agcaagaaaa |
| 481 | gcctgaagtg gaaggacatc acccctcagg agatgaaaaa gttcctggga ctgatcgttc |
| 541 | tgatgggaca ggtgcggaag gacagaaggg atgattactg gacaaccgaa ccttggaccg |
| 601 | agacccctta ctttggcaag accatgacca gagacagatt cagacagatc tggaaagcct |
| 661 | ggcacttcaa caacaatgct gatatgctga acgagtctga tagactgtgt aaagtgcggc |
| 721 | cagtgttgga ttacttcgtg cctaagttca tcaacatcta taagcctcac cagcagctga |
| 781 | gcctggatga aggcatcgtg ccctggcggg gcagactgtt cttcagagtg tacaatgctg |
| 841 | gcaagatcgt caaatacggc atcctggtgc gccttctgtg cgagagcgat acaggctaca |
| 901 | tctgtaatat ggaaatctac tgcggcgagg gcaaaagact gctggaaacc atccagaccg |
| 961 | tcgtttcccc ttataccgac agctggtacc acatctacat ggacaactac tacaattctg |
| 1021 | tggccaactg cgaggccctg atgaagaaca agtttagaat ctgcggcaca atcagaaaaa |
| 1081 | acagaggcat ccctaaggac ttccagacca tctctctgaa gaagggcgaa accaagttca |
| 1141 | tcagaaagaa cgacatcctg ctccaagtgt ggcagtccaa gaaacccgtg tacctgatca |
| 1201 | gcagcatcca tagcgccgag atggaagaaa gccagaacat cgacagaaca agcaagaaga |
| 1261 | agatcgtgaa gcccaatgct ctgatcgact acaacaagca catgaaaggc gtggaccggg |
| 1321 | ccgaccagta cctgtcttat tactctatcc tgagaagaac agtgaaatgg accaagagac |
| 1381 | tggccatgta catgatcaat tgcgccctgt tcaacagcta cgccgtgtac aagtccgtgc |
| 1441 | gacaaagaaa aatgggattc aagatgttcc tgaagcagac agccatccac tggctgacag |
| 1501 | acgacattcc tgaggacatg gacattgtgc cagatctgca acctgtgccc agcacctctg |
| 1561 | gtatgagagc taagcctccc accagcgatc ctccatgtag actgagcatg gacatgcgga |
| 1621 | agcacaccct gcaggccatc gtcggcagcg gcaagaagaa gaacatcctt agacggtgca |
| 1681 | gggtgtgcag cgtgcacaag ctgcggagcg agactcggta catgtgcaag ttttgcaaca |
| 1741 | ttcccctgca caagggagcc tgcttcgaga agtaccacac cctgaagaat tactagAACC |
| 1801 | AGCCTCAAGA ACACCCGAAT GGAGTCTCTA AGCTACATAA TACCAACTTA CACTTTACAA |
| 1861 | AATGTTGTCC CCCAAAATGT AGCCATTCGT ATCTGCTCCT AATAAAAAGA AAGTTTCTTC |
| 1921 | ACaaaaaaaa aaaaaaaaaa aaaaaaaaaa aaaaaa |
| SEQ ID NO: 2: amino acid sequence of hyperactive helper (572 amino acids) | |
| 1 | MAQHSDYPDD EFRADKLSNY SCDSDLENAS TSDEDSSDDE VMVRPRTLRR RRISSSSSDS |
| 61 | ESDIEGGREE WSHVDNPPVL EDFLGHQGLN TDAVINNIED AVKLFIGDDF FEFLVEESNR |
| 121 | YYNQNRNNFK LSKKSLKWKD ITPQEMKKFL GLIVLMGQVR KDRRDDYWTT EPWTETPYFG |
| 181 | KTMTRDRFRQ IWKAWHFNNN ADIVNESDRL CKVRPVLDYF VPKFINIYKP HQQLSLDEGI |
| 241 | VPWRGRLFFR VYNAGKIVKY GILVRLLCES DTGYICNMEI YCGEGKRLLE TIQTVVSPYT |
| 301 | DSWYHIYMDN YYNSVANCEA LMKNKFRICG TIRKNRGIPK DFQTISLKKG ETKFIRKNDI |
| 361 | LLQVWQSKKP VYLISSIHSA EMEESQNIDR TSKKKIVKPN ALIDYNKHMK GVDRADQYLS |
| 421 | YYSILRRTVK WTKRLAMYMI NCALFNSYAV YKSVRQRKMG FKMFLKQTAI HWLTDDIPED |
| 481 | MDIVPDLQPV PSTSGMRAKP PTSDPPCRLS MDMRKHTLQA IVGSGKKKNI LRRCRVCSVH |
| 541 | KLRSETRYMC KFCNIPLHKG ACFEKYHTLK NY |
| SEQ ID NO: 11: nucleotide sequence encoding hyperactive helper (SEQ ID NO: 2) | |
| (1719 nt) | |
| 1 | ATGGCCCAGC ACAGCGACTA CCCCGACGAC GAGTTCAGAG CCGATAAGCT GAGTAACTAC |
| 61 | AGCTGCGACA GCGACCTGGA AAACGCCAGC ACATCCGACG AGGACAGCTC TGACGACGAG |
| 121 | GTGATGGTGC GGCCCAGAAC CCTGAGACGG AGAAGAATCA GCAGCTCTAG CAGCGACTCT |
| 181 | GAATCCGACA TCGAGGGCGG CCGGGAAGAG TGGAGCCACG TGGACAACCC TCCTGTTCTG |
| 241 | GAAGATTTTC TGGGCCATCA GGGCCTGAAC ACCGACGCCG TGATCAACAA CATCGAGGAT |
| 301 | GCCGTGAAGC TGTTCATAGG AGATGATTTC TTIGAGTTCC TGGTCGAGGA ATCCAACCGC |
| 361 | TATTACAACC AGAATAGAAA CAACTTCAAG CTGAGCAAGA AAAGCCTGAA GTGGAAGGAC |
| 421 | ATCACCCCTC AGGAGATGAA AAAGTTCCTG GGACTGATCG TTCTGATGGG ACAGGTGCGG |
| 481 | AAGGACAGAA GGGATGATTA CTGGACAACC GAACCTTGGA CCGAGACCCC TTACTTTGGC |
| 541 | AAGACCATGA CCAGAGACAG ATTCAGACAG ATCTGGAAAG CCTGGCACTT CAACAACAAT |
| 601 | GCTGATATCG TGAACGAGTC TGATAGACTG TGTAAAGTGC GGCCAGTGTT GGATTACTTC |
| 661 | GTGCCTAAGT TCATCAACAT CTATAAGCCT CACCAGCAGC TGAGCCTGGA TGAAGGCATC |
| 721 | GTGCCCTGGC GGGGCAGACT GTTCTTCAGA GTGTACAATG CTGGCAAGAT CGTCAAATAC |
| 781 | GGCATCCTGG TGCGCCTTCT GTGCGAGAGC GATACAGGCT ACATCTGTAA TATGGAAATC |
| 841 | TACTGCGGCG AGGGCAAAAG ACTGCTGGAA ACCATCCAGA CCGTCGTTTC CCCTTATACC |
| 901 | GACAGCTGGT ACCACATCTA CATGGACAAC TACTACAATT CTGTGGCCAA CTGCGAGGCC |
| 961 | CTGATGAAGA ACAAGTTTAG AATCTGCGGC ACAATCAGAA AAAACAGAGG CATCCCTAAG |
| 1021 | GACTTCCAGA CCATCTCTCT GAAGAAGGGC GAAACCAAGT TCATCAGAAA GAACGACATC |
| 1081 | CTGCTCCAAG TGTGGCAGTC CAAGAAACCC GTGTACCTGA TCAGCAGCAT CCATAGCGCC |
| 1141 | GAGATGGAAG AAAGCCAGAA CATCGACAGA ACAAGCAAGA AGAAGATCGT GAAGCCCAAT |
| 1201 | GCTCTGATCG ACTACAACAA GCACATGAAA GGCGTGGACC GGGCCGACCA GTACCTGTCT |
| 1261 | TATTACTCTA TCCTGAGAAG AACAGTGAAA TGGACCAAGA GACTGGCCAT GTACATGATC |
| 1321 | AATTGCGCCC TGTTCAACAG CTACGCCGTG TACAAGTCCG TGCGACAAAG AAAAATGGGA |
| 1381 | TTCAAGATGT TCCTGAAGCA GACAGCCATC CACTGGCTGA CAGACGACAT TCCTGAGGAC |
| 1441 | ATGGACATTG TGCCAGATCT GCAACCTGTG CCCAGCACCT CTGGTATGAG AGCTAAGCCT |
| 1501 | CCCACCAGCG ATCCTCCATG TAGACTGAGC ATGGACATGC GGAAGCACAC CCTGCAGGCC |
| 1561 | ATCGTCGGCA GCGGCAAGAA GAAGAACATC CTTAGACGGT GCAGGGTGTG CAGCGTGCAC |
| 1621 | AAGCTGCGGA GCGAGACTCG GTACATGTGC AAGTTTTGCA ACATTCCCCT GCACAAGGGA |
| 1681 | GCCTGCTTCG AGAAGTACCA CACCCTGAAG AATTACTAG |
| SEQ ID NO: 3: hyperactive helper Left ITR (157 bp) | |
| The left ITR retains recognition activity when the underlined nucleotides | |
| are deleted (80 bp). | |
| 1 | ttaacacttg gattgcggga aacgagttaa gtcggctcgc gtgaattgcg cgtactccgc |
| 61 | gggagccgtc ttaactcggt tcatatagat ttgcggtgga gtgcgggaaa cgtgtaaact |
| 121 | cgggccgatt gtaactgcgt attaccaaat atttgtt |
| SEQ ID NO: 4: hyperactive helper Right ITR (212 bp) | |
| The right ITR retains recognition activity when the underlined nucleotides | |
| are deleted (80 bp). | |
| 1 | aattatttat gtactgaata gataaaaaaa tgtctgtgat tgaataaatt ttcatttttt |
| 61 | acacaagaaa ccgaaaattt catttcaatc gaacccatac ttcaaaagat ataggcattt |
| 121 | taaactaact ctgattttgc gcgggaaacc taaataattg cccgcgccat cttatatttt |
| 181 | ggcgggaaat tcacccgaca ccgtagtgtt aa |
| SEQ ID NO: 5: nucleotide sequence of dead Cas9 DNA BINDING protein (5004 bp) | |
| 1 | ATGGACAAGA AGTACTCCAT TGGGCTCGCT ATCGGCACAA ACAGCGTCGG CTGGGCCGTC |
| 61 | ATTACGGACG AGTACAAGGT GCCGAGCAAA AAATTCAAAG TTCTGGGCAA TACCGATCGC |
| 121 | CACAGCATAA AGAAGAACCT CATTGGCGCC CTCCTGTTCG ACTCCGGGGA GACGGCCGAA |
| 181 | GCCACGCGGC TCAAAAGAAC AGCACGGCGC AGATATACCC GCAGAAAGAA TCGGATCTGC |
| 241 | TACCTGCAGG AGATCTTTAG TAATGAGATG GCTAAGGTGG ATGACTCTTT CTTCCATAGG |
| 301 | CTGGAGGAGT CCTTTTTGGT GGAGGAGGAT AAAAAGCACG AGCGCCACCC AATCTTTGGC |
| 361 | AATATCGTGG ACGAGGTGGC GTACCATGAA AAGTACCCAA CCATATATCA TCTGAGGAAG |
| 421 | AAGCTTGTAG ACAGTACTGA TAAGGCTGAC TTGCGGTTGA TCTATCTCGC GCTGGCGCAT |
| 481 | ATGATCAAAT TTCGGGGACA CTTCCTCATC GAGGGGGACC TGAACCCAGA CAACAGCGAT |
| 541 | GTCGACAAAC TCTTTATCCA ACTGGTTCAG ACTTACAATC AGCTTTTCGA AGAGAACCCG |
| 601 | ATCAACGCAT CCGGAGTTGA CGCCAAAGCA ATCCTGAGCG CTAGGCTGTC CAAATCCCGG |
| 661 | CGGCTCGAAA ACCTCATCGC ACAGCTCCCT GGGGAGAAGA AGAACGGCCT GTTTGGTAAT |
| 721 | CTTATCGCCC TGTCACTCGG GCTGACCCCC AACTTTAAAT CTAACTTCGA CCTGGCCGAA |
| 781 | GATGCCAAGC TTCAACTGAG CAAAGACACC TACGATGATG ATCTCGACAA TCTGCTGGCC |
| 841 | CAGATCGGCG ACCAGTACGC AGACCTTTTT TTGGCGGCAA AGAACCTGTC AGACGCCATT |
| 901 | CTGCTGAGTG ATATTCTGCG AGTGAACACG GAGATCACCA AAGCTCCGCT GAGCGCTAGT |
| 961 | ATGATCAAGC GCTATGATGA GCACCACCAA GACTTGACTT TGCTGAAGGC CCTTGTCAGA |
| 1021 | CAGCAACTGC CTGAGAAGTA CAAGGAAATT TTCTTCGATC AGTCTAAAAA TGGCTACGCC |
| 1081 | GGATACATTG ACGGCGGAGC AAGCCAGGAG GAATTTTACA AATTTATTAA GCCCATCTTG |
| 1141 | GAAAAAATGG ACGGCACCGA GGAGCTGCTG GTAAAGCTTA ACAGAGAAGA TCTGTTGCGC |
| 1201 | AAACAGCGCA CTTTCGACAA TGGAAGCATC CCCCACCAGA TTCACCTGGG CGAACTGCAC |
| 1261 | GCTATCCTCA GGCGGCAAGA GGATTTCTAC CCCTTTTTGA AAGATAACAG GGAAAAGATT |
| 1321 | GAGAAAATCC TCACATTTCG GATACCCTAC TATGTAGGCC CCCTCGCCCG GGGAAATTCC |
| 1381 | AGATTCGCGT GGATGACTCG CAAATCAGAA GAGACCATCA CTCCCTGGAA CTTCGAGGAA |
| 1441 | GTCGTGGATA AGGGGGCCTC TGCCCAGTCC TTCATCGAAA GGATGACTAA CTTTGATAAA |
| 1501 | AATCTGCCTA ACGAAAAGGT GCTTCCTAAA CACTCTCTGC TGTACGAGTA CTTCACAGTT |
| 1561 | TATAACGAGC TCACCAAGGT CAAATACGTC ACAGAAGGGA TGAGAAAGCC AGCATTCCTG |
| 1621 | TCTGGAGAGC AGAAGAAAGC TATCGTGGAC CTCCTCTTCA AGACGAACCG GAAAGTTACC |
| 1681 | GTGAAACAGC TCAAAGAAGA CTATTTCAAA AAGATTGAAT GTTTCGACTC TGTTGAAATC |
| 1741 | AGCGGAGTGG AGGATCGCTT CAACGCATCC CTGGGAACGT ATCACGATCT CCTGAAAATC |
| 1801 | ATTAAAGACA AGGACTTCCT GGACAATGAG GAGAACGAGG ACATTCTTGA GGACATTGTC |
| 1861 | CTCACCCTTA CGTTGTTTGA AGATAGGGAG ATGATTGAAG AACGCTTGAA AACTTACGCT |
| 1921 | CATCTCTTCG ACGACAAAGT CATGAAACAG CTCAAGAGGC GCCGATATAC AGGATGGGGG |
| 1981 | CGGCTGTCAA GAAAACTGAT CAATGGGATC CGAGACAAGC AGAGTGGAAA GACAATCCTG |
| 2041 | GATTTTCTTA AGTCCGATGG ATTTGCCAAC CGGAACTTCA TGCAGTTGAT CCATGATGAC |
| 2101 | TCTCTCACCT TTAAGGAGGA CATCCAGAAA GCACAAGTTT CTGGCCAGGG GGACAGTCTT |
| 2161 | CACGAGCACA TCGCTAATCT TGCAGGTAGC CCAGCTATCA AAAAGGGAAT ACTGCAGACC |
| 2221 | GTTAAGGTCG TGGATGAACT CGTCAAAGTA ATGGGAAGGC ATAAGCCCGA GAATATCGTT |
| 2281 | ATCGAGATGG CCCGAGAGAA CCAAACTACC CAGAAGGGAC AGAAGAACAG TAGGGAAAGG |
| 2341 | ATGAAGAGGA TTGAAGAGGG TATAAAAGAA CTGGGGTCCC AAATCCTTAA GGAACACCCA |
| 2401 | GTTGAAAACA CCCAGCTTCA GAATGAGAAG CTCTACCTGT ACTACCTGCA GAACGGCAGG |
| 2461 | GACATGTACG TGGATCAGGA ACTGGACATC AATCGGCTCT CCGACTACGA CGTGGCTGCT |
| 2521 | ATCGTGCCCC AGTCTTTTCT CAAAGATGAT TCTATTGATA ATAAAGTGTT GACAAGATCC |
| 2581 | GATAAAGCTA GAGGGAAGAG TGATAACGTC CCCTCAGAAG AAGTTGTCAA GAAAATGAAA |
| 2641 | AATTATTGGC GGCAGCTGCT GAACGCCAAA CTGATCACAC AACGGAAGTT CGATAATCTG |
| 2701 | ACTAAGGCTG AACGAGGTGG CCTGTCTGAG TTGGATAAAG CCGGCTTCAT CAAAAGGCAG |
| 2761 | CTTGTTGAGA CACGCCAGAT CACCAAGCAC GTGGCCCAAA TTCTCGATTC ACGCATGAAC |
| 2821 | ACCAAGTACG ATGAAAATGA CAAACTGATT CGAGAGGTGA AAGTTATTAC TCTGAAGTCT |
| 2881 | AAGCTGGTCT CAGATTTCAG AAAGGACTTT CAGTTTTATA AGGTGAGAGA GATCAACAAT |
| 2941 | TACCACCATG CGCATGATGC CTACCTGAAT GCAGTGGTAG GCACTGCACT TATCAAAAAA |
| 3001 | TATCCCAAGC TTGAATCTGA ATTTGTTTAC GGAGACTATA AAGTGTACGA TGTTAGGAAA |
| 3061 | ATGATCGCAA AGTCTGAGCA GGAAATAGGC AAGGCCACCG CTAAGTACTT CTTTTACAGC |
| 3121 | AATATTATGA ATTTTTTCAA GACCGAGATT ACACTGGCCA ATGGAGAGAT TCGGAAGCGA |
| 3181 | CCACTTATCG AAACAAACGG AGAAACAGGA GAAATCGTGT GGGACAAGGG TAGGGATTTC |
| 3241 | GCGACAGTCC GGAAGGTCCT GTCCATGCCG CAGGTGAACA TCGTTAAAAA GACCGAAGTA |
| 3301 | CAGACCGGAG GCTTCTCCAA GGAAAGTATC CTCCCGAAAA GGAACAGCGA CAAGCTGATC |
| 3361 | GCACGCAAAA AAGATTGGGA CCCCAAGAAA TACGGCGGAT TCGATTCTCC TACAGTCGCT |
| 3421 | TACAGTGTAC TGGTTGTGGC CAAAGTGGAG AAAGGGAAGT CTAAAAAACT CAAAAGCGTC |
| 3481 | AAGGAACTGC TGGGCATCAC AATCATGGAG CGATCAAGCT TCGAAAAAAA CCCCATCGAC |
| 3541 | TTTCTGGAGG CGAAAGGATA TAAAGAGGTC AAAAAAGACC TCATCATTAA GCTTCCCAAG |
| 3601 | TACTCTCTCT TTGAGCTTGA AAACGGCCGG AAACGAATGC TCGCTAGTGC GGGCGAGCTG |
| 3661 | CAGAAAGGTA ACGAGCTGGC ACTGCCCTCT AAATACGTTA ATTTCTTGTA TCTGGCCAGC |
| 3721 | CACTATGAAA AGCTCAAAGG GTCTCCCGAA GATAATGAGC AGAAGCAGCT GTTCGTGGAA |
| 3781 | CAACACAAAC ACTACCTTGA TGAGATCATC GAGCAAATAA GCGAATTCTC CAAAAGAGTG |
| 3841 | ATCCTCGCCG ACGCTAACCT CGATAAGGTG CTTTCTGCTT ACAATAAGCA CAGGGATAAG |
| 3901 | CCCATCAGGG AGCAGGCAGA AAACATTATC CACTTGTTTA CTCTGACCAA CTTGGGCGCG |
| 3961 | CCTGCAGCCT TCAAGTACTT CGACACCACC ATAGACAGAA AGCGGTACAC CTCTACAAAG |
| 4021 | GAGGTCCTGG ACGCCACACT GATTCATCAG TCAATTACGG GGCTCTATGA AACAAGAATC |
| 4081 | GACCTCTCTC AGCTCGGTGG AGAC |
| SEQ ID NO: 6: amino acid sequence of dead Cas9 DNA BINDING protein (1368 | |
| amino acids) | |
| 1 | MDKKYSIGLA IGTNSVGWAV ITDEYKVPSK KFKVLGNTDR HSIKKNLIGA LLFDSGETAE |
| 61 | ATRLKRTARR RYTRRKNRIC YLQEIFSNEM AKVDDSFFHR LEESFLVEED KKHERHPIFG |
| 121 | NIVDEVAYHE KYPTIYHLRK KLVDSTDKAD LRLIYLALAH MIKFRGHFLI EGDLNPDNSD |
| 181 | VDKLFIQLVQ TYNQLFEENP INASGVDAKA ILSARLSKSR RLENLIAQLP GEKKNGLFGN |
| 241 | LIALSLGLTP NFKSNFDLAE DAKLQLSKDT YDDDLDNLLA QIGDQYADLF LAAKNLSDAI |
| 301 | LLSDILRVNT EITKAPLSAS MIKRYDEHHQ DLTLLKALVR QQLPEKYKEI FFDQSKNGYA |
| 361 | GYIDGGASQE EFYKFIKPIL EKMDGTEELL VKLNREDLLR KQRTFDNGSI PHQIHLGELH |
| 421 | AILRRQEDFY PFLKDNREKI EKILTFRIPY YVGPLARGNS RFAWMTRKSE ETITPWNFEE |
| 481 | VVDKGASAQS FIERMTNFDK NLPNEKVLPK HSLLYEYFTV YNELTKVKYV TEGMRKPAFL |
| 541 | SGEQKKAIVD LLFKTNRKVT VKQLKEDYFK KIECFDSVEI SGVEDRFNAS LGTYHDLLKI |
| 601 | IKDKDFLDNE ENEDILEDIV LTLTLFEDRE MIEERLKTYA HLFDDKVMKQ LKRRRYTGWG |
| 661 | RLSRKLINGI RDKQSGKTIL DFLKSDGFAN RNFMQLIHDD SLTFKEDIQK AQVSGQGDSL |
| 721 | HEHIANLAGS PAIKKGILQT VKVVDELVKV MGRHKPENIV IEMARENQTT QKGQKNSRER |
| 781 | MKRIEEGIKE LGSQILKEHP VENTQLQNEK LYLYYLQNGR DMYVDQELDI NRLSDYDVAA |
| 841 | IVPQSFLKDD SIDNKVLTRS DKARGKSDNV PSEEVVKKMK NYWRQLLNAK LITQRKFDNL |
| 901 | TKAERGGLSE LDKAGFIKRQ LVETRQITKH VAQILDSRMN TKYDENDKLI REVKVITLKS |
| 961 | KLVSDFRKDF QFYKVREINN YHHAHDAYLN AVVGTALIKK YPKLESEFVY GDYKVYDVRK |
| 1021 | MIAKSEQEIG KATAKYFFYS NIMNFFKTEI TLANGEIRKR PLIETNGETG EIVWDKGRDF |
| 1081 | ATVRKVLSMP QVNIVKKTEV QTGGFSKESI LPKRNSDKLI ARKKDWDPKK YGGFDSPTVA |
| 1141 | YSVLVVAKVE KGKSKKLKSV KELLGITIME RSSFEKNPID FLEAKGYKEV KKDLIIKLPK |
| 1201 | YSLFELENGR KRMLASAGEL QKGNELALPS KYVNFLYLAS HYEKLKGSPE DNEQKQLFVE |
| 1261 | QHKHYLDEII EQISEFSKRV ILADANLDKV LSAYNKHRDK PIREQAENII HLFTLTNLGA |
| 1321 | PAAFKYFDTT IDRKRYTSTK EVLDATLIHQ SITGLYETRI DLSQLGGD |
| SEQ ID NO: 12: amino acid sequence of E. coli TnsD (508 amino acids) | |
| 1 | MRNFPVPYSN ELIYSTIARA GVYQGIVSPK QLLDEVYGNR KVVATLGLPS HLGVIARHLH |
| 61 | QTGRYAVQQL IYEHTLFPLY APFVGKERRD EAIRLMEYQA QGAVHLMLGV AASRVKSDNR |
| 121 | FRYCPDCVAL QLNRYGEAFW QRDWYLPALP YCPKHGALVF FDRAVDDHRH QFWALGHTEL |
| 181 | LSDYPKDSLS QLTALAAYIA PLLDAPRAQE LSPSLEQWTL FYQRLAQDLG LTKSKHIRHD |
| 241 | LVAERVRQTF SDEALEKLDL KLAENKDTCW LKSIFRKHRK AFSYLQHSIV WQALLPKLTV |
| 301 | IEALQQASAL TEHSITTRPV SQSVQPNSED LSVKHKDWQQ LVHKYQGIKA ARQSLEGGVL |
| 361 | YAWLYRHDRD WLVHWNQQHQ QERLAPAPRV DWNQRDRIAV RQLLRIIKRL DSSLDHPRAT |
| 421 | SSWLLKQTPN GTSLAKNLQK LPLVALCLKR YSESVEDYQI RRISQAFIKL KQEDVELRRW |
| 481 | RLLRSATLSK ERITEEAQRF LEMVYGEE |
| SEQ ID NO: 501: Myositis lucifugus (hyperactive helper) nucleotide sequence(NO). | |
| 1716 bp | |
| 1 | ATGGCCCAGC ACAGCGACTA CCCCGACGAC GAGTTCAGAG CCGATAAGCT GAGTAACTAC |
| 61 | AGCTGCGACA GCGACCTGGA AAACGCCAGC ACATCCGACG AGGACAGCTC TGACGACGAG |
| 121 | GTGATGGTGC GGCCCAGAAC CCTGAGACGG AGAAGAATCA GCAGCTCTAG CAGCGACTCT |
| 181 | GAATCCGACA TCGAGGGCGG CCGGGAAGAG TGGAGCCACG TGGACAACCC TCCTGTTCTG |
| 241 | GAAGATTTTC TGGGCCATCA GGGCCTGAAC ACCGACGCCG TGATCAACAA CATCGAGGAT |
| 301 | GCCGTGAAGC TGTTCATAGG AGATGATTTC TTTGAGTTCC TGGTCGAGGA ATCCAACCGC |
| 361 | TATTACAACC AGAATAGAAA CAACTTCAAG CTGAGCAAGA AAAGCCTGAA GTGGAAGGAC |
| 421 | ATCACCCCTC AGGAGATGAA AAAGTTCCTG GGACTGATCG TTCTGATGGG ACAGGTGCGG |
| 481 | AAGGACAGAA GGGATGATTA CTGGACAACC GAACCTTGGA CCGAGACCCC TTACTTTGGC |
| 541 | AAGACCATGA CCAGAGACAG ATTCAGACAG ATCTGGAAAG CCTGGCACTT CAACAACAAT |
| 601 | GCTGATATCG TGAACGAGTC TGATAGACTG TGTAAAGTGC GGCCAGTGTT GGATTACTTC |
| 661 | GTGCCTAAGT TCATCAACAT CTATAAGCCT CACCAGCAGC TGAGCCTGGA TGAAGGCATC |
| 721 | GTGCCCTGGC GGGGCAGACT GTTCTTCAGA GTGTACAATG CTGGCAAGAT CGTCAAATAC |
| 781 | GGCATCCTGG TGCGCCTTCT GTGCGAGAGC GATACAGGCT ACATCTGTAA TATGGAAATC |
| 841 | TACTGCGGCG AGGGCAAAAG ACTGCTGGAA ACCATCCAGA CCGTCGTTTC CCCTTATACC |
| 901 | GACAGCTGGT ACCACATCTA CATGGACAAC TACTACAATT CTGTGGCCAA CTGCGAGGCC |
| 961 | CTGATGAAGA ACAAGTTTAG AATCTGCGGC ACAATCAGAA AAAACAGAGG CATCCCTAAG |
| 1021 | GACTTCCAGA CCATCTCTCT GAAGAAGGGC GAAACCAAGT TCATCAGAAA GAACGACATC |
| 1081 | CTGCTCCAAG TGTGGCAGTC CAAGAAACCC GTGTACCTGA TCAGCAGCAT CCATAGCGCC |
| 1141 | GAGATGGAAG AAAGCCAGAA CATCGACAGA ACAAGCAAGA AGAAGATCGT GAAGCCCAAT |
| 1201 | GCTCTGATCG ACTACAACAA GCACATGAAA GGCGTGGACC GGGCCGACCA GTACCTGTCT |
| 1261 | TATTACTCTA TCCTGAGAAG AACAGTGAAA TGGACCAAGA GACTGGCCAT GTACATGATC |
| 1321 | AATTGCGCCC TGTTCAACAG CTACGCCGTG TACAAGTCCG TGCGACAAAG AAAAATGGGA |
| 1381 | TTCAAGATGT TCCTGAAGCA GACAGCCATC CACTGGCTGA CAGACGACAT TCCTGAGGAC |
| 1441 | ATGGACATTG TGCCAGATCT GCAACCTGTG CCCAGCACCT CTGGTATGAG AGCTAAGCCT |
| 1501 | CCCACCAGCG ATCCTCCATG TAGACTGAGC ATGGACATGC GGAAGCACAC CCTGCAGGCC |
| 1561 | ATCGTCGGCA GCGGCAAGAA GAAGAACATC CTTAGACGGT GCAGGGTGTG CAGCGTGCAC |
| 1621 | AAGCTGCGGA GCGAGACTCG GTACATGTGC AAGTTTTGCA ACATTCCCCT GCACAAGGGA |
| 1681 | GCCTGCTTCG AGAAGTACCA CACCCTGAAG AATTAC |
| SEQ ID NO: 502: Myositis lucifugus (hyperactive helper) amino acid sequence(NO). | |
| 572 aa | |
| 1 | MAQHSDYPDD EFRADKLSNY SCDSDLENAS TSDEDSSDDE VMVRPRTLRR RRISSSSSDS |
| 61 | ESDIEGGREE WSHVDNPPVL EDFLGHQGLN TDAVINNIED AVKLFIGDDF FEFLVEESNR |
| 121 | YYNQNRNNFK LSKKSLKWKD ITPQEMKKFL GLIVLMGQVR KDRRDDYWTT EPWTETPYFG |
| 181 | KTMTRDRFRQ IWKAWHFNNN ADIVNESDRL CKVRPVLDYF VPKFINIYKP HQQLSLDEGI |
| 241 | VPWRGRLFFR VYNAGKIVKY GILVRLLCES DTGYICNMEI YCGEGKRLLE TIQTVVSPYT |
| 301 | DSWYHIYMDN YYNSVANCEA LMKNKFRICG TIRKNRGIPK DFQTISLKKG ETKFIRKNDI |
| 361 | LLQVWQSKKP VYLISSIHSA EMEESQNIDR TSKKKIVKPN ALIDYNKHMK GVDRADQYLS |
| 421 | YYSILRRTVK WTKRLAMYMI NCALFNSYAV YKSVRQRKMG FKMFLKQTAI HWLTDDIPED |
| 481 | MDIVPDLQPV PSTSGMRAKP PTSDPPCRLS MDMRKHTLQA IVGSGKKKNI LRRCRVCSVH |
| 541 | KLRSETRYMC KFCNIPLHKG ACFEKYHTLK NY |
| SEQ ID NO: 503: N-terminal deletion Myositis lucifugus (hyperactive helper) | |
| nucleotide sequence (N1; nucleotide 4-105 deletion). 1614 bp | |
| 1 | ATGAGCTCTG ACGACGAGGT GATGGTGCGG CCCAGAACCC TGAGACGGAG AAGAATCAGC |
| 61 | AGCTCTAGCA GCGACTCTGA ATCCGACATC GAGGGCGGCC GGGAAGAGTG GAGCCACGTG |
| 121 | GACAACCCTC CTGTTCTGGA AGATTTTCTG GGCCATCAGG GCCTGAACAC CGACGCCGTG |
| 181 | ATCAACAACA TCGAGGATGC CGTGAAGCTG TTCATAGGAG ATGATTTCTT TGAGTTCCTG |
| 241 | GTCGAGGAAT CCAACCGCTA TTACAACCAG AATAGAAACA ACTTCAAGCT GAGCAAGAAA |
| 301 | AGCCTGAAGT GGAAGGACAT CACCCCTCAG GAGATGAAAA AGTTCCTGGG ACTGATCGTT |
| 361 | CTGATGGGAC AGGTGCGGAA GGACAGAAGG GATGATTACT GGACAACCGA ACCTTGGACC |
| 421 | GAGACCCCTT ACTTTGGCAA GACCATGACC AGAGACAGAT TCAGACAGAT CTGGAAAGCC |
| 481 | TGGCACTTCA ACAACAATGC TGATATCGTG AACGAGTCTG ATAGACTGTG TAAAGTGCGG |
| 541 | CCAGTGTTGG ATTACTTCGT GCCTAAGTTC ATCAACATCT ATAAGCCTCA CCAGCAGCTG |
| 601 | AGCCTGGATG AAGGCATCGT GCCCTGGCGG GGCAGACTGT TCTTCAGAGT GTACAATGCT |
| 661 | GGCAAGATCG TCAAATACGG CATCCTGGTG CGCCTTCTGT GCGAGAGCGA TACAGGCTAC |
| 721 | ATCTGTAATA TGGAAATCTA CTGCGGCGAG GGCAAAAGAC TGCTGGAAAC CATCCAGACC |
| 781 | GTCGTTTCCC CTTATACCGA CAGCTGGTAC CACATCTACA TGGACAACTA CTACAATTCT |
| 841 | GTGGCCAACT GCGAGGCCCT GATGAAGAAC AAGTTTAGAA TCTGCGGCAC AATCAGAAAA |
| 901 | AACAGAGGCA TCCCTAAGGA CTTCCAGACC ATCTCTCTGA AGAAGGGCGA AACCAAGTTC |
| 961 | ATCAGAAAGA ACGACATCCT GCTCCAAGTG TGGCAGTCCA AGAAACCCGT GTACCTGATC |
| 1021 | AGCAGCATCC ATAGCGCCGA GATGGAAGAA AGCCAGAACA TCGACAGAAC AAGCAAGAAG |
| 1081 | AAGATCGTGA AGCCCAATGC TCTGATCGAC TACAACAAGC ACATGAAAGG CGTGGACCGG |
| 1141 | GCCGACCAGT ACCTGTCTTA TTACTCTATC CTGAGAAGAA CAGTGAAATG GACCAAGAGA |
| 1201 | CTGGCCATGT ACATGATCAA TTGCGCCCTG TTCAACAGCT ACGCCGTGTA CAAGTCCGTG |
| 1261 | CGACAAAGAA AAATGGGATT CAAGATGTTC CTGAAGCAGA CAGCCATCCA CTGGCTGACA |
| 1321 | GACGACATTC CTGAGGACAT GGACATTGTG CCAGATCTGC AACCTGTGCC CAGCACCTCT |
| 1381 | GGTATGAGAG CTAAGCCTCC CACCAGCGAT CCTCCATGTA GACTGAGCAT GGACATGCGG |
| 1441 | AAGCACACCC TGCAGGCCAT CGTCGGCAGC GGCAAGAAGA AGAACATCCT TAGACGGTGC |
| 1501 | AGGGTGTGCA GCGTGCACAA GCTGCGGAGC GAGACTCGGT ACATGTGCAA GTTTTGCAAC |
| 1561 | ATTCCCCTGC ACAAGGGAGC CTGCTTCGAG AAGTACCACA CCCTGAAGAA TTAC |
| 1621 | AAGCTGCGGA |
| SEQ ID NO: 504: Myositis lucifugus (hyperactive helper) amino acid sequence | |
| (N1, amino acid 2-35 deletion). 538 aa | |
| 1 | MSSDDEVMVR PRTLRRRRIS SSSSDSESDI EGGREEWSHV DNPPVLEDFL GHQGLNTDAV |
| 61 | INNIEDAVKL FIGDDFFEFL VEESNRYYNQ NRNNFKLSKK SLKWKDITPQ EMKKFLGLIV |
| 121 | LMGQVRKDRR DDYWTTEPWT ETPYFGKTMT RDRFRQIWKA WHFNNNADIV NESDRLCKVR |
| 181 | PVLDYFVPKF INIYKPHQQL SLDEGIVPWR GRLFFRVYNA GKIVKYGILV RLLCESDTGY |
| 241 | ICNMEIYCGE GKRLLETIQT VVSPYTDSWY HIYMDNYYNS VANCEALMKN KFRICGTIRK |
| 301 | NRGIPKDFQT ISLKKGETKF IRKNDILLQV WQSKKPVYLI SSIHSAEMEE SQNIDRTSKK |
| 361 | KIVKPNALID YNKHMKGVDR ADQYLSYYSI LRRTVKWTKR LAMYMINCAL FNSYAVYKSV |
| 421 | RQRKMGFKMF LKQTAIHWLT DDIPEDMDIV PDLQPVPSTS GMRAKPPTSD PPCRLSMDMR |
| 481 | KHTLQAIVGS GKKKNILRRC RVCSVHKLRS ETRYMCKFCN IPLHKGACFE KYHTLKNY |
| SEQ ID NO: 505: N-terminal deletion Myositis lucifugus (hyperactive helper) | |
| nucleotide sequence (N2; nucleotide 4-135 deletion). 1584 bp | |
| 1 | ATGAGAACCC TGAGACGGAG AAGAATCAGC AGCTCTAGCA GCGACTCTGA ATCCGACATC |
| 61 | GAGGGCGGCC GGGAAGAGTG GAGCCACGTG GACAACCCTC CTGTTCTGGA AGATTTTCTG |
| 121 | GGCCATCAGG GCCTGAACAC CGACGCCGTG ATCAACAACA TCGAGGATGC CGTGAAGCTG |
| 181 | TTCATAGGAG ATGATTTCTT TGAGTTCCTG GTCGAGGAAT CCAACCGCTA TTACAACCAG |
| 241 | AATAGAAACA ACTTCAAGCT GAGCAAGAAA AGCCTGAAGT GGAAGGACAT CACCCCTCAG |
| 301 | GAGATGAAAA AGTTCCTGGG ACTGATCGTT CTGATGGGAC AGGTGCGGAA GGACAGAAGG |
| 361 | GATGATTACT GGACAACCGA ACCTTGGACC GAGACCCCTT ACTTTGGCAA GACCATGACC |
| 421 | AGAGACAGAT TCAGACAGAT CTGGAAAGCC TGGCACTTCA ACAACAATGC TGATATCGTG |
| 481 | AACGAGTCTG ATAGACTGTG TAAAGTGCGG CCAGTGTTGG ATTACTTCGT GCCTAAGTTC |
| 541 | ATCAACATCT ATAAGCCTCA CCAGCAGCTG AGCCTGGATG AAGGCATCGT GCCCTGGCGG |
| 601 | GGCAGACTGT TCTTCAGAGT GTACAATGCT GGCAAGATCG TCAAATACGG CATCCTGGTG |
| 661 | CGCCTTCTGT GCGAGAGCGA TACAGGCTAC ATCTGTAATA TGGAAATCTA CTGCGGCGAG |
| 721 | GGCAAAAGAC TGCTGGAAAC CATCCAGACC GTCGTTTCCC CTTATACCGA CAGCTGGTAC |
| 781 | CACATCTACA TGGACAACTA CTACAATTCT GTGGCCAACT GCGAGGCCCT GATGAAGAAC |
| 841 | AAGTTTAGAA TCTGCGGCAC AATCAGAAAA AACAGAGGCA TCCCTAAGGA CTTCCAGACC |
| 901 | ATCTCTCTGA AGAAGGGCGA AACCAAGTTC ATCAGAAAGA ACGACATCCT GCTCCAAGTG |
| 961 | TGGCAGTCCA AGAAACCCGT GTACCTGATC AGCAGCATCC ATAGCGCCGA GATGGAAGAA |
| 1021 | AGCCAGAACA TCGACAGAAC AAGCAAGAAG AAGATCGTGA AGCCCAATGC TCTGATCGAC |
| 1081 | TACAACAAGC ACATGAAAGG CGTGGACCGG GCCGACCAGT ACCTGTCTTA TTACTCTATC |
| 1141 | CTGAGAAGAA CAGTGAAATG GACCAAGAGA CTGGCCATGT ACATGATCAA TTGCGCCCTG |
| 1201 | TTCAACAGCT ACGCCGTGTA CAAGTCCGTG CGACAAAGAA AAATGGGATT CAAGATGTTC |
| 1261 | CTGAAGCAGA CAGCCATCCA CTGGCTGACA GACGACATTC CTGAGGACAT GGACATTGTG |
| 1321 | CCAGATCTGC AACCTGTGCC CAGCACCTCT GGTATGAGAG CTAAGCCTCC CACCAGCGAT |
| 1381 | CCTCCATGTA GACTGAGCAT GGACATGCGG AAGCACACCC TGCAGGCCAT CGTCGGCAGC |
| 1441 | GGCAAGAAGA AGAACATCCT TAGACGGTGC AGGGTGTGCA GCGTGCACAA GCTGCGGAGC |
| 1501 | GAGACTCGGT ACATGTGCAA GTTTTGCAAC ATTCCCCTGC ACAAGGGAGC CTGCTTCGAG |
| 1561 | AAGTACCACA CCCTGAAGAA TTAC |
| SEQ ID NO: 506: Myositis lucifugus (hyperactive helper) amino acid sequence | |
| (N2, amino acid 2-45 deletion). 528 aa | |
| 1 | MRTLRRRRIS SSSSDSESDI EGGREEWSHV DNPPVLEDFL GHQGLNTDAV INNIEDAVKL |
| 61 | FIGDDFFEFL VEESNRYYNQ NRNNFKLSKK SLKWKDITPQ EMKKFLGLIV LMGQVRKDRR |
| 121 | DDYWTTEPWT ETPYFGKTMT RDRFRQIWKA WHFNNNADIV NESDRLCKVR PVLDYFVPKF |
| 181 | INIYKPHQQL SLDEGIVPWR GRLFFRVYNA GKIVKYGILV RLLCESDTGY ICNMEIYCGE |
| 241 | GKRLLETIQT VVSPYTDSWY HIYMDNYYNS VANCEALMKN KFRICGTIRK NRGIPKDFQT |
| 301 | ISLKKGETKF IRKNDILLQV WQSKKPVYLI SSIHSAEMEE SQNIDRTSKK KIVKPNALID |
| 361 | YNKHMKGVDR ADQYLSYYSI LRRTVKWTKR LAMYMINCAL FNSYAVYKSV RQRKMGFKMF |
| 421 | LKQTAIHWLT DDIPEDMDIV PDLQPVPSTS GMRAKPPTSD PPCRLSMDMR KHTLQAIVGS |
| 481 | GKKKNILRRC RVCSVHKLRS ETRYMCKFCN IPLHKGACFE KYHTLKNY |
| SEQ ID NO: 507: N-terminal deletion Myositis lucifugus (hyperactive helper) | |
| nucleotide sequence (N3; nucleotide 4-204 deletion). 1515 bp | |
| 1 | ATGGAAGAGT GGAGCCACGT GGACAACCCT CCTGTTCTGG AAGATTTTCT GGGCCATCAG |
| 61 | GGCCTGAACA CCGACGCCGT GATCAACAAC ATCGAGGATG CCGTGAAGCT GTTCATAGGA |
| 121 | GATGATTTCT TTGAGTTCCT GGTCGAGGAA TCCAACCGCT ATTACAACCA GAATAGAAAC |
| 181 | AACTTCAAGC TGAGCAAGAA AAGCCTGAAG TGGAAGGACA TCACCCCTCA GGAGATGAAA |
| 241 | AAGTTCCTGG GACTGATCGT TCTGATGGGA CAGGTGCGGA AGGACAGAAG GGATGATTAC |
| 301 | TGGACAACCG AACCTTGGAC CGAGACCCCT TACTTTGGCA AGACCATGAC CAGAGACAGA |
| 361 | TTCAGACAGA TCTGGAAAGC CTGGCACTTC AACAACAATG CTGATATCGT GAACGAGTCT |
| 421 | GATAGACTGT GTAAAGTGCG GCCAGTGTTG GATTACTTCG TGCCTAAGTT CATCAACATC |
| 481 | TATAAGCCTC ACCAGCAGCT GAGCCTGGAT GAAGGCATCG TGCCCTGGCG GGGCAGACTG |
| 541 | TTCTTCAGAG TGTACAATGC TGGCAAGATC GTCAAATACG GCATCCTGGT GCGCCTTCTG |
| 601 | TGCGAGAGCG ATACAGGCTA CATCTGTAAT ATGGAAATCT ACTGCGGCGA GGGCAAAAGA |
| 661 | CTGCTGGAAA CCATCCAGAC CGTCGTTTCC CCTTATACCG ACAGCTGGTA CCACATCTAC |
| 721 | ATGGACAACT ACTACAATTC TGTGGCCAAC TGCGAGGCCC TGATGAAGAA CAAGTTTAGA |
| 781 | ATCTGCGGCA CAATCAGAAA AAACAGAGGC ATCCCTAAGG ACTTCCAGAC CATCTCTCTG |
| 841 | AAGAAGGGCG AAACCAAGTT CATCAGAAAG AACGACATCC TGCTCCAAGT GTGGCAGTCC |
| 901 | AAGAAACCCG TGTACCTGAT CAGCAGCATC CATAGCGCCG AGATGGAAGA AAGCCAGAAC |
| 961 | ATCGACAGAA CAAGCAAGAA GAAGATCGTG AAGCCCAATG CTCTGATCGA CTACAACAAG |
| 1021 | CACATGAAAG GCGTGGACCG GGCCGACCAG TACCTGTCTT ATTACTCTAT CCTGAGAAGA |
| 1081 | ACAGTGAAAT GGACCAAGAG ACTGGCCATG TACATGATCA ATTGCGCCCT GTTCAACAGC |
| 1141 | TACGCCGTGT ACAAGTCCGT GCGACAAAGA AAAATGGGAT TCAAGATGTT CCTGAAGCAG |
| 1201 | ACAGCCATCC ACTGGCTGAC AGACGACATT CCTGAGGACA TGGACATTGT GCCAGATCTG |
| 1261 | CAACCTGTGC CCAGCACCTC TGGTATGAGA GCTAAGCCTC CCACCAGCGA TCCTCCATGT |
| 1321 | AGACTGAGCA TGGACATGCG GAAGCACACC CTGCAGGCCA TCGTCGGCAG CGGCAAGAAG |
| 1381 | AAGAACATCC TTAGACGGTG CAGGGTGTGC AGCGTGCACA AGCTGCGGAG CGAGACTCGG |
| 1441 | TACATGTGCA AGTTTTGCAA CATTCCCCTG CACAAGGGAG CCTGCTTCGA GAAGTACCAC |
| 1501 | ACCCTGAAGA ATTAC |
| SEQ ID NO: 508: Myositis lucifugus (hyperactive helper) amino acid sequence | |
| (N3, amino acid 2-68 deletion) 505 aa | |
| 1 | MEEWSHVDNP PVLEDFLGHQ GLNTDAVINN IEDAVKLFIG DDFFEFLVEE SNRYYNQNRN |
| 61 | NFKLSKKSLK WKDITPQEMK KFLGLIVLMG QVRKDRRDDY WTTEPWTETP YFGKTMTRDR |
| 121 | FRQIWKAWHF NNNADIVNES DRLCKVRPVL DYFVPKFINI YKPHQQLSLD EGIVPWRGRL |
| 181 | FFRVYNAGKI VKYGILVRLL CESDTGYICN MEIYCGEGKR LLETIQTVVS PYTDSWYHIY |
| 241 | MDNYYNSVAN CEALMKNKFR ICGTIRKNRG IPKDFQTISL KKGETKFIRK NDILLQVWQS |
| 301 | KKPVYLISSI HSAEMEESQN IDRTSKKKIV KPNALIDYNK HMKGVDRADQ YLSYYSILRR |
| 361 | TVKWTKRLAM YMINCALFNS YAVYKSVRQR KMGFKMFLKQ TAIHWLTDDI PEDMDIVPDL |
| 421 | QPVPSTSGMR AKPPTSDPPC RLSMDMRKHT LQAIVGSGKK KNILRRCRVC SVHKLRSETR |
| 481 | YMCKFCNIPL HKGACFEKYH TLKNY |
| SEQ ID NO: 509: N-terminal deletion Myositis lucifugus (hyperactive helper) | |
| nucleotide sequence (N4; nucleotide 4-267 deletion). 1452 bp | |
| 1 | ATGAACACCG ACGCCGTGAT CAACAACATC GAGGATGCCG TGAAGCTGTT CATAGGAGAT |
| 61 | GATTTCTTTG AGTTCCTGGT CGAGGAATCC AACCGCTATT ACAACCAGAA TAGAAACAAC |
| 121 | TTCAAGCTGA GCAAGAAAAG CCTGAAGTGG AAGGACATCA CCCCTCAGGA GATGAAAAAG |
| 181 | TTCCTGGGAC TGATCGTTCT GATGGGACAG GTGCGGAAGG ACAGAAGGGA TGATTACTGG |
| 241 | ACAACCGAAC CTTGGACCGA GACCCCTTAC TTTGGCAAGA CCATGACCAG AGACAGATTC |
| 301 | AGACAGATCT GGAAAGCCTG GCACTTCAAC AACAATGCTG ATATCGTGAA CGAGTCTGAT |
| 361 | AGACTGTGTA AAGTGCGGCC AGTGTTGGAT TACTTCGTGC CTAAGTTCAT CAACATCTAT |
| 421 | AAGCCTCACC AGCAGCTGAG CCTGGATGAA GGCATCGTGC CCTGGCGGGG CAGACTGTTC |
| 481 | TTCAGAGTGT ACAATGCTGG CAAGATCGTC AAATACGGCA TCCTGGTGCG CCTTCTGTGC |
| 541 | GAGAGCGATA CAGGCTACAT CTGTAATATG GAAATCTACT GCGGCGAGGG CAAAAGACTG |
| 601 | CTGGAAACCA TCCAGACCGT CGTTTCCCCT TATACCGACA GCTGGTACCA CATCTACATG |
| 661 | GACAACTACT ACAATTCTGT GGCCAACTGC GAGGCCCTGA TGAAGAACAA GTTTAGAATC |
| 721 | TGCGGCACAA TCAGAAAAAA CAGAGGCATC CCTAAGGACT TCCAGACCAT CTCTCTGAAG |
| 781 | AAGGGCGAAA CCAAGTTCAT CAGAAAGAAC GACATCCTGC TCCAAGTGTG GCAGTCCAAG |
| 841 | AAACCCGTGT ACCTGATCAG CAGCATCCAT AGCGCCGAGA TGGAAGAAAG CCAGAACATC |
| 901 | GACAGAACAA GCAAGAAGAA GATCGTGAAG CCCAATGCTC TGATCGACTA CAACAAGCAC |
| 961 | ATGAAAGGCG TGGACCGGGC CGACCAGTAC CTGTCTTATT ACTCTATCCT GAGAAGAACA |
| 1021 | GTGAAATGGA CCAAGAGACT GGCCATGTAC ATGATCAATT GCGCCCTGTT CAACAGCTAC |
| 1081 | GCCGTGTACA AGTCCGTGCG ACAAAGAAAA ATGGGATTCA AGATGTTCCT GAAGCAGACA |
| 1141 | GCCATCCACT GGCTGACAGA CGACATTCCT GAGGACATGG ACATTGTGCC AGATCTGCAA |
| 1201 | CCTGTGCCCA GCACCTCTGG TATGAGAGCT AAGCCTCCCA CCAGCGATCC TCCATGTAGA |
| 1261 | CTGAGCATGG ACATGCGGAA GCACACCCTG CAGGCCATCG TCGGCAGCGG CAAGAAGAAG |
| 1321 | AACATCCTTA GACGGTGCAG GGTGTGCAGC GTGCACAAGC TGCGGAGCGA GACTCGGTAC |
| 1381 | ATGTGCAAGT TTTGCAACAT TCCCCTGCAC AAGGGAGCCT GCTTCGAGAA GTACCACACC |
| 1441 | CTGAAGAATT AC |
| SEQ ID NO: 510: Myositis lucifugus (hyperactive helper) amino acid sequence | |
| (N4, amino acid 2-89 deletion). 484 aa | |
| 1 | MNTDAVINNI EDAVKLFIGD DFFEFLVEES NRYYNQNRNN FKLSKKSLKW KDITPQEMKK |
| 61 | FLGLIVLMGQ VRKDRRDDYW TTEPWTETPY FGKTMTRDRF RQIWKAWHFN NNADIVNESD |
| 121 | RLCKVRPVLD YFVPKFINIY KPHQQLSLDE GIVPWRGRLF FRVYNAGKIV KYGILVRLLC |
| 181 | ESDTGYICNM EIYCGEGKRL LETIQTVVSP YTDSWYHIYM DNYYNSVANC EALMKNKFRI |
| 241 | CGTIRKNRGI PKDFQTISLK KGETKFIRKN DILLQVWQSK KPVYLISSIH SAEMEESQNI |
| 301 | DRTSKKKIVK PNALIDYNKH MKGVDRADQY LSYYSILRRT VKWTKRLAMY MINCALFNSY |
| 361 | AVYKSVRQRK MGFKMFLKQT AIHWLTDDIP EDMDIVPDLQ PVPSTSGMRA KPPTSDPPCR |
| 421 | LSMDMRKHTL QAIVGSGKKK NILRRCRVCS VHKLRSETRY MCKFCNIPLH KGACFEKYHT |
| 481 | LKNY |
| SEQ ID NO: 511: C-terminal deletion Myositis lucifugus (hyperactive helper) | |
| nucleotide sequence (C1; nucleotide 1663-1716 deletion). 1662 bp | |
| 1 | ATGGCCCAGC ACAGCGACTA CCCCGACGAC GAGTTCAGAG CCGATAAGCT GAGTAACTAC |
| 61 | AGCTGCGACA GCGACCTGGA AAACGCCAGC ACATCCGACG AGGACAGCTC TGACGACGAG |
| 121 | GTGATGGTGC GGCCCAGAAC CCTGAGACGG AGAAGAATCA GCAGCTCTAG CAGCGACTCT |
| 181 | GAATCCGACA TCGAGGGCGG CCGGGAAGAG TGGAGCCACG TGGACAACCC TCCTGTTCTG |
| 241 | GAAGATTTTC TGGGCCATCA GGGCCTGAAC ACCGACGCCG TGATCAACAA CATCGAGGAT |
| 301 | GCCGTGAAGC TGTTCATAGG AGATGATTTC TTTGAGTTCC TGGTCGAGGA ATCCAACCGC |
| 361 | TATTACAACC AGAATAGAAA CAACTTCAAG CTGAGCAAGA AAAGCCTGAA GTGGAAGGAC |
| 421 | ATCACCCCTC AGGAGATGAA AAAGTTCCTG GGACTGATCG TTCTGATGGG ACAGGTGCGG |
| 481 | AAGGACAGAA GGGATGATTA CTGGACAACC GAACCTTGGA CCGAGACCCC TTACTTTGGC |
| 541 | AAGACCATGA CCAGAGACAG ATTCAGACAG ATCTGGAAAG CCTGGCACTT CAACAACAAT |
| 601 | GCTGATATCG TGAACGAGTC TGATAGACTG TGTAAAGTGC GGCCAGTGTT GGATTACTTC |
| 661 | GTGCCTAAGT TCATCAACAT CTATAAGCCT CACCAGCAGC TGAGCCTGGA TGAAGGCATC |
| 721 | GTGCCCTGGC GGGGCAGACT GTTCTTCAGA GTGTACAATG CTGGCAAGAT CGTCAAATAC |
| 781 | GGCATCCTGG TGCGCCTTCT GTGCGAGAGC GATACAGGCT ACATCTGTAA TATGGAAATC |
| 841 | TACTGCGGCG AGGGCAAAAG ACTGCTGGAA ACCATCCAGA CCGTCGTTTC CCCTTATACC |
| 901 | GACAGCTGGT ACCACATCTA CATGGACAAC TACTACAATT CTGTGGCCAA CTGCGAGGCC |
| 961 | CTGATGAAGA ACAAGTTTAG AATCTGCGGC ACAATCAGAA AAAACAGAGG CATCCCTAAG |
| 1021 | GACTTCCAGA CCATCTCTCT GAAGAAGGGC GAAACCAAGT TCATCAGAAA GAACGACATC |
| 1081 | CTGCTCCAAG TGTGGCAGTC CAAGAAACCC GTGTACCTGA TCAGCAGCAT CCATAGCGCC |
| 1141 | GAGATGGAAG AAAGCCAGAA CATCGACAGA ACAAGCAAGA AGAAGATCGT GAAGCCCAAT |
| 1201 | GCTCTGATCG ACTACAACAA GCACATGAAA GGCGTGGACC GGGCCGACCA GTACCTGTCT |
| 1261 | TATTACTCTA TCCTGAGAAG AACAGTGAAA TGGACCAAGA GACTGGCCAT GTACATGATC |
| 1321 | AATTGCGCCC TGTTCAACAG CTACGCCGTG TACAAGTCCG TGCGACAAAG AAAAATGGGA |
| 1381 | TTCAAGATGT TCCTGAAGCA GACAGCCATC CACTGGCTGA CAGACGACAT TCCTGAGGAC |
| 1441 | ATGGACATTG TGCCAGATCT GCAACCTGTG CCCAGCACCT CTGGTATGAG AGCTAAGCCT |
| 1501 | CCCACCAGCG ATCCTCCATG TAGACTGAGC ATGGACATGC GGAAGCACAC CCTGCAGGCC |
| 1561 | ATCGTCGGCA GCGGCAAGAA GAAGAACATC CTTAGACGGT GCAGGGTGTG CAGCGTGCAC |
| 1621 | AAGCTGCGGA GCGAGACTCG GTACATGTGC AAGTTTTGCA AC |
| SEQ ID NO: 512: Myositis lucifugus (hyperactive helper) amino acid sequence | |
| (C1, amino acid 555-572 deletion). 554 aa | |
| 1 | MAQHSDYPDD EFRADKLSNY SCDSDLENAS TSDEDSSDDE VMVRPRTLRR RRISSSSSDS |
| 61 | ESDIEGGREE WSHVDNPPVL EDFLGHQGLN TDAVINNIED AVKLFIGDDF FEFLVEESNR |
| 121 | YYNQNRNNFK LSKKSLKWKD ITPQEMKKFL GLIVLMGQVR KDRRDDYWTT EPWTETPYFG |
| 181 | KTMTRDRFRQ IWKAWHFNNN ADIVNESDRL CKVRPVLDYF VPKFINIYKP HQQLSLDEGI |
| 241 | VPWRGRLFFR VYNAGKIVKY GILVRLLCES DTGYICNMEI YCGEGKRLLE TIQTVVSPYT |
| 301 | DSWYHIYMDN YYNSVANCEA LMKNKFRICG TIRKNRGIPK DFQTISLKKG ETKFIRKNDI |
| 361 | LLQVWQSKKP VYLISSIHSA EMEESQNIDR TSKKKIVKPN ALIDYNKHMK GVDRADQYLS |
| 421 | YYSILRRTVK WTKRLAMYMI NCALFNSYAV YKSVRQRKMG FKMFLKQTAI HWLTDDIPED |
| 481 | MDIVPDLQPV PSTSGMRAKP PTSDPPCRLS MDMRKHTLQA IVGSGKKKNI LRRCRVCSVH |
| 541 | KLRSETRYMC KFCN |
| SEQ ID NO: 513: C-terminal deletion Myositis lucifugus (hyperactive helper) | |
| nucleotide sequence (C2; nucleotide 1588-1716 deletion). 1587 bp | |
| 1 | ATGGCCCAGC ACAGCGACTA CCCCGACGAC GAGTTCAGAG CCGATAAGCT GAGTAACTAC |
| 61 | AGCTGCGACA GCGACCTGGA AAACGCCAGC ACATCCGACG AGGACAGCTC TGACGACGAG |
| 121 | GTGATGGTGC GGCCCAGAAC CCTGAGACGG AGAAGAATCA GCAGCTCTAG CAGCGACTCT |
| 181 | GAATCCGACA TCGAGGGCGG CCGGGAAGAG TGGAGCCACG TGGACAACCC TCCTGTTCTG |
| 241 | GAAGATTTTC TGGGCCATCA GGGCCTGAAC ACCGACGCCG TGATCAACAA CATCGAGGAT |
| 301 | GCCGTGAAGC TGTTCATAGG AGATGATTTC TTTGAGTTCC TGGTCGAGGA ATCCAACCGC |
| 361 | TATTACAACC AGAATAGAAA CAACTTCAAG CTGAGCAAGA AAAGCCTGAA GTGGAAGGAC |
| 421 | ATCACCCCTC AGGAGATGAA AAAGTTCCTG GGACTGATCG TTCTGATGGG ACAGGTGCGG |
| 481 | AAGGACAGAA GGGATGATTA CTGGACAACC GAACCTTGGA CCGAGACCCC TTACTTTGGC |
| 541 | AAGACCATGA CCAGAGACAG ATTCAGACAG ATCTGGAAAG CCTGGCACTT CAACAACAAT |
| 601 | GCTGATATCG TGAACGAGTC TGATAGACTG TGTAAAGTGC GGCCAGTGTT GGATTACTTC |
| 661 | GTGCCTAAGT TCATCAACAT CTATAAGCCT CACCAGCAGC TGAGCCTGGA TGAAGGCATC |
| 721 | GTGCCCTGGC GGGGCAGACT GTTCTTCAGA GTGTACAATG CTGGCAAGAT CGTCAAATAC |
| 781 | GGCATCCTGG TGCGCCTTCT GTGCGAGAGC GATACAGGCT ACATCTGTAA TATGGAAATC |
| 841 | TACTGCGGCG AGGGCAAAAG ACTGCTGGAA ACCATCCAGA CCGTCGTTTC CCCTTATACC |
| 901 | GACAGCTGGT ACCACATCTA CATGGACAAC TACTACAATT CTGTGGCCAA CTGCGAGGCC |
| 961 | CTGATGAAGA ACAAGTTTAG AATCTGCGGC ACAATCAGAA AAAACAGAGG CATCCCTAAG |
| 1021 | GACTTCCAGA CCATCTCTCT GAAGAAGGGC GAAACCAAGT TCATCAGAAA GAACGACATC |
| 1081 | CTGCTCCAAG TGTGGCAGTC CAAGAAACCC GTGTACCTGA TCAGCAGCAT CCATAGCGCC |
| 1141 | GAGATGGAAG AAAGCCAGAA CATCGACAGA ACAAGCAAGA AGAAGATCGT GAAGCCCAAT |
| 1201 | GCTCTGATCG ACTACAACAA GCACATGAAA GGCGTGGACC GGGCCGACCA GTACCTGTCT |
| 1261 | TATTACTCTA TCCTGAGAAG AACAGTGAAA TGGACCAAGA GACTGGCCAT GTACATGATC |
| 1321 | AATTGCGCCC TGTTCAACAG CTACGCCGTG TACAAGTCCG TGCGACAAAG AAAAATGGGA |
| 1381 | TTCAAGATGT TCCTGAAGCA GACAGCCATC CACTGGCTGA CAGACGACAT TCCTGAGGAC |
| 1441 | ATGGACATTG TGCCAGATCT GCAACCTGTG CCCAGCACCT CTGGTATGAG AGCTAAGCCT |
| 1501 | CCCACCAGCG ATCCTCCATG TAGACTGAGC ATGGACATGC GGAAGCACAC CCTGCAGGCC |
| 1561 | ATCGTCGGCA GCGGCAAGAA GAAGAAC |
| SEQ ID NO: 514: Myositis lucifugus (hyperactive helper) amino acid sequence (C2, | |
| amino acid 530-572 deletion). 529 aa | |
| 1 | MAQHSDYPDD EFRADKLSNY SCDSDLENAS TSDEDSSDDE VMVRPRTLRR RRISSSSSDS |
| 61 | ESDIEGGREE WSHVDNPPVL EDFLGHQGLN TDAVINNIED AVKLFIGDDF FEFLVEESNR |
| 121 | YYNQNRNNFK LSKKSLKWKD ITPQEMKKFL GLIVLMGQVR KDRRDDYWTT EPWTETPYFG |
| 181 | KTMTRDRFRQ IWKAWHFNNN ADIVNESDRL CKVRPVLDYF VPKFINIYKP HQQLSLDEGI |
| 241 | VPWRGRLFFR VYNAGKIVKY GILVRLLCES DTGYICNMEI YCGEGKRLLE TIQTVVSPYT |
| 301 | DSWYHIYMDN YYNSVANCEA LMKNKFRICG TIRKNRGIPK DFQTISLKKG ETKFIRKNDI |
| 361 | LLQVWQSKKP VYLISSIHSA EMEESQNIDR TSKKKIVKPN ALIDYNKHMK GVDRADQYLS |
| 421 | YYSILRRTVK WTKRLAMYMI NCALFNSYAV YKSVRQRKMG FKMFLKQTAI HWLTDDIPED |
| 481 | MDIVPDLQPV PSTSGMRAKP PTSDPPCRLS MDMRKHTLQA IVGSGKKKN |

### NUMBERED EMBODIMENTS

1. A composition comprising
(A) a helper enzyme or a nucleic acid encoding the helper enzyme, wherein the helper enzyme comprises an amino acid sequence having at least about 80% sequence identity to SEQ ID NO: 9 or SEQ ID NO: 2 and has an alanine residue at position 2 of SEQ ID NO: 9 or SEQ ID NO: 2 or a position corresponding thereto;
(B) composition comprising (a) a helper enzyme or a nucleic acid encoding the helper enzyme and (b) a targeting element or a nucleic acid encoding the targeting element and a linker connecting the helper enzyme and the targeting element, wherein: the helper enzyme comprises an amino acid sequence having at least about 80% sequence identity to SEQ ID NO: 9 or SEQ ID NO: 2 and has a non-polar aliphatic amino acid at position 2 of SEQ ID NO: 9 or SEQ ID NO: 2 or a position corresponding thereto and one or more of S8X₁ of SEQ ID NO: 9 or SEQ ID NO: 2 or a position corresponding thereto, wherein X₁ is selected from alanine (A), glycine (G), valine (V), leucine (L), isoleucine (I), and proline (P); C13X₂ of SEQ ID NO: 9 or SEQ ID NO: 2 or a position corresponding thereto, wherein X₂ is selected from lysine (K), arginine (R), and histidine (H); and N125X₃ of SEQ ID NO: 9 or SEQ ID NO: 2 or a position corresponding thereto, wherein X₃ is selected from is selected from lysine (K), arginine (R), and histidine (H); the targeting element is or comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof; and the linker comprises less than about 25 amino acids or 75 nucleotides; or
(C) composition comprising (a) a helper enzyme or a nucleic acid encoding the helper enzyme and (b) a targeting element or a nucleic acid encoding the targeting element, wherein: the helper enzyme comprises an amino acid sequence having at least about 80% sequence identity to SEQ ID NO: 9 or SEQ ID NO: 2 and has a non-polar aliphatic amino acid at position 2 of SEQ ID NO: 9 or a position corresponding thereto and one or more of S8X₁ of SEQ ID NO: 9 or SEQ ID NO: 2 or a position corresponding thereto, wherein X₁ is selected from alanine (A), glycine (G), valine (V), leucine (L), isoleucine (I), and proline (P); C13X₂ of SEQ ID NO: 9 or SEQ ID NO: 2 or a position corresponding thereto, wherein X₂ is selected from lysine (K), arginine (R), and histidine (H); and N125X₃ of SEQ ID NO: 9 or SEQ ID NO: 2 or a position corresponding thereto, wherein X₃ is selected from is selected from lysine (K), arginine (R), and histidine (H); the targeting element is or comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof; and wherein the targeting element directs the helper enzyme to one or more nucleic acids sites that are upstream and/or downstream of the TTAA integration sites and within about 5 to about 30 base pairs of the TTAA integration sites or within about 15 to about 19 base pairs of the TTAA integration sites.

2. The composition of Embodiment 1, wherein the helper enzyme comprises an amino acid sequence of at least about 90% identity to SEQ ID NO: 9 or SEQ ID NO: 2.

3. The composition of Embodiment 1, wherein the helper enzyme comprises an amino acid sequence of at least about 93% identity to SEQ ID NO: 9 or SEQ ID NO: 2.

4. The composition of Embodiment 1, wherein the helper enzyme comprises an amino acid sequence of at least about 95% identity to SEQ ID NO: 9 or SEQ ID NO: 2.

5. The composition of Embodiment 1, wherein the helper enzyme comprises an amino acid sequence of at least about 98% identity to SEQ ID NO: 9 or SEQ ID NO: 2.

6. The composition of Embodiment 1, wherein the helper enzyme comprises an amino acid sequence of at least about 99% identity to SEQ ID NO: 9 or SEQ ID NO: 2.

7. The composition of any one of Embodiments 1-6, wherein the helper enzyme has one or more mutations which confer hyperactivity.

8. The composition of any one of Embodiments 1-7, wherein the helper enzyme has one or more amino acid substitutions selected from S8X₁ and/or C13X₂ or substitutions at positions corresponding thereto.

9. The composition of Embodiment 8, wherein the helper enzyme has S8X₁ and C13X₂ substitutions or substitutions at positions corresponding thereto.

10. The composition of Embodiment 8 or Embodiment 9, wherein X₁ is selected from G, A, V, L, I, and P and X₂ is selected from K, R, and H.

11. The composition of any one of Embodiments 8-10, wherein: X₁ is P and X₂ is R.

12. The composition of any one of Embodiments 1-11, wherein the helper enzyme comprises an amino acid sequence of SEQ ID NO: 2.

13. The composition of any one of Embodiments 1-12, wherein the nucleic acid that encodes the helper enzyme has a nucleotide sequence of SEQ ID NO: 11 or a codon-optimized form thereof.

14. The composition of any one of Embodiments 1-13, wherein the helper enzyme comprises at least one substitution at positions selected from TABLE 1 and/or TABLE 2 or positions corresponding thereto, which correspond positions of SEQ ID NO: 9 or SEQ ID NO: 2.

15. The composition of any one of Embodiments 1-14, wherein the helper enzyme comprises at least one substitution at positions selected from: 164, 165, 168, 286, 287, 310, 331, 333, 334, 336, 338, 349, 350, 368, 369, 416, or positions corresponding thereto relative to SEQ ID NO: 9 or SEQ ID NO: 2.

16. The composition of any one of Embodiments 1-14, wherein the helper enzyme comprises at least one substitution at positions selected from: R164N, D165N, W168V, W168A, K286A, R287A, N310A, T331A, R333A, K334A, R336A, I338A, K349A, K350A, K368A, K369A, D416A, D416N, or positions corresponding thereto relative to SEQ ID NO: 9 or SEQ ID NO: 2.

17. The composition of any one of Embodiments 1-15, wherein the helper enzyme comprises at least one substitution at position corresponding to: 331, 333, and/or 416 or positions corresponding thereto relative to SEQ ID NO: 9 or SEQ ID NO: 2.

18. The composition of Embodiment 17, wherein the substitution is selected from G, A, V, N, and Q.

19. The composition of any one of Embodiments 1-16, wherein the helper enzyme comprises at least one substitution at selected from: W168V, T331A, R333A, and/or D416N, or positions corresponding thereto, wherein the positions are relative to SEQ ID NO: 9 or SEQ ID NO: 2.

20. The composition of any one of Embodiments 1-17, wherein the helper enzyme comprises a deletion of about 30, or about 40, or about 50, or about 60, or about 70, or about 80, or about 90, or about 100 amino acids from an N-terminus of the polypeptide having an amino acid sequence of SEQ ID NO: 9 or SEQ ID NO: 2.

21. The composition of any one of Embodiments 1-17, wherein the helper enzyme comprises a deletion at positions about 1-35, or about 1-45, or about 1-55, or about 1-65, or about 1-75, or about 1-85, or about 1-95, or about 1-105 or positions corresponding thereto, wherein the positions are relative to SEQ ID NO: 9 or SEQ ID NO: 502, or the helper enzyme comprises an N-terminal deletion, optionally at positions about 1-34, or about 1-45, or about 1-68, or about 1-89 or positions corresponding thereto, wherein the positions are relative to SEQ ID NO: 9 or SEQ ID NO: 502, or the helper enzyme comprises a C-terminal deletion, optionally at positions about 555-573 or about 530-573 or positions corresponding thereto, wherein the positions are relative to SEQ ID NO: 9 or SEQ ID NO: 502, wherein the deletion comprises an N or C terminal deletion, wherein the N or C terminal deletion yields reduced or ablated off-target effects of the helper enzyme compared to the helper enzyme without the N or C terminal deletion, wherein the helper enzyme comprising the N terminal deletion is N2, wherein the helper enzyme comprising the N terminal deletion is or comprises SEQ ID NO: 506, wherein the mutant with an N or C terminal deletion is further fused to a DNA binder, wherein the DNA binder comprises TALEs, ZnF, and/or both.

22. The composition of any one of Embodiments 1-19, wherein the helper enzyme has increased activity relative to a helper enzyme comprising an amino acid sequence of SEQ ID NO: 9 or SEQ ID NO: 2 or functional equivalent thereof.

23. The composition of any one of Embodiments 1-20, wherein the helper enzyme is excision positive.

24. The composition of any one of Embodiments 1-21, wherein the helper enzyme is integration deficient.

25. The composition of any one of Embodiments 14-22, wherein the helper enzyme has decreased integration activity relative to a helper enzyme comprising an amino acid sequence of SEQ ID NO: 9 or SEQ ID NO: 2 or functional equivalent thereof.

26. The composition of any one of Embodiments 14-23, wherein the helper enzyme has increased excision activity relative to a helper enzyme comprising an amino acid sequence of SEQ ID NO: 9 or SEQ ID NO: 2 or functional equivalent thereof.

27. The composition of any one of Embodiments 1-26, wherein the helper enzyme comprises a targeting element.

28. The composition of any one of Embodiments 1-27, wherein the helper enzyme is capable of inserting a donor comprising a transgene in a genomic safe harbor site (GSHS).

29. The composition of Embodiment 28, wherein the binding of a GSHS of a nucleic acid molecule in a mammalian cell is with high target specificity, relative to a control.

30. The composition of Embodiment 29, wherein the control is a composition comprising a helper enzyme comprising an amino acid sequence of SEQ ID NO: 9 or SEQ ID NO: 2 or a nucleic acid comprising a nucleotide sequence of SEQ ID NO: 10 or a codon-optimized form thereof, and/or wherein the control is a composition comprising a helper enzyme comprising an amino acid sequence of SEQ ID NO: 2 or SEQ ID NO: 2 or a nucleic acid comprising a nucleotide sequence of SEQ ID NO: 11 or a codon-optimized form thereof.

31. The composition of any one of Embodiments 27-30, wherein the targeting element is able to direct a transposition machinery to the GSHS of a nucleic acid molecule in a mammalian cell.

32. The composition of any one of Embodiments 27-31, wherein the GSHS is in an open chromatin location in a chromosome.

33. The composition of any one of Embodiments 27-32, wherein the GSHS is selected from adeno-associated virus site 1 (AAVS1), chemokine (C-C motif) receptor 5 (CCR5) gene, HIV-1 coreceptor, and human Rosa26 locus.

34. The composition of any one of Embodiments 27-33, wherein the GSHS is an adeno-associated virus site 1 (AAVS1).

35. The composition of any one of Embodiments 27-34, wherein the GSHS is a human Rosa26 locus.

36. The composition of any one of Embodiments 27-35, wherein the GSHS is located on human chromosome 2, 4, 6, 10, 11, 17, 22, or X.

37. The composition of any one of Embodiments 27-36, wherein the GSHS is selected from TABLES 3-17.

38. The composition of any one of Embodiments 27-37, wherein the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

39. The composition of any one of Embodiments 27-38, wherein the targeting element is or comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof.

40. The composition of Embodiment 39, wherein the targeting element comprises a TALE DBD.

41. The composition of Embodiment 40, wherein the TALE DBD comprises one or more repeat sequences.

42. The composition of Embodiment 41, wherein the TALE DBD comprises about 14, or about 15, or about, 16, or about 17, or about 18, or about 18.5 repeat sequences.

43. The composition of Embodiment 41 or Embodiment 42, wherein the repeat sequences each independently comprises about 33 or 34 amino acids.

44. The composition of Embodiment 43, wherein the repeat sequences each independently comprises a repeat variable di-residue (RVD) at residue 12 or 13 of the 33 or 34 amino acids, respectively.

45. The composition of Embodiment 44, wherein the RVD recognizes one base pair in a target nucleic acid sequence.

46. The composition of Embodiment 43 or Embodiment 44, wherein the RVD recognizes a C residue in the target nucleic acid sequence and is selected from HD, N(gap), HA, ND, and HI.

47. The composition of Embodiment 43 or Embodiment 44, wherein the RVD recognizes a G residue in the target nucleic acid sequence and is selected from NN, NH, NK, HN, and NA.

48. The composition of Embodiment 43 or Embodiment 44, wherein the RVD recognizes an A residue in the target nucleic acid sequence and is selected from NI and NS.

49. The composition of Embodiment 43 or Embodiment 44, wherein the RVD recognizes a T residue in the target nucleic acid sequence and is selected from NG, HG, H(gap), and IG.

50. The composition of Embodiment 39-49, wherein the TALE DBD targets one or more of GSHS sites selected from TABLES 8-12 and TABLE 20.

51. The composition of any one of Embodiments 39-50, wherein the TALE DBD comprises one or more of RVD selected from TABLES 8-12 and TABLE 20, or variants thereof comprising about 1, about 2, about 3, about 4, about 5, about 6, about 7, about 8, about 9, about 10, about 11, about 12, about 13, about 14, about 15, about 16, about 17, about 18, about 19, or about 20 mutations.

52. The composition of Embodiment 39, wherein the targeting element comprises a Cas9 enzyme associated with a gRNA.

53. The composition of Embodiment 52, wherein the Cas9 enzyme associated with a gRNA comprises a catalytically inactive dCas9 associated with a gRNA.

54. The composition of Embodiment 53, wherein catalytically inactive dCas9 comprises at least about 90%, or at least about 95%, or at least about 96%, or at least about 97%, or at least about 98%, or at least about 99% identity to an amino acid sequence of SEQ ID NO: 6 or a nucleic acid comprising a nucleotide sequence of SEQ ID NO: 5 or a codon-optimized form thereof.

55. The composition of any one of Embodiments 39 or 52-54, wherein the targeting element comprises a Cas12 enzyme associated with a gRNA.

56. The composition of Embodiment 55, wherein the targeting element comprises a catalytically inactive Cas12 associated with a gRNA, optionally wherein the catalytically inactive Cas12 is dCas12j or dCas12a.

57. The composition of any one of Embodiments 39 or 52-54, wherein the targeting element comprises a TnsC, TnsB, TnsA, TniQ, Cas6, Cas7, Cas8 enzyme associated with a gRNA.

58. The composition of any one of Embodiments 39 or 52-54, wherein the targeting element comprises a TnsD.

59. The composition of Embodiments 39 or 52-56, wherein the guide RNA is selected from TABLES 3-7 and TABLE 19, or variants thereof comprising about 1, about 2, about 3, about 4, about 5, about 6, about 7, about 8, about 9, about 10, about 11, about 12, about 13, about 14, about 15, about 16, about 17, about 18, about 19, or about 20 mutations.

60. The composition of Embodiments 39 or 52-56, wherein the guide RNA targets one or more sites selected from TABLES 3-7 and TABLE 19.

61. The composition of Embodiment 39, wherein the zinc finger comprises one of the sequences selected from TABLES 13-17, or variants thereof comprising about 99, about 98, about 97, about 95, about 94, about 93, about 92, about 91, about 90, about 89, about 88, about 87, about 86, about 85, about 84, about 83, about 82, about 81, about 80 percent identity to the sequence.

62. The composition of Embodiment 39, wherein the zinc finger targets one or more sites selected from TABLES 13-17.

63. The composition of any one of Embodiments 39-62, wherein the targeting element comprises a nucleic acid binding component of a gene-editing system.

64. The composition of any one of Embodiments 39-63, wherein the helper enzyme or variant thereof and the targeting element are connected.

65. The composition of Embodiment 64, wherein the helper enzyme and the targeting element are fused to one another or linked via a linker to one another.

66. The composition of Embodiment 64, wherein the linker is a flexible linker.

67. The composition of Embodiment 66, wherein the flexible linker is substantially comprised of glycine and serine residues, optionally wherein the flexible linker comprises (Gly₄Ser)ₙ, or (GSS)ₙ where n is an integer from 1-12.

68. The composition of Embodiment 67, wherein the flexible linker is of about 20, or about 30, or about 40, or about 50, or about 60 amino acid residues.

69. The composition of Embodiment 68, wherein the helper enzyme is directly fused to the N-terminus of the targeting element and, optionally, wherein the targeting element is or comprises dCas9 enzyme.

70. The composition of any one of Embodiments 1-69, wherein the helper enzyme or variant thereof is able to directly or indirectly cause transposition of a target gene.

71. The composition of any one of Embodiments 1-70, wherein the helper enzyme or variant thereof is able to directly or indirectly interact and/or form a complex with one or more proteins or nucleic acids.

72. The composition of any one of the preceding Embodiments, wherein a nucleic acid encoding the helper enzyme capable of targeted genomic integration by transposition comprises an intein, optionally NpuN (Intein-N) (SEQ ID NO: 423) and/or NpuC (Intein-C) (SEQ ID NO: 424), or a variant thereof.

73. The composition of Embodiment 72, wherein the nucleic acid encodes the helper enzyme in the form of first and second portions with the intein encoded between the first and second portions, such that the first and second portions are fused into a functional helper enzyme upon post-translational excision of the intein from the helper enzyme.

74. The composition of Embodiment 72 or Embodiment 73, wherein the intein is suitable for linking the helper enzyme and the targeting element.

75. The composition of any one of the preceding Embodiments, wherein a nucleic acid encoding the helper enzyme capable of targeted genomic integration by transposition comprises a dimerization enhancer.

76. The composition of Embodiment 75, wherein the nucleic acid encodes the helper enzyme in the form of first and second portions with the dimerization enhancer encoded between the first and second portions, such that the first and second portions are fused into a functional helper enzyme upon post-translational excision of the dimerization enhancer from the helper enzyme.

77. The composition of Embodiment 75 or Embodiment 76, wherein the dimerization enhancer is suitable for linking the helper enzyme and the targeting element.

78. The composition of any one of Embodiments 75-77, wherein the dimerization enhancer is selected from: a protein comprising a SH3 domain, biotin, avidin, or a rapamycin binder, optionally, wherein the rapamycin binder is FKBP12 or mTOR, or a variant thereof.

79. The composition of any one of Embodiments 1-78, further comprising a nucleic acid encoding a donor comprising a transgene to be integrated, optionally wherein the transgene is defective or substantially absent in a disease state.

80. The composition of Embodiment 79, wherein the transgene comprises a cargo nucleic acid sequence and a first and a second donor end sequences.

81. The composition of Embodiment 80, wherein the cargo nucleic acid sequence is flanked by the first and the second donor end sequences.

82. The composition of Embodiment 80 or Embodiment 81, wherein the donor end sequences are selected from nucleotide sequences of SEQ ID NO: 3 and/or SEQ ID NO: 4, or a nucleotide sequence having at least about 90% identity thereto.

83. The composition of any one of Embodiments 80-82, wherein the end sequences include at least one repeat from a nucleotide sequence having at least about 90% identity to the nucleotide sequence of SEQ ID NO: 3. 84. The composition of Embodiment 83, wherein the at least one repeat from the nucleotide sequence having at least about 90% identity to the nucleotide sequence of SEQ ID NO: 3 is positioned at the 5′ end of the donor.

85. The composition of any one of Embodiments 80-84, wherein the end sequences can further include at least one repeat from a nucleotide sequence having at least about 90% identity to the nucleotide sequence of SEQ ID NO: 4.

86. The composition of any one of Embodiments 81-85, wherein the at least one repeat from the nucleotide sequence having at least about 90% identity to the nucleotide sequence of SEQ ID NO: 4 is positioned at the 3′ end of the donor.

87. The composition of any one of Embodiments 1-86, wherein the helper enzyme or variant thereof is incorporated into a vector or a vector-like particle.

88. The composition of any one of Embodiments 1-87, wherein the vector or a vector-like particle comprises one or more expression cassettes.

89. The composition of Embodiment 88, wherein the vector or a vector-like particle comprises one expression cassette.

90. The composition of Embodiment 89, wherein the expression cassette further comprises the helper enzyme or variant thereof, the transgene, the donor end sequences, or a combination thereof.

91. The composition of Embodiment 90, wherein the helper enzyme or variant thereof, the transgene, the donor end sequences, or a combination thereof are incorporated into one or more vectors or vector-like particles.

92. The composition of Embodiment 90, wherein the helper enzyme or variant thereof, the transgene, the donor end sequences, or combination thereof are incorporated into a same vector or vector-like particle.

93. The composition of Embodiment 90, wherein the helper enzyme or variant thereof, the transgene, the donor end sequences, or combination thereof is incorporated into different vectors or vector-like particles.

94. The composition of any one of Embodiments 87-93, wherein the vector or vector-like particle is nonviral.

95. The composition of any one of Embodiments 79-94, wherein the donor is under the control of at least one tissue-specific promoter.

96. The composition of Embodiment 95, wherein the at least one tissue-specific promoter is a single promoter.

97. The composition of Embodiment 95, wherein the at least one tissue-specific promoter is under the control of a dual promoter or a tandem promoter.

98. The composition of any one of Embodiments 79-97, wherein the transgene to be integrated comprises at least one gene of interest.

99. The composition of any one of Embodiments 79-98, wherein the transgene to be integrated comprises one gene of interest.

100. The composition of any one of Embodiments 79-98, wherein the transgene to be integrated comprises two or more genes of interest.

101. The composition of any one of Embodiments 79-100, wherein the at least one gene of interest comprises peptides for linking genes of interest.

102. The composition of Embodiment 101, wherein the peptides are 2A self-cleaving peptides, or functional variants thereof, wherein the 2A self-cleaving peptide is optionally selected from P2A, E2A, F2A, and T2A, or derivative thereof.

103. The composition of any one of Embodiments 79-102, wherein the at least one gene of interest is linked to polynucleotide comprising a sequence comprising a 5′-miRNA, a sense and antisense miRNA pair, and/or a 3′-miRNA.

104. The composition of any one of Embodiments 1-103, wherein the composition comprises DNA, RNA, or both.

105. The composition of any one of Embodiments 1-104, wherein the helper enzyme or variant thereof is in the form of RNA.

106. A host cell comprising the composition any one of Embodiments 1-105. 107. The composition of any one of Embodiments 1-105, wherein the composition is encapsulated in a lipid nanoparticle (LNP).

108. The composition of any one of Embodiments 1-105, wherein the polynucleotide encoding the helper enzyme or variant thereof and the polynucleotide encoding the donor are in the form of the same LNP, optionally in a co-formulation.

109. The composition of Embodiment 107 or Embodiment 108, wherein the LNP comprises one or more lipids selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy(polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC) and/or comprising of one or more molecules selected from polyethylenimine (PEI) and poly(lactic-co-glycolic acid) (PLGA), and N-Acetylgalactosamine (GalNAc).

110. A method for inserting a gene into the genome of a cell, comprising contacting a cell with the composition of any one of Embodiments 1-105 or 107-109 or host cell of Embodiment 106.

111. The method of Embodiment 110, further comprising contacting the cell with a polynucleotide encoding a donor DNA.

112. The method of Embodiment 110 or Embodiment 111, wherein the donor comprises a gene encoding a complete polypeptide.

113. The method of any one of Embodiments 110-112, wherein the donor comprises a gene which is defective or substantially absent in a disease state.

114. A method for treating a disease or disorder ex vivo, comprising contacting a cell with the composition of any one of Embodiments 1-105 or 107-109 or host cell of Embodiment 106 and administering the cell to a subject in need thereof.

115. A method for treating a disease or disorder in vivo, comprising administering the composition of any one of Embodiments 1-105 or 107-109 or host cell of Embodiment 106 to a subject in need thereof.

This invention is further illustrated by the following non-limiting examples.

### EXAMPLES

Hereinafter, the present disclosure will be described in further detail with reference to examples. These examples are illustrative purposes only and are not to be construed to limit the scope of the present invention. In addition, various modifications and variations can be made without departing from the technical scope of the present invention.

### Example 1—Bioengineering the MLT Transposase Protein for Site-Specific Targeting and Heterodimerization

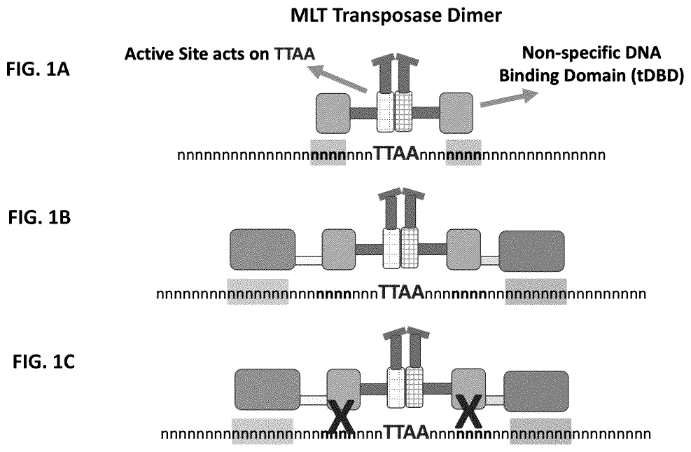

FIG. 1A-FIG. 1C depict the concepts of bioengineering the MLT transposase protein of the present disclosure for site-specific targeting and hetrodimerization. As shown in FIG. 1A, the unengineered MLT transposase dimer binds the target DNA TTAA and flanking non-TTAA (nnnn) phosphodiester backbone (sequence independent). As shown in FIG. 1B, the recruitment to a site-specific TTAA is directed by fusing (i.e., linking) protein sequence-specific DNA binding domains that recognize target DNA sequences flanking the TTAA. Such DNA binding domains encompass, without limitation, TALE, ZnF, and Cas. In FIG. 1C, mutations (depicted as “X” in the figure) in the intrinsic DNA binding domains decrease MLT transposase interactions with target DNA non-TTAA which flank the TTAA but leave excision and TTAA use intact (Exc+, Int−).

FIG. 1A-FIG. 1C depict the bioengineering strategy to eliminate or reduce the intrinsic non-specific DNA binding of MLT transposase by mutagenesis and substitute site-specific, single synthetic DNA binder (e.g., without limitation, TALE, ZF, Cas, etc.) linked to homodimers or two synthetic binders linker to each heterodimer. This targeting strategy permits the insertion of a DNA element (GOI) at a single TTAA.

### Example 2—Types of Covalent and Non-Covalent Linkers

This example shows the discovery of DNA binding proteins (e.g., without limitations, TALE and Cas9), linkers, and fusion sites that target specific TTAA.

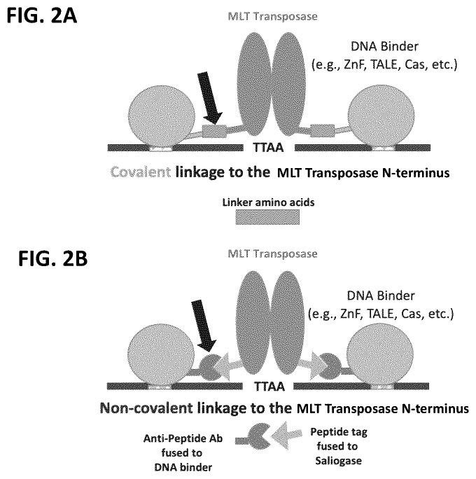

FIG. 2A-FIG. 2B depict the types of covalent and non-covalent linkers that are used to directly fuse (i.e., link) protein sequence-specific DNA binding domains (e.g., without limitation, TALE, ZnF, Cas) that recognize target DNA sequences flanking the TTAA. In FIG. 2A, the arrow shows covalent linker that fuses DNA binders to the N-terminus of MLT transposase. The linkers are strings of amino acids of varying lengths and flexibility. In FIG. 2B, the arrows show non-covalent linkers that an antipeptide antibody (Ab) fused to a DNA binder and a peptide tag fused to the N-terminus of MLT transposase. These components can be changed where the antipeptide Ab is fused to MLT transposase and the peptide tag is fused to the DNA binder.

FIG. 2A-FIG. 2B depict two different types of linkers used to bioengineer synthetic DNA binders and allow the flexibility to bind to nearby flanking recognition sites. The distance of the recognition site from the TTAA was determined empirically to be 15-19 bp using non-covalent and covalent (4×, original) linkers.

### Example 3—A 5-Step Plasmid Landing Pad Assay in HEK293 Cells to Identify Site-Specific Targeting Using MLT Transposase or Other Mobile Elements

This example demonstrates, inter alia, the development of landing pad assay in HEK293 and show site- and sequence-specific targeting.

FIG. 3 depicts a 5-step plasmid landing pad assay in HEK293 cells to identify site-specific targeting using MLT transposase or other mobile elements (e.g., without limitation, recombinases, integrases, transposases).

Step 1 involves transfection of HEK293 cells using a donor DNA with CMV driving the 5-half (left) of GFP followed by a splice-donor (SD) site, MLT transposase fusion helpers with various linkers and DNA binding fusions linked to the N-terminus of MLT transposase, and a plasmid landing pad (reporter plasmid) with site specific DNA binding recognition sites flanking a TTAA followed by a splice acceptor site (SA) and the 3-half (right) half of GFP.

Step 2 shows the mechanism of splicing and integration into the landing pad after transfection.

In Step 3, the left and right halves of GFP are joined and the SA and SD are spliced out thus turning on GFP (GFP readout).

Step 4 is the PCR amplification step to identify targeting.

Step 5 uses Amplicon-Seq to analyze integration in specific sequence regions.

FIG. 3 depicts plasmid cell-based assay to assess integration patterns. Step 1 to Step 3 involves transfection of HEK293 cells using a donor plasmid, reporter plasmid, and bioengineered MLT transposase. The integration readout is GFP expression by splicing the 5′-left GFP region to the 3-right GFP region. Step 4 and Step 5 uses PCR and sequencing to analyze integrants. The DNA is extracted and the insertions or amplified using oligonucleotide primers within donor insert and outside the landing pad. Briefly the cell pellets are prepared for lysis using Viagen DirectCell according to manufacturer's protocol. Proteinase K powder (0.4 mg/ml) and 90 μl of buffer is added to each pellet and rotated for 3 hrs at 55° C. The mixture is heat inactivated for 45 min at 85° C. and 1.0 μl of lysate is used as a genomic DNA template. 1 μl of lysis was used for genomic PCR template. Forward (outside landing pad) and reverse primers (within insert) with barcodes are added to a 20 μl master mix in a 20 μl reaction containing 10 μl KOD ONE BLUE, 7.8 μl water and 0.6 μl each primer (10 uM). The PCR mixture is hot started at 95° C. for 30 seconds followed by 32 PCR cycles (denaturation 95° C. for 10 seconds, annealing at 60° C. for 5 seconds, and extension for 68° C. for 5 seconds). Plasmid cell-based assay was used to assess integration patterns. Step 5 uses Amplicon-Seq to analyze integration in specific sequence regions. The ultra-deep sequencing of PCR products (amplicons) used oligonucleotide barcodes designed to capture the regions of interest, followed by next-generation sequencing (NGS). Briefly, the remaining 11 μl of the PCR reaction is cleaned using the Zymo DNA Clean & Concentrator, according to manufacturer's protocol. The DNA is quantified and diluted to 20 ng/μl and samples with unique barcodes are mixed in equal amounts and analyzed by NGS. The bioinformatic output by internal amplicon seq analysis software shows the flanking sequence, position on reporter, number of reads, percent insertion at each TTAA site.

### Example 4—PCR Amplification to Identify Targeting

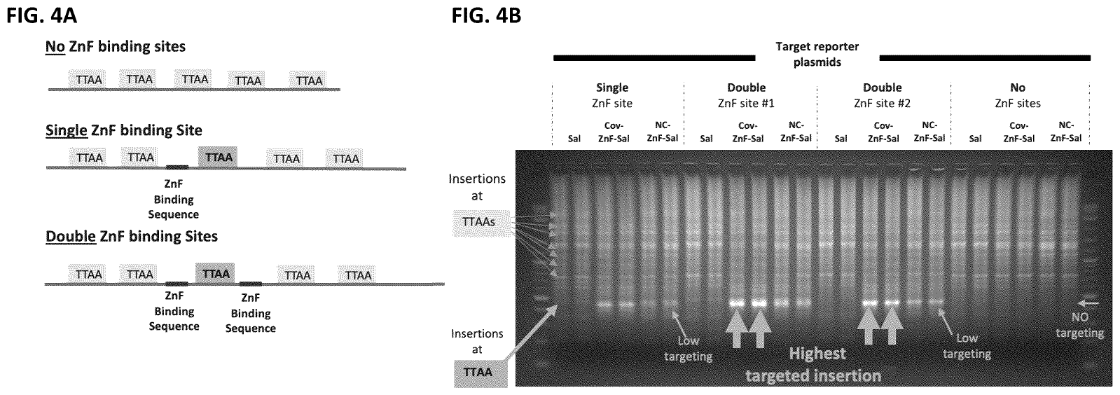

FIG. 4A-FIG. 4B depict PCR amplification to identify targeting Step 4 in FIG. 3. In FIG. 4A, a landing pad with no DNA binding recognition sites (zinc fingers (ZnF) in this case, but could be TALE, Cas, etc.) is used as a negative control. Landing pads with DNA binding recognition sites (ZnF in this case, but could be TALE, Cas, etc.) on one or both sides of the target TTAA are analyzed for targeting. In FIG. 4B, a 2% agarose gel shows the PCR products using both covalent (Cov) and non-covalent (NC) linkers (shown in FIG. 2A and FIG. 2B) and landing pads with a single, double or no ZnF recognition sites. There are no unique PCR products when unengineered MLT transposase (labeled as “Sal” in the figure) or landing pads without DNA binding recognition sites are used. Targeted PCR products are seen using MLT transposase fusion proteins using both Cov and NC linkers. The highest targeted insertions are seen using covalently linked MLT transposase fusions when there are two flanking DNA binding recognition sites.

FIG. 4A-FIG. 4B depict the PCR readout of the plasmid cell-based assay to assess integration patterns using the methodology described for FIG. 3. The 2% agarose gel show a specific targeted band (465 bp) when synthetic DNA binders are fused to the N-terminus of MLT transposase and their recognition site flank a targeted TTAA. This gel shows site-specific targeting of a single TTAA.

### Example 5—Sequence-Specific Targeting as Shown by Amplicon-Seq Results

This example shows that landing pads of the present disclosure enable Amplicon-seq to show high efficiency targeting (e.g., without limitations, 42%) using covalent linkers and flanking DNA binding recognition sites that were within 15-19 base pairs of the target TTAA.

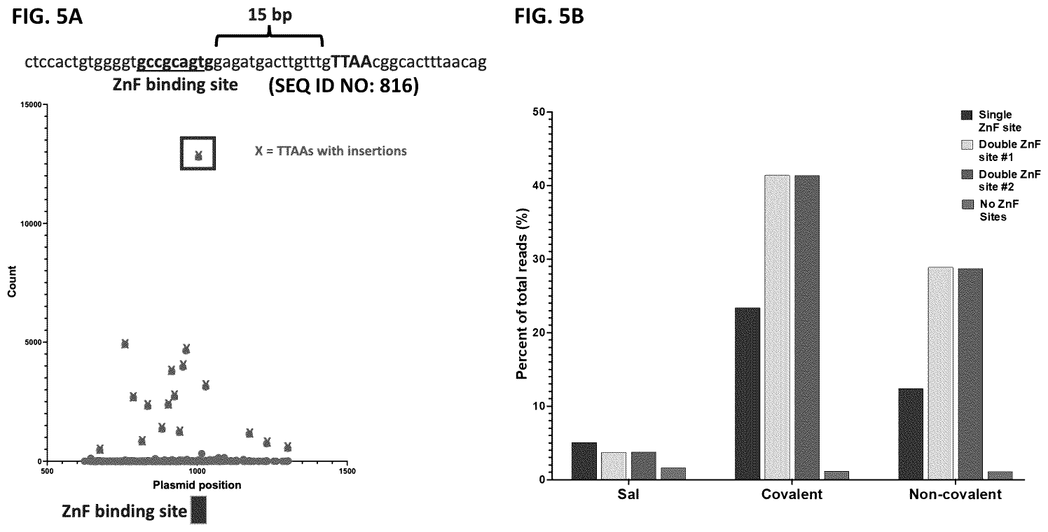

FIG. 5A-FIG. 5B depict Step 5 Amplicon-Seq results showing sequence-specific targeting at 15 base pairs (also occurs at 19 bp, data not shown) from the DNA binding recognition site (SEQ ID NO: 816). FIG. 5A depicts Next Generation sequencing results show on-target insertion (boxed) at 15 base pairs from the targeted TTAA with few off-targets within 350 bp on either side of the TTAA. FIG. 5B depicts a bar graph showing that covalent linker and a landing pad with flanking DNA binding recognition sites has about a 42% targeting efficiency (42% of total reads) compared to a single site landing pad (24%). Non-covalent linkers with a landing pad with flanking DNA binding recognition sites had a 29% efficiency with the least with a single DNA binding recognition site (12%).

FIG. 5A-FIG. 5B depict frequent site-specific targeting of a single TTAA with minimal off target integration in the surrounding 500 bp region (SEQ ID NO: 816). The distance of the targeted TTAA insertion was 15 bp from the DNA binding recognition site. The integration frequency increased two-fold when recognition sites were placed flanking the targeted TTAA. Covalent linkers (4× and Original) showed to most efficient single-site integration. This data shows, inter alia, that MLT transposase can target a single TTAA site when synthetic DNA binders are fused to the N-terminus of MLT transposase and recognition sites are placed 15 bp from the target TTAA.

### Example 6—Design of Transposon System

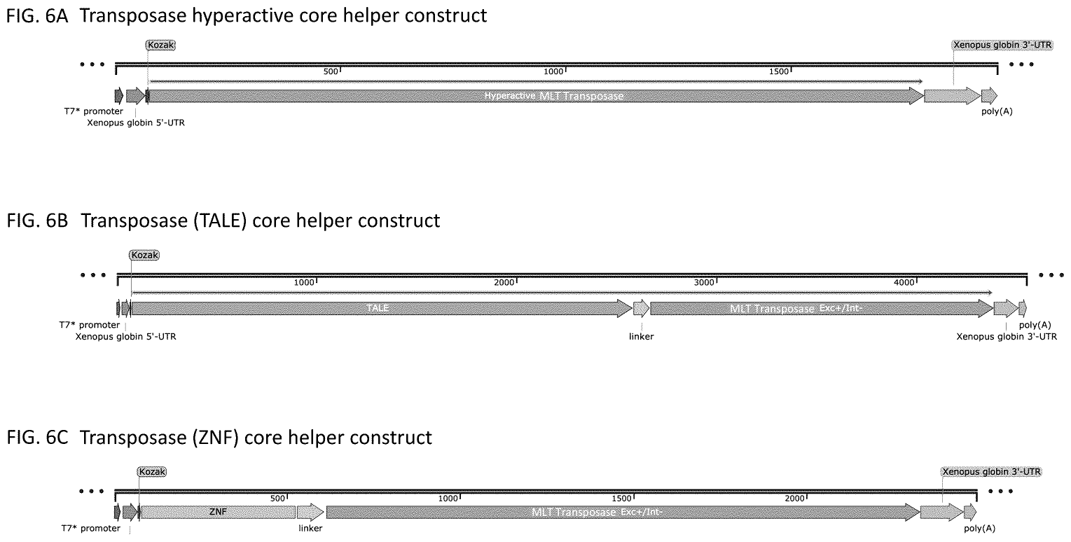

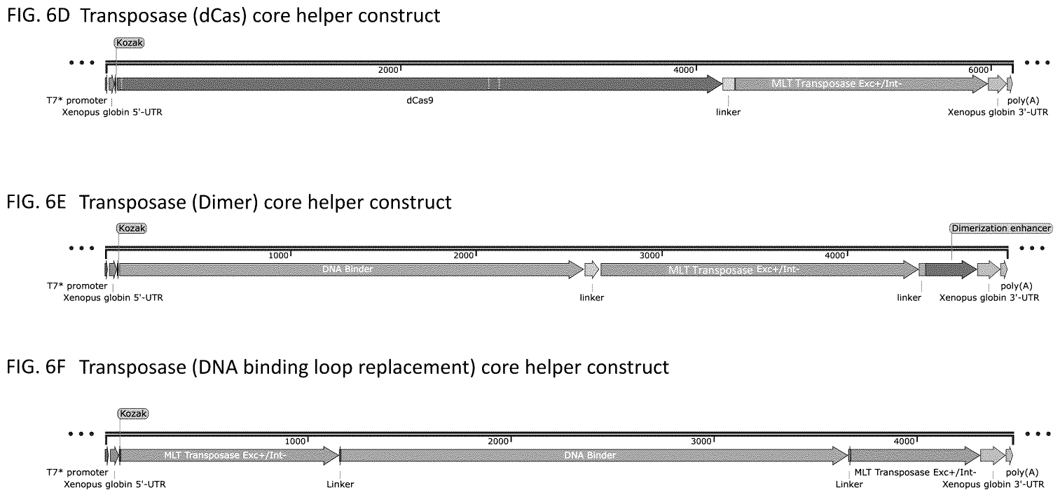

FIG. 6A-FIG. 6F depict six illustrative bioengineered RNA helper constructs that are contained in a replication backbone (e.g., plasmid or miniplasmid) with a T7 promoter (cap dependent), beta-globin 5′-UTR, and a helper enzyme with 2 or more mutations in the Myotis lucifugus helper (SEQ ID NO: 1, SEQ ID NO: 2, SEQ ID NO: 9, SEQ ID NO: 11) followed by a beta-globin 3′-UTR, and a poly-alanine tail (FIG. 6A). TALEs (FIG. 6B, TABLE 8-TABLE 12), ZnF (FIG. 6C, TABLE 13-TABLE 17), or a dead Cas9 (dCas9) binding protein (FIG. 6D, SEQ ID NO: 5, SEQ ID NO: 6) with guide RNAs (TABLE 3-TABLE 7) were linked to the N-terminus to target the specific TTAA sites at hROSA 26, AAVS1, chromosome 4, chromosome 22, and chromosome X loci. FIG. 6E depicts a construct with a dimerization enhancer. The dimerization enhancer may be selected from, without limitation, SH3, biotin, avidin, and rapamycin binders. The dimerization enhancer can be replaced with an intein. FIG. 6F depicts a construct that interrupts the natural DNA binding loop present in MLT (Y281-P339) and renders the helper enzyme Exc+/Int−. The extrinsic DNA binder that is inserted in the DNA binding loop binds to a target that is within 50 bp from a site-specific TTAA in the genome.

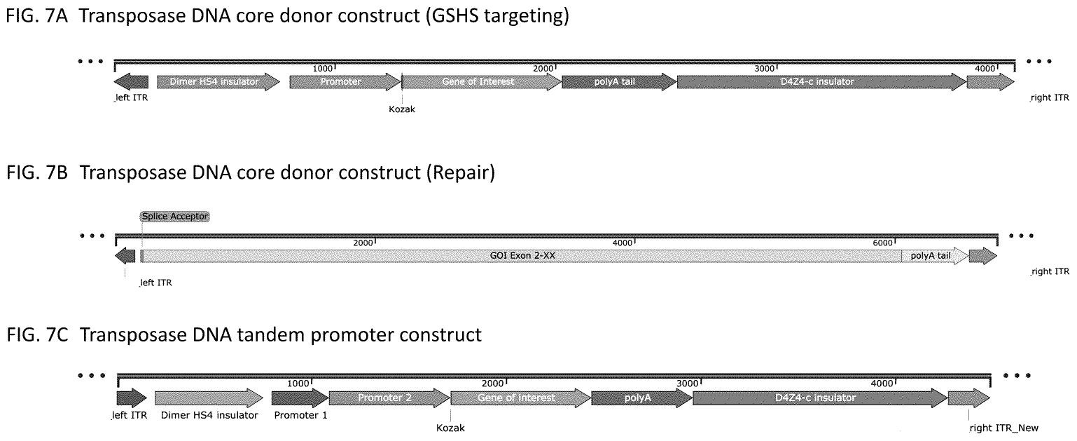

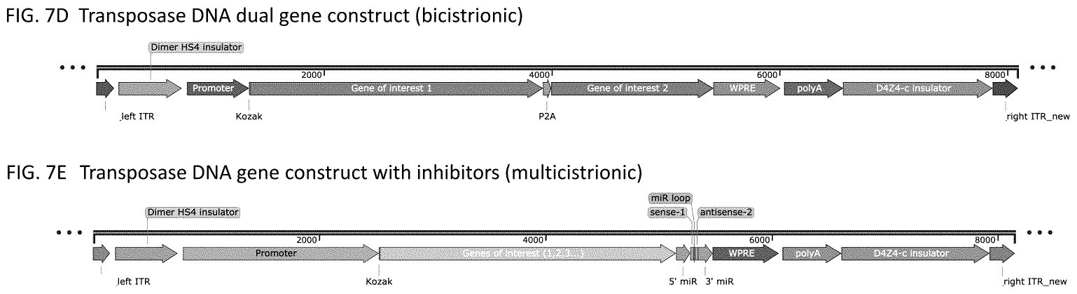

FIG. 7A depicts an illustrative core donor construct that is contained in a replication backbone (e.g., plasmid or miniplasmid) with a promoter driving a gene of interest (GOI) with a polyA tail flanked by two insulators and ITRs. The inverted terminal repeat (ITR) recognition sequences are included at the 5′-(SEQ ID NO: 3) and 3-ends (SEQ ID NO: 4). This construct is used for targeting genomic safe harbor sites (GSHS) or other loci.

FIG. 7B depicts an illustrative core donor construct that is contained in a replication backbone (e.g., plasmid or miniplasmid) with a splice acceptor site for exon 2 and other exons of a gene of interest (GOI) followed by a polyA tail and flanked by ITRs. The inverted terminal repeat (ITR) recognition sequences are included at the 5′-(SEQ ID NO: 3) and 3-ends (SEQ ID NO: 4). This construct is used for targeting endogenous genes in the first intron to repair downstream mutations.

FIG. 7C depicts an illustrative core donor construct that is contained in a replication backbone (e.g., plasmid or miniplasmid) with tandem promoters to affect expression in different tissues (e.g., without limitation, liver specific promoter, cardiac specific promoter) and a gene(s) of interest (GOI) followed by a polyA tail and flanked by ITRs. The inverted terminal repeat (ITR) recognition sequences are included at the 5′-(SEQ ID NO: 3) and 3-ends (SEQ ID NO: 4). This construct is used to differentially promote expression of genes in different organs, tissues or cell types.

FIG. 7D depicts an illustrative core donor construct that is contained in a replication backbone (e.g., plasmid or miniplasmid) with two or more genes of interest (GOI) linked by 2A “self-cleaving” peptides and followed by WPRE and a polyA tail. The construct is flanked by ITRs. The inverted terminal repeat (ITR) recognition sequences are included at the 5′-(SEQ ID NO: 3) and 3-ends (SEQ ID NO: 4). This construct is used for delivering multiple genes or genetic factors.

FIG. 7E depicts an illustrative core donor construct that is contained in a replication backbone (e.g., plasmid or miniplasmid) with a promoter(s) driving the expression of two or more genes as in FIG. 2D and linked to a sequence consisting of a 5′-miRNA, a sense and antisense miRNA pair, and completed with the 3-miRNA. The construct is followed by WPRE and flanked by ITRs. The inverted terminal repeat (ITR) recognition sequences are included at the 5′-(SEQ ID NO: 3) and 3-ends (SEQ ID NO: 4). This construct combines protein replacement and miRNA to inhibit other related protein expression. The sense and anti-sense miRNA pair regulate the sense miRNAs, probably via modulating the chromatin architectures of the resided genomic loci. See Brown, T., Howe, F. S., Murray, S. C., Wouters, M., Lorenz, P., Seward, E., . . . Mellor, J. (2018). Antisense transcription-dependent chromatin signature modulates sense transcript dynamics. Mol Syst Biol, 14(2), e8007; Murray, S. C., Haenni, S., Howe, F. S., Fischl, H., Chocian, K., Nair, A., & Mellor, J. (2015). Sense and antisense transcription are associated with distinct chromatin architectures across genes. Nucleic Acids Res, 43(16), 7823-7837.

### Example 7—Identification of Excision Positive and Integration Negative Mutants

FIG. 8 depicts the results of integration and excision assays on mutants by amino acid residue. Number denotes the position of the amino acid residue relative to SEQ ID NO: 2. The excision assay is a PCR-based assay to test for excision of the donor DNA. A HEK293 cell line that expresses GFP at a known genomic site was transfected with helper plasmid alone to excise the donor GFP DNA at the genomic locus by recognizing the end sequences. For the integration assay, HEK293 cells were plated in 12-well size plates the day before transfection. The day of the transfection the media was exchanged 1 hour and 30 min before the transfection was performed. A 3:1 ratio of X-tremeGENE™ 9 DNA Transfection Reagent protocol reagent was used to co-transfect a donor plasmid containing GFP and a helper plasmid in duplicate using 600 ng of DNA each. Forty-eight (48) hrs after the transfection the cells were analyzed by flow cytometry to count the percentage of GFP expressing cells to measure transient transfection efficiency. The cells were gated to distinguish them from debris and 20,000 cells were counted. The cultures were grown for 15-20 days without antibiotic. Cells were passaged ⅔ times per week. Flow cytometry was used to count the percentage of GFP expressing cells to measure integration efficiency at 2 weeks. The final integration efficiency was calculated by dividing the 2-week percentage of GFP cells by the percentage of GFP cell at 48 hr. The excision assay was performed by measuring the percentage of GFP cells in a cell line with a known GFP donor integration. The cells were grown to 80% confluency and analyzed by flow cytometry to count the percentage of GFP expressing cells as a baseline measurement. This percentage was used as the standard (i.e., 100%). X-tremeGENE™ 9 DNA Transfection Reagent protocol reagent was used to transfect helper plasmid in duplicate using 600 ng of DNA. The cells were gated to distinguish them from debris and 20,000 cells were counted. Forty-eight (48) hrs after the transfection the cells were analyzed by flow cytometry to count the percentage of GFP expressing cells. The cells were gated to distinguish them from debris and 20,000 cells were counted. The final integration efficiency was calculated by the baseline percentage of GFP cells by the percentage of GFP cells at 48 hrs.

Excision positive (EXC+) and integration deficient (INT−) mutants are shown in TABLE 1 and TABLE 2, respectively.

TABLE 1
Hyperactive helper mutants with excision activity

| MUTANT 1 | MUTANT 2 | MUTANT 3 | % GFP ≥20 | % GFP 10-19 | % GFP <10 |
|---|---|---|---|---|---|
| W168V | | | X | | |
| W168A | D416A | | X | | |
| K286A | D416A | | X | | |
| K287A | | | X | | |
| R333A | D416A | | X | | |
| K334A | | | X | | |
| K334A | D416A | | X | | |
| R336A | D416A | | X | | |
| K349A | | | X | | |
| K349A | D416A | | X | | |
| K350A | | | X | | |
| K350A | D416A | | X | | |
| K368A | | | X | | |
| K368A | D416A | | X | | |
| K369A | | | X | | |
| K369A | D416A | | X | | |
| D416A | | | X | | |
| D416A* | | | X | | |
| W168A | | | | X | |
| K286A | | | | X | |
| N310A | | | | X | |
| N310A | D416A | | | X | |
| K369A | | | | X | |
| R164N | | | | | X |
| D165N | | | | | X |
| K286A | R287A | | | | X |
| K286A | N310A | | | | X |
| K286A | K369A | | | | X |
| R287A | N310A | | | | X |
| R287A | K369A | | | | X |
| R287A | N310A | K369A | | | X |
| T331A | | | | | X |
| R333A | | | | | X |
| R336A | | | | | X |
| I338A | | | | | X |

*D416A HAS ONE MUTATION ONLY AND DOES NOT INLCUDE THE HYPERACTIVE HELPER MUTANTS S8P/C13R.

TABLE 2
Hyperactive helper Integration deficient mutants

| MUTANT 1 | MUTANT 2 | MUTANT 3 | % GFP <3 | % GFP 3-10 | % GFP ≥10 |
|---|---|---|---|---|---|
| R164N | | | X | | |
| D165N | | | X | | |
| W168V† | | | X | | |
| K286A | R287A | | X | | |
| K286A | N310A | | X | | |
| K286A | K369A | | X | | |
| R287A | N310A | K369A | X | | |
| T331A† | | | X | | |
| R333A† | | | X | | |
| D416N*† | | | X | | |
| W168A | D416A | | | X | |
| K286A | | | | X | |
| K287A | N310A | | X | | |
| N310A | | | | X | |
| R336A | | | | X | |
| I338A | | | | X | |
| K369A | | | | X | |
| K286A | | | | | X |
| K286A | D416A | | | | X |
| R287A | | | | | X |
| K287A | | | | | X |
| N310A | D416A | | | | X |
| R333A | D416A | | | | X |
| K334A | | | | | X |
| K334A | D416A | | | | X |
| R336A | D416A | | | | X |
| K349A | | | | | X |
| K349 | D416A | | | | X |
| K350A | | | | | X |
| K350A | D416A | | | | X |
| K368A | | | | | X |
| K368A | D416A | | | | X |
| K369A | | | | X | |
| K369A | D416A | | | | X |
| D416A | | | | | X |

*D416N HAS ONE MUTATION ONLY AND DOES NOT INCLUDE THE HYPERACTIVE HELPER MUTANTS S8P/C13R. D416N ENHANCES INTEGRATION AND EXCISION WHEN COMBINED WITH OTHER MUTANTS (FIG. 20).
†EXCISION+/INTEGRATION− (EXC+/INT−) MUTANTS

### Example 8—Identification Deletion Mutants and Fusion Protein Mutants

FIG. 9 depicts the integration and excision activity of deletion mutants. Number denotes the position of the amino acid residue relative to SEQ ID NO: 2. N-terminus deletions of the first 68 amino acid residues retain excision and integration activity with no activity after the deletion of the first 89 amino acid residues. Deletion of the C-terminus after amino acid residue 530 caused a loss of both excision and integration activity. Addition of an HA-tag did not alter the results.

FIG. 10 depicts the integration and excision activity of fusion proteins mutants. Number denotes the position of the amino acid residue relative to SEQ ID NO: 2. Fusion of TALEs and dCas9 on the N-terminus of the helper enzyme by a linker caused a loss of excision and integration activity. Post-translational protein splicing by an intein of a TALE and dCas9 showed a retention of both excision and integration activity.

### Example 9—Construction of Targeting Elements Directed to TTAA Sites in hROSA26, AAVS1, Chromosome 4, Chromosome 22, and Chromosome X Targeted by guideRNAs, TALES, and ZnF

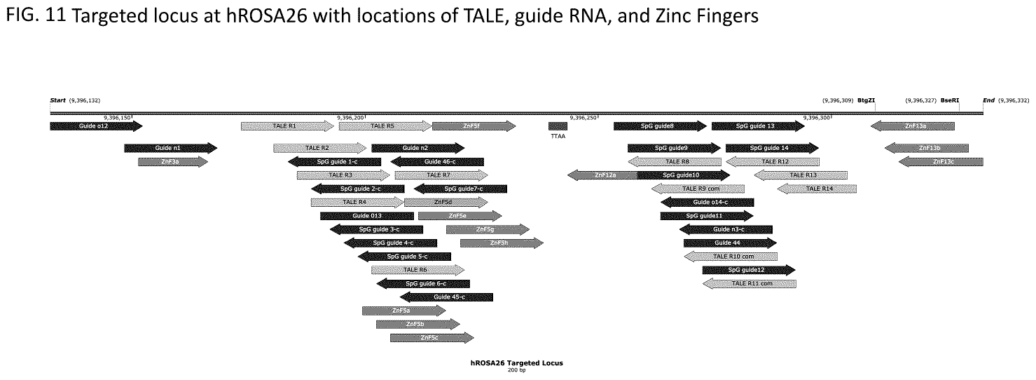

FIG. 11 depicts the TTAA site in hROSA26 (hg38 chr3:9,396,133-9,396,305) that is targeted by guideRNAs (TABLE 3), TALES (TABLE 8), and ZnF (TABLE 13).

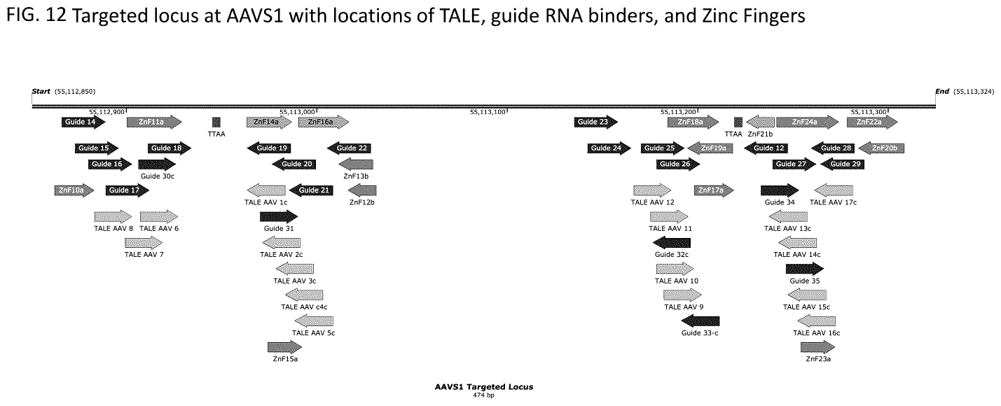

FIG. 12 depicts two TTAA sites in AAVS1 (hg38 chr19:55,112,851-55,113,324) that are targeted by guideRNAs (TABLE 4) or TALES (TABLE 9), and ZnF (TABLE 14).

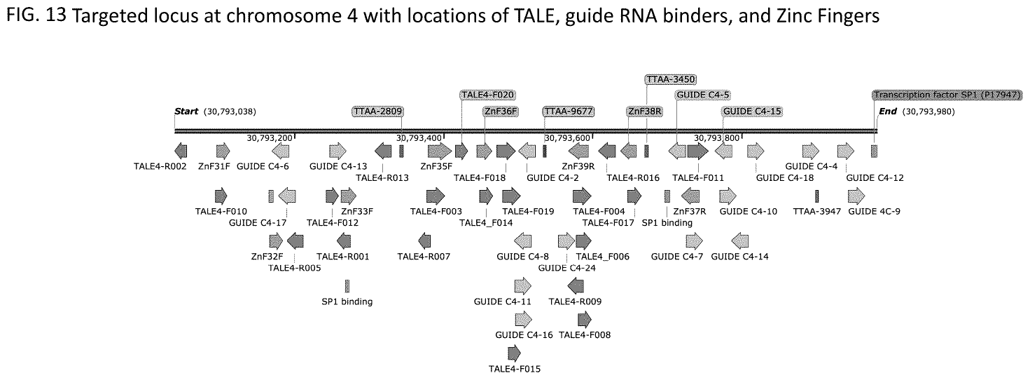

FIG. 13 depicts two TTAA sites in Chromosome 4 (hg38 chr4:30,793,534-30,875,476) that are targeted by guideRNAs (TABLE 5) or TALES (TABLE 10), and ZnF (TABLE 15).

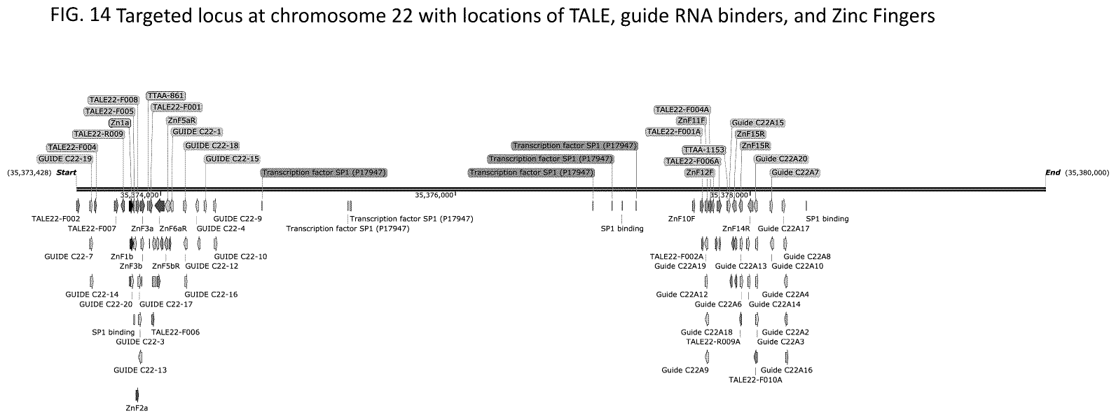

FIG. 14 depicts two TTAA sites in Chromosome 22 (hg38 chr22:35,370,000-35,380,000) that are targeted by guideRNAs (TABLE 6) or TALES (TABLE 11), and ZnF (TABLE 16).

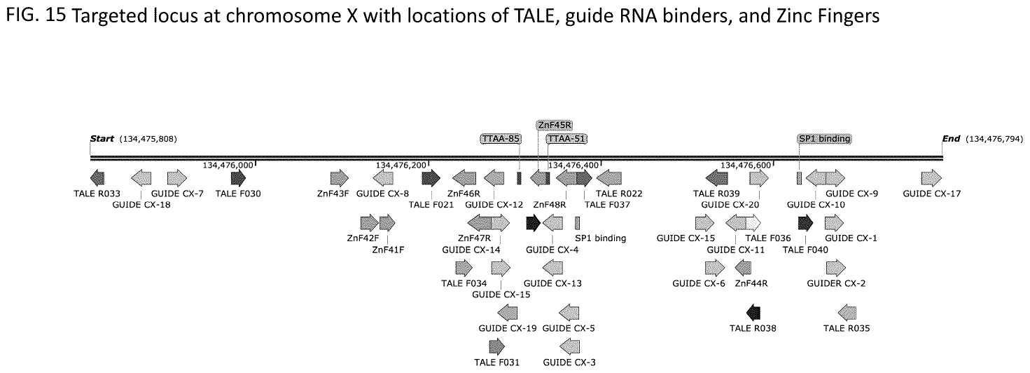

FIG. 15 depicts two TTAA sites in Chromosome X (hg38 chrX:134,419,661-134,541,172) that are targeted by guideRNAs (TABLE 7) or TALES (TABLE 12), and ZnF (TABLE 17).

TABLE 3
Guide RNA sequences targeting the genomic safe
harbor site, hROSA26.

| HROSA26 GUIDE NO. | DNA SEQUENCE |
|---|---|
| GUIDE 44 | AATCGAGAAGCGACTCGACA |
| GUIDE 45-C | GTCCCTGGGCGTTGCCCTGC |
| GUIDE 46-C | CCCTGGGCGTTGCCCTGCAG |
| SPG GUIDE1-C | GAGTGAGCAGCTGTAAGATT |
| SPG GUIDE2-C | CAGGGGAGTGAGCAGCTGTA |
| SPG GUIDE3-C | CCTGCAGGGGAGTGAGCAGC |
| SPG GUIDE4-C | TGCCCTGCAGGGGAGTGAGC |
| SPG GUIDE5-C | CGTTGCCCTGCAGGGGAGTG |
| SPG GUIDE6-C | TGGGCGTTGCCCTGCAGGGG |
| SPG GUIDE7-C | TTGGTCCCTGGGCGTTGCCC |
| SPG GUIDE8 | AAGAATCCCGCCCATAATCG |
| SPG GUIDE9 | AATCCCGCCCATAATCGAGA |
| SPG GUIDE10 | TCCCGCCCATAATCGAGAAG |
| SPG GUIDE11 | CCCATAATCGAGAAGCGACT |
| SPG GUIDE12 | GAGAAGCGACTCGACATGGA |
| SPG GUIDE13 | GAAGCGACTCGACATGGAGG |
| SPG GUIDE14 | GCGACTCGACATGGAGGCGA |
| GUIDE N1 | CCGTGGGAAGATAAACTAAT |
| GUIDE N2 | TCCCCTGCAGGGCAACGCCC |
| GUIDE N3-C | GTCGAGTCGCTTCTCGATTA |
| GUIDE O12 | CGACACCAACTCTAGTCCGT |
| GUIDE O13 | CAGCTGCTCACTCCCCTGCA |
| GUIDE O14-C | AGTCGCTTCTCGATTATGGG |

TABLE 4
Guide RNA sequences targeting the genomic safe
harbor site, AAVS1.

| AAVS1 GUIDE NO. | DNA SEQUENCE |
|---|---|
| AAV GUIDE 12 | ACCCTTGGAAGGACCTGGCTGGG |
| AAV GUIDE 13c | TCCGAGCTTGACCCTTGGAA |
| AAV GUIDE 14 | GGAGCCACGAAAACAGATCCAGG |
| AAV GUIDE 14c | TGGTTTCCGAGCTTGACCCT |
| AAV GUIDE 15 | GGAGCCACGAAAACAGATCCAGG |
| AAV GUIDE 16 | AGATCCAGGGACACGGTGCTAGG |
| AAV GUIDE 17 | GACACGGTGCTAGGACAGTGGGG |
| AAV GUIDE 18 | GAAAATGACCCAACAGCCTCTGG |
| AAV GUIDE 19 | GCCTGGCCGGCCTGACCACTGGG |
| AAV GUIDE 20 | CTGAGCACTGAAGGCCTGGCCGG |
| AAV GUIDE 21 | TGGTTTCCACTGAGCACTGAAGG |
| AAV GUIDE 22 | GGTGCTTTCCTGAGGACCGATAG |
| AAV GUIDE 23 | GCGCTTCCAGTGCTCAGACTAGG |
| AAV GUIDE 24 | CAGTGCTCAGACTAGGGAAGAGG |
| AAV GUIDE 25 | GCCCCTCCTCCTTCAGAGCCAGG |
| AAV GUIDE 26 | TCCTTCAGAGCCAGGAGTCCTGG |
| AAV GUIDE 27 | CCAAGGGTCAAGCTCGGAAACCA |
| AAV GUIDE 28 | CTGCAGAGTATCTGCTGGGGTGG |
| AAV GUIDE 29 | CGTTCCTGCAGAGTATCTGCTGG |
| AAV GUIDE 30c | GTGGGGAAAATGACCCAACA |
| AAV GUIDE 31 | GAAGGCCTGGCCGGCCTGAC |
| AAV GUIDE 32c | ACTCCTGGCTCTGAAGGAGG |
| AAV GUIDE 33c | GGGCTGGGGGCCAGGACTCC |
| AAV GUIDE 34 | GTCCTTCCAAGGGTCAAGCT |
| AAV GUIDE 35 | TCAAGCTCGGAAACCACCCC |

TABLE 5
Guide RNA sequences targeting chromosome 4 TTAA
hotspot [hg38 chr4:30, 793, 533-30, 793, 537
(9677); chr4:30, 875, 472-30, 875, 476 (8948)].

| CHR4 GUIDE NO. | DNA SEQUENCE |
|---|---|
| Guide C4-1 | ATTGTCTTCACTAAACCCGTTGG |
| Guide C4-2 | TAAACCCGTTGGGAATACAATGG |
| Guide C4-3 | TTGTCTTCACTAAACCCGTTGGG |
| Guide C4-4 | TGATTCATAGGAGTCTATTAAGG |
| Guide C4-5 | TTACATATGCTTCGAGTTTGTGG |
| Guide C4-6 | ACTCTTAAGGTAGGACTAATTGG |
| Guide C4-7 | TATGTGTGCAATAGCGTTAAAGG |
| Guide C4-8 | CGTTGGGAATACAATGGCTTAGG |
| Guide C4-9 | TCACAATGGAACTCTGCCTTTGG |
| Guide C4-10 | GACCACAAATCAATGCCCAAAGG |
| Guide C4-11 | CTAAGCCATTGTATTCCCAACGG |
| Guide C4-12 | AGCATTCTGGAGTGTCACAATGG |
| Guide C4-13 | CAATAGCCCACTTTAATACTAGG |
| Guide C4-14 | CTTTATCCAAGTGAATCCTTTGG |
| Guide C4-15 | GGCATTGATTTGTGGTCATTTGG |
| Guide C4-16 | TAAGCCATTGTATTCCCAACGGG |
| Guide C4-17 | AATACAATCACTCTTAAGGTAGG |
| Guide C4-18 | GAAGTACCTTTCACTATTTTGGG |
| Guide C4-19 | CAAGCAACAAATGACTTCTAAGG |
| Guide C4-20 | TTTGAATACAATCACTCTTAAGG |
| Guide C4A1 | ACAAACGGACTACGTAAACTTGG |
| Guide C4A2 | ACAAGATGTGAACACGACGATGG |
| Guide C4A3 | GTTGCACCGTTGATTCCTTCAGG |
| Guide C4A4 | AGTAATATTGAATTAGGGCGTGG |
| Guide C4A5 | CCTGATGTTGGCTCGACATTAGG |
| Guide C4A6 | CTTTGTTGGGTCTTAGCTTAAGG |
| Guide C4A7 | TCGGAACAGCTCCTTCCTGAAGG |
| Guide C4A8 | AGTAGTTTCTGAGGTCATGTTGG |
| Guide C4A9 | CTTGAAAATACGATGATGTGAGG |
| Guide C4A10 | GCATTAATCTAGAGAGAGGGAGG |
| Guide C4A11 | GGGTCATGTTAGAATTCATGTGG |
| Guide C4A12 | TGATGCATTAATCTAGAGAGAGG |
| Guide C4A13 | ACATCATCGTATTTTCAAGTTGG |
| Guide C4A14 | CTAGCTGACAAACATGTGAGTGG |
| Guide C4A15 | AACATGACCCAAGTGAGTCCAGG |
| Guide C4A16 | GATTCCGTATTTGCTTTGTTGGG |
| Guide C4A17 | TACGATGATGTGAGGAAATAAGG |
| Guide C4A18 | GTAATATGTCTAAGTACTGATGG |
| Guide C4A19 | GTAAAGTGAGCTGGTTCATTAGG |
| Guide C4A20 | ACTAGAGTCCTTAAGAAGGGGGG |

CHOPCHOP algorithm

TABLE 6
Guide RNA sequences targeting chromosome 22 TTAA
hotspot. [hg38 chr22:35, 373, 912-35, 373, 916
(861); chr22:35, 377, 843-35, 377, 847 (1153)].

| CHR22 GUIDE NO. | DNA SEQUENCE |
|---|---|
| Guide C22-1 | ATAACACGTGAGCCGTCCTAAGG |
| Guide C22-2 | GGAAGACTTTTCTCTATACGAGG |
| Guide C22-3 | GCATTCCTTTCATCCATGGCAGG |
| Guide C22-4 | GACATATGGTTATAAAAATCAGG |
| Guide C22-5 | GGAGTGCAGTCCCTGACATATGG |
| Guide C22-6 | GTGGGTTAGGGTGGTTAACTGGG |
| Guide C22-7 | AGGTGCAAAAAGGTTGCTGTGGG |
| Guide C22-8 | CGTGACAAGGCAAAGTGGCGTGG |
| Guide C22-9 | GAAGGACTGCCCCTGACGTCAGG |
| Guide C22-10 | CTGCCCCTGACGTCAGGAGTTGG |
| Guide C22-11 | TGTGGGTTAGGGTGGTTAACTGG |
| Guide C22-12 | ACCCTTTTAGAGTTTTCTGCTGG |
| Guide C22-13 | AACTTCCTGCCATGGATGAAAGG |
| Guide C22-14 | GCAAAAAGGTTGCTGTGGGTTGG |
| Guide C22-15 | AATTTGGGGGTAGATAGGCATGG |
| Guide C22-16 | AGAAAACTCTAAAAGGGTATAGG |
| Guide C22-17 | ATTAGCATTCCTTTCATCCATGG |
| Guide C22-18 | CCCAGCAGAAAACTCTAAAAGGG |
| Guide C22-19 | CAGGTGCAAAAAGGTTGCTGTGG |
| Guide C22-20 | GCAAGAGATGAAATTCCATATGG |
| Guide C22A1 | GGGCTGTTCTAACGAAGTCTGGG |
| Guide C22A2 | TGTCCATTCAGCGACCCTAGAGG |
| Guide C22A3 | GGCTGTTCTAACGAAGTCTGGGG |
| Guide C22A4 | GTCCATTCAGCGACCCTAGAGGG |
| Guide C22A5 | GGGGCTGTTCTAACGAAGTCTGG |
| Guide C22A6 | GGCTGAATCAGCATGCGAAAGGG |
| Guide C22A7 | TTCCAATGGGGGGCATAGCCTGG |
| Guide C22A8 | TACCCTCTAGGGTCGCTGAATGG |
| Guide C22A9 | ATCCTCTTGGGCCTTATAAGAGG |
| Guide C22A10 | GGCCAGGCTATGCCCCCCATTGG |
| Guide C22A11 | CTAGAGGACCAGAACAACTCTGG |
| Guide C22A12 | TCCCTCTTATAAGGCCCAAGAGG |
| Guide C22A13 | AGGCTGAATCAGCATGCGAAAGG |
| Guide C22A14 | GGACCAGAACAACTCTGGCCTGG |
| Guide C22A15 | GGGCTTTTATTTGGCCCAGCAGG |
| Guide C22A16 | GTCGCTGAATGGACAGACTCTGG |
| Guide C22A17 | CTCATGAGTTTTACCCTCTAGGG |
| Guide C22A18 | TCCTCTTGGGCCTTATAAGAGGG |
| Guide C22A19 | TCTTGGGCCTTATAAGAGGGAGG |
| Guide C22A20 | TAGAACAGCCCCCCACACAGTGG |

TABLE 7
Guide RNA sequences targeting chromosome X (HPRT)
TTAA hotspot. [hg38 chrX:134, 476, 304-134, 476,
307 (85); chrX:134,476, 337-134, 476, 340 (51)].

| CHRX GUIDE NO. | DNA SEQUENCE |
|---|---|
| Guide CX-1 | GTTACGTTATGACTAATCTTTGG |
| Guide CX-2 | TACGTTATGACTAATCTTTGGGG |
| Guide CX-3 | GGAAGTAGTGTTATGATGTATGG |
| Guide CX-4 | GTTATGATGTATGGGCATAAAGG |
| Guide CX-5 | GAAGTAGTGTTATGATGTATGGG |
| Guide CX-6 | ATAGCTGCTGGCAGTATAACTGG |
| Guide CX-7 | GCATCACAACATTGACACTGTGG |
| Guide CX-8 | AAGGCGAGTTTCTACAAAGATGG |
| Guide CX-9 | TTACGTTATGACTAATCTTTGGG |
| Guide CX-10 | CAAGACTGATTAAGACTGATGGG |
| Guide CX-11 | AGCAGCAATGTATTAAAGGCTGG |
| Guide CX-12 | CTACAGGATTGATGTAAACATGG |
| Guide CX-13 | TGGGCATAAAGGGTTTTAATGGG |
| Guide CX-14 | ACATCAATCCTGTAGGTGATTGG |
| Guide CX-15 | ATTCTAGTCATTATAGCTGCTGG |
| Guide CX-16 | CATCAATCCTGTAGGTGATTGGG |
| Guide CX-17 | GTTATAAGATCAATTCTGAGTGG |
| Guide CX-18 | GGCAGACTGTGGATCAAAAGTGG |
| Guide CX-19 | ATGGCTGCCCAATCACCTACAGG |
| Guide CX-20 | TCAAAGCATGTACTTAGAGTTGG |

TABLE 8
TALE sequences targeting the genomic safe harbor site, hROSA26.

| NAME | DNA SEQUENCE | RVD AMINO ACID CODE |
|---|---|---|
| R1 | TCGCCCCTCAAATCTTACAG | HD NH HD HD HD HD NG HD NI NI NI NG HD NG NG NI HD NI NH |
| R2 | TCAAATCTTACAGCTGCTCA | HD NI NI NI NG HD NG NG NI HD NI NH HD NG NH HD NG HD NI |
| R3 | TCTTACAGCTGCTCACTCCC | HD NG NG NI HD NI NH HD NG NH HD NG HD NI HD NG HD HD HD |
| R4 | TACAGCTGCTCACTCCCCTG | NI HD NI NH HD NG NH HD NG HD NI HD NG HD HD HD HD NG NH |
| R5 | TGCTCACTCCCCTGCAGGGC | NH HD NG HD NI HD NG HD HD HD HD NG NH HD NI NH NH NH HD |
| R6 | TCCCCTGCAGGGCAACGCCC | HD HD HD HD NG NH HD NI NH NH NH HD NI NI HD NH HD HD HD |
| R7 | TGCAGGGCAACGCCCAGGGA | NH HD NI NH NH NH HD NI NI HD NH HD HD HD NI NH NH NH NI |
| R8 | TCTCGATTATGGGCGGGATT | HD NG HD NH NI NG NG NI NG NH NH NH HD NH NH NH NI NG NG |
| R9 | TCGCTTCTCGATTATGGGCG | HD NH HD NG NG HD NG HD NH NI NG NG NI NG NH NH NH HD NH |
| R10 | TGTCGAGTCGCTTCTCGATT | NH NG HD NH NI NH NG HD NH HD NG NG HD NG HD NH NI NG NG |
| R11 | TCCATGTCGAGTCGCTTCTC | HD HD NI NG NH NG HD NH NI NH NG HD NH HD NG NG HD NG HD |
| R12 | TCGCCTCCATGTCGAGTCGC | HD NH HD HD NG HD HD NI NG NH NG HD NH NI NH NG HD NH HD |
| R13 | TCGTCATCGCCTCCATGTCG | HD NH NG HD NI NG HD NH HD HD NG HD HD NI NG NH NG HD NH |
| R14 | TGATCTCGTCATCGCCTCCA | NH NI NG HD NG HD NH NG HD NI NG HD NH HD HD NG HD HD NI |

TABLE 9
TALE sequences targeting the genomic safe harbor site, AAVS1.

| NAME | DNA SEQUENCE | RVD AMINO ACID CODE |
|---|---|---|
| AAV1c | TGGCCGGCCTGACCACTGGG | NH NH HD HD NH NH HD HD NG NH NI HD HD NI HD NG NH NH NH |
| AAV2c | TGAAGGCCTGGCCGGCCTGA | NH NI NI NH NH HD HD NG NH NH HD HD NH NH HD HD NG NH NI |
| AAV3c | TGAGCACTGAAGGCCTGGCC | NH NI NH HD NI HD NG NH NI NI NH NH HD HD NG NH NH HD HD |
| AAV4c | TCCACTGAGCACTGAAGGCC | HD HD NI HD NG NH NI NH HD NI HD NG NH NI NI NH NH HD HD |
| AAV5c | TGGTTTCCACTGAGCACTGA | NH NH NG NG NG HD HD NI HD NG NH NI NH HD NI HD NG NH NI |
| AAV6 | TGGGGAAAATGACCCAACAG | NH NH NH NH NI NI NI NI NG NH NI HD HD HD NI NI HD NI NH |
| AAV7 | TAGGACAGTGGGGAAAATGA | NI NH NH NI HD NI NH NG NH NH NH NH NI NI NI NI NG NH NI |
| AAV8 | TCCAGGGACACGGTGCTAGG | HD HD NI NH NH NH NI HD NI HD NH NH NG NH HD NG NI NH NH |
| AAV9 | TCAGAGCCAGGAGTCCTGGC | HD NI NH NI NH HD HD NI NH NH NI NH NG HD HD NG NH NH HD |
| AAV10 | TCCTTCAGAGCCAGGAGTCC | HD HD NG NG HD NI NH NI NH HD HD NI NH NH NI NH NG HD HD |
| AAV11 | TCCTCCTTCAGAGCCAGGAG | HD HD NG HD HD NG NG HD NI NH NI NH HD HD NI NH NH NI NH |
| AAV12 | TCCAGCCCCTCCTCCTTCAG | HD HD NI NH HD HD HD HD NG HD HD NG HD HD NG NG HD NI NH |
| AAV13c | TCCGAGCTTGACCCTTGGAA | HD HD NH NI NH HD NG NG NH NI HD HD HD NG NG NH NH NI NI |
| AAV14c | TGGTTTCCGAGCTTGACCCT | NH NH NG NG NG HD HD NH NI NH HD NG NG NH NI HD HD HD NG |
| AAV15c | TGGGGTGGTTTCCGAGCTTG | NH NH NH NH NG NH NH NG NG NG HD HD NH NI NH HD NG NG NH |
| AAV16c | TCTGCTGGGGTGGTTTCCGA | HD NG NH HD NG NH NH NH NH NG NH NH NG NG NG HD HD NH NI |
| AAV17c | TGCAGAGTATCTGCTGGGGT | NH HD NI NH NI NH NG NI NG HD NG NH HD NG NH NH NH NH NG |

TABLE 10
TALE sequences targeting the chromosome 4 hotspot. [hg38 chr4:30, 793, 533-30, 793, 537
(9677); chr4:30, 875, 472-30, 875, 476 (8948)].

| NAME | DNA SEQUENCE | RVD AMINO ACID CODE |
|---|---|---|
| TALE4-R001 | TCTTCCTAGTATTAAAGT | HD NG NG HD HD NG NI NH NG NI NG NG NI NI NI |
| | | NH NG |
| TALE4-R002 | TCCTTAATATTACCAGT | HD HD NG NG NI NI NG NI NG NG NI HD HD NI NH |
| | | NG |
| TALE4-F003 | TACCAAGCTGAAATGACACAAAAGT | NI HD HD NI NI NH HD NG NH NI NI NI NG NH NI |
| | | HD NI HD NI NI NI NI NH NG |
| TALE4-F004 | TGGCTGTGTCACATACCAGCAGAAT | NH NH HD NG NH NG NH NG HD NI HD NI NG NI HD |
| | | HD NI NH HD NI NH NI NI NG |
| TALE4-F005 | TGTTAATTTGAATACAATCACT | NH NG NG NI NI NG NG NG NH NI NI NG NI HD NI |
| | | NI NG HD NI HD NG |
| TALE4-F006 | TGTGTCACATACCAGCAGAAT | NH NG NH NG HD NI HD NI NG NI HD HD NI NH HD |
| | | NI NH NI NI NG |
| TALE4-R007 | TGGTAACTACTAATTT | NH NH NG NI NI HD NG NI HD NG NI NI NG NG NG |
| TALE4-F008 | TGTCACATACCAGCAGAAT | NH NG HD NI HD NI NG NI HD HD NI NH HD NI NH |
| | | NI NI NG |
| TALE4-R009 | TGTGACACAGCCATCAACAAT | NH NG NH NI HD NI HD NI NH HD HD NI NG HD NI |
| | | NI HD NI NI NG |
| TALE4-F010 | TCCTTTGATGAACAGT | HD HD NG NG NG NH NI NG NH NI NI HD NI NH NG |
| TALE4-F011 | TGTGTGCAATAGCGTTAAAGGAACTACAT | NH NG NH NG NH HD NI NI NG NI NH HD NH NG NG |
| | | NI NI NI NH NH NI NI HD NG NI HD NI NG |
| TALE4-F012 | TCTTTCAATAGCCCACT | HD NG NG NG HD NI NI NG NI NH HD HD HD NI HD |
| | | NG |
| TALE4-R013 | TCTCAAATGACAAGAGCACAGT | HD NG HD NI NI NI NG NH NI HD NI NI NH NI NH |
| | | HD NI HD NI NH NG |
| TALE4-F014 | TACCAGTTAATTAGCACT | NI HD HD NI NH NG NG NI NI NG NG NI NH HD NI |
| | | HD NG |
| TALE4-F015 | TGTTGTGACCTAAGCCAT | NH NG NG NH NG NH NI HD HD NG NI NI NH HD HD |
| | | NI NG |
| TALE4-R016 | TCTCATGTTTTAAAGTCAAGAAT | HD NG HD NI NG NH NG NG NG NG NI NI NI NH NG |
| | | HD NI NI NH NI NI NG |
| TALE4-F017 | TCCTGAATTCAGAACAGAT | HD HD NG NH NI NI NG NG HD NI NH NI NI HD NI |
| | | NH NI NG |
| TALE4-F018 | TAGCATGATGTTTCATGTTGTGACCT | NI NH HD NI NG NH NI NG NH NG NG NG HD NI NG |
| | | NH NG NG NH NG NH NI HD HD NG |
| TALE4-F019 | TGTTTCATGTTGTGACCTAAGCCAT | NH NG NG NG HD NI NG NH NG NG NH NG NH NI HD |
| | | HD NG NI NI NH HD HD NI NG |
| TALE4-F020 | TACAACAGTCTATTTCAT | NI HD NI NI HD NI NH NG HD NG NI NG NG NG HD |
| | | NI NG |

TABLE 11
TALE sequences targeting the chromosome 22 hotspot. [hg38 chr22:35, 373, 912-35, 373,
916 (861); chr22:35, 377, 843-35, 377, 847 (1153)].

| NAME | DNA SEQUENCE | RVD AMINO ACID CODE |
|---|---|---|
| TALE22F- | TCTTCCTAGTCTCTTCTCTACCCAGT | HD NG NG HD HD NG NI NH NG HD NG HD NG NG HD NG |
| R001 | | HD NG NI HD HD HD NI NH NG |
| TALE22- | TACACTCCAGCCTGGGAAACAGAGT | NI HD NI HD NG HD HD NI NH HD HD NG NH NH NH NI |
| F002 | | NI NI HD NI NH NI NH NG |
| TALE22- | TCTTTTCCTTAGGACGGCT | HD NG NG NG NG HD HD NG NG NI NH NH NI HD NH NH |
| F003 | | HD NG |
| TALE22- | TCGCTCAGGCCTGTCAT | HD NH HD NG HD NI NH NH HD HD NG NH NG HD NI NG |
| F004 | | |
| TALE22- | TCCATATGGAAGACTT | HD HD NI NG NI NG NH NH NI NI NH NI HD NG NG |
| F005 | | |
| TALE22- | TACCCAGTTAACCACCCT | NI HD HD HD NI NH NG NG NI NI HD HD NI HD HD HD |
| F006 | | NG |
| TALE22- | TGGCGCATGCCTGTAATCCCAGCTACT | NH NH HD NH HD NI NG NH HD HD NG NH NG NI NI NG |
| F007 | | HD HD HD NI NH HD NG NI HD NG |
| TALE22- | TATACGAGGAGAAAATTAGCATTCCT | NI NG NI HD NH NI NH NH NI NH NI NI NI NI NG NG |
| F008 | | NI NH HD NI NG NG HD HD NG |
| TALE22- | TCTGCCTCCCAGGTTCACGCAAT | HD NG NH HD HD NG HD HD HD NI NH NH NG NG HD NI |
| R009 | | HD NH HD NI NI NG |
| TALE22- | TGCCTTGTCACGTTTTCACAGT | NH HD HD NG NG NH NG HD NI HD NH NG NG NG NG HD |
| F010 | | NI HD NI NH NG |
| TALE22- | TGTCACCTTCTGTATGTGCAACCAT | NH NG HD NI HD HD NG NG HD NG NH NG NI NG NH NG |
| F001A | | NH HD NI NI HD HD NI NG |
| TALE22- | TCTGTATGTGCAACCAT | HD NG NH NG NI NG NH NG NH HD NI NI HD HD NI NG |
| F002A | | |
| TALE22- | TAGTCAAGCAACAGGAT | NI NH NG HD NI NI NH HD NI NI HD NI NH NH NI NG |
| R03A | | |
| TALE22- | TCCAAGATAATTCCCCAT | HD HD NI NI NH NI NG NI NI NG NG HD HD HD HD NI |
| F004A | | NG |
| TALE22- | TCTGCAAGATCCTTTT | HD NG NH HD NI NI NH NI NG HD HD NG NG NG NG |
| F005A | | |
| TALE22- | TGCTATGTAAGGTAGCAAAAAGGTAACCT | NH HD NG NI NG NH NG NI NI NH NH NG NI NH HD NI |
| F006A | | NI NI NI NI NH NH NG NI NI HD HD NG |
| TALE22- | TCTCTCTCCTCCTGCT | HD NG HD NG HD NG HD HD NG HD HD NG NH HD NG |
| R007A | | |
| TALE22- | TCCAAATGCTATTCTCTCT | HD HD NI NI NI NG NH HD NG NI NG NG HD NG HD NG |
| R008A | | HD NG |
| TALE22- | TGCTGATTCAGCCTCCT | NH HD NG NH NI NG NG HD NI NH HD HD NG HD HD NG |
| R009A | | |
| TALE22- | TAGAACAGCCCCCCACACAGT | NI NH NI NI HD NI NH HD HD HD HD HD HD NI HD NI |
| F010A | | HD NI NH NG |

TABLE 12
TALE sequences targeting the chromosome X (HPRT) hotspot.

| NAME | DNA SEQUENCE | RVD AMINO ACID CODE |
|---|---|---|
| TALE F002 | TTTAGCAGATGCATCAGC | NG NG NI NH HD NI NH NI NG NH HD NI NG HD NI NH HD |
| TALE F003 | TGACCAGGGGCATGTCCTGG | NH NI HD HD NI NH NH NH NH HD NI NG NH NG HD HD NG |
| | | NH NH |
| TALE F004 | TGGTCCACCTACCTGAAAATG | HD NI NI NH NH NI NH NG NG HD NG NH NH HD NG NH NH |
| | | NH NG HD |
| TALE F007 | TGTCCCACAGGTATTACGGGC | NH NH HD HD HD NI HD NI NH NH NG NI NG NG NI HD NH |
| | | NH NG HD |
| TALE F008 | TACGGGCCAACCTGACAATAC | NI HD NH NH NH HD HD NI NI HD HD NG NH NI HD NI NI |
| | | NG NI HD |
| TALE F009 | TGAGCTTTGGGGACTGAAAGA | NH NI NH HD NG NG NG NH NH NH NH NI HD NG NH NI NI |
| | | NI NH NI |
| TALE R002 | CTGGCATAATCTTTTCCCCCA | NH NH NH NH NH NI NI NI NI NH NI NG NG NI NG NH HD |
| | | HD NI NH |
| TALE R003 | CCAGCCTCCTGGCCATGTGCA | NG HD NI HD NI NG NH NH HD HD NI NH NH NI NH NH HD |
| | | NG NH NH |
| TALE R004 | GGCCATGTGCACAGGGGCTGA | HD NI NH HD HD HD HD NG NH NG NH HD NI HD NI NG NH |
| | | NH HD HD |
| TALE R005 | CTGATATGTGAAGGTTTAGCA | NH HD NG NI NI NI HD HD NG NG HD NI HD NI NG NI NG |
| | | HD NI NH |
| TALE R007 | TGACCAGGCGTGGTGGCTCAC | NH NI HD HD NI NH NH HD NH NG NH NH NG NH NH HD NG |
| | | HD NI HD |
| TALE F020* | TATAGACATTTTCACT | NI NG NI NH NI HD NI NG NG NG NG HD NI HD NG |
| TALE F021* | TCTACATTTAACTATCAACCT | HD NG NI HD NI NG NG NG NI NI HD NG NI NG HD NI NI |
| | | HD HD NG |
| TALE F030* | TCGTGCAAACGTTTGAT | HD NH NG NH HD NI NI NI HD NH NG NG NG NH NI NG |
| TALE F031* | TACATCAATCCTGTAGGT* | NI HD NI NG HD NI NI NG HD HD NG NH NG NI NH NH NG |
| TALE F034* | TCTATTTTAGTGACCCAAGT | HD NG NI NG NG NG NG NI NH NG NH NI HD HD HD NI NI |
| | | NH NG |
| TALE F036* | TAGAGTCAAAGCATGTACT | NI NH NI NH NG HD NI NI NI NH HD NI NG NH NG NI HD |
| | | NG |
| TALE F037* | TCCTACCCATAAGCTCCT | HD HD NG NI HD HD HD NI NG NI NI NH HD NG HD HD NG |
| TALE F040* | TCCCCATCCCCATCAGT | HD HD HD HD NI NG HD HD HD HD NI NG HD NI NH NG |
| TALE R022* | TCTTTAATTCAAGCAAGACTTTAACAAGT | HD NG NG NG NI NI NG NG HD NI NI NH HD NI NI NH NI |
| | | HD NG NG NG NI NI HD NI NI NH NG |
| TALE R033* | TGCAGTCCCCTTTCTT | NH HD NI NH NG HD HD HD HD NG NG NG HD NG NG |
| TALE R035* | TCTGCACAAATCCCCAAAGAT | HD NG NH HD NI HD NI NI NI NG HD HD HD HD NI NI NI |
| | | NH NI NG |
| TALE R038* | TACATGCTTTGACTCT | NI HD NI NG NH HD NG NG NG NH NI HD NG HD NG |
| TALE R039* | TGGCCAGTTATACTGCCAGCAGCTATAAT | NH NH HD HD NI NH NG NG NI NG NI HD NG NH HD HD NI |
| | | NH HD NI NH HD NG NI NG NI NI NG |

*TALES near hotspots with 85 and 51 hits.

TABLE 13
Zinc finger sequences targeting the genomic safe harbor site, hROSA26.

| hROSA | | | | |
|---|---|---|---|---|
| 26 | | | | |
| TTAA | NAME | TARGET | SCORE | ZFP AMINO ACID CODE |
| 5′ | ZnF3a | TGG GAA GAT | 58.64 | LEPGEKPYKCPECGKSFSQNSTLTEHQRTHTGEKPYKCPECGKSFSQRANLRAHQ |
| | | AAA CTA | | RTHTGEKPYKCPECGKSFSTSGNLVRHQRTHTGEKPYKCPECGKSFSQSSNLVRH |
| | | | | QRTHTGEKPYKCPECGKSFSRSDHLTTHQRTHTGKKTS |
| 5′ | ZnF5a | ACT CCC CTG | 56.25 | LEPGEKPYKCPECGKSFSDSGNLRVHQRTHTGEKPYKCPECGKSFSDPGHLVRHQ |
| | | CAG GGC AAC | | RTHTGEKPYKCPECGKSFSRADNLTEHQRTHTGEKPYKCPECGKSFSRNDALTEH |
| | | | | QRTHTGEKPYKCPECGKSFSSKKHLAEHQRTHTGEKPYKCPECGKSFSTHLDLIR |
| | | | | HQRTHTGKKTS |
| 5′ | ZnF5b | CCC CTG CAG | 56.25 | LEPGEKPYKCPECGKSFSDCRDLARHQRTHTGEKPYKCPECGKSFSDSGNLRVHQ |
| | | GGC AAC GCC | | RTHTGEKPYKCPECGKSFSDPGHLVRHQRTHTGEKPYKCPECGKSFSRADNLTEH |
| | | | | QRTHTGEKPYKCPECGKSFSRNDALTEHQRTHTGEKPYKCPECGKSFSSKKHLAE |
| | | | | HQRTHTGKKTS |
| 5′ | ZnF5c | CTG CAG GGC | 60.58 | LEPGEKPYKCPECGKSFSRADNLTEHQRTHTGEKPYKCPECGKSFSDCRDLARHQ |
| | | AAC GCC CAG | | RTHTGEKPYKCPECGKSFSDSGNLRVHQRTHTGEKPYKCPECGKSFSDPGHLVRH |
| | | | | QRTHTGEKPYKCPECGKSFSRADNLTEHQRTHTGEKPYKCPECGKSFSRNDALTE |
| | | | | HQRTHTGKKTS |
| 5′ | ZnF5d | CAG GGC AAC | 58.08 | LEPGEKPYKCPECGKSFSQRAHLERHQRTHTGEKPYKCPECGKSFSRADNLTEHQ |
| | | GCC CAG GGA | | RTHTGEKPYKCPECGKSFSDCRDLARHQRTHTGEKPYKCPECGKSFSDSGNLRVH |
| | | | | QRTHTGEKPYKCPECGKSFSDPGHLVRHQRTHTGEKPYKCPECGKSFSRADNLTE |
| | | | | HQRTHTGKKTS |
| 5′ | ZnF5e | GGC AAC GCC | 57.32 | LEPGEKPYKCPECGKSFSTSHSLTEHQRTHTGEKPYKCPECGKSFSQRAHLERHQ |
| | | CAG GGA CCA | | RTHTGEKPYKCPECGKSFSRADNLTEHQRTHTGEKPYKCPECGKSFSDCRDLARH |
| | | | | QRTHTGEKPYKCPECGKSFSDSGNLRVHQRTHTGEKPYKCPECGKSFSDPGHLVR |
| | | | | HQRTHTGKKTS |
| 5′ | ZnF5f | AAC GCC CAG | 54.99 | LEPGEKPYKCPECGKSFSHRTTLTNHQRTHTGEKPYKCPECGKSFSTSHSLTEHQ |
| | | GGA CCA AGT | | RTHTGEKPYKCPECGKSFSQRAHLERHQRTHTGEKPYKCPECGKSFSRADNLTEH |
| | | | | QRTHTGEKPYKCPECGKSFSDCRDLARHQRTHTGEKPYKCPECGKSFSDSGNLRV |
| | | | | HQRTHTGKKTS |
| 5′ | ZnF5g | GCC CAG GGA | 55.31 | LEPGEKPYKCPECGKSFSREDNLHTHQRTHTGEKPYKCPECGKSFSHRTTLTNHQ |
| | | CCA AGT TAG | | RTHTGEKPYKCPECGKSFSTSHSLTEHQRTHTGEKPYKCPECGKSFSQRAHLERH |
| | | | | QRTHTGEKPYKCPECGKSFSRADNLTEHQRTHTGEKPYKCPECGKSFSDCRDLAR |
| | | | | HQRTHTGKKTS |
| 5′ | ZnF5h | CAG GGA CCA | 50.76 | LEPGEKPYKCPECGKSFSSKKHLAEHQRTHTGEKPYKCPECGKSFSREDNLHTHQ |
| | | AGT TAG CCC | | RTHTGEKPYKCPECGKSFSHRTTLTNHQRTHTGEKPYKCPECGKSFSTSHSLTEH |
| | | | | QRTHTGEKPYKCPECGKSFSQRAHLERHQRTHTGEKPYKCPECGKSFSRADNLTE |
| | | | | HQRTHTGKKTS |
| 3′ | ZnF12a | GCC TAG GCA | 59.09 | LEPGEKPYKCPECGKSFSQSSNLVRHQRTHTGEKPYKCPECGKSFSQRANLRAHQ |
| | | AAA GAA | | RTHTGEKPYKCPECGKSFSQSGDLRRHQRTHTGEKPYKCPECGKSFSREDNLHTH |
| | | | | QRTHTGEKPYKCPECGKSFSDCRDLARHQRTHTGKKTS |
| 3′ | ZnF13a | CGC GAG GAG | 57.19 | LEPGEKPYKCPECGKSFSRSDHLTNHQRTHTGEKPYKCPECGKSFSRSDHLTNHQ |
| | | GAA AGG AGG | | RTHTGEKPYKCPECGKSFSQSSNLVRHQRTHTGEKPYKCPECGKSFSRSDNLVRH |
| | | | | QRTHTGEKPYKCPECGKSFSRSDNLVRHQRTHTGEKPYKCPECGKSFSHTGHLLE |
| | | | | HQRTHTGKKTS |
| 3′ | ZnF13b | GAG GAG GAA | 57.80 | LEPGEKPYKCPECGKSFSRSDNLVRHQRTHTGEKPYKCPECGKSFSRSDHLTNHQ |
| | | AGG AGG GAG | | RTHTGEKPYKCPECGKSFSRSDHLTNHQRTHTGEKPYKCPECGKSFSQSSNLVRH |
| | | | | QRTHTGEKPYKCPECGKSFSRSDNLVRHQRTHTGEKPYKCPECGKSFSRSDNLVR |
| | | | | HQRTHTGKKTS |
| 3′ | ZnF13c | GAG GAA AGG | 57.61 | LEPGEKPYKCPECGKSFSDPGHLVRHQRTHTGEKPYKCPECGKSFSRSDNLVRHQ |
| | | AGG GAG GGC | | RTHTGEKPYKCPECGKSFSRSDHLTNHQRTHTGEKPYKCPECGKSFSRSDHLTNH |
| | | | | QRTHTGEKPYKCPECGKSFSQSSNLVRHQRTHTGEKPYKCPECGKSFSRSDNLVR |
| | | | | HQRTHTGKKTS |

No Sequences have Target site overlap (TSO) . The first and last 4 amino acid residues may be omitted from the amino acid code. Available on the world wide web at scripps. edu/barbas/zfdesign/searchsequence. php

TABLE 14
Zinc finger sequences targeting the genomic safe harbor site, AAVS1.

| AAVS1 | | | | |
|---|---|---|---|---|
| TTAA | NAME | TARGET | SCORE | ZFP AMINO ACID CODE |
| 5′ | ZnF11a | TAG GAC AGT GGG GAA AAT GAC | 57.08 | LEPGEKPYKCPECGKSFSDCRDLARHQRTHTGEKPYKCPECG |
| | | CCA ACA GCC | | KSFSSPADLTRHQRTHTGEKPYKCPECGKSFSTSHSLTEHQR |
| | | | | THTGEKPYKCPECGKSFSDPGNLVRHQRTHTGEKPYKCPECG |
| | | | | KSFSTTGNLTVHQRTHTGEKPYKCPECGKSFSQSSNLVRHQR |
| | | | | THTGEKPYKCPECGKSFSRSDKLVRHQRTHTGEKPYKCPECG |
| | | | | KSFSHRTTLTNHQRTHTGEKPYKCPECGKSFSDPGNLVRHQR |
| | | | | THTGEKPYKCPECGKSFSREDNLHTHQRTHTGKKTS |
| 5′ | ZnF10a | AGA GGG AGC CAC GAA AAC AGA | 56.91 | LEPGEKPYKCPECGKSFSQLAHLRAHQRTHTGEKPYKCPECG |
| | | | | KSFSDSGNLRVHQRTHTGEKPYKCPECGKSFSQSSNLVRHQR |
| | | | | THTGEKPYKCPECGKSFSSKKALTEHQRTHTGEKPYKCPECG |
| | | | | KSFSERSHLREHQRTHTGEKPYKCPECGKSFSRSDKLVRHQR |
| | | | | THTGEKPYKCPECGKSFSQLAHLRAHQRTHTGKKTS |
| 3′ | ZnF12b | GCA GAT AGC CAG GAG | 59.97 | LEPGEKPYKCPECGKSFSRSDNLVRHQRTHTGEKPYKCPECG |
| | | | | KSFSRADNLTEHQRTHTGEKPYKCPECGKSFSERSHLREHQR |
| | | | | THTGEKPYKCPECGKSFSTSGNLVRHQRTHTGEKPYKCPECG |
| | | | | KSFSQSGDLRRHQRTHTGKKTS |
| 3′ | ZnF13b | AGA TAG CCA GGA GTC CTT | 56.80 | LEPGEKPYKCPECGKSFSTTGALTEHQRTHTGEKPYKCPECG |
| | | | | KSFSDPGALVRHQRTHTGEKPYKCPECGKSFSQRAHLERHQR |
| | | | | THTGEKPYKCPECGKSFSTSHSLTEHQRTHTGEKPYKCPECG |
| | | | | KSFSREDNLHTHQRTHTGEKPYKCPECGKSFSQLAHLRAHQR |
| | | | | THTGKKTS |
| 5′ | ZnF14a | CCC AGT GGT CAG GCC GGC CAG | 61.78 | LEPGEKPYKCPECGKSFSDCRDLARHQRTHTGEKPYKCPECG |
| | | GCC | | KSFSRADNLTEHQRTHTGEKPYKCPECGKSFSDPGHLVRHQR |
| | | | | THTGEKPYKCPECGKSFSDCRDLARHQRTHTGEKPYKCPECG |
| | | | | KSFSRADNLTEHQRTHTGEKPYKCPECGKSFSTSGHLVRHQR |
| | | | | THTGEKPYKCPECGKSFSHRTTLTNHQRTHTGEKPYKCPECG |
| | | | | KSFSSKKHLAEHQRTHTGKKTS |
| 5′ | ZnF15a | GGC CGG CCA GGC CTT CAG | 58.15 | LEPGEKPYKCPECGKSFSRADNLTEHQRTHTGEKPYKCPECG |
| | | | | KSFSTTGALTEHQRTHTGEKPYKCPECGKSFSDPGHLVRHQR |
| | | | | THTGEKPYKCPECGKSFSTSHSLTEHQRTHTGEKPYKCPECG |
| | | | | KSFSRSDKLTEHQRTHTGEKPYKCPECGKSFSDPGHLVRHQR |
| | | | | THTGKKTS |
| 5′ | ZnF16a | AGT GCT CAG TGG AAA CCA CGA | 58.65 | LEPGEKPYKCPECGKSFSDPGNLVRHQRTHTGEKPYKCPECG |
| | | AAG GAC | | KSFSRKDNLKNHQRTHTGEKPYKCPECGKSFSQSGHLTEHQR |
| | | | | THTGEKPYKCPECGKSFSTSHSLTEHQRTHTGEKPYKCPECG |
| | | | | KSFSQRANLRAHQRTHTGEKPYKCPECGKSFSRSDHLTTHQR |
| | | | | THTGEKPYKCPECGKSFSRADNLTEHQRTHTGEKPYKCPECG |
| | | | | KSFSTSGELVRHQRTHTGEKPYKCPECGKSFSHRTTLTNHQR |
| | | | | THTGKKTS |
| 5′ | ZnF17a | TGG CCC CCA GCC CCT CCT GCC | 60.89 | LEPGEKPYKCPECGKSFSDCRDLARHQRTHTGEKPYKCPECG |
| | | | | KSFSTKNSLTEHQRTHTGEKPYKCPECGKSFSTKNSLTEHQR |
| | | | | THTGEKPYKCPECGKSFSDCRDLARHQRTHTGEKPYKCPECG |
| | | | | KSFSTSHSLTEHQRTHTGEKPYKCPECGKSFSSKKHLAEHQR |
| | | | | THTGEKPYKCPECGKSFSRSDHLTTHQRTHTGKKTS |
| 5′ | ZnF18a | AGA GCC AGG AGT CCT GGC CCC | 57.23 | LEPGEKPYKCPECGKSFSSKKHLAEHQRTHTGEKPYKCPECG |
| | | CAG CCC | | KSFSRADNLTEHQRTHTGEKPYKCPECGKSFSSKKHLAEHQR |
| | | | | THTGEKPYKCPECGKSFSDPGHLVRHQRTHTGEKPYKCPECG |
| | | | | KSFSTKNSLTEHQRTHTGEKPYKCPECGKSFSHRTTLTNHQR |
| | | | | THTGEKPYKCPECGKSFSRSDHLTNHQRTHTGEKPYKCPECG |
| | | | | KSFSDCRDLARHQRTHTGEKPYKCPECGKSFSQLAHLRAHQR |
| | | | | THTGKKTS |
| 3′ | ZnF19a | GCA GGA GGG GCT GGG GGC CAG | 59.93 | LEPGEKPYKCPECGKSFSDPGNLVRHQRTHTGEKPYKCPECG |
| | | GAC | | KSFSRADNLTEHQRTHTGEKPYKCPECGKSFSDPGHLVRHQR |
| | | | | THTGEKPYKCPECGKSFSRSDKLVRHQRTHTGEKPYKCPECG |
| | | | | KSFSTSGELVRHQRTHTGEKPYKCPECGKSFSRSDKLVRHQR |
| | | | | THTGEKPYKCPECGKSFSQRAHLERHQRTHTGEKPYKCPECG |
| | | | | KSFSQSGDLRRHQRTHTGKKTS |
| 3′ | ZnF20b | ATA GCC CTG GGC CCA CGG CTT | 59.53 | LEPGEKPYKCPECGKSFSSRRTCRAHQRTHTGEKPYKCPECG |
| | | CGT | | KSFSTTGALTEHQRTHTGEKPYKCPECGKSFSRSDKLTEHQR |
| | | | | THTGEKPYKCPECGKSFSTSHSLTEHQRTHTGEKPYKCPECG |
| | | | | KSFSDPGHLVRHQRTHTGEKPYKCPECGKSFSRNDALTEHQR |
| | | | | THTGEKPYKCPECGKSFSDCRDLARHQRTHTGEKPYKCPECG |
| | | | | KSFSQKSSLIAHQRTHTGKKT |
| 3′ | ZnF21b | GAA GGA CCT GGC TGG | 55.22 | LEPGEKPYKCPECGKSFSRSDHLTTHQRTHTGEKPYKCPECG |
| | | | | KSFSDPGHLVRHQRTHTGEKPYKCPECGKSFSTKNSLTEHQR |
| | | | | THTGEKPYKCPECGKSFSQRAHLERHQRTHTGEKPYKCPECG |
| | | | | KSFSQSSNLVRHQRTHTGKKTS |
| 5′ | ZnF22a | GCA GGA ACG AAG CCG TGG GCC | 56.47 | LEPGEKPYKCPECGKSFSDPGHLVRHQRTHTGEKPYKCPECG |
| | | CAG GGC | | KSFSRADNLTEHQRTHTGEKPYKCPECGKSFSDCRDLARHQR |
| | | | | THTGEKPYKCPECGKSFSRSDHLTTHQRTHTGEKPYKCPECG |
| | | | | KSFSRNDTLTEHQRTHTGEKPYKCPECGKSFSRKDNLKNHQR |
| | | | | THTGEKPYKCPECGKSFSRTDTLRDHQRTHTGEKPYKCPECG |
| | | | | KSFSQRAHLERHQRTHTGEKPYKCPECGKSFSQSGDLRRHQR |
| | | | | THTGKKTS |
| 5′ | ZnF23a | GGA AAC CAC CCC AGC AGA | 52.63 | LEPGEKPYKCPECGKSFSQLAHLRAHQRTHTGEKPYKCPECG |
| | | | | KSFSERSHLREHQRTHTGEKPYKCPECGKSFSSKKHLAEHQR |
| | | | | THTGEKPYKCPECGKSFSSKKALTEHQRTHTGEKPYKCPECG |
| | | | | KSFSDSGNLRVHQRTHTGEKPYKCPECGKSFSQRAHLERHQR |
| | | | | THTGKKTS |
| 5′ | ZnF24a | AAG GGT CAA GCT CGG AAA CCA | 55.09 | LEPGEKPYKCPECGKSFSQKSSLIAHQRTHTGEKPYKCPECG |
| | | CCC CAG CAG ATA | | KSFSRADNLTEHQRTHTGEKPYKCPECGKSFSRADNLTEHQR |
| | | | | THTGEKPYKCPECGKSFSSKKHLAEHQRTHTGEKPYKCPECG |
| | | | | KSFSTSHSLTEHQRTHTGEKPYKCPECGKSFSQRANLRAHQR |
| | | | | THTGEKPYKCPECGKSFSRSDKLTEHQRTHTGEKPYKCPECG |
| | | | | KSFSTSGELVRHQRTHTGEKPYKCPECGKSFSQSGNLTEHQR |
| | | | | THTGEKPYKCPECGKSFSTSGHLVRHQRTHTGEKPYKCPECG |
| | | | | KSFSRKDNLKNHQRTHTGKKTS |

No Sequences have Target site overlap (TSO) . The first and last 4 amino acid residues may be omitted from the amino acid code. Available on the world wide web at scripps. edu/barbas/zfdesign/searchsequence. php

TABLE 15
Zinc finger sequences targeting the chromosome 4 hotspot. [hg38 chr4:30, 793, 533-30, 793, 537
(9677); chr4:30, 875, 472-30, 875, 476 (8948)].

| Chr4 | | | | |
|---|---|---|---|---|
| TTAA | NAME | TARGET | SCORE | ZFP AMINO ACID CODE |
| 5′ | ZnF31F | CTTTGATGAACAGTCACA | 58.41 | LEPGEKPYKCPECGKSFSSPADLTRHQRTHTGEKPYKCPECG |
| | | | | KSFSDPGALVRHQRTHTGEKPYKCPECGKSFSSPADLTRHQR |
| | | | | THTGEKPYKCPECGKSFSQAGHLASHQRTHTGEKPYKCPECG |
| | | | | KSFSQAGHLASHQRTHTGEKPYKCPECGKSFSTTGALTEHQR |
| | | | | THTGKKTS |
| 5′ | ZnF32F | CTTCCAATTAGTCCTACC | 55.84 | LEPGEKPYKCPECGKSFSDKKDLTRHQRTHTGEKPYKCPECG |
| | | | | KSFSTKNSLTEHQRTHTGEKPYKCPECGKSFSHRTTLTNHQR |
| | | | | THTGEKPYKCPECGKSFSHKNALQNHQRTHTGEKPYKCPECG |
| | | | | KSFSTSHSLTEHQRTHTGEKPYKCPECGKSFSTTGALTEHQR |
| | | | | THTGKKTS |
| 5′ | ZnF33F | ATACTAGGAAGAAATACAATA | 57.27 | LEPGEKPYKCPECGKSFSQKSSLIAHQRTHTGEKPYKCPECG |
| | | | | KSFSSPADLTRHQRTHTGEKPYKCPECGKSFSTTGNLTVHQR |
| | | | | THTGEKPYKCPECGKSFSQLAHLRAHQRTHTGEKPYKCPECG |
| | | | | KSFSQRAHLERHQRTHTGEKPYKCPECGKSFSQNSTLTEHQR |
| | | | | THTGEKPYKCPECGKSFSQKSSLIAHQRTHTGKKTS |
| 5′ | ZnF34F | GCTCTTGTCATTTGAGAT | 57.38 | LEPGEKPYKCPECGKSFSTSGNLVRHQRTHTGEKPYKCPECG |
| | | | | KSFSQAGHLASHQRTHTGEKPYKCPECGKSFSHKNALQNHQR |
| | | | | THTGEKPYKCPECGKSFSDPGALVRHQRTHTGEKPYKCPECG |
| | | | | KSFSTTGALTEHQRTHTGEKPYKCPECGKSFSTSGELVRHQR |
| | | | | THTGKKTS |
| 5′ | ZnF35F | CCAAGCTGAAATGACACAAAAGTTAAA | 58.23 | LEPGEKPYKCPECGKSFSRKDNLKNHQRTHTGEKPYKCPECG |
| | | ACAAAG | | KSFSSPADLTRHQRTHTGEKPYKCPECGKSFSQRANLRAHQR |
| | | | | THTGEKPYKCPECGKSFSTSGSLVRHQRTHTGEKPYKCPECG |
| | | | | KSFSQRANLRAHQRTHTGEKPYKCPECGKSFSSPADLTRHQR |
| | | | | THTGEKPYKCPECGKSFSDPGNLVRHQRTHTGEKPYKCPECG |
| | | | | KSFSTTGNLTVHQRTHTGEKPYKCPECGKSFSQAGHLASHQR |
| | | | | THTGEKPYKCPECGKSFSERSHLREHQRTHTGEKPYKCPECG |
| | | | | KSFSTSHSLTEHQRTHTGKKTS |
| 5′ | ZnF36F | CTTATACCAGTTAATTAGCAC | 49.93 | LEPGEKPYKCPECGKSFSSKKALTEHQRTHTGEKPYKCPECG |
| | | | | KSFSREDNLHTHQRTHTGEKPYKCPECGKSFSTTGNLTVHQR |
| | | | | THTGEKPYKCPECGKSFSTSGSLVRHQRTHTGEKPYKCPECG |
| | | | | KSFSTSHSLTEHQRTHTGEKPYKCPECGKSFSQKSSLIAHQR |
| | | | | THTGEKPYKCPECGKSFSTTGALTEHQRTHTGKKTS |
| 3′ | ZnF37R | AACGCTATTGCACACATAGTTACA | 57.67 | LEPGEKPYKCPECGKSFSSPADLTRHQRTHTGEKPYKCPECG |
| | | | | KSFSTSGSLVRHQRTHTGEKPYKCPECGKSFSQKSSLIAHQR |
| | | | | THTGEKPYKCPECGKSFSSKKALTEHQRTHTGEKPYKCPECG |
| | | | | KSFSQSGDLRRHQRTHTGEKPYKCPECGKSFSHKNALQNHQR |
| | | | | THTGEKPYKCPECGKSFSTSGELVRHQRTHTGEKPYKCPECG |
| | | | | KSFSDSGNLRVHQRTHTGKKTS |
| 3′ | ZnF38R | TGAATTCAGGAACAAAGTATA | 53. | LEPGEKPYKCPECGKSFSQKSSLIAHQRTHTGEKPYKCPECG |
| | | | 21 | KSFSHRTTLTNHQRTHTGEKPYKCPECGKSFSQSGNLTEHQR |
| | | | | THTGEKPYKCPECGKSFSQSSNLVRHQRTHTGEKPYKCPECG |
| | | | | KSFSRADNLTEHQRTHTGEKPYKCPECGKSFSHKNALQNHQR |
| | | | | THTGEKPYKCPECGKSFSQAGHLASHQRTHTGKKTS |
| 3′ | ZnF39R | GCTGGTATGTGACACAGCCATCAACAA | 50. | LEPGEKPYKCPECGKSFSQSGNLTEHQRTHTGEKPYKCPECG |
| | | | 63 | KSFSQSGNLTEHQRTHTGEKPYKCPECGKSFSTSGNLTEHQR |
| | | | | THTGEKPYKCPECGKSFSERSHLREHQRTHTGEKPYKCPECG |
| | | | | KSFSSKKALTEHQRTHTGEKPYKCPECGKSFSQAGHLASHQR |
| | | | | THTGEKPYKCPECGKSFSRRDELNVHQRTHTGEKPYKCPECG |
| | | | | KSFSTSGHLVRHQRTHTGEKPYKCPECGKSFSTSGELVRHQR |
| | | | | THTGKKTS |

No Sequences have Target site overlap (TSO) . The first and last 4 amino acid residues may be omitted from the amino acid code. Available on the world wide web at scripps.edu/barbas/zfdesign/searchsequence. php

TABLE 16
Zinc finger sequences targeting the chromosome 22 hotspot.

| Chr | | | | |
|---|---|---|---|---|
| 22 | | | | |
| TTAA | NAME | TARGET | SCORE | ZFP |
| 5′ | ZnF1a | CTTCCTGAAAGCAAGAGAT | 57.34 | LEPGEKPYKCPECGKSFSTTGNLTVHQRTHTGEKPYKCPECGKSFSQAGHLASH |
| | | GAAAT | | QRTHTGEKPYKCPECGKSFSQLAHLRAHQRTHTGEKPYKCPECGKSFSRKDNLK |
| | | | | NHQRTHTGEKPYKCPECGKSFSERSHLREHQRTHTGEKPYKCPECGKSFSQSSN |
| | | | | LVRHQRTHTGEKPYKCPECGKSFSTKNSLTEHQRTHTGEKPYKCPECGKSFSTT |
| | | | | GALTEHQRTHTGKKTS |
| 5′ | ZnF1b | CTGAAAGCAAGAGATGAAA | 58.92 | LEPGEKPYKCPECGKSFSTSHSLTEHQRTHTGEKPYKCPECGKSFSHKNALQNH |
| | | TTCCA | | QRTHTGEKPYKCPECGKSFSQSSNLVRHQRTHTGEKPYKCPECGKSFSTSGNLV |
| | | | | RHQRTHTGEKPYKCPECGKSFSQLAHLRAHQRTHTGEKPYKCPECGKSFSQSGD |
| | | | | LRRHQRTHTGEKPYKCPECGKSFSQRANLRAHQRTHTGEKPYKCPECGKSFSRN |
| | | | | DALTEHQRTHTGKKTS |
| 5′ | ZnF2a | ATACGAGGAGAAAATTAGC | 51.25 | LEPGEKPYKCPECGKSFSTSGNLTEHQRTHTGEKPYKCPECGKSFSREDNLHTH |
| | | AT | | QRTHTGEKPYKCPECGKSFSTTGNLTVHQRTHTGEKPYKCPECGKSFSQSSNLV |
| | | | | RHQRTHTGEKPYKCPECGKSFSQRAHLERHQRTHTGEKPYKCPECGKSFSQSGH |
| | | | | LTEHQRTHTGEKPYKCPECGKSFSQKSSLIAHQRTHTGKKTS |
| 5′ | ZnF3a | CATCCATGGCAGGAAGTTG | 58.67 | LEPGEKPYKCPECGKSFSRNDALTEHQRTHTGEKPYKCPECGKSFSTTGNLTVH |
| | | AAGCCAAAATAAATCTG | | QRTHTGEKPYKCPECGKSFSQKSSLIAHQRTHTGEKPYKCPECGKSFSQRANLR |
| | | | | AHQRTHTGEKPYKCPECGKSFSDCRDLARHQRTHTGEKPYKCPECGKSFSQSSN |
| | | | | LVRHQRTHTGEKPYKCPECGKSFSTSGSLVRHQRTHTGEKPYKCPECGKSFSQS |
| | | | | SNLVRHQRTHTGEKPYKCPECGKSFSRADNLTEHQRTHTGEKPYKCPECGKSFS |
| | | | | RSDHLTTHQRTHTGEKPYKCPECGKSFSTSHSLTEHQRTHTGEKPYKCPECGKS |
| | | | | FSTSGNLTEHQRTHTGKKTS |
| 5′ | ZnF3b | ATGGCAGGAAGTTGAAGCC | 54.14 | LEPGEKPYKCPECGKSFSQRANLRAHQRTHTGEKPYKCPECGKSFSTTGNLTVH |
| | | AAAATAAA | | QRTHTGEKPYKCPECGKSFSQSGNLTEHQRTHTGEKPYKCPECGKSFSERSHLR |
| | | | | EHQRTHTGEKPYKCPECGKSFSQAGHLASHQRTHTGEKPYKCPECGKSFSHRTT |
| | | | | LTNHQRTHTGEKPYKCPECGKSFSQRAHLERHQRTHTGEKPYKCPECGKSFSQS |
| | | | | GDLRRHQRTHTGEKPYKCPECGKSFSRRDELNVHQRTHTGKKTS |
| 3′ | ZnF5aR | GAAAAGAAGACTCAAGGAA | 55.40 | LEPGEKPYKCPECGKSFSSKKALTEHQRTHTGEKPYKCPECGKSFSQRANLRAH |
| | | ACAGAGCCAAACAC | | QRTHTGEKPYKCPECGKSFSDCRDLARHQRTHTGEKPYKCPECGKSFSQLAHLR |
| | | | | AHQRTHTGEKPYKCPECGKSFSDSGNLRVHQRTHTGEKPYKCPECGKSFSQRAH |
| | | | | LERHQRTHTGEKPYKCPECGKSFSQSGNLTEHQRTHTGEKPYKCPECGKSFSTH |
| | | | | LDLIRHQRTHTGEKPYKCPECGKSFSRKDNLKNHQRTHTGEKPYKCPECGKSFS |
| | | | | RKDNLKNHQRTHTGEKPYKCPECGKSFSQSSNLVRHQRTHTGKKTS |
| 3′ | ZnF5bR | AGGAAACAGAGCCAAACAC | 54.66 | LEPGEKPYKCPECGKSFSSPADLTRHQRTHTGEKPYKCPECGKSFSTTGALTEH |
| | | TTACA | | QRTHTGEKPYKCPECGKSFSSPADLTRHQRTHTGEKPYKCPECGKSFSQSGNLT |
| | | | | EHQRTHTGEKPYKCPECGKSFSERSHLREHQRTHTGEKPYKCPECGKSFSRADN |
| | | | | LTEHQRTHTGEKPYKCPECGKSFSQRANLRAHQRTHTGEKPYKCPECGKSFSRS |
| | | | | DHLTNHQRTHTGKKTS |
| 3′ | ZnF6aR | ATGCAGATTTGGACACAGA | 58.57 | LEPGEKPYKCPECGKSFSRSDKLVRHQRTHTGEKPYKCPECGKSFSSRRTCRAH |
| | | GTAGTAAACTGTGAAAACG | | QRTHTGEKPYKCPECGKSFSRSDHLTTHQRTHTGEKPYKCPECGKSFSRKDNLK |
| | | TGACAAGGCAAAGTGGCGT | | NHQRTHTGEKPYKCPECGKSFSQSGDLRRHQRTHTGEKPYKCPECGKSFSRKDN |
| | | GGG | | LKNHQRTHTGEKPYKCPECGKSFSDPGNLVRHQRTHTGEKPYKCPECGKSFSSR |
| | | | | RTCRAHQRTHTGEKPYKCPECGKSFSQRANLRAHQRTHTGEKPYKCPECGKSFS |
| | | | | QAGHLASHQRTHTGEKPYKCPECGKSFSRNDALTEHQRTHTGEKPYKCPECGKS |
| | | | | FSQRANLRAHQRTHTGEKPYKCPECGKSFSHRTTLTNHQRTHTGEKPYKCPECG |
| | | | | KSFSHRTTLTNHQRTHTGEKPYKCPECGKSFSRADNLTEHQRTHTGEKPYKCPE |
| | | | | CGKSFSSPADLTRHQRTHTGEKPYKCPECGKSFSRSDHLTTHQRTHTGEKPYKC |
| | | | | PECGKSFSHKNALQNHQRTHTGEKPYKCPECGKSFSRADNLTEHQRTHTGEKPY |
| | | | | KCPECGKSFSRRDELNVHQRTHTGKKTS |
| 3′ | ZnF6bR | GGACACAGAGTAGTAAAC | 55.80 | LEPGEKPYKCPECGKSFSDSGNLRVHQRTHTGEKPYKCPECGKSFSQSSSLVRH |
| | | | | QRTHTGEKPYKCPECGKSFSQSSSLVRHQRTHTGEKPYKCPECGKSFSQLAHLR |
| | | | | AHQRTHTGEKPYKCPECGKSFSSKKALTEHQRTHTGEKPYKCPECGKSFSQRAH |
| | | | | LERHQRTHTGKKTS |
| 5′ | ZnF10F | AAAGCTAGCAGCATGGCA | 57.55 | LEPGEKPYKCPECGKSFSQSGDLRRHQRTHTGEKPYKCPECGKSFSRRDELNVH |
| | | | | QRTHTGEKPYKCPECGKSFSERSHLREHQRTHTGEKPYKCPECGKSFSERSHLR |
| | | | | EHQRTHTGEKPYKCPECGKSFSTSGELVRHQRTHTGEKPYKCPECGKSFSQRAN |
| | | | | LRAHQRTHTGKKTS |
| 5′ | ZnF11F | CCTCTTATAAGGCCCAAGA | 52.55 | LEPGEKPYKCPECGKSFSQKSSLIAHQRTHTGEKPYKCPECGKSFSRSDHLTNH |
| | | GGATA | | QRTHTGEKPYKCPECGKSFSRKDNLKNHQRTHTGEKPYKCPECGKSFSSKKHLA |
| | | | | EHQRTHTGEKPYKCPECGKSFSRSDHLTNHQRTHTGEKPYKCPECGKSFSQKSS |
| | | | | LIAHQRTHTGEKPYKCPECGKSFSTTGALTEHQRTHTGEKPYKCPECGKSFSTK |
| | | | | NSLTEHQRTHTGKKTS |
| 5′ | ZnF12F | CAACATCCTTGACTTAATC | 55.00 | LEPGEKPYKCPECGKSFSSKKALTEHQRTHTGEKPYKCPECGKSFSTTGNLTVH |
| | | AC | | QRTHTGEKPYKCPECGKSFSTTGALTEHQRTHTGEKPYKCPECGKSFSQAGHLA |
| | | | | SHQRTHTGEKPYKCPECGKSFSTKNSLTEHQRTHTGEKPYKCPECGKSFSTSGN |
| | | | | LTEHQRTHTGEKPYKCPECGKSFSQSGNLTEHQRTHTGKKTS |
| 5′ | ZnF13F | GGTAGCAAAAAGGTAACC | 46.33 | LEPGEKPYKCPECGKSFSDKKDLTRHQRTHTGEKPYKCPECGKSFSQSSSLVRH |
| | | | | QRTHTGEKPYKCPECGKSFSRKDNLKNHQRTHTGEKPYKCPECGKSFSQRANLR |
| | | | | AHQRTHTGEKPYKCPECGKSFSERSHLREHQRTHTGEKPYKCPECGKSFSTSGH |
| | | | | LVRHQRTHTGKKTS |
| 3′ | ZnF14R | TGGGGTGCAAGAGGCCAGG | 61.28 | LEPGEKPYKCPECGKSFSDPGALVRHQRTHTGEKPYKCPECGKSFSRNDALTEH |
| | | CCAGAGTTGTTCTGGTC | | QRTHTGEKPYKCPECGKSFSTSGSLVRHQRTHTGEKPYKCPECGKSFSTSGSLV |
| | | | | RHQRTHTGEKPYKCPECGKSFSQLAHLRAHQRTHTGEKPYKCPECGKSFSDCRD |
| | | | | LARHQRTHTGEKPYKCPECGKSFSRADNLTEHQRTHTGEKPYKCPECGKSFSDP |
| | | | | GHLVRHQRTHTGEKPYKCPECGKSFSQLAHLRAHQRTHTGEKPYKCPECGKSFS |
| | | | | QSGDLRRHQRTHTGEKPYKCPECGKSFSTSGHLVRHQRTHTGEKPYKCPECGKS |
| | | | | FSRSDHLTTHQRTHTGKKTS |
| 3′ | ZnF15R | CGCATGCTGATTCAGCCTC | 58.41 | LEPGEKPYKCPECGKSFSDPGNLVRHQRTHTGEKPYKCPECGKSFSTKNSLTEH |
| | | CTGAC | | QRTHTGEKPYKCPECGKSFSTKNSLTEHQRTHTGEKPYKCPECGKSFSRADNLT |
| | | | | EHQRTHTGEKPYKCPECGKSFSHKNALQNHQRTHTGEKPYKCPECGKSFSRNDA |
| | | | | LTEHQRTHTGEKPYKCPECGKSFSRRDELNVHQRTHTGEKPYKCPECGKSFSHT |
| | | | | GHLLEHQRTHTGKKTS |
| 3′ | ZnF14R | AGTCAAGCAACAGGATGA | 50.89 | LEPGEKPYKCPECGKSFSQAGHLASHQRTHTGEKPYKCPECGKSFSQRAHLERH |
| | | | | QRTHTGEKPYKCPECGKSFSSPADLTRHQRTHTGEKPYKCPECGKSFSQSGDLR |
| | | | | RHQRTHTGEKPYKCPECGKSFSQSGNLTEHQRTHTGEKPYKCPECGKSFSHRTT |
| | | | | LTNHQRTHTGKKTS |
| 3′ | ZnF15R | GTCAAGCAACAGGATGATC | 59.22 | LEPGEKPYKCPECGKSFSHKNALQNHQRTHTGEKPYKCPECGKSFSTSGELVRH |
| | | CAAATGCTATT | | QRTHTGEKPYKCPECGKSFSTTGNLTVHQRTHTGEKPYKCPECGKSFSTSHSLT |
| | | | | EHQRTHTGEKPYKCPECGKSFSTSGNLVRHQRTHTGEKPYKCPECGKSFSTSGN |
| | | | | LVRHQRTHTGEKPYKCPECGKSFSRADNLTEHQRTHTGEKPYKCPECGKSFSQS |
| | | | | GNLTEHQRTHTGEKPYKCPECGKSFSRKDNLKNHQRTHTGEKPYKCPECGKSFS |
| | | | | DPGALVRHQRTHTGKKTS |

No Sequences have Target site overlap (TSO) . The first and last 4 amino acid residues may be omitted from the amino acid code. Available on the world wide web at scripps. edu/barbas/zfdesign/searchsequence. php

TABLE 17
Zinc finger sequences targeting the chromosome X (HPRT) hotspot. [hg38 chrX: 134, 476, 304-
chrX:134, 476, 337-134, 476, 340 (51)].

| ChrX | | | | |
|---|---|---|---|---|
| TTAA | NAME | TARGET | SCORE | ZFP AMINO ACID CODE |
| 5′ | ZnF41F | GTAGAAACTCGCCTTATG | 54.04 | LEPGEKPYKCPECGKSFSRRDELNVHQRTHTGEKPYKCPECG |
| | | | | KSFSTTGALTEHQRTHTGEKPYKCPECGKSFSHTGHLLEHQR |
| | | | | THTGEKPYKCPECGKSFSTHLDLIRHQRTHTGEKPYKCPECG |
| | | | | KSFSQSSNLVRHQRTHTGEKPYKCPECGKSFSQSSSLVRHQR |
| | | | | THTGKKTS |
| 5′ | ZnF42F | TGAATGAGTCCTGTCCATCTT | 55.08 | LEPGEKPYKCPECGKSFSTTGALTEHQRTHTGEKPYKCPECG |
| | | | | KSFSTSGNLTEHQRTHTGEKPYKCPECGKSFSDPGALVRHQR |
| | | | | THTGEKPYKCPECGKSFSTKNSLTEHQRTHTGEKPYKCPECG |
| | | | | KSFSHRTTLTNHQRTHTGEKPYKCPECGKSFSRRDELNVHQR |
| | | | | THTGEKPYKCPECGKSFSQAGHLASHQRTHTGKKTS |
| 5′ | ZnF43F | AAGATTAGAACAAATGTCCAG | 60.20 | LEPGEKPYKCPECGKSFSRADNLTEHQRTHTGEKPYKCPECG |
| | | | | KSFSDPGALVRHQRTHTGEKPYKCPECGKSFSTTGNLTVHQR |
| | | | | THTGEKPYKCPECGKSFSSPADLTRHQRTHTGEKPYKCPECG |
| | | | | KSFSQLAHLRAHQRTHTGEKPYKCPECGKSFSHKNALQNHQR |
| | | | | THTGEKPYKCPECGKSFSRKDNLKNHQRTHTGKKTS |
| 3′ | ZnF44R | ACTCTAAGCAGCAATGTA | 59.94 | LEPGEKPYKCPECGKSFSQSSSLVRHQRTHTGEKPYKCPECG |
| | | | | KSFSTTGNLTVHQRTHTGEKPYKCPECGKSFSERSHLREHQR |
| | | | | THTGEKPYKCPECGKSFSERSHLREHQRTHTGEKPYKCPECG |
| | | | | KSFSQNSTLTEHQRTHTGEKPYKCPECGKSFSTHLDLIRHQR |
| | | | | THTGKKTS |
| 5′ | ZnF45R | TGGGATAGTGAAAATGTC | 57.10 | LEPGEKPYKCPECGKSFSDPGALVRHQRTHTGEKPYKCPECG |
| | | | | KSFSTTGNLTVHQRTHTGEKPYKCPECGKSFSQSSNLVRHQR |
| | | | | THTGEKPYKCPECGKSFSHRTTLTNHQRTHTGEKPYKCPECG |
| | | | | KSFSTSGNLVRHQRTHTGEKPYKCPECGKSFSRSDHLTTHQR |
| | | | | THTGKKTS |
| 5′ | ZnF46R | AAAACTTGGGTCACTAAAATAGATGAT | 61.20 | LEPGEKPYKCPECGKSFSTSGNLVRHQRTHTGEKPYKCPECG |
| | | | | KSFSTSGNLVRHQRTHTGEKPYKCPECGKSFSQKSSLIAHQR |
| | | | | THTGEKPYKCPECGKSFSQRANLRAHQRTHTGEKPYKCPECG |
| | | | | KSFSTHLDLIRHQRTHTGEKPYKCPECGKSFSDPGALVRHQR |
| | | | | THTGEKPYKCPECGKSFSRSDHLTTHQRTHTGEKPYKCPECG |
| | | | | KSFSTHLDLIRHQRTHTGEKPYKCPECGKSFSQRANLRAHQR |
| | | | | THTGKKTS |
| 5′ | ZnF47R | AAACATGGAAAAGGTCAAAAACTTGGG | 43.59 | LEPGEKPYKCPECGKSFSRSDKLVRHQRTHTGEKPYKCPECG |
| | | | | KSFSTTGALTEHQRTHTGEKPYKCPECGKSFSQRANLRAHQR |
| | | | | THTGEKPYKCPECGKSFSQSGNLTEHQRTHTGEKPYKCPECG |
| | | | | KSFSTSGHLVRHQRTHTGEKPYKCPECGKSFSQRANLRAHQR |
| | | | | THTGEKPYKCPECGKSFSQRAHLERHQRTHTGEKPYKCPECG |
| | | | | KSFSTSGNLTEHQRTHTGEKPYKCPECGKSFSQRANLRAHQR |
| | | | | THTGKKTS |
| 3′ | ZnF48R | AATGACTAGAATGAAGTCCTACTG | 59.44 | LEPGEKPYKCPECGKSFSRNDALTEHQRTHTGEKPYKCPECG |
| | | | | KSFSQNSTLTEHQRTHTGEKPYKCPECGKSFSDPGALVRHQR |
| | | | | THTGEKPYKCPECGKSFSQSSNLVRHQRTHTGEKPYKCPECG |
| | | | | KSFSTTGNLTVHQRTHTGEKPYKCPECGKSFSREDNLHTHQR |
| | | | | THTGEKPYKCPECGKSFSDPGNLVRHQRTHTGEKPYKCPECG |
| | | | | KSFSTTGNLTVHQRTHTGKKTS |

No Sequences have Target site overlap (TSO) . The first and last 4 amino acid residues may be omitted from the amino acid code. Available on the world wide web at scripps. edu/barbas/zfdesign/searchsequence. php

### Example 10—Hyperactive Helper Enzymes with N or C Terminal Deletions

Hyperactive helper enzymes were tested for excision and integration frequencies by deleting either N or C termini at various positions and various lengths. Without wishing to be bound by theory, structural rationale for deleting the N- and C-termini amino acid residues in MLT helper are shown in TABLE 18.

TABLE 18
Illustrative and non-limiting structural rationale for
deleting the N- and C-termini amino acid residues.

| Deletion | Amino Acid | |
|---|---|---|
| Name | Deleted* | Illustrative Rationale |
| N1 | 1-Asp35 | Retains the beta sheet |
| N2 | 1-Pro45 | Removes predicted beta sheet based on |
| | | homology with piggyBac (pB) |
| N3 | 1-Arg68 | Not conserved |
| N4 | 1-Leu89 | Beginning of homology with solved pB |
| | | structure |
| C1 | Ile555-572 | Not conserved |
| C2 | Ile530-572 | Removes conserved cysteines |

*Numbering of amino acids is relative to SEQ ID NO: 502

FIG. 16 depicts the results of excision and integration assays on MLT helper that contains different deletions at the N- and C-termini. Bars represent % GFP cells measured by flow cytometry. MLT NO was used as a positive control known for high excision activity. Stuffer DNA (MLT Neg) that did not show expression served as negative controls. Abbreviations of test conditions are found in TABLE 18. For each sample, the left histogram is excision, and the right is integration.

The excision assay was performed by measuring the percentage of GFP cells in a cell line with a known GFP donor integration. The cells were grown to 80% confluency and analyzed by flow cytometry to count the percentage of GFP expressing cells as a baseline measurement. This percentage was used as the standard (i.e., 100%). X-tremeGENE™ 9 DNA Transfection Reagent protocol reagent was used to transfect helper plasmid in duplicate using 600 ng of DNA. The cells were gated to distinguish them from debris and 20,000 cells were counted. Forty-eight (48) hrs after the transfection the cells were analyzed by flow cytometry to count the percentage of GFP expressing cells. The cells were gated to distinguish them from debris and 20,000 cells were counted. The final integration efficiency was calculated by the baseline percentage of GFP cells by the percentage of GFP cells at 48 hr. For the integration assay, HEK293 cells were plated in 12-well size plates the day before transfection. The day of the transfection the media was exchanged 1 hour and 30 min before the transfection was performed. A 3:1 ratio of X-tremeGENE™ 9 DNA Transfection Reagent protocol reagent was used to co-transfect a donor plasmid containing GFP and a helper plasmid in duplicate using 600 ng of DNA each. Forty-eight (48) hrs after the transfection the cells were analyzed by flow cytometry to count the percentage of GFP expressing cells to measure transient transfection efficiency. The cells were gated to distinguish them from debris and 20,000 cells were counted. The cultures were grown for 15-20 days without antibiotic. Cells were passaged ⅔ times per week. Flow cytometry was used to count the percentage of GFP expressing cells to measure integration efficiency at 2 weeks. The final integration efficiency was calculated by dividing the 2-week percentage of GFP cells by the percentage of GFP cell at 48 hrs.

Moreover, truncated mutants of MLT were further fused to DNA binders to test for effects on excision and integration activities. FIG. 17 depicts the effects of fusing DNA binders on the N-terminus of MLT. DNA binder comprises TALEs, ZnF, and/or both. Specifically, FIG. 17 uses ZFs as DNA binders. Abbreviations of test conditions are found in TABLE 18. For each sample, the left histogram is excision, and the right is integration.

Additional experiments were performed to compare the integration pattern between the full length MLT and either an N- or C-terminal deleted mutant. FIGS. 18A-18C show comparison of integration pattern between full length MLT and N-terminal deleted [2-45aa] MLT (“N2”). FIG. 18A depicts a reduction in the number of integration sites in N-terminus deletions (N2). FIG. 18B shows the differences in the epigenetic profile in the MLT N2 mutant compared to hyperactive piggyBac (pB) and MLT. The heat map shows a shift from a strong association with promoters, transcription start sites to (H3K4me3 and H3K4me1), enhancers (H3K27ac) and gene bodies (H3K9me3 and H3K36me3) for pB and MLT compared to a weak signal for such sites with the N2 mutant. FIG. 18C depicts that the TTAA integration site is the main sequence for integration by the MLT N-terminus deletion mutant, N2.

The results from FIGS. 18A-18C demonstrates that MLT transposase N-terminus deletion mutants (e.g., without limitation, N2) of the present disclosure show a favorable integration and/or epigenetic profile.

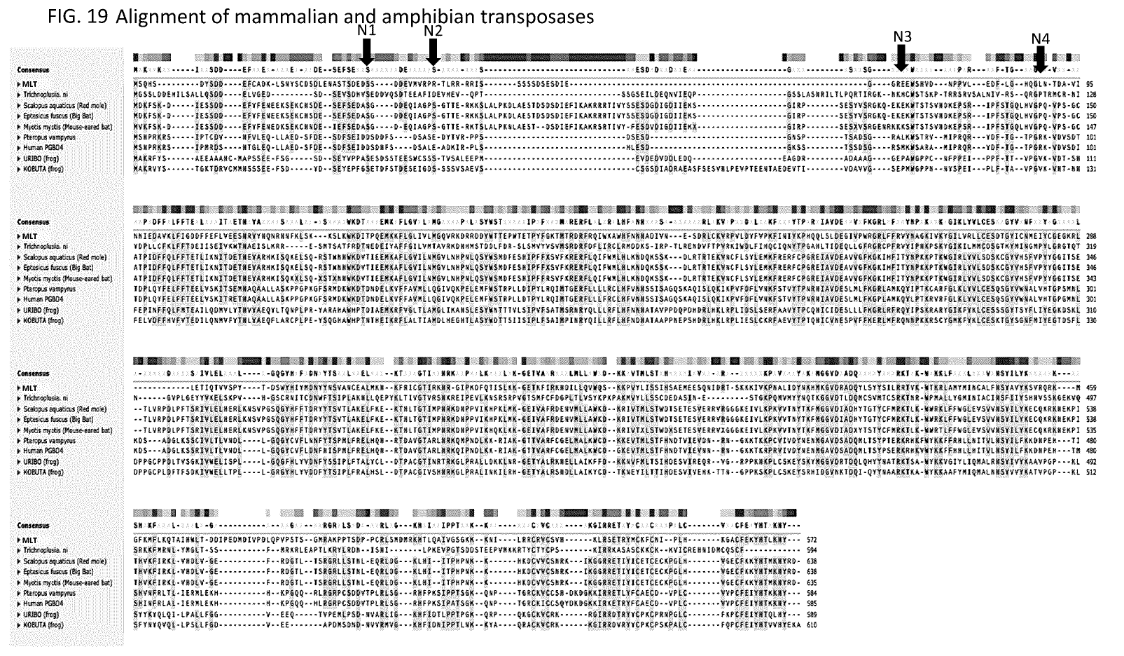

FIG. 19 depicts the alignment of mammalian and amphibian transposases. The arrows show the positions of the MLT N-terminus deletions and their alignment to other transposases.

The experiments described above show, inter alia, Exc+/Int− frequencies from different MLT variants with N or C terminal truncations. The results suggest that deletion of either N- or C-termini can result in MLT mutants with good excision activity. N-terminal deletion appears to yield mutants with decreased integration. On the other hand, C-terminal deletion appears to yield reduced excision and no integration. Without wishing to be bound by theory, the decreased integration may reflect the inability of the helper enzyme to interact with chromatin proteins. Moreover, without wishing to be bound by theory, the observation that C-terminal deletion resulted in decreased excision and no integration may reflect the helper enzyme's inability to form a dimer. In summary, the results show that the engineering of MLT for deletion in either N or C terminus produces variants with high excision and low intrinsic target binding abilities.

### Example 11—Increasing Excision by an Addition of MLT Transposase Mutants

FIG. 20 depicts that the addition of MLT transposase D416N mutants to MLT transposase containing 2 or more mutants increases excision by ˜5-fold.

FIG. 20 depicts the ability of the D416N mutants to increase excision and integration of MLT transposase mutants with little or no activity. The significance of the finding is, inter alia, that D416N can increase excision activity to create EXC+INT− mutants that, when fused to synthetic DNA binders, will only integrate at single chromosomal TTAA genomic location. Dark bars are excision, whereas light bars are integration.

Integration assay in HEK293 cells. HEK293 cells were plated in 12-well size plates the day before transfection at a density of 2.5×10⁶ cells/well. The day of the transfection the media was exchanged 1 hour and 30 min before the transfection was performed. The X-tremeGENE™ 9 DNA Transfection Reagent 9 DNA (Roche, cat #: 06365787001protocol was used in accordance with the manufacturer's instructions. A nucleic acid ratio of 600 ng:600 ng/12-well plate in was transfected in triplicate (e.g., three wells on the same plate) with a positive and control and donor only negative control. Forty-eight hours after the transfection the cells were analyzed by flow cytometry and the % of GFP expressing cells was used to measure transient transfection efficiency. Cells were passaged twice a week for 17 days. Flow cytometry, count % of GFP expressing cells was used to measure integration efficiency at 17 days. Gating was conservative, using live cells belonging to an obvious bright population; dim cells were excluded. Integration efficiency was calculated by dividing 17-day % GFP cells by the 48-hour % GFP cells to calculate final integration efficiency.

Excision assay in HEK293 cells. HEK293 cells were plated in 12-well size plates the day before transfection at a density of 2.5×10⁶ cells/well. The day of the transfection the media was exchanged 1 hour and 30 min before the transfection was performed. The X-tremeGENE™ 9 DNA Transfection Reagent 9 DNA (Roche, cat #: 06365787001protocol was used in accordance with the manufacturer's instructions. A nucleic acid ratio of 600 ng:600 ng/12-well plate in was transfected in triplicate (e.g., three wells on the same plate) with a positive and control and donor only negative control. A specialized HEK293 reporter cell line that expresses GFP if the helper plasmid active was used to detect excision (i.e., excises a DNA element that activates GFP). After 20 passages, an earlier aliquot of the cell line was used. Cells were cultured for 4 days. Flow cytometry, count % of GFP expressing cells was used to measure excision efficiency at 4 days. Gating was conservative, using live cells belonging to an obvious bright population; dim cells were excluded. Excision efficiency was calculated by % GFP cells.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A composition comprising (a) a helper enzyme or a nucleic acid encoding the helper enzyme and (b) a targeting element or a nucleic acid encoding the targeting element and (c) a linker connecting the helper enzyme and the targeting element, wherein:
the helper enzyme comprises an amino acid sequence having at least about 80% sequence identity to SEQ ID NO: 9 and has a non-polar aliphatic amino acid at position 2 of SEQ ID NO: 9 or a position corresponding thereto and one or more of
S8X₁ of SEQ ID NO: 9 or a position corresponding thereto, wherein X₁ is selected from alanine (A), glycine (G), valine (V), leucine (L), isoleucine (I), and proline (P);
C13X₂ of SEQ ID NO: 9 or a position corresponding thereto, wherein X₂ is selected from lysine (K), arginine (R), and histidine (H); and
N125X₃ of SEQ ID NO: 9 or a position corresponding thereto, wherein X₃ is selected from is selected from lysine (K), arginine (R), and histidine (H);
the targeting element is or comprises one or more of a Cas enzyme, which is optionally catalytically inactive and which is optionally associated with a guide RNA (gRNA), a transcription activator-like effector (TALE) DNA binding domain (DBD), a Zinc finger (ZF), a catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof; and
the linker comprises less than about 25 amino acids or 75 nucleotides.

2. A composition comprising (a) a helper enzyme or a nucleic acid encoding the helper enzyme and (b) a targeting element or a nucleic acid encoding the targeting element, wherein:
the helper enzyme comprises an amino acid sequence having at least about 80% sequence identity to SEQ ID NO: 9 and has a non-polar aliphatic amino acid at position 2 of SEQ ID NO: 9 or a position corresponding thereto and one or more of
S8X₁ of SEQ ID NO: 9 or a position corresponding thereto, wherein X₁ is selected from alanine (A), glycine (G), valine (V), leucine (L), isoleucine (I), and proline (P);
C13X₂ of SEQ ID NO: 9 or a position corresponding thereto, wherein X₂ is selected from lysine (K), arginine (R), and histidine (H); and
N125X₃ of SEQ ID NO: 9 or a position corresponding thereto, wherein X₃ is selected from is selected from lysine (K), arginine (R), and histidine (H);
the targeting element is or comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof; and
wherein the targeting element directs the helper enzyme to one or more nucleic acids sites that are upstream and/or downstream of the TTAA integration sites and within about 5 to about 30 base pairs of the TTAA integration sites or within about 15 to about 19 base pairs of the TTAA integration sites
and optionally a linker connecting the helper enzyme and the targeting element, the linker comprises less than about 25 amino acids or 75 nucleotides.

3. The composition of claim 1 or claim 2, wherein the non-polar aliphatic amino acid is selected from alanine (A), glycine (G), valine (V), leucine (L), isoleucine (I), and proline (P).

4. The composition of any one of claims 1-3, wherein the linker comprises about 10 amino acids to about 20 amino acids or about 12 amino acids to about 15 amino acids, or about 30 nucleotides to about 60 nucleotides or about 36 nucleotides to about 45 nucleotides.

5. The composition of any one of claims 1-4, wherein the linker is substantially comprised of glycine (G) and serine (S) residues.

6. The composition of any one of claims 1-5, wherein the linker is or comprises (GSS)₄ or in the case of insertion of a DNA binder (TALE, ZnF) in an intrinsic DNA binding loop, the linker is (GS)1 on either side of the DNA binder (TALE, ZnF).

7. The composition of any one of claims 1-6, wherein the linker connects the targeting element to the N-terminus of the helper enzyme or connects the targeting element within the helper enzyme.

8. The composition of any one of claims 1-7, wherein the helper enzyme is suitable of inserting a donor nucleic acid comprising a transgene in a genomic safe harbor site (GSHS) and/or wherein the targeting element is suitable for directing the helper enzyme to a GSHS.

9. The composition of claim 8 wherein the GSHS is in an open chromatin location in a chromosome.

10. The composition of claim 8 or 9, wherein the GSHS is selected from adeno-associated virus site 1 (AAVS1), chemokine (C-C motif) receptor 5 (CCR5) gene, HIV-1 coreceptor, and human Rosa26 locus.

11. The composition of any one of claims 8-10, wherein the GSHS comprises one or more TTAA integration sites.

12. The composition of any one of claims 8-11, wherein the targeting element directs the helper enzyme to either one or more nucleic acid sites that are upstream and/or downstream of the TTAA integration sites or to the TTAA integration sites.

13. The composition of any one of claims 8-12, wherein the targeting element directs the helper enzyme to one or more nucleic acid sites that are upstream and/or downstream of the TTAA integration sites and within about 5 to about 30 base pairs of the TTAA integration sites or within about 15 to about 19 base pairs of the TTAA integration sites.

14. The composition of any one of claims 8-13, wherein the targeting element directs the helper enzyme to two nucleic acid sites of the TTAA integration sites, wherein a first site is upstream of TTAA and within about 5 to about 30 base pairs or about 15 to about 19 base pairs of the TTAA and a second site is downstream of TTAA and within about 5 to about 30 base pairs or about 15 to about 19 base pairs of the TTAA.

15. The composition of any one of claims 1-14, wherein the helper enzyme comprises an amino acid sequence having at least about 90% sequence identity to SEQ ID NO: 9.

16. The composition of any one of claims 1-14, wherein the helper enzyme comprises an amino acid sequence having at least about 95% sequence identity or at least about 98% sequence identity to SEQ ID NO: 9.

17. The composition of any one of claims 1-14, wherein a donor DNA and a helper RNA are transfected at a donor DNA to helper RNA ratio of about 1 to about 4, or about 1 to about 2, or about 1 to about 1.

18. The composition of any one of claims 1-17, wherein:
a. the helper enzyme comprises an N- or C-terminal deletion, optionally at positions 1-35, or 1-45, or 1-55, or 1-65, or 1-75, or 1-85, or 1-95, or 1-105 or positions corresponding thereto, wherein the positions are relative to SEQ ID NO: 9;
b. the helper enzyme comprises an N-terminal deletion, optionally at positions 1-34, or 1-45, or 1-68, or 1-89 or positions corresponding thereto, wherein the positions are relative to SEQ ID NO: 9; and/or
c. the helper enzyme comprises a C-terminal deletion, optionally at positions 555-573 or 530-573 or positions corresponding thereto, wherein the positions are relative to SEQ ID NO: 9.

19. The composition of claim 18, wherein the N- or C-terminal deletion yields reduced or ablated off-target effects of the helper enzyme compared to the helper enzyme without the N- or C-terminal deletion.

20. The composition of claim 18 or 19, wherein the helper enzyme comprising the N-terminal deletion is or comprises an amino acid sequence of SEQ ID NO: 506, or a sequence having at least about 80%, or at least about 90%, or at least about 95%, or at least about 98% identity thereto.

21. The composition of any one of claims 1-20, wherein the helper enzyme comprises at least one substitution at position D416, or a position corresponding thereto relative to SEQ ID NO: 9.

22. The composition of claim 21, wherein the substitution at position D416 or a position corresponding thereto relative to SEQ ID NO: 9 is a polar and positively charged hydrophilic residue optionally selected from arginine (R) and lysine (K), a polar and neutral of charge hydrophilic residue selected from asparagine (N), glutamine (Q), serine (S), threonine (T), proline (P), and cysteine (C).

23. The composition of claim 22, wherein the substitution at position D416 or a position corresponding thereto relative to SEQ ID NO: 9 is asparagine (N).

24. The composition of any one of claims 1-20, wherein the helper enzyme comprises at least one substitution at selected from the mutations of FIG. 8, FIG. 20, TABLE 1, and/or TABLE 2.

25. The composition of anyone of claims 1-24, wherein the composition is a nucleic acid, optionally an RNA.

26. The composition of anyone of claims 1-25, wherein the composition further comprises a donor nucleic acid or is suitable for insertion of a donor nucleic acid, optionally wherein the donor nucleic acid is a transposon.

27. A method for inserting a gene into the genome of a cell, comprising contacting a cell with the composition of any one of claims 1-26.

28. A method for treating a disease or disorder ex vivo, comprising contacting a cell with the composition of any one of claims 1-26 and administering the cell to a subject in need thereof.

29. A method for treating a disease or disorder in vivo, comprising administering the composition of any one of claims 1-26 to a subject in need thereof.

30. A method for identifying site-specific targeting to a nucleic acid by a helper enzyme and a targeting element, comprising:
(a) transfecting a cell with a donor plasmid, the helper enzyme and a targeting element, and a reporter plasmid, wherein:
the donor plasmid comprises a first fragment of a reporter gene under the control of a promoter and a splice-donor site (SD);
the reporter plasmid comprises a landing pad for the targeting element comprising site specific DNA binding recognition sites flanking a TTAA followed by a splice acceptor site (SA) and a second fragment of a reporter gene; and
(b) splicing and integrating into the landing pad, to permit the reconstitution of the reporter gene from the fragments thereof and thereby causing a reporter redout.

31. The method of claim 30, further comprising (c) amplifying the donor plasmid to identify targeting.

32. The method of claim 31, further comprising (d) sequencing the amplified product to analyze integration in specific sequence regions.

33. The method of any one of claims 30-32, wherein the amplifying is via PCR.

34. The method of any one of claims 30-33, wherein the sequencing is amplicon sequencing.

35. The method of any one of claims 30-34, wherein the cell is a HEK293 cell.

36. The method of any one of claims 30-35, wherein the reporter gene encodes a fluorescent protein.

37. The method of any one of claims 30-36, wherein the fluorescent protein is or comprises a monomeric red fluorescent protein (mRFP).

38. The method of claim 37 wherein the mRFP is selected from mCherry, DsRed, mRFP1, mStrawberry, mOrange, and dTomato.

39. The method of any one of claims 30-36, wherein the fluorescent protein is or comprises a green fluorescent protein (GFP).

40. The method of any one of claims 30-39, wherein the reporter redout is fluorescence.

41. The method of any one of claims 30-40, wherein the promoter is selected from cytomegalovirus (CMV), CMV enhancer fused to the chicken β-actin (CAG), chicken β-actin (CBA), simian vacuolating virus 40 (SV40), p glucuronidase (GUSB), polyubiquitin C gene (UBC), elongation-factor 1α subunit (EF-1α), and phosphoglycerate kinase (PGK).

42. The method of any one of claims 30-41, wherein the helper enzyme is a recombinase, integrase or a transposase.

43. The method of any one of claims 30-42, wherein the helper enzyme is a mammal-derived transposase.

44. The method of any one of claims 30-43, wherein the helper enzyme is derived from Bombyx mori, Xenopus tropicalis, Trichoplusia ni, Myotis lucifugus, Rhinolophus ferrumequinum, Rousettus aegyptiacus, Phyllostomus discolor, Myotis myotis, Pteropus vampyrus, Pipistrellus kuhlii, troglodytes, Molossus molossus, or Homo sapiens.

45. The method of any one of claims 30-44, wherein the helper enzyme comprises an amino acid sequence having at least about 80% sequence identity to SEQ ID NO: 9 and has a non-polar aliphatic amino acid at position 2 of SEQ ID NO: 9 or a position corresponding thereto and one or more of
S8X₁ of SEQ ID NO: 9 or a position corresponding thereto, wherein X₁ is selected from alanine (A), glycine (G), valine (V), leucine (L), isoleucine (I), and proline (P);
C13X₂ of SEQ ID NO: 9 or a position corresponding thereto, wherein X₂ is selected from lysine (K), arginine (R), and histidine (H); and
N125X₃ of SEQ ID NO: 9 or a position corresponding thereto, wherein X₃ is selected from is selected from lysine (K), arginine (R), and histidine (H).

46. The method of any one of claims 30-45, wherein the targeting element is or comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof.

47. The method of any one of claims 30-46, wherein the SA and SD are spliced out of the donor plasmid in step (b).

48. The method of any one of claims 30-47, wherein the method is substantially as in FIG. 3.
